(19) United States
(12) Patent Application Publication
(10) Pub. No.: US 2011/0237538 A1
(43) Pub. Date: Sep. 29, 2011

(54) TREATMENT OF LYSOSOMAL STORAGE DISORDERS AND OTHER PROTEOSTATIC DISEASES

(75) Inventors: Olivier De Moor, Oxford (GB); Graeme Horne, Wantage (GB); Penny Jane Middleton, Didcot (GB); Frank Schroer, Wantage (GB); Stephen Paul Wren, Aylesbury (GB); Maria Ines Passos Eleuterio, Didcot (GB); Renate Van Well, Didcot (GB); Colin Richard Dorgan, Abingdon (GB); Francis Xavier Wilson, Welwyn Garden City (GB); Robert Nash, Ystrad Meurig (GB); Richard Storer, Folkestone (GB); Graham Michael Wynne, Bicester (GB); Alan Geoffrey Roach, Ashwell (GB); Akane Kawamura, Oxford (GB); Jonathon Mark Tinsley, Didcot (GB)

(73) Assignee: SUMMIT CORPORATION PLC, Oxfordshire (GB)

(21) Appl. No.: 13/057,654

(22) PCT Filed: Aug. 4, 2009
(86) PCT No.: PCT/GB09/01918

§ 371 Date: Jun. 16, 2011

(30) Foreign Application Priority Data
Aug. 6, 2008 (GB) 0814322.4
Sep. 24, 2008 (GB) 0817446.8
Oct. 1, 2008 (GB) 0817859.2
Oct. 24, 2008 (GB) 0819523.2
Oct. 24, 2008 (GB) 0819543.0
Apr. 9, 2009 (GB) 0906175.5
Apr. 9, 2009 (GB) 0906179.7
May 20, 2009 (GB) 0908661.2
May 20, 2009 (GB) 0908666.1

(51) Int. Cl. A61K 31/7028; A61K 31/407; A61K 31/445; A61K 31/439; A61K 31/55; A61K 31/40; A61K 31/404; A61K 31/47; A61K 31/495; A61P 3/10; A61P 9/00; A61P 25/00; A61P 19/02; A61P 35/00

(52) U.S. Cl. 514/53; 514/23; 514/413; 514/315; 514/304; 514/212.01; 514/408; 514/415; 514/311; 514/252.12

(57) ABSTRACT

Described are various compounds, in particular iminosugars, and methods for the treatment of proteostatic diseases, in particular lysosomal storage disorders. The compound may be a pharmacoperone of an enzyme selected from: (a) Acid alpha-glucosidase; (b) Acid beta-glucosidase; (c) glucocerebrosidase; (d) alpha-Galactosidase A; (e) Acid beta-galactosidase; (f) beta-Hexosaminidase A; (g) beta-Hexosaminidase B; (h) Acid sphingomyelinase; (i) Galactocerebrosidase; (j) Acid ceramidase; (k) Arylsulfatase A; (l) alpha-L-Iduronidase; (m) Iduronate-2-sulfatase; (n) Heparan N-sulfatase; (o) alpha-N-Acetylglucosaminidase; (p) Acetyl-CoA: alpha-glucosaminide N-acetyltransferase; (q) N-Acetylglucosamine-6-sulfate sulfatase; (r) N-Acetylgalactosamine-6-sulfate sulfatase; (s) Acid beta-galactosidase; (t) Arylsulfatase B; (u) beta-Glucuronidase; (v) Acid alpha-mannosidase; (w) Acid beta-mannosidase; (x) Acid alpha-L-fucosidase; (y) Sialidase; and (z) alpha-N-acetylgalactosaminidase.

### FIELD OF THE INVENTION

This invention relates to certain compounds, in particular iminosugars, and to methods for the treatment of proteostatic diseases, and in particular lysosomal storage disorders, based on the use of these compounds.

### BACKGROUND OF THE INVENTION

### Lysosomal storage disorders

Lysosomal storage disorders (LSDs) are a group of diseases which arise from abnormal metabolism of various substrates, including glycosphingolipids, glycogen, mucopolysaccharides and glycoproteins. More than fifty disorders have been identified that are caused by mutations in metabolic enzymes that are required for the degradation of such compounds. Many of them are neuronopathic and so may produce severe neurological impairment.

The metabolism of the substrates normally occurs in the lysosome and the process is regulated in a stepwise process by various degradative enzymes. Therefore, a deficiency in any one enzyme activity can perturb the entire process and result in the accumulation of particular substrates. Listed below are a number of lysosomal storage disorders and the corresponding defective enzymes:

Pompe disease: Acid alpha-glucosidase

Gaucher disease: Acid beta-glucosidase or glucocerebrosidase

Fabry disease: alpha-Galactosidase A

GMI-gangliosidosis: Acid beta-galactosidase

Tay-Sachs disease: beta-Hexosaminidase A

Sandhoff disease: beta-Hexosaminidase B

Niemann-Pick disease: Acid sphingomyelinase

Krabbe disease: Galactocerebrosidase

Farber disease: Acid ceramidase

Metachromatic leukodystrophy: Arylsulfatase A

Hurler-Scheie disease: alpha-L-Iduronidase

Hunter disease: Iduronate-2-sulfatase

Sanfilippo disease A: Heparan N-sulfatase

Sanfilippo disease B: alpha-N-Acetylglucosaminidase

Sanfilippo disease C: Acetyl-CoA: alpha-glucosaminide N-acetyltransferase

Sanfilippo disease D: N-Acetylglucosamine-6-sulfate sulfatase

Morquio disease A: N-Acetylgalactosamine-6-sulfate sulfatase

Morquio disease B: Acid beta-galactosidase

Maroteaux-Lamy disease: Arylsulfatase B

Sly disease: beta-Glucuronidase

alpha-Mannosidosis: Acid alpha-mannosidase

beta-Mannosidosis: Acid beta-mannosidase

Fucosidosis: Acid alpha-L-fucosidase

Sialidosis: Sialidase

Schindler-Kanzaki disease: alpha-N-acetylgalactosaminidase

Enzyme replacement therapy (ERT) and bone marrow transplantation are currently being used to treat these disorders. Cell- and gene-based therapies are also under investigation. However, all of these treatment regimens have severe drawbacks: for example, ERT is limited by the inability of the enzymes to cross the blood/brain barrier and so is ineffective in ameliorating the neurological deficits commonly associated with LSDs.

There is therefore great interest in the development of small molecules for treating LSDs and iminosugars have emerged as an important class of drugs for the treatment of LSDs.

Various iminosugars are known to act to modify the underlying metabolic dysfunction in LSDs by: (a) inhibiting enyzyme(s) involved in the biosynthesis of the accumulating substrate, thereby preventing pathological levels of accumulation (substrate reduction therapy). Here, the aim is to reduce the rate of biosynthesis of accumulating substrate to offset the catabolic defect, restoring the balance between the rate of biosynthesis and the rate of catabolism; and/or (b) promoting (or augmenting residual) endogenous enzymic activity by effecting proper folding and/or trafficking of a mutant enzyme by acting as molecular chaperones (pharmacoperones) in a treatment modality known as chaperone-mediated therapy (CMT).

Recent reviews of substrate reduction therapies based on the use of various iminosugars include Butters (2007) Iminosugar inhibitors for substrate reduction therapy for the lysosomal glycosphingolipidoses, in Iminosugars From Synthesis to Therapeutic Applications: Compain, Philippe/Martin, Olivier R. (eds.) ISBN-13: 978-0-470-03391-3-John Wiley & Sons, pages 249-268 as well as in the Table set out in Chapter 14.8 thereof (the disclosure of which is hereby incorporated by reference).

In the case of pharmacoperone-based therapies, attention has focused on iminosugars that bind to the active site of the mutant enzyme. Such therapies are often referred to as “Active-Site-Specific Chaperone” (ASSC) therapies. These treatments exploit the fact that small molecules can serve as molecular scaffolding and cause otherwise-misfolded mutant proteins to fold, and/or route correctly within the cell or its organelles. Molecules which can act in this way have been dubbed “chemical chaperones”, “pharmaceutical chaperones” or “pharmacoperones”. In particular, competitive inhibitors of the mutant enzymes implicated in various lysosomal storage disorders can, at subinhibitory concentrations, act as “Active-Site-Specific Chaperones” (ASSCs) by either inducing or stabilizing the proper conformation of the mutant enzyme by specific binding to the catalytic site. In this approach, the correctly folded mutant enzyme is secreted out of the ER where the ASSC (now in the presence of highly concentrated substrate and so at a subinhibitory concentration) is displaced to allow function of the enzyme (the dynamic exchange of ASSC as a competitive inhibitor and the enzyme's substrate being dependent on their relative concentrations). This area is reviewed, for example, by Fan (2007) Iminosugars as active-site-specific chaperones for the treatment of lysosomal storage disorders, in Iminosugars From Synthesis to Therapeutic Applications: Compain, Philippe/Martin, Olivier R. (eds.) ISBN-13: 978-0-470-03391-3-John Wiley & Sons), pages 225-247.

Various iminosugars have been identified as ASSCs and their specific binding to the catalytic active site of an enzyme implicated in lysosomal storage diseases exploited to form the basis of a new form of therapy dubbed active-site-specific chaperone therapy (see e.g. U.S. Pat. No. 6,583,158, U.S. Pat. No. 6,589,964 and U.S. Pat. No. 6,599,919). ASSC therapy uses low concentrations of potent enzyme inhibitors to enhance the folding and activity of mutant proteins in specific LSDs. This approach was first tested in Fabry disease, where 1-deoxy-galactononjirimycin (DGJ), an inhibitor of alpha-galactosidase A, was used to enhance the residual alpha-galactosidase activity in cell lines from Fabry disease patients (see U.S. Pat. No. 6,274,597 and U.S. Pat. No. 6,583,158). The ASSC strategy has been extended to other lysosomal storage diseases, including Gaucher disease and GMI-gangliosidosis.

ASSC therapy is now currently under development for several LSDs, including Gaucher disease, and offers several advantages over ERT or substrate deprivation therapy. Most notably, since the active site inhibitors used in ASSC are specific for the disease-causing enzyme, the therapy is targeted to a single protein and metabolic pathway, unlike substrate deprivation therapy that inhibits an entire synthetic pathway.

Like substrate deprivation therapy, the small molecule inhibitors for ASSC have the potential of crossing the blood brain barrier and could be used to treat neurological LSD forms. Moreover, in addition to enhancing the activity of the deficient enzymes associated with the LSDs, the ASSCs have also been demonstrated to enhance the activity of the corresponding wild-type enzyme (see U.S. Pat. No. 6,589,964) and so can be used adjunctively with enzyme replacement therapy in LSD patients.

### Proteostatic Diseases

Proteostasis (sometimes referred to as protein homeostasis) is the regulation of the concentration, conformation (tertiary structure), binding interactions (quaternary structure) and location of the individual proteins that constitute the proteome of an organism. Proteostasis is therefore essential for maintaining normal cellular function and so ultimately determines the health status of the organism as a whole.

Proteostasis is effected by numerous distinct but interacting regulated processes dubbed the proteostasis network. This comprises many diverse components, including transcription and translation factors, the protein quality control complex, degradative enzymes, organic and inorganic solutes, small molecule ligands, chaperones, cochaperones, folding enzymes, the ubuiquitin proteasome system (UPS) and constituents of the intra- and intercellular transport machinery. Proteostatic mechanisms operating within the proteostasis network therefore include transcription, translation and posttranslational modification, quality control, the heat shock response (HSR), the unfolded protein response (UPR), as well as protein folding, trafficking, aggregation, disaggregation and degradation. These processes together control fundamental aspects of cell, tissue and organismal development and environmental adaptation, as well as the response to intrinsic and extrinsic challenges (such as aging, neoplasia, genetic abnormalities and infection).

While the role of transcription and translation in governing the concentration of any given protein has been intensively studied and is well-characterized, the mechanisms regulating protein folding, trafficking, aggregation, disaggregation and degradation are less well-understood. However, it is now known that a given protein does not exist in just one structure (its native state) but can assume many different conformations, some of which have biological functions and each of which may exhibit quite different types of interaction with the proteostatsis network in general (and degradative pathways in particular). Posttranslational modification, folding, trafficking, aggregation, disaggregation and degradation all contribute to regulating the relative concentrations of the different protein conformers. Small changes in the concentration of any one conformer can profoundly change its solution state, rate of degradation, trafficking and deposition.

Over the past two years there has been a growing recognition of the role of proteostatic deficiencies in a wide variety of diseases (see e.g. Balch et al. (2008) Science 319: 916-919 and Morimoto (2008) Genes & Development 22: 1427-1438). Such diseases are collectively referred to as proteostatic diseases: they include aggregative and misfolding proteostatic diseases. Aggregative proteostatic disease, typically associated with the accumulation of proteotoxic species, includes prion diseases and a wide range of neurodegenerative disorders (e.g. Parkinson's disease, Alzheimer's disease and Huntington's disease). Misfolding proteostatic diseases include lysosomal storage disorders, certain forms of diabetes, emphysema, cancer and cystic fibrosis.

As demonstrated by Mu et al. (2008) Cell 134: 769-781, pharmacological intervention at the level of the proteostasis network by the administration of proteostasis regulators constitutes a potentially new and extremely powerful approach to the treatment of a wide range of protein folding diseases.

### SUMMARY OF THE INVENTION

According to a first aspect of the present invention there is provided a compound of Formula (1)

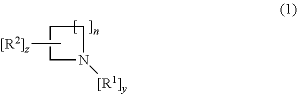

in which
n represents an integer from 1 to 7, provided that where n>1 the ring may also contain at least one unsaturated C—C bond
z represents an integer from 1 to (n+2)
y represents 1 or 2
R¹ represents H; C1-15 alkyl, C1-15 alkenyl or C1-15 alkynyl, optionally substituted with one or more R²; oxygen or an oxygen containing group such that the compound is an N-oxide; C(O)OR³; C(O)NR³R⁴; SO₂NR³; OH, OR³, or formyl
R² represents OH; OR³; ═O; NH₂, N₃; SH; SOₓR³; halo; CN; NO₂; NR³R⁴; (NR³)NR³R⁴; NH(NR³)NR³R⁴; CO₂R⁴; OC(O)R³; CONR³R⁴; NR⁴C(O)R³; NR⁴SO₂R³; P(O)(OR³)₂; C1-15 alkyl or alkenyl optionally substituted with one or more OH, OR³, ═O, NH₂, N₃, SH, SOₓR³, halo, CN, NO₂, NR³R⁴, (NR³)NR³R⁴, NH(NR³)NR³R⁴, CO₂R⁴, OC(O)R³, CONR³R⁴, NR⁴C(O)R³, NR⁴SO₂R³, P(O)(OR³)₂, aryl or carbocyclyl groups; carbocyclyl or aryl, either of which is optionally substituted with one or more OH, OR³, ═O, NH₂, N₃, SH, SOₓR³, halo, CN, NO₂, NR³R⁴, (NR³)NR³R⁴, NH(NR³)NR³R⁴, CO₂R⁴, OC(O)R³, CONR³R⁴, NR⁴C(O)R³, NR⁴SO₂R³, P(O)(OR³)₂, C1-9 alkyl optionally substituted with one or more OH, OR³, ═O, NH₂, N₃, halo, CN, NO₂, NR³R⁴, CO₂R⁴, CONR³R⁴, aryl or carbocyclyl groups; O-glycosyl; C-glycosyl; O-sulfate; O-phosphate or a group which together with the endocyclic carbon forms a spiro ring, with the provisos that: (a) two OH groups may not be attached to the same endocyclic carbon atom; (b) where there is only one R² substituent it contains an oxygen atom directly bonded to an endocyclic carbon atom; and (c) where z>1 any two R² substituents may together form an optionally heterocyclic ring (for example a carbocycle, cyclic ether or acetal)
R³ represents H; C1-6 alkyl, optionally substituted with one or more OH; aryl or C1-3 alkyl optionally substituted with aryl; SiR⁴₃ and
R⁴ represents H; C1-6 alkyl, optionally substituted with one or more OH
R³ and R⁴ may optionally form a 4 to 8 membered ring, containing one or more O, SOₓ or NR³ groups
x represents an integer from 0 to 2
   or a pharmaceutically acceptable salt or derivative thereof, for the treatment of a lysosomal storage disorder or a proteostatic disease.

In a second aspect, the invention provides a compound of Formula (2)

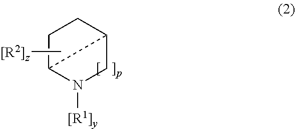

in which
p represents an integer from 1 to 2
z represents an integer from 1 to (p+7)
y represents 1 or 2
the broken line represents a bridge containing 2 or 3 carbon atoms between any two different ring carbon atoms, any or all of which bridge or bridgehead carbon atoms being optionally substituted with R²
R¹ represents H; C1-15 alkyl, C1-15 alkenyl or C1-15 alkynyl, optionally substituted with one or more R²; oxygen or an oxygen containing group such that the compound is an N-oxide; C(O)OR³; C(O)NR³R⁴; SO₂NR³; OH, OR³, or formyl
R² represents OH; OR³; ═O; NH₂; N₃; SH; SOₓR³; halo; CN; NO₂; NR³R⁴; (NR³)NR³R⁴; NH(NR³)NR³R⁴; CO₂R⁴; OC(O)R³; CONR³R⁴; NR⁴C(O)R³; NR⁴SO₂R³; P(O)(OR³)₂; C1-15 alkyl or alkenyl optionally substituted with one or more OH, OR³, ═O, NH₂, N₃, SH, SOₓR³, halo, CN, NO₂, NR³R⁴, (NR³)NR³R⁴, NH(NR³)NR³R⁴, CO₂R⁴, OC(O)R³, CONR³R⁴, NR⁴C(O)R³, NR⁴SO₂R³, P(O)(OR³)₂, aryl or carbocyclyl groups; carbocyclyl or aryl, either of which is optionally substituted with one or more OH, OR³, ═O, NH₂, N₃, SH, SOₓR³, halo, CN, NO₂, NR³R⁴, (NR³)NR³R⁴, NH(NR³)NR³R⁴, CO₂R⁴, OC(O)R³, CONR³R⁴, NR⁴C(O)R³, NR⁴SO₂R³, P(O)(OR³)₂, C1-9 alkyl optionally substituted with one or more OH, OR³, ═O, NH₂, N₃, halo, CN, NO₂, NR³R⁴, CO₂R⁴, CONR³R⁴, aryl or carbocyclyl groups; O-glycosyl; C-glycosyl; O-sulfate; O-phosphate or a group which together with the endocyclic carbon forms a spiro ring, with the provisos that: (a) two OH groups may not be attached to the same endocyclic carbon atom; (b) where there is only one R² substituent it contains an oxygen atom directly bonded to an endocyclic carbon atom; and (c) where z>1 any two R² substituents may together form an optionally heterocyclic ring (for example a carbocycle, cyclic ether or acetal)
R³ represents H; C1-6 alkyl, optionally substituted with one or more OH; aryl or C1-3 alkyl optionally substituted with aryl; SiR⁴₃ and
R⁴ represents H; C1-6 alkyl, optionally substituted with one or more OH
R³ and R⁴ may optionally form a 4 to 8 membered ring, containing one or more O, SOₓ or NR³ groups
x represents an integer from 0 to 2
   or pharmaceutically acceptable salt or derivative thereof, for the treatment of a lysosomal storage disorder or a proteostatic disease.

In a third aspect, the invention provides a compound of Formula (3)

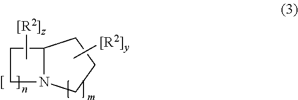

in which
n represents an integer from 1 to 7, for example 1 to 5, provided that where n>1 the ring may also contain at least one unsaturated C—C bond
m represents an integer from 1 to 3 and the ring may also contain at least one unsaturated C—C bond
z represents an integer from 0 to (n+2), provided that where z=0 then y≦1
y represents an integer from 0 to (m+2), provided that where y=0 then z≦1 the endocyclic nitrogen atom may be bonded to an oxygen or an oxygen containing group such that the compound is an N-oxide,
R² represents OH; OR³; ═O; NH₂; N₃; SH; SOₓR³; halo; CN; NO₂; NR³R⁴; (NR³)NR³R⁴; NH(NR³)NR³R⁴; CO₂R⁴; OC(O)R³; CONR³R⁴; NR⁴C(O)R³; NR⁴SO₂R³; P(O)(OR³)₂; C1-15 alkyl or alkenyl optionally substituted with one or more OH, OR³, ═O, NH₂, N₃, SH, SOₓR³, halo, CN, NO₂, NR³R⁴, (NR³)NR³R⁴, NH(NR³)NR³R⁴, CO₂R⁴, OC(O)R³, CONR³R⁴, NR⁴C(O)R³, NR⁴SO₂R³, P(O)(OR³)₂, aryl or carbocyclyl groups; carbocyclyl or aryl, either of which is optionally substituted with one or more OH, OR³, ═O, NH₂, N₃, SH, SOₓR³, halo, CN, NO₂, NR³R⁴, (NR³)NR³R⁴, NH(NR³)NR³R⁴, CO₂R⁴, OC(O)R³, CONR³R⁴, NR⁴C(O)R³, NR⁴SO₂R³, P(O)(OR³)₂, C1-9 alkyl optionally substituted with one or more OH, OR³, ═O, NH₂, N₃, halo, CN, NO₂, NR³R⁴, CO₂R⁴, CONR³R⁴, aryl or carbocyclyl groups; O-glycosyl; C-glycosyl; O-sulfate; O-phosphate or a group which together with the endocyclic carbon forms a Spiro ring, with the provisos that: (a) two OH groups may not be attached to the same endocyclic carbon atom; (b) where there is only one R² substituent it contains an oxygen atom directly bonded to an endocyclic carbon atom; and (c) where z>1 any two R² substituents may together form an optionally heterocyclic ring (for example a carbocycle, cyclic ether or acetal)
R³ represents H; C1-6 alkyl, optionally substituted with one or more OH; aryl or C1-3 alkyl optionally substituted with aryl; SiR⁴₃ and
R⁴ represents H; C1-6 alkyl, optionally substituted with one or more OH
R³ and R⁴ may optionally form a 4 to 8 membered ring, containing one or more O, SOₓ or NR³ groups
x represents an integer from 0 to 2
optionally wherein the compound has three, four or more rings
   or pharmaceutically acceptable salt or derivative thereof, for the treatment of a lysosomal storage disorder or a proteostatic disease.

In a further aspect, the invention provides an iminosugar as herein defined for the treatment of a lysosomal storage disease or a proteostatic disease.

In a yet further aspect, the invention provides a compound selected from compounds 1 to 892 of Table 1 for the treatment of a lysosomal storage disease or a proteostatic disease, or a pharmaceutically acceptable salt or derivative thereof.

In a yet further aspect, the compound for use according to the invention is (1R,2S,3S,4S,5R,6S)-5-amino-6-hydroxymethyl)cyclohexane-1,2,3,4-tetraol (Compound A), or a pharmaceutically acceptable salt or derivative thereof.

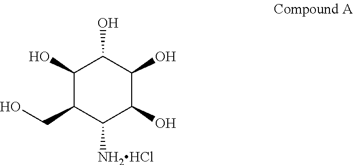

Other aspects and preferred embodiments of the invention are defined and described in the claims set out below.

The lysosomal storage disease may be selected from any of those diseases listed below:
Pompe disease (including infantile and late-onset forms)
Gaucher disease (including Type 1, Type 2 and Type 3 Gaucher disease)
Fabry disease
GMI-gangliosidosis
Tay-Sachs disease
Sandhoff disease
Niemann-Pick disease
Krabbe disease
Farber disease
Metachromatic leukodystrophy
Hurler-Scheie disease
Hunter disease
Sanfilippo disease A
Sanfilippo disease B
Sanfilippo disease C
Sanfilippo disease D
Morquio disease A
Morquio disease B
Maroteaux-Lamy disease
Sly disease
alpha-Mannosidosis
beta-Mannosidosis
Fucosidosis
Sialidosis
Schindler-Kanzaki disease

In preferred embodiments, the lysosomal storage disease is selected from: (a) Pompe disease; (b) Gaucher disease; and (c) Fabry disease.

In particularly preferred embodiments, the lysosomal disease is selected from Type 1, Type 2 and Type 3 Gaucher disease.

The iminosugar may be a pharmacoperone of an enzyme selected from: (a) Acid alpha-glucosidase; (b) Acid beta-glucosidase; (c) glucocerebrosidase; (d) alpha-Galactosidase A; (e) Acid beta-galactosidase; (f) beta-Hexosaminidase A; (g) beta-Hexosaminidase B; (h) Acid sphingomyelinase; (i) Galactocerebrosidase; (j) Acid ceramidase; (k) Arylsulfatase A; (l) alpha-L-Iduronidase; (m) Iduronate-2-sulfatase; (n) Heparan N-sulfatase; (o) alpha-N-Acetylglucosaminidase; (p) Acetyl-CoA: alpha-glucosaminide N-acetyltransferase; (q) N-Acetylglucosamine-6-sulfate sulfatase; (r) N-Acetylgalactosamine-6-sulfate sulfatase; (s) Acid beta-galactosidase; (t) Arylsulfatase B; (u) beta-Glucuronidase; (v) Acid alpha-mannosidase; (w) Acid beta-mannosidase; (x) Acid alpha-L-fucosidase; (y) Sialidase; and (z) alpha-N-acetylgalactosaminidase.

The compounds and/or iminosugars of the invention may act in synergy with lysosomal enzymes in enzyme replacement therapy, since the compounds may stabilize the replacement enzyme in situ (and so increase its half life) and/or relieve the inhibitory effects of substrate accumulation in the lysosome (so increasing the therapeutic activity of the replacement enzyme). This may provide the possibility of dose sparing of the replacement enzyme and/or the adoption of a more sustainable dosage regimen.

The invention also contemplates adjunctive use of the compounds of the invention with various adjunctive agents. The adjunctive agent may be selected from:
(a) a lysosomal enzyme; and/or
(b) a pharmacoperone of a lysosomal enzyme; and/or
(c) an inhibitor of a lysosomal enzyme; and/or
(d) a cell expressing a lysosomal enzyme; and/or
(e) nucleic acid encoding a lysosomal enzyme.

Thus, in another aspect, the invention provides a composition comprising a compound or iminosugar of the invention and an adjunctive agent selected from those listed above.

In such embodiments, the lysosomal enzyme may be selected from: (a) Acid alpha-glucosidase; (b) Acid beta-glucosidase; (c) glucocerebrosidase; (d) alpha-Galactosidase A; (e) Acid beta-galactosidase; (f) beta-Hexosaminidase A; (g) beta-Hexosaminidase B; (h) Acid sphingomyelinase; (i) Galactocerebrosidase; (j) Acid ceramidase; (k) Arylsulfatase A; (l) alpha-L-Iduronidase; (m) Iduronate-2-sulfatase; (n) Heparan N-sulfatase; (o) alpha-N-Acetylglucosaminidase; (p) Acetyl-CoA: alpha-glucosaminide N-acetyltransferase; (q) N-Acetylglucosamine-6-sulfate sulfatase; (r) N-Acetylgalactosamine-6-sulfate sulfatase; (s) Acid beta-galactosidase; (t) Arylsulfatase B; (u) beta-Glucuronidase; (v) Acid alpha-mannosidase; (w) Acid beta-mannosidase; (x) Acid alpha-L-fucosidase; (y) Sialidase; and (z) alpha-N-acetylgalactosaminidase.

In embodiments where the adjunctive agent is a lysosomal enzyme (for example, a lysosomal enzyme selected from (a)-(z), above), the lysosomal enzyme is preferably recombinant, for example being produced by the expression of heterologous DNA in a prokaryotic or eukaryotic host cell. In such embodiments, the lysosomal enzyme may have a modified primary amino acid sequence, for example containing N- and/or C-terminal tag sequences (for example, mannose-terminated recombinant glucocerebrosidase). Alternatively (or in addition), the lysosomal enzyme may be a truncated form of the wild type enzyme. It may be glycosylated, unglycosylated or deglycosylated.

In embodiments where the adjunctive agent is a pharmacoperone of a lysosomal enzyme, the pharmacoperone may be selected from the pharmacoperones described by Fan (2007) Iminosugars as active-site-specific chaperones for the treatment of lysosomal storage disorders, in Iminosugars From Synthesis to Therapeutic Applications: Compain, Philippe/Martin, Olivier R. (eds.) ISBN-13: 978-0-470-03391-3-John Wiley & Sons, pages 225-247 as well as in the Table set out in Chapter 14.8 thereof (the disclosure of which is hereby incorporated by reference).

In embodiments where the adjunctive agent is an inhibitor of the lysosomal enzyme, the inhibitor is preferably suitable for substrate reduction therapy of a lysosomal storage disorder, for example being selected from the inhibitors described in Butters (2007) Iminosugar inhibitors for substrate reduction therapy for the lysosomal glycosphingolipidoses, in Iminosugars From Synthesis to Therapeutic Applications: Compain, Philippe/Martin, Olivier R. (eds.) ISBN-13: 978-0-470-03391-3-John Wiley & Sons, pages 249-268 as well as in the Table set out in Chapter 14.8 thereof (the disclosure of which is hereby incorporated by reference).

In another aspect, the invention provides a pharmaceutical kit of parts comprising a compound of the invention in combination with a lysosomal enzyme, pharmacoperone of a lysosomal enzyme, cell expressing a lysosomal enzyme and/or nucleic acid encoding a lysosomal enzyme (as described above). The kit may also further comprise instructions for use in the treatment of a lysosomal disease.

In the compositions of the invention the compound of the invention and the adjunctive therapeutic(s) may act in a complementary or synergistic fashion. Particularly preferred are compositions and methods comprising both the compound or iminosugar of the invention and a lysosomal enzyme for combination enzyme replacement therapy.

### DETAILED DESCRIPTION OF THE INVENTION

All publications, patents, patent applications and other references mentioned herein are hereby incorporated by reference in their entireties for all purposes as if each individual publication, patent or patent application were specifically and individually indicated to be incorporated by reference and the content thereof recited in full.

### DEFINITIONS AND GENERAL PREFERENCES

Where used herein and unless specifically indicated otherwise, the following terms are intended to have the following meanings in addition to any broader (or narrower) meanings the terms might enjoy in the art:

Unless otherwise required by context, the use herein of the singular is to be read to include the plural and vice versa. The term “a” or “an” used in relation to an entity is to be read to refer to one or more of that entity. As such, the terms “a” (or “an”), “one or more,” and “at least one” are used interchangeably herein.

As used herein, the term “comprise,” or variations thereof such as “comprises” or “comprising,” are to be read to indicate the inclusion of any recited integer (e.g. a feature, element, characteristic, property, method/process step or limitation) or group of integers (e.g. features, element, characteristics, properties, method/process steps or limitations) but not the exclusion of any other integer or group of integers. Thus, as used herein the term “comprising” is inclusive or open-ended and does not exclude additional, unrecited integers or method/process steps.

The phrase “consisting essentially of” is used herein to require the specified integer(s) or steps as well as those which do not materially affect the character or function of the claimed invention.

As used herein, the term “consisting” is used to indicate the presence of the recited integer (e.g. a feature, element, characteristic, property, method/process step or limitation) or group of integers (e.g. features, element, characteristics, properties, method/process steps or limitations) alone.

As used herein, the term “disease” is used to define any abnormal condition that impairs physiological function and is associated with specific symptoms. The term is used broadly to encompass any disorder, illness, abnormality, pathology, sickness, condition or syndrome in which physiological function is impaired irrespective of the nature of the aetiology (or indeed whether the aetiological basis for the disease is established). It therefore encompasses conditions arising from infection, trauma, injury, surgery, radiological ablation, poisoning or nutritional deficiencies.

The term “proteostatic disease” is a term of art used to define a set of diseases mediated, at least in part, by deficiencies in proteostasis. The term therefore covers aggregative and misfolding proteostatic diseases, including in particular neurodegenerative disorders (e.g. Parkinson's disease, Alzheimer's disease and Huntington's disease), lysosomal storage disorders, diabetes, emphysema, cancer and cystic fibrosis.

The term “proteostasis regulator” is a term of art that defines an agent capable of modulating the conformation, concentration, binding interactions and/or location(s) of the constituent proteins of the proteome via the pathways of the proteostasis network.

The term “proteostasis network” is a term of art which defines the various conserved pathways which effect proteostasis, including transcription, translation and posttranslational modification, quality control, the heat shock response (HSR), the unfolded protein response (UPR), as well as protein folding, trafficking, aggregation, disaggregation and degradation.

As used herein, the term “treatment” or “treating” refers to an intervention (e.g. the administration of an agent to a subject) which cures, ameliorates or lessens the symptoms of a disease or removes (or lessens the impact of) its cause(s) (for example, the reduction in accumulation of pathological levels of lysosomal enzymes). In this case, the term is used synonymously with the term “therapy”.

Additionally, the terms “treatment” or “treating” refers to an intervention (e.g. the administration of an agent to a subject) which prevents or delays the onset or progression of a disease or reduces (or eradicates) its incidence within a treated population. In this case, the term treatment is used synonymously with the term “prophylaxis”.

The term “intervention” is a term of art used herein to define any agency which effects a physiological change at any level. Thus, the intervention may comprise the induction or repression of any physiological process, event, biochemical pathway or cellular/biochemical event. The interventions of the invention typically effect (or contribute to) the treatment (i.e. therapy or prophylaxis as herein defined) of a disease and typically involve the administration of an agent to a subject.

The term “metabolic syndrome” is used herein to define conditions characterized by the presence of three or more of the following symptoms: central obesity (waist measurement of more than 40 inches for men and more than 35 inches for women); high levels of triglycerides (150 mg/dL or higher); low levels of HDL (below 40 mg/dL for men and below 50 mg/dL for women) and high blood pressure (130/85 mm Hg or higher).

The term therefore includes conditions defined in accordance with the definition of metabolic syndrome by the World Health Organization: (a) fasting plasma glucose above 6.1 mmol/L; (b) blood pressure above 140/90 mm Hg; and (c) one or more of the following: (i) plasma triglycerides above 1.7 mmol/L; (ii) HDL below 0.9 and 1.0 mmol/L (for men and women, respectively); (iii) a body mass index above 30 kg/m².

References herein to the treatment of metabolic syndrome are to be interpreted to include the treatment of any or all of the disorders associated with metabolic syndrome, including in particular obesity (e.g. central obesity) and elevated serum triglycerides.

References herein to the treatment of type 1 or type 2 diabetes are to be interpreted to include the treatment of type 1 and type 2 diabetes per se as well as pre-diabetes (incipient diabetes) and insulin resistance.

The term “pre-diabetes” or “incipient diabetes” defines conditions in which elevated levels of glucose or glycosylated haemoglobin are present in the absence of diabetes.

In this context “subject” (which is to be read to include “individual”, “animal”, “patient” or “mammal” where context permits) defines any subject, particularly a mammalian subject, for whom treatment is indicated. Mammalian subjects include, but are not limited to, humans, domestic animals, farm animals, zoo animals, sport animals, pet animals such as dogs, cats, guinea pigs, rabbits, rats, mice, horses, cattle, cows; primates such as apes, monkeys, orangutans, and chimpanzees; canids such as dogs and wolves; felids such as cats, lions, and tigers; equids such as horses, donkeys, and zebras; food animals such as cows, pigs, and sheep; ungulates such as deer and giraffes; rodents such as mice, rats, hamsters and guinea pigs; and so on. In preferred embodiments, the subject is a human.

As used herein, an effective amount or a therapeutically effective amount of a compound defines an amount that can be administered to a subject without excessive toxicity, irritation, allergic response, or other problem or complication, commensurate with a reasonable benefit/risk ratio, but one that is sufficient to provide the desired effect, e.g. the treatment or prophylaxis manifested by a permanent or temporary improvement in the subject's condition. The amount will vary from subject to subject, depending on the age and general condition of the individual, mode of administration and other factors. Thus, while it is not possible to specify an exact effective amount, those skilled in the art will be able to determine an appropriate “effective” amount in any individual case using routine experimentation and background general knowledge. A therapeutic result in this context includes eradication or lessening of symptoms, reduced pain or discomfort, prolonged survival, improved mobility and other markers of clinical improvement. A therapeutic result need not be a complete cure.

As used herein, a “prophylactically effective amount” refers to an amount effective, at dosages and for periods of time necessary, to achieve the desired prophylactic result. Typically, since a prophylactic dose is used in subjects prior to or at an earlier stage of disease, the prophylactically effective amount will be less than the therapeutically effective amount.

The term “adjunctive” as applied to the use of the compounds of the invention in therapy or prophylaxis defines uses in which the compound is administered together with one or more other drugs, interventions, regimens or treatments (such as surgery and/or irradiation). Such adjunctive therapies may comprise the concurrent, separate or sequential administration/application of the materials of the invention and the other treatment(s). Thus, in some embodiments, adjunctive use of the materials of the invention is reflected in the formulation of the pharmaceutical compositions of the invention. For example, adjunctive use may be reflected in a specific unit dosage, or in formulations in which the compound of the invention is present in admixture with the other drug(s) with which it is to be used adjunctively (or else physically associated with the other drug(s) within a single unit dose). In other embodiments, adjunctive use of the compounds or compositions of the invention may be reflected in the composition of the pharmaceutical kits of the invention, wherein the compound of the invention is co-packaged (e.g. as part of an array of unit doses) with the other drug(s) with which it is to be used adjunctively. In yet other embodiments, adjunctive use of the compounds of the invention may be reflected in the content of the information and/or instructions co-packaged with the compound relating to formulation and/or posology.

As used herein, the term “combination”, as applied to two or more compounds and/or agents (also referred to herein as the components), is intended to define material in which the two or more compounds/agents are associated. The terms “combined” and “combining” in this context are to be interpreted accordingly.

The association of the two or more compounds/agents in a combination may be physical or non-physical. Examples of physically associated combined compounds/agents include:
compositions (e.g. unitary formulations) comprising the two or more compounds/agents in admixture (for example within the same unit dose);
compositions comprising material in which the two or more compounds/agents are chemically/physicochemically linked (for example by crosslinking, molecular agglomeration or binding to a common vehicle moiety);
compositions comprising material in which the two or more compounds/agents are chemically/physicochemically co-packaged (for example, disposed on or within lipid vesicles, particles (e.g. micro- or nanoparticles) or emulsion droplets);
pharmaceutical kits, pharmaceutical packs or patient packs in which the two or more compounds/agents are co-packaged or co-presented (e.g. as part of an array of unit doses);

Examples of non-physically associated combined compounds/agents include:
material (e.g. a non-unitary formulation) comprising at least one of the two or more compounds/agents together with instructions for the extemporaneous association of the at least one compound/agent to form a physical association of the two or more compounds/agents;
material (e.g. a non-unitary formulation) comprising at least one of the two or more compounds/agents together with instructions for combination therapy with the two or more compounds/agents;
material comprising at least one of the two or more compounds/agents together with instructions for administration to a patient population in which the other(s) of the two or more compounds/agents have been (or are being) administered;
material comprising at least one of the two or more compounds/agents in an amount or in a form which is specifically adapted for use in combination with the other(s) of the two or more compounds/agents.

As used herein, the term “combination therapy” is intended to define therapies which comprise the use of a combination of two or more compounds/agents (as defined above). Thus, references to “combination therapy”, “combinations” and the use of compounds/agents “in combination” in this application may refer to compounds/agents that are administered as part of the same overall treatment regimen. As such, the posology of each of the two or more compounds/agents may differ: each may be administered at the same time or at different times. It will therefore be appreciated that the compounds/agents of the combination may be administered sequentially (e.g. before or after) or simultaneously, either in the same pharmaceutical formulation (i.e. together), or in different pharmaceutical formulations (i.e. separately). Simultaneously in the same formulation is as a unitary formulation whereas simultaneously in different pharmaceutical formulations is non-unitary. The posologies of each of the two or more compounds/agents in a combination therapy may also differ with respect to the route of administration.

As used herein, the term “pharmaceutical kit” defines an array of one or more unit doses of a pharmaceutical composition together with dosing means (e.g. measuring device) and/or delivery means (e.g. inhaler or syringe), optionally all contained within common outer packaging. In pharmaceutical kits comprising a combination of two or more compounds/agents, the individual compounds/agents may unitary or non-unitary formulations. The unit dose(s) may be contained within a blister pack. The pharmaceutical kit may optionally further comprise instructions for use.

As used herein, the term “pharmaceutical pack” defines an array of one or more unit doses of a pharmaceutical composition, optionally contained within common outer packaging. In pharmaceutical packs comprising a combination of two or more compounds/agents, the individual compounds/agents may unitary or non-unitary formulations. The unit dose(s) may be contained within a blister pack. The pharmaceutical pack may optionally further comprise instructions for use.

As used herein, the term “patient pack” defines a package, prescribed to a patient, which contains pharmaceutical compositions for the whole course of treatment. Patient packs usually contain one or more blister pack(s). Patient packs have an advantage over traditional prescriptions, where a pharmacist divides a patient's supply of a pharmaceutical from a bulk supply, in that the patient always has access to the package insert contained in the patient pack, normally missing in patient prescriptions. The inclusion of a package insert has been shown to improve patient compliance with the physician's instructions.

The combinations of the invention may produce a therapeutically efficacious effect relative to the therapeutic effect of the individual compounds/agents when administered separately. The term iminosugar defines a saccharide analogue in which the ring oxygen is replaced by a nitrogen. The term is used herein sensu lato to include isoiminosugars, these being aza-carba analogues of sugars in which the C1 carbon is replaced by nitrogen and the ring oxygen is replaced by a carbon atom, as well as azasugars in which an endocyclic carbon is replaced with a nitrogen atom. 1-Azasugars (with the N in the anomeric position) in which the ring oxygen is substituted with a carbon atom are isoiminosugars (as herein defined), but 1-azasugars in which the ring oxygen remains unsubstituted (oxazines) or is substituted with a nitrogen atom (hydrazines) are also of particular importance. In all cases, one or more endocyclic carbon atoms may be substituted with a sulphur, oxygen or nitrogen atom.

As used herein, the term polyhydroxylated iminosugar defines a class of oxygenated iminosugars. Typically these have at least 2, 3, 4, 5, 6 or 7 (preferably 3, 4 or 5) hydroxyl groups (or alkyl groups with one or more hydroxy substituent(s)) on the ring system nucleus.

The term iminosugar acid defines mono- or bicyclic sugar acid analogues in which the ring oxygen is replaced by a nitrogen. The term N-acid ISA defines an iminosugar acid in which the carboxylic acid group is located on the ring nitrogen.

Preferred ISAs are selected from the following structural classes: piperidine (including (poly)hydroxypipecolic acids); pyrroline; pyrrolidine (including (poly)hydroxyprolines); pyrrolizidine; indolizidine and nortropane.

As used herein, the term polyhydroxylated as applied to iminosugar acids defines an ISA having at least 2 (preferably at least 3) hydroxyl groups (or alkyl groups with one or more hydroxy substituent(s)) on the ring system nucleus.

As used herein, the term bicyclic polyhydroxylated iminosugar defines a class of highly oxygenated iminosugars having a double or fused ring nucleus (i.e. having two or more cyclic rings in which two or more atoms are common to two adjoining rings). Typically, such iminosugars have at least 3, 4, 5, 6 or 7 (preferably 3, 4 or 5) free hydroxyl groups on the ring system nucleus.

The term pharmacoperone is a term of art (from “pharmacological chaperone”) used to define a class of biologically active small molecules (sometimes also referred to in the art as “chemical chaperones”) that serve as molecular scaffolds, causing otherwise misfolded mutant proteins to fold and route correctly within the cell.

The term ligand as used herein in relation to the compounds of the invention is intended to define those compounds which can act as binding partners for a biological target molecule in vivo (for example, an enzyme or receptor, such as a PRR). Such ligands therefore include those which bind (or directly physically interact) with the target in vivo irrespective of the physiological consequences of that binding. Thus, the ligands of the invention may bind the target as part of a cellular signalling cascade in which the target forms a part. Alternatively, they may bind the target in the context of some other aspect of cellular physiology. In the latter case, the ligands may for example bind the target at the cell surface without triggering a signalling cascade, in which case the binding may affect other aspects of cell function. Thus, the ligands of the invention may bind the target and thereby result in an increase in the concentration of functional target at the cell surface (for example mediated via an increase in target stability, absolute receptor numbers and/or target activity). Alternatively, the iminosugar ligands may bind target (or target precursors) intracellularly, in which case they may act as molecular chaperones to increase the expression of active target.

The term PRR ligand as used herein in relation to the compounds for use according to the invention defines compounds which can act as binding partners for a PRR. Such compounds therefore include those which bind (or directly physically interact) with a PRR in vivo irrespective of the physiological consequences of that binding. Thus, the ligands of the invention may bind a PRR as part of a cellular signalling cascade in which the PRR forms a part. Alternatively, they may bind PRR in the context of some other aspect of cellular physiology. In the latter case, the ligands may for example bind PRR at the cell surface without triggering a signalling cascade, in which case the binding may affect other aspects of cell function. Thus, the ligands of the invention may bind PRRs and thereby result in an increase in the concentration of functional PRR at the cell surface (for example mediated via an increase in PRR stability, absolute receptor numbers and/or PRR activity). Alternatively, the ligands may bind PRR (or PRR precursors) intracellularly, in which case they may act as molecular chaperones to increase the expression of active PRR.

In preferred embodiments, the PRR ligands of the invention are PRR agonists. The term agonist is used herein in relation to the PRR ligands of the invention to define a subclass of ligands which productively bind PRR to trigger the cellular signalling cascade of which the PRR forms a part.

The term bioisostere (or simply isostere) is a term of art used to define drug analogues in which one or more atoms (or groups of atoms) have been substituted with replacement atoms (or groups of atoms) having similar steric and/or electronic features to those atoms which they replace. The substitution of a hydrogen atom or a hydroxyl group with a fluorine atom is a commonly employed bioisosteric replacement. Sila-substitution (C/Si-exchange) is a relatively recent technique for producing isosteres. This approach involves the replacement of one or more specific carbon atoms in a compound with silicon (for a review, see Tacke and Zilch (1986) Endeavour, New Series 10: 191-197). The sila-substituted isosteres (silicon isosteres) may exhibit improved pharmacological properties, and may for example be better tolerated, have a longer half-life or exhibit increased potency (see for example Englebienne (2005) Med. Chem., 1(3): 215-226). Similarly, replacement of an atom by one of its isotopes, for example hydrogen by deuterium, may also lead to improved pharmacological properties, for example leading to longer half-life (see for example Kushner et al (1999) Can J Physiol Pharmacol. 77(2):79-88). In its broadest aspect, the present invention contemplates all bioisosteres (and specifically, all silicon bioisosteres) of the compounds of the invention.

In its broadest aspect, the present invention contemplates all optical isomers, racemic forms and diastereoisomers of the compounds described herein. Those skilled in the art will appreciate that, owing to the asymmetrically substituted carbon atoms present in the compounds of the invention, the compounds may be produced in optically active and racemic forms. If a chiral centre or another form of isomeric centre is present in a compound of the present invention, all forms of such isomer or isomers, including enantiomers and diastereoisomers, are intended to be covered herein. Compounds of the invention containing a chiral centre (or multiple chiral centres) may be used as a racemic mixture, an enantiomerically enriched mixture, or the racemic mixture may be separated using well-known techniques and an individual enantiomer may be used alone. Thus, references to the compounds (e.g. iminosugars) of the present invention encompass the products as a mixture of diastereoisomers, as individual diastereoisomers, as a mixture of enantiomers as well as in the form of individual enantiomers.

Therefore, the present invention contemplates all optical isomers and racemic forms thereof of the compounds of the invention, and unless indicated otherwise (e.g. by use of dash-wedge structural formulae) the compounds shown herein are intended to encompass all possible optical isomers of the compounds so depicted. In cases where the stereochemical form of the compound is important for pharmaceutical utility, the invention contemplates use of an isolated eutomer.

The terms derivative and pharmaceutically acceptable derivative as applied to the compounds of the invention define compounds which are obtained (or obtainable) by chemical derivatization of the parent compound of the invention. The pharmaceutically acceptable derivatives are therefore suitable for administration to or use in contact with the tissues of humans without undue toxicity, irritation or allergic response (i.e. commensurate with a reasonable benefit/risk ratio). Preferred derivatives are those obtained (or obtainable) by alkylation, esterification or acylation of the parent compounds.

The pharmaceutically acceptable derivatives of the invention may retain some or all of the biological activities described herein. In some cases, the biological activity (e.g. chaperone activity) is increased by derivatization. The derivatives may act as pro-drugs, and one or more of the biological activities described herein (e.g. pharmacoperones activity) may arise only after in vivo processing. Particularly preferred pro-drugs are ester derivatives which are esterified at one or more of the free hydroxyls and which are activated by hydrolysis in vivo. Derivatization may also augment other biological activities of the compound, for example bioavailability and/or glycosidase inhibitory activity and/or glycosidase inhibitory profile. For example, derivatization may increase glycosidase inhibitory potency and/or specificity and/or CNS penetration (e.g. penetration of the blood-brain barrier).

The term pharmaceutically acceptable salt as applied to the iminosugars of the invention defines any non-toxic organic or inorganic acid addition salt of the free base which are suitable for use in contact with the tissues of humans and lower animals without undue toxicity, irritation, allergic response and which are commensurate with a reasonable benefit/risk ratio. Suitable pharmaceutically acceptable salts are well known in the art. Examples are the salts with inorganic acids (for example hydrochloric, hydrobromic, sulphuric and phosphoric acids), organic carboxylic acids (for example acetic, propionic, glycolic, lactic, pyruvic, malonic, succinic, fumaric, malic, tartaric, citric, ascorbic, maleic, hydroxymaleic, dihydroxymaleic, benzoic, phenylacetic, 4-aminobenzoic, 4-hydroxybenzoic, anthranilic, cinnamic, salicylic, 2-phenoxybenzoic, 2-acetoxybenzoic and mandelic acid) and organic sulfonic acids (for example methanesulfonic acid and p-toluenesulfonic acid).

These salts and the free base compounds can exist in either a hydrated or a substantially anhydrous form. Crystalline forms, including all polymorphic forms, of the iminosugars of the invention are also contemplated and in general the acid addition salts of the compounds are crystalline materials which are soluble in water and various hydrophilic organic solvents and which in comparison to their free base forms, demonstrate higher melting points and an increased solubility.

In the present specification the term “alkyl” defines a straight or branched saturated hydrocarbon chain. The term “C₁-C₆ alkyl” refers to a straight or branched saturated hydrocarbon chain having one to six carbon atoms. The term “C₁-C₉ alkyl” refers to a straight or branched saturated hydrocarbon chain having one to nine carbon atoms. The term “C₁-C₁₅ alkyl” refers to a straight or branched saturated hydrocarbon chain having one to fifteen carbon atoms. Preferred is C₁-C₆ alkyl. Examples include methyl, ethyl, n-propyl, isopropyl, t-butyl, n-hexyl. The alkyl groups of the invention may be optionally substituted by one or more halogen atoms.

In the present specification the term “alkenyl” defines a straight or branched hydrocarbon chain having containing at least one carbon-carbon double bond. The term “C₁-C₆ alkenyl” refers to a straight or branched unsaturated hydrocarbon chain having one to six carbon atoms. The term “C₁-C₉ alkenyl” refers to a straight or branched unsaturated hydrocarbon chain having one to nine carbon atoms. The term “C₁-C₁₅ alkenyl” refers to a straight or branched unsaturated hydrocarbon chain having one to fifteen carbon atoms. Preferred is C₁-C₆ alkenyl. Examples include ethenyl, 2-propenyl, and 3-hexenyl. The alkenyl groups of the invention may be optionally substituted by one or more halogen atoms.

In the present specification the term “alkynyl” defines a straight or branched hydrocarbon chain having containing at least one carbon-carbon triple bond. The term “C₁-C₆ alkynyl” refers to a straight or branched unsaturated hydrocarbon chain having one to six carbon atoms. The term “C₁-C₉ alkynyl” refers to a straight or branched unsaturated hydrocarbon chain having one to nine carbon atoms. The term “C₁-C₁₅ alkynyl” refers to a straight or branched unsaturated hydrocarbon chain having one to fifteen carbon atoms. Preferred is C₁-C₆ alkynyl. Examples include ethynyl, 2-propynyl, and 3-hexynyl. The alkynyl groups of the invention may be optionally substituted by one or more halogen atoms.

As used herein, the term “carbocyclyl” means a mono- or polycyclic residue containing 3 or more (e.g. 3-10 or 3-8) carbon atoms. The carbocyclyl residues of the invention may be optionally substituted by one or more halogen atoms. Mono- and bicyclic carbocyclyl residues are preferred. The carbocyclyl residues can be saturated or partially unsaturated.

Saturated carbocyclyl residues are preferred and are referred to herein as “cycloalkyls” and the term “cycloalkyl” is used herein to define a saturated 3 to 14 membered carbocyclic ring including fused bicyclic or tricyclic systems. Examples of such groups include cyclopropyl, cyclobutyl, cyclopentyl, cyclohexyl and also bridged systems such as norbornyl and adamantyl. The cycloalkyl residues of the invention may be optionally substituted by one or more halogen atoms.

In the present specification the term “aryl” defines a 5-14 (e.g. 5-10) membered aromatic mono-, bi- or tricyclic group at least one ring of which is aromatic. Thus, bicyclic aryl groups may contain only one aromatic ring. As used herein, the term “aryl” includes heteroaryls containing heteroatoms (e.g. nitrogen, sulphur and/or oxygen) being otherwise as defined above. The aryl groups of the invention may optionally be substituted by one or more halogen atoms. Examples of aromatic moieties are benzene, naphthalene, imidazole and pyridine.

In the present specification, “halo” refers to fluoro, chloro, bromo or iodo.

### Medical Uses of the Compounds of the Invention

The invention finds general application in the treatment of any proteostatic disease. Accordingly, the compounds of the invention may be used for the treatment of both aggregative and misfolding proteostatic diseases, including prion diseases, various amyloidoses and neurodegenerative disorders (e.g. Parkinson's disease, Alzheimer's disease and Huntington's disease), lysosomal storage disorders, certain forms of diabetes, emphysema, cancer and cystic fibrosis. These medical uses are described in further detail below.

The folding and maintenance of proteins in a correctly folded, active (or native) form is essential to normal cellular function. The role of protein misfolding in a wide variety of human diseases is an emerging field of research and several previously unrelated diseases (including prion diseases, diabetes, cystic fibrosis, lysosomal storage diseases (LSDs), Parkinson's disease (PD), Alzheimer's disease (AD), Huntington's disease (HD), amyloidosis and cancer) are now known to involve proteotoxicity arising from misfolded protein. The proteotoxicity attendant on misfolding may arise from a variety of causes, including aggregation and deposition leading to tissue damage (e.g. in the amyloidoses) and inappropriate trafficking or clearance (e.g. in cystic fibrosis and the LSDs), leading to enzymic deficits.

The compounds of the invention find broad application in the treatment of protein misfolding diseases in general, including in particular the protein misfolding diseases described below:

### Protein Aggregation Diseases

### Amyloidoses

Certain proteins can assume a non-native, misfolded β-pleated sheet conformation which accumulates as amyloid fibrils (sometimes referred to as amyloid deposits or plaques) in organs and/or tissues, causing disease. These diseases are collectively known as amyloidoses.

Amyloidoses can be classified clinically as primary, secondary, familial, systemic or isolated. Primary amyloidosis appears without any preceding disease, for example arising from immune cell dysfunction such as multiple myeloma and other immunocyte dyscrasias. Secondary amyloidosis is a sequela of an existing disorder (typically a chronic inflammatory disease). Familial amyloidosis (which includes neuropathic, cardiopathic and or nephropathic forms) arises from an inherited mutation and can now be identified by DNA tests. Systemic forms involve amyloid deposition in plural tissues and/or organs (although the brain is almost never directly involved in systemic amyloidosis), while isolated (or localized) amyloidosis involves a single organ, tissue type or system. Thus, recognized clinical forms include ocular amyloidosis and central nervous system amyloidosis.

Amyloidoses can also be classified according to the chemical type of the amyloid protein. The amyloidoses are referred to with a capital A (for amyloid) followed by an abbreviation for the fibril protein. Thus, AA amyloidosis is characterized by extracellular deposition of fibrils that are composed of fragments of serum amyloid A (SAA) protein, a major acute-phase reactant protein, produced predominantly by hepatocytes. Similarly, AL amyloidosis (also called primary amyloidosis or light chain amyloidosis) is characterized by extracellular deposition of fibrils that are composed of an immunoglobulin light chain or light chain fragment, while ATTR amyloidosis is characterized by extracellular deposition of fibrils consisting of the transport protein transthyretin (TTR). Aβ amyloidosis (which appears in Alzheimer's disease) is characterized by extracellular deposition of fibrils that are composed of β-protein precursor.

Symptoms, prognosis and clinical setting differ greatly between amyloid types. Although about 23 different proteins are known to form amyloid in humans, only a few are associated with clinically significant amyloidosis. The various amyloid proteins and the type of amyloidosis and clinical setting in which they are involved are shown in the table below:

| Amyloidosis | Amyloid | |
|---|---|---|
| type | protein type | Principal Clinical Setting(s) |
| Systemic | Immunoglobulin | Plasma cell disorders |
| | light chains | |
| | Transthyretin | Familial amyloid polyneuropathies; |
| | (TTR) | senile cardiac amyloidosis |
| | Serum amyloid A | Amyloid A (AA) amyloidosis; |
| | | Inflammation-associated amyloidosis; |
| | | familial mediterranean fever |
| | β2-microglobulin | Dialysis-associated amyloidosis |
| | Immunoglobulin | Systemic amyloidosis |
| | heavy chains | |
| | Fibrinogen | Familial systemic amyloidosis |
| | alpha chain | |
| Hereditary | Apolipoprotein AI | Familial systemic amyloidosis |
| | Apolipoprotein AII | Familial systemic amyloidosis |
| | Lysozyme | Familial systemic amyloidosis |
| | β-protein precursor | Alzheimer's disease; Down's |
| | | syndrome; hereditary cerebral |
| | | hemorrhage with amyloidosis (Dutch) |
| Central | Prion protein | Creutzfeldt-Jakob disease; Gerstmann- |
| nervous | (AScr or PrP- | Sträussler-Scheinker disease; |
| system | 27) | fatal familial insomnia |
| | Cystatin C | hereditary cerebral hemorrhage with |
| | | amyloidosis (Icelandic) |
| | ABri precursor | Familial dementia (British) |
| | protein | |
| | ADan precursor | Familial dementia (Danish) |
| | protein | |
| | Gelsolin | Familial amyloidosis (Finnish) |
| Ocular | Lactoferrin | Familial corneal amyloidosis |
| | Keratoepithelin | Familial corneal dystrophies |
| | Calcitonin | Medullary thyroid carcinoma |
| | Amylin | Insulinoma; type 2 diabetes |
| Localized | Atrial natriuretic | Atrial amyloidosis |
| | factor | |
| | Prolactin | Pituitary amyloid |
| | Keratin | Cutaneous amyloidosis |
| | Medin | Senile aortic amyloidosis |

Amyloid A (AA) amyloidosis is the most common form of systemic amyloidosis worldwide. It occurs in the course of a chronic inflammatory disease of either infectious or noninfectious aetiology, hereditary periodic fevers and with certain neoplasms such as Hodgkin disease and renal cell carcinoma.

Thus, the compounds of the invention find application in the treatment of any of the various amyloidoses described above (and in particular those listed in the above table).

### Synucleinopathies

Synucleinopathies comprise a diverse group of neurodegenerative diseases characterized by the presence of lesions composed of aggregates of conformational and posttranslational modifications of α-synuclein in certain populations of neurons and glia. Abnormal filamentous aggregates of misfolded α-synuclein protein are the major components of Lewy bodies, dystrophic (Lewy) neurites, and the Papp-Lantos filaments in oligodendroglia and neurons in multiple system atrophy linked to degeneration of affected brain regions. In contrast to the extracellular amyloid plaques found in the brains of Alzheimer's patients, Lewy bodies are intracellular.

The synucleinopathies include Lewy body diseases (LBDs), dementia with Lewy bodies, multiple system atrophy (MSA), Hallervorden-Spatz disease, Parkinson's disease (PD), the Lewy body variant of Alzheimer's disease (LBVAD), neurodegeneration with brain iron accumulation type-1 (NBIA-1), pure autonomic failure, neuroaxonal dystrophy, amytrophic lateral sclerosis and Pick disease and various tauopathies.

Thus, the compounds of the invention find application in the treatment of Lewy body diseases (LBDs), dementia with Lewy bodies, multiple system atrophy (MSA), Hallervorden-Spatz disease, Parkinson's disease (PD), the Lewy body variant of Alzheimer's disease (LBVAD), neurodegeneration with brain iron accumulation type-1 (NBIA-1), pure autonomic failure, neuroaxonal dystrophy, amytrophic lateral sclerosis and Pick disease and various tauopathies.

### Expanded CAG Repeat Diseases

Certain protein aggregation diseases stem from the expansion of CAG repeats in particular genes with the encoded proteins having corresponding polyglutamine tracts which lead to aggregation and accumulation in the nuclei and cytoplasm of neurons. Aggregated amino-terminal fragments of mutant huntingtin are toxic to neuronal cells and are thought to mediate neurodegeneration.

An example is Huntington's disease (HD). Huntington's disease (HD) is characterized by selective neuronal cell death primarily in the cortex and striatum. It is caused by a CAG repeat expansion in the first exon of the huntingtin gene, which encodes a large protein of unknown function. The CAG repeat is highly polymorphic and varies from 6 to 39 repeats in normal individuals and from 35 to 180 repeats in HD cases.

In addition to HD, CAG expansions have been found in at least seven other inherited neurodegenerative disorders, including for example spinal and bulbar muscular atrophy (SBMA), Kennedy's disease, some forms of amyotrophic lateral sclerosis (ALS), dentatorubral pallidoluysian atrophy (DRPLA) and spinocerebellar ataxia (SCA) types 1, 2, 3, 6 and 7.

Thus, the compounds of the invention find application in the treatment of HD, SBMA, Kennedy's disease, ALS, DRPLA and SCA (e.g. types 1, 2, 3, 6 and 7).

### Tauopathies

The tauopathies are a group of diverse dementias and movement disorders which have as a common pathological feature the presence of intracellular accumulations of abnormal filaments of tau protein. Examples include Down's Syndrome (DS), Corticobasal Degeneration (CBD), Frontotemporal Dementia with Parkinsonism linked to Chromosome 17 (FTDP17), Pick Disease (PiD) and Progressive Supranuclear Palsy (PSP).

### Other Aggregation Diseases

Dominant mutations in Cu,Zn-superoxide dismutase (SOD1) cause a familial form of amyotrophic lateral sclerosis (fALS). A growing body of evidence suggests that the familial form of ALS (fALS) is caused by destabilization of the native structure of SOD1 leading to aggregation.

### Protein Folding (Conformational) Diseases

Factors that tilt the balance between correctly folded proteins and misfolded proteins are common causes of disease. The balance can be perturbed as the result of an age-related reduction in the efficiency of the quality control system and/or the acquisition or inheritance of mutations in the primary sequence of the encoding gene. Both lead to incorrect folding, lost activity, improper trafficking and/or misfolding outside the endoplasmic reticulum and cytoplasm.

### Lysosomal Storage Diseases

All lysosomal storage disorders (LSDs) are single gene diseases in which a mutant lysosomal enzyme is aberrantly expressed, processed or translocated in a manner which eliminates or reduces enzyme activity resulting in defective lysosomal acid hydrolysis of endogenous macromolecules and their consequent accumulation. This accumulation leads to tissue enlargement together with a fundamental perturbation of cellular physiology (including ER and oxidative stress) which can lead to severe systemic symptoms (including neurological deficits).

Listed below are a number of lysosomal storage disorders and the corresponding defective enzymes:
Pompe disease: Acid alpha-glucosidase
Gaucher disease: Acid beta-glucosidase or glucocerebrosidase
Fabry disease: alpha-Galactosidase A
GMI-gangliosidosis: Acid beta-galactosidase
Tay-Sachs disease: beta-Hexosaminidase A
Sandhoff disease: beta-Hexosaminidase B
Niemann-Pick disease: Acid sphingomyelinase
Krabbe disease: Galactocerebrosidase
Farber disease: Acid ceramidase
Metachromatic leukodystrophy: Arylsulfatase A
Hurler-Scheie disease: alpha-L-Iduronidase
Hunter disease: Iduronate-2-sulfatase
Sanfilippo disease A: Heparan N-sulfatase
Sanfilippo disease B: alpha-N-Acetylglucosaminidase
Sanfilippo disease C: Acetyl-CoA: alpha-glucosaminide N-acetyltransferase
Sanfilippo disease D: N-Acetylglucosamine-6-sulfate sulfatase
Morquio disease A: N-Acetylgalactosamine-6-sulfate sulfatase
Morquio disease B: Acid beta-galactosidase
Maroteaux-Lamy disease: Arylsulfatase B
Sly disease: beta-Glucuronidase
alpha-Mannosidosis: Acid alpha-mannosidase
beta-Mannosidosis: Acid beta-mannosidase
Fucosidosis: Acid alpha-L-fucosidase
Sialidosis: Sialidase
Schindler-Kanzaki disease: alpha-N-acetylgalactosaminidase

Thus, the compounds of the invention may be used for the treatment of an LSD selected from:
Pompe disease (including infantile and late-onset forms)
Gaucher disease (including Type 1, Type 2 and Type 3 Gaucher disease)
Fabry disease
GMI-gangliosidosis
Tay-Sachs disease
Sandhoff disease
Niemann-Pick disease
Krabbe disease
Farber disease
Metachromatic leukodystrophy
Hurler-Scheie disease
Hunter disease
Sanfilippo disease A
Sanfilippo disease B
Sanfilippo disease C
Sanfilippo disease D
Morquio disease A
Morquio disease B
Maroteaux-Lamy disease
Sly disease
alpha-Mannosidosis
beta-Mannosidosis
Fucosidosis
Sialidosis
Schindler-Kanzaki disease

In preferred embodiments, the lysosomal storage disease is selected from: (a) Pompe disease; (b) Gaucher disease; and (c) Fabry disease.

In particularly preferred embodiments, the lysosomal disease is selected from Type 1, Type 2 and Type 3 Gaucher disease.

### Cystic Fibrosis

The cystic fibrosis transmembrane conductance regulator (CFTR) is a chloride ion channel important in creating sweat, digestive juices and mucus. Cystic fibrosis occurs when there is a mutation in the CFTR gene leading to reduced ion channel activity (via increased clearance of the misfolded CFTR proteins).

Thus, the compounds of the invention find application in the treatment of cystic fibrosis.

### Emphysema

Misfolding/trafficking of alpha 1 anti-trypsin can cause emphysema, especially in childhood. Thus, the compounds of the invention can (at least partially) restore proper folding and/or trafficking of alpha 1 anti-trypsin and so find application in the treatment of emphysema, particularly childhood emphysema.

### Endoplasmic Reticulum Stress-Induced Diseases

The endoplasmic reticulum (ER) fulfills multiple cellular functions, including protein folding and the transport of proteins destined for extracellular compartments. Many disorders cause accumulation of unfolded proteins in the ER, triggering the unfolded protein response (UPR). The UPR functions to increase folding capacity and retrograde transport of misfolded proteins into the cytosol for proteasome-dependent degradation. However, chronic or excessive ER stress triggers apoptosis.

There is a growing recognition that ER stress underlies a wide range of different diseases, all caused (at least in part) by ER stress coupled with an aberrant UPR.

### Diabetes, Insulin Resistance, Obesity and Metabolic Syndrome

It has recently been recognized that aberrant UPR leads to ER stress and ultimately β-cell failure that contributes to the development of type II diabetes, insulin resistance and metabolic syndrome.

Thus, the compounds of the invention find application in the treatment of insulin resistance. Insulin resistance is characterized by a reduced action of insulin in skeletal muscle, adipocytes and hepatocytes so that normal amounts of insulin become inadequate to produce a normal insulin response from the cells of these tissues. In adipocytes, insulin resistance results in hydrolysis of stored triglycerides, leading to elevated free fatty acids in the blood plasma. In muscle, insulin resistance reduces glucose uptake while in hepatocytes it reduces glucose storage. In both of the latter cases an elevation of blood glucose concentrations results. High plasma levels of insulin and glucose due to insulin resistance often progresses to metabolic syndrome and type 2 diabetes.

The invention finds application in the treatment of metabolic syndrome. The disorder is also known as (metabolic) syndrome X, insulin resistance syndrome, Reaven's syndrome and CHAOS.

The invention finds application in the treatment of diseases associated with metabolic syndrome, including for example: fatty liver (often progressing to non-alcoholic fatty liver disease), polycystic ovarian syndrome, hemochromatosis (iron overload) and acanthosis nigricans (dark skin patches).

The invention finds application in the treatment of Type 2 diabetes. Type 2 diabetes is a chronic disease that is characterised by persistently elevated blood glucose levels (hyperglycaemia). Insulin resistance together with impaired insulin secretion from the pancreatic β-cells characterizes the disease. The progression of insulin resistance to type 2 diabetes is marked by the development of hyperglycaemia after eating when pancreatic β-cells become unable to produce adequate insulin to maintain normal blood sugar levels (euglycemia)).

The invention finds application in the treatment of Type 1 diabetes (or insulin dependent diabetes). Type 1 diabetes is characterized by loss of the insulin-producing beta cells of the islets of Langerhans in the pancreas, leading to a deficiency of insulin. The main cause of this beta cell loss is a T-cell mediated autoimmune attack. There is no known preventative measure that can be taken against type 1 diabetes, which comprises up to 10% of diabetes mellitus cases in North America and Europe. Most affected people are otherwise healthy and of a healthy weight when onset occurs. Sensitivity and responsiveness to insulin are usually normal, especially in the early stages.

The invention also finds application in the treatment of insulin resistance, various forms of diabetes, metabolic syndrome, obesity, wasting syndromes (for example, cancer associated cachexia), myopathies, gastrointestinal disease, growth retardation, hypercholesterolemia, atherosclerosis and age-associated metabolic dysfunction.

The invention may also be used for the treatment of conditions associated with metabolic syndrome, obesity and/or diabetes, including for example hyperglycaemia, glucose intolerance, hyperinsulinaemia, glucosuria, metabolic acidosis, cataracts, diabetic neuropathy, diabetic nephropathy, diabetic retinopathy, macular degeneration, glomerulosclerosis, diabetic cardiomyopathy, insulin resistance, impaired glucose metabolism, arthritis, hypertension, hyperlipidemia, osteoporosis, osteopenia, bone loss, brittle bone syndromes, acute coronary syndrome, infertility, short bowel syndrome, chronic fatigue, eating disorders, intestinal motility dysfunction and sugar metabolism dysfunction.

### Infectious Disease and Cancer

The compounds of the present invention can decreasing protein folding and or protein trafficking capacity, and since elevated enhanced folding and trafficking capacity is required for bacterial and viral replication and assembly as well as tumour cell growth and replication, the compounds of the invention find application in the treratment of infectious disease and cancer.

### Age-Onset Proteotoxicity Diseases

Age-associated decrease in cellular proteostasis capacity together with increasing protein damage leads to a plethora of age-onset diseases with a proteotoxic component. Thus, the compounds of the invention find application in the treatment of age-onset diseases, including for example various dementias and neurodegenerative diseases (e.g. AD, PD, ALS and HD), cancer, heart disease and autoimmune diseases (e.g. rheumatoid arthritis and diabetes).

### Compounds for Use According to the Invention

Certain compounds as described below (e.g. those compounds of Formula (1), (2) or (3) described in Section A(I) and/or the iminosugars described in Section A(II)) are novel.

According to the invention, those compounds which are novel are claimed as compounds per se, together with processes for their preparation, compositions containing them, as well as their use as pharmaceuticals (for example in any of the particular medical uses described herein).

Moreover, to the extent that certain of the compounds as described below (e.g. those compounds of Formula (1), (2) or (3) described in Section A(I) and/or the iminosugars described in Section A(II)) are known as such but not as pharmaceuticals, those compounds are claimed for use as pharmaceuticals (for example in any of the particular medical uses described herein).

### A. Structural Considerations

### (I) Compounds of Formula (1), (2) or (3)

The compounds for use according to the invention may comprise a nucleus selected from those shown below and numbered (1), (2) and (3):

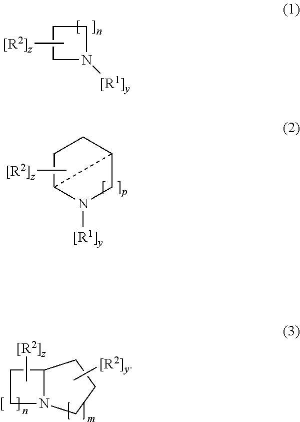

### (i) Compounds of Formula (1)

The compounds for use according to the invention may be of Formula (1)

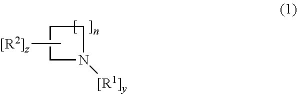

in which
n represents an integer from 1 to 7, provided that where n>1 the ring may also contain at least one unsaturated C—C bond
z represents an integer from 1 to (n+2)
y represents 1 or 2
R¹ represents H; C1-15 alkyl, C1-15 alkenyl or C1-15 alkynyl, optionally substituted with one or more R²; oxygen or an oxygen containing group such that the compound is an N-oxide; C(O)OR³; C(O)NR³R⁴; SO₂NR³; OH, OR³, or formyl
R² represents OH; OR³; ═O; NH₂; N₃; SH; SOₓR³; halo; CN; NO₂; NR³R⁴; (NR³)NR³R⁴; NH(NR³)NR³R⁴; CO₂R⁴; OC(O)R³; CONR³R⁴; NR⁴C(O)R³; NR⁴SO₂R³; P(O)(OR³)₂; C1-15 alkyl or alkenyl optionally substituted with one or more OH, OR³, ═O, NH₂, N₃, SH, SOₓR³, halo, CN, NO₂, NR³R⁴, (NR³)NR³R⁴, NH(NR³)NR³R⁴, CO₂R⁴, OC(O)R³, CONR³R⁴, NR⁴C(O)R³, NR⁴SO₂R³, P(O)(OR³)₂, aryl or carbocyclyl groups; carbocyclyl or aryl, either of which is optionally substituted with one or more OH, OR³, ═O, NH₂, N₃, SH, SOₓR³, halo, CN, NO₂, NR³R⁴, (NR³)NR³R⁴, NH(NR³)NR³R⁴, CO₂R⁴, OC(O)R³, CONR³R⁴, NR⁴C(O)R³, NR⁴SO₂R³, P(O)(OR³)₂, C1-9 alkyl optionally substituted with one or more OH, OR³, ═O, NH₂, N₃, halo, CN, NO₂, NR³R⁴, CO₂R⁴, CONR³R⁴, aryl or carbocyclyl groups; O-glycosyl; C-glycosyl; O-sulfate; O-phosphate or a group which together with the endocyclic carbon forms a spiro ring, with the provisos that: (a) two OH groups may not be attached to the same endocyclic carbon atom; (b) where there is only one R² substituent it contains an oxygen atom directly bonded to an endocyclic carbon atom; and (c) where z>1 any two R² substituents may together form an optionally heterocyclic ring (for example a carbocycle, cyclic ether or acetal)
R³ represents H; C1-6 alkyl, optionally substituted with one or more OH; aryl or C1-3 alkyl optionally substituted with aryl; SiR⁴₃ and
R⁴ represents H; C1-6 alkyl, optionally substituted with one or more OH
R³ and R⁴ may optionally form a 4 to 8 membered ring, containing one or more O, SOₓ or NR³ groups
x represents an integer from 0 to 2
   or a pharmaceutically acceptable salt or derivative thereof.

In preferred embodiments, the compound of Formula (1) is selected from any one of the Formulae shown below:

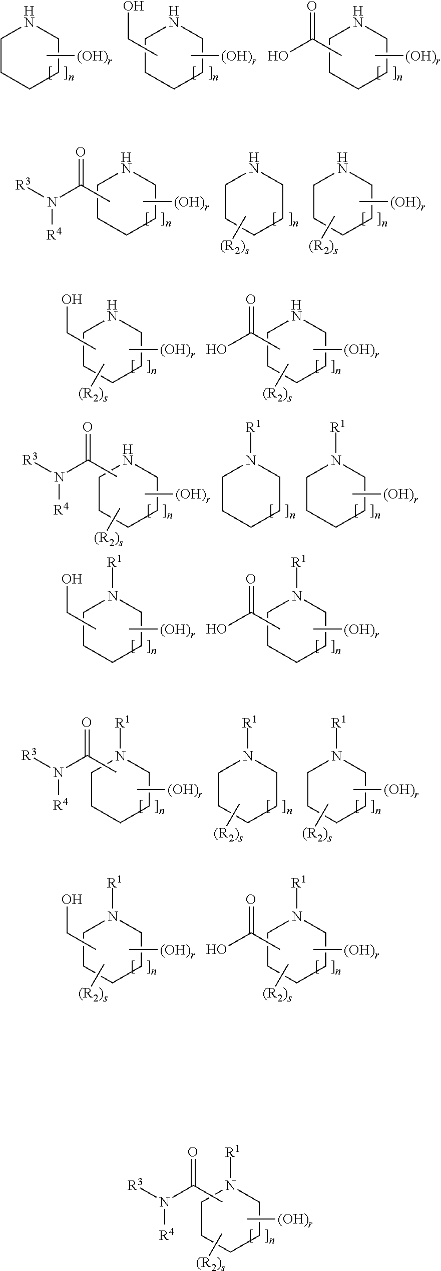

wherein:
r represents an integer from 1 to (n+4)
s represents an integer from 1 to (n+4)
n represents an integer from 0 to 2
R¹ represents C1-9 alkyl, optionally substituted with up to 6 OH, NR³R⁴, aryl, O—C1-3 alkyl, O—C1-3 alkenyl, CO₂H, NH(NH)NH₂, CONR³R⁴; C(O)OR³; C(O)NR³R⁴; SO₂NR³
R² represents ═O; C1-9 alkyl, C1-9 alkenyl, aryl, optionally substituted with up to 6 OH, NR³R⁴, aryl, O—C1-3 alkyl, CONR³R⁴, C(O)OR³; C(O)NR³R⁴; SO₂NR³; NH(NH)NH₂; NR⁴C(O)R³; NR⁴SO₂R³; N₃, F; Cl
R³ represents H; C1-6 alkyl, optionally substituted with up to 4 OH; aryl or C1-3 alkyl optionally substituted with aryl
R⁴ represents H; C1-6 alkyl, optionally substituted with up to 4 OH
R³ and R⁴ may optionally form a 4 to 8 membered ring, containing 0 to 1 O, S or NR³ groups.

### (ii) Compounds of Formula (21

The compounds for use according to the invention may be of Formula (2)

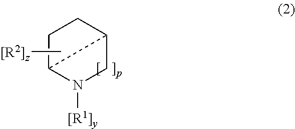

in which
p represents an integer from 1 to 2
z represents an integer from 1 to (p+7)
y represents 1 or 2
the broken line represents a bridge containing 2 or 3 carbon atoms between any two different ring carbon atoms, any or all of which bridge or bridgehead carbon atoms being optionally substituted with R²
R¹ represents H; C1-15 alkyl, C1-15 alkenyl or C1-15 alkynyl, optionally substituted with one or more R²; oxygen or an oxygen containing group such that the compound is an N-oxide; C(O)OR³; C(O)NR³R⁴; SO₂NR³; OH, OR³, or formyl
R² represents OH; OR³; ═O; NH₂; N₃, SH; SOₓR³; halo; CN; NO₂; NR³R⁴; (NR³)NR³R⁴; NH(NR³)NR³R⁴; CO₂R⁴; OC(O)R³; CONR³R⁴; NR⁴C(O)R³; NR⁴SO₂R³;
P(O)(OR³)₂; C1-15 alkyl or alkenyl optionally substituted with one or more OH, OR³, ═O, NH₂, N₃, SH, SOₓR³, halo, CN, NO₂, NR³R⁴, (NR³)NR³R⁴, NH(NR³)NR³R⁴, CO₂R⁴, OC(O)R³, CONR³R⁴, NR⁴C(O)R³, NR⁴SO₂R³, P(O)(OR³)₂, aryl or carbocyclyl groups; carbocyclyl or aryl, either of which is optionally substituted with one or more OH, OR³, ═O, NH₂, N₃, SH, SOₓR³, halo, CN, NO₂, NR³R⁴, (NR³)NR³R⁴, NH(NR³)NR³R⁴, CO₂R⁴, OC(O)R³, CONR³R⁴, NR⁴C(O)R³, NR⁴SO₂R³, P(O)(OR³)₂, C1-9 alkyl optionally substituted with one or more OH, OR³, ═O, NH₂, N₃, halo, CN, NO₂, NR³R⁴, CO₂R⁴, CONR³R⁴, aryl or carbocyclyl groups; O-glycosyl; C-glycosyl; O-sulfate; O-phosphate or a group which together with the endocyclic carbon forms a Spiro ring, with the provisos that: (a) two OH groups may not be attached to the same endocyclic carbon atom; (b) where there is only one R² substituent it contains an oxygen atom directly bonded to an endocyclic carbon atom; and (c) where z>1 any two R² substituents may together form an optionally heterocyclic ring (for example a carbocycle, cyclic ether or acetal)
R³ represents H; C1-6 alkyl, optionally substituted with one or more OH; aryl or C1-3 alkyl optionally substituted with aryl; SiR⁴₃ and
R⁴ represents H; C1-6 alkyl, optionally substituted with one or more OH
R³ and R⁴ may optionally form a 4 to 8 membered ring, containing one or more O, SOₓ or NR³ groups
x represents an integer from 0 to 2
   or pharmaceutically acceptable salt or derivative thereof.

In preferred embodiments, the compound of Formula (2) is selected from any one of the Formulae shown below:

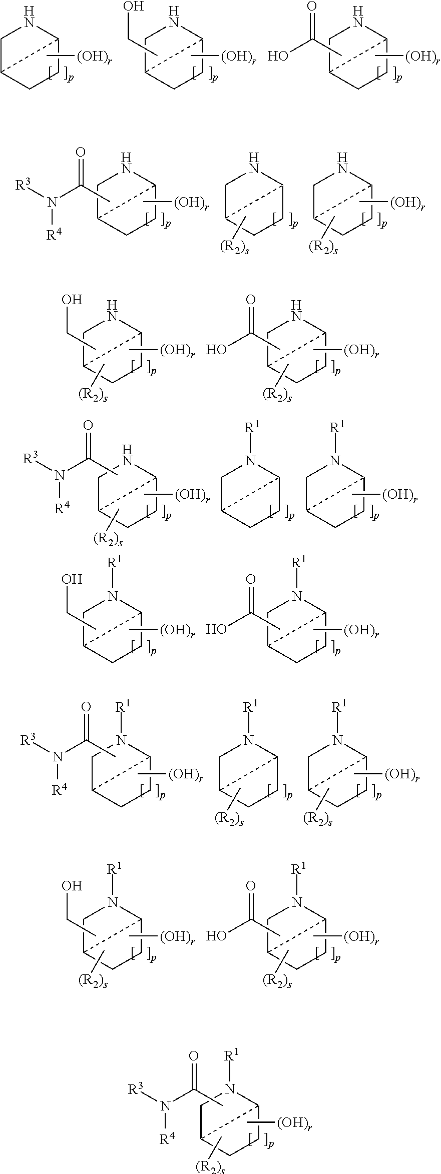

wherein:
r represents an integer from 1 to (n+4)
s represents an integer from 1 to (n+4)
p represents an integer from 1 to 2
R¹ represents C1-9 alkyl, optionally substituted with up to 6 OH, NR³R⁴, aryl, O—C1-3 alkyl, O—C1-3 alkenyl, CO₂H, NH(NH)NH₂, CONR³R⁴; C(O)OR³; C(O)NR³R⁴; SO₂NR³
R² represents ═O; C1-9 alkyl, C1-9 alkenyl, aryl, optionally substituted with up to 6 OH, NR³R⁴, aryl, O—C1-3 alkyl, CONR³R⁴, C(O)OR³; C(O)NR³R⁴; SO₂NR³; NH(NH)NH₂; NR⁴C(O)R³; NR⁴SO₂R³; N₃; F; Cl
R³ represents H; C1-6 alkyl, optionally substituted with up to 4 OH; aryl or C1-3 alkyl optionally substituted with aryl
R⁴ represents H; C1-6 alkyl, optionally substituted with up to 4 OH
R³ and R⁴ may optionally form a 4 to 8 membered ring, containing 0 to 1 O, S or NR³ groups.
   (iii) Compounds of Formula (3)

The compounds for use according to the invention may be of Formula (3)

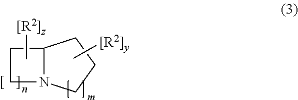

in which
n represents an integer from 1 to 7, for example 1 to 5, provided that where n>1 the ring may also contain at least one unsaturated C—C bond
m represents an integer from 1 to 3 and the ring may also contain at least one unsaturated C—C bond
z represents an integer from 0 to (n+2), provided that where z=0 then y≧1
y represents an integer from 0 to (m+2), provided that where y=0 then z≧1 the endocyclic nitrogen atom may be bonded to an oxygen or an oxygen containing group such that the compound is an N-oxide,
R² represents OH; OR³; ═O; NH₂; N₃; SH; SOₓR³; halo; CN; NO₂; NR³R⁴; (NR³)NR³R⁴; NH(NR³)NR³R⁴; CO₂R⁴; OC(O)R³; CONR³R⁴; NR⁴C(O)R³; NR⁴SO₂R³; P(O)(OR³)₂; C1-15 alkyl or alkenyl optionally substituted with one or more OH, OR³, ═O, NH₂, N₃, SH, SOₓR³, halo, CN, NO₂, NR³R⁴, (NR³)NR³R⁴, NH(NR³)NR³R⁴, CO₂R⁴, OC(O)R³, CONR³R⁴, NR⁴C(O)R³, NR⁴SO₂R³, P(O)(OR³)₂, aryl or carbocyclyl groups; carbocyclyl or aryl, either of which is optionally substituted with one or more OH, OR³, ═O, NH₂, N₃, SH, SOₓR³, halo, CN, NO₂, NR³R⁴, (NR³)NR³R⁴, NH(NR³)NR³R⁴, CO₂R⁴, OC(O)R³, CONR³R⁴, NR⁴C(O)R³, NR⁴SO₂R³, P(O)(OR³)₂, C1-9 alkyl optionally substituted with one or more OH, OR³, ═O, NH₂, N₃, halo, CN, NO₂, NR³R⁴, CO₂R⁴, CONR³R⁴, aryl or carbocyclyl groups; O-glycosyl; C-glycosyl; O-sulfate; O-phosphate or a group which together with the endocyclic carbon forms a spiro ring, with the provisos that: (a) two OH groups may not be attached to the same endocyclic carbon atom; (b) where there is only one R² substituent it contains an oxygen atom directly bonded to an endocyclic carbon atom; and (c) where z>1 any two R² substituents may together form an optionally heterocyclic ring (for example a carbocycle, cyclic ether or acetal)
R³ represents H; C1-6 alkyl, optionally substituted with one or more OH; aryl or C1-3 alkyl optionally substituted with aryl; SiR⁴₃ and
R⁴ represents H; C1-6 alkyl, optionally substituted with one or more OH
R³ and R⁴ may optionally form a 4 to 8 membered ring, containing one or more O, SOₓ or NR³ groups
x represents an integer from 0 to 2
optionally wherein the compound has three, four or more rings
   or pharmaceutically acceptable salt or derivative thereof.

In preferred embodiments, the compound of Formula (3) is selected from any one of the Formulae shown below:

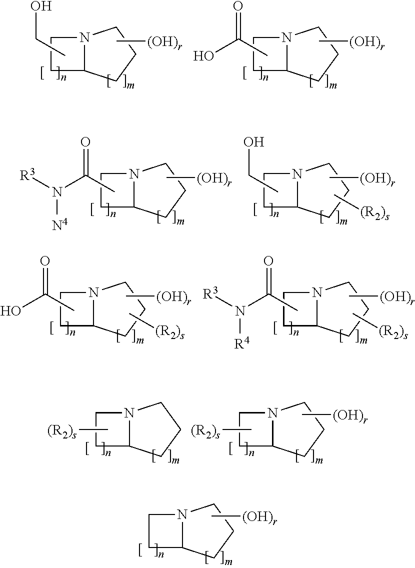

wherein:
r represents an integer from 1 to (n+m+4)
s represents an integer from 1 to (n+m+4)
n represents an integer from 1 to 3
m represents an integer from 1 to 3
R² represents ═O; C1-9 alkyl, C1-9 alkenyl, aryl, optionally substituted with up to 6 OH, NR³R⁴, aryl, O—C1-3 alkyl, CONR³R⁴, C(O)OR³; C(O)NR³R⁴; SO₂NR³; NH(NH)NH₂; NR⁴C(O)R³; NR⁴SO₂R³; N₃; F; Cl
R³ represents H; C1-6 alkyl, optionally substituted with up to 4 OH; aryl or C1-3 alkyl optionally substituted with aryl
R⁴ represents H; C1-6 alkyl, optionally substituted with up to 4 OH
R³ and R⁴ may optionally form a 4 to 8 membered ring, containing 0 to 1 O, S or NR³ groups the endocyclic nitrogen atom may be bonded to an oxygen or an oxygen containing group such that the compound is an N-oxide.

In all of the above compounds, one or more endocyclic carbon atoms may be substituted with a sulphur, oxygen or nitrogen atom.

It will be appreciated that the compounds of Formula (1), (2) and (3) may comprise compounds having three, four or more rings.

Preferred are compounds of Formula (1), (2) or (3) which are polyhydroxylated, having 2, 3 or more hydroxyl residues on the ring system nucleus.

Also preferred are oligomers (e.g. dimers, trimers etc.) of the above-defined compounds. Such compounds may be di- and/or oligosaccharide mimetics (as described below), and they may be linked, for example, at C6 and C2, 3 or 4. Oligomers of the above-defined compounds are preferably imino-C-disaccharides and analogues as described in Section II(b)(vi), below.

Certain compounds of Formula (1), (2) or (3) are novel. According to the invention, those compounds of Formula (1), (2) or (3) which are novel are claimed as compounds per se, together with processes for their preparation, compositions containing them, as well as their use as pharmaceuticals (for example in any of the particular medical uses described herein).

Moreover, to the extent that certain of the compounds falling within the scope of Formula (1), (2) or (3) are known, as such, but not as pharmaceuticals, those compounds are claimed for use as pharmaceuticals (for example in any of the particular medical uses described herein).

The compounds of Formula (1), (2) or (3) may be, but not necessarily are, iminosugars as defined in Section A(II) (below).

### (II) Iminosugars

The compounds for use according to the invention may be iminosugars, as hereinbefore defined.

Thus, the compounds for use according to the invention may be selected from:
iminosugars sensu stricto, being saccharide analogues in which the ring oxygen is replaced by a nitrogen; or
isoiminosugars, being aza-carba analogues of sugars in which the C1 carbon is replaced by nitrogen and the ring oxygen is replaced by a carbon atom; and
azasugars in which an endocyclic carbon is replaced with a nitrogen atom.

In embodiments where the iminosugar for use according to the invention is an azasugar as defined above, then the iminosugar may be selected from:
1-azasugars in which the N is in the anomeric position;
oxazines in which the ring oxygen remains unsubstituted; and
hydrazines in which the ring oxygen is substituted with a nitrogen atom.

In all of the above iminosugars, one or more endocyclic carbon atoms may be substituted with a sulphur, oxygen or nitrogen atom.

The iminosugars for use according to the invention may be of Formula (1), (2) or (3) as defined in Section A(I) (above).

The iminosugars as defined above for use according to the invention may be of any structural class or subclass, including the classes described below:

### (a) Principal Structural Iminosugar Classes

The compounds for use according to the invention may be an iminosugar (as herein defined). The iminosugars for use according to the invention may be of a structural class selected from:
(a) a piperidine;
(b) a pyrroline;
(c) a pyrrolidine;
(d) a pyrrolizidine;
(e) an indolizidine;
(f) a quinolizidine;
(g) a nortropane;
(h) ring-open iminosugars;
(i) 5,7 fused;
(j) an azepane;
(k) an azetidine;
(l) mixtures of any two or more of (a) to (k).

The iminosugars of any of the foregoing structural classes may be polyhydroxylated, as hereinbefore defined. As used herein, the term polyhydroxylated piperidine iminosugar defines an oxygenated iminosugar (e.g. having at least 2 (preferably at least 3) free hydroxyl groups (or alkyl groups with one or more OH substituents) on the ring system nucleus) that comprises the nucleus:

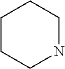

As used herein, the term polyhydroxylated pyrrolidine iminosugar defines an oxygenated iminosugar (e.g. having at least 2 (preferably at least 3) free hydroxyl groups (or alkyl groups with one or more OH substituents) on the ring system nucleus) that comprises the nucleus:

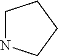

As used herein, the term polyhydroxylated pyrrolizidine iminosugar defines an oxygenated iminosugar (e.g. having at least 3, 4, 5, 6 or 7 (preferably 3, 4 or 5) free hydroxyl groups (or alkyl groups with one or more OH substituents) on the ring system nucleus) that comprises the nucleus:

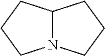

As used herein, the term polyhydroxylated indolizidine iminosugar defines an oxygenated iminosugar (e.g. having at least 3, 4, 5, 6 or 7 (preferably 3, 4 or 5) free hydroxyl groups (or alkyl groups with one or more OH substituents) on the ring system nucleus) that comprises the nucleus:

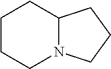

As used herein, the term polyhydroxylated quinolizidine iminosugar defines an oxygenated iminosugar (e.g. having at least 3, 4, 5, 6 or 7 (preferably 3, 4, 5 or 6) free hydroxyl groups (or alkyl groups with one or more OH substituents) on the ring system nucleus) that comprises the nucleus:

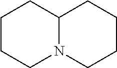

In each of the above iminosugar nuclei, it is to be understood that one or more endocyclic carbon atoms may be substituted with a sulphur, oxygen or nitrogen atom.

### (i) Piperidine Iminosugars

Piperidine iminosugars comprise the nucleus:

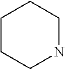

Preferred are polyhydroxylated piperidine iminosugars as hereinbefore defined comprising the above nucleus and having at least 2 (preferably at least 3) hydroxyl groups (or alkyl groups with one or more hydroxy substituent(s)) on the ring system nucleus.

### (ii) Pyrroline Iminosugars

Pyrroline iminosugars comprise one of the following three nuclei:

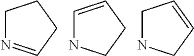

Preferred are polyhydroxylated pyrroline iminosugars as hereinbefore defined having at least 2 hydroxyl groups (or alkyl groups with one or more hydroxy substituent(s)) on the ring system nucleus.

(iii) Pyrrolidine Iminosugars

Pyrrolidine iminosugars comprise the nucleus:

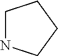

Preferred are polyhydroxylated pyrrolidine iminosugars as hereinbefore defined comprising the above nucleus and having at least 2 (for example at least 3) hydroxyl groups (or alkyl groups with one or more hydroxy substituent(s)) on the ring system nucleus.

### (iv) Pyrrolizidine Iminosugars

Pyrrolizidine iminosugars comprise the nucleus:

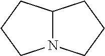

Preferred are polyhydroxylated pyrrolizidine iminosugars as hereinbefore defined comprising the above nucleus and having at least 2, 3, 4, 5, 6 or 7 (preferably 3, 4 or 5) hydroxyl groups (or alkyl groups with one or more hydroxy substituent(s)) on the ring system nucleus.

### (v) Indolizidine Iminosugars

Indolizidine iminosugars comprise the nucleus:

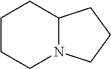

Preferred are polyhydroxylated indolizidine iminosugars as hereinbefore defined comprising the above nucleus and having at least 2, 3, 4, 5, 6 or 7 (preferably 3, 4 or 5) hydroxyl groups (or alkyl groups with one or more hydroxy substituent(s)) on the ring system nucleus.

### (vi) Quinolizidine Iminosugars

Quinolizidine iminosugars comprise the nucleus:

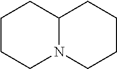

Preferred are polyhydroxylated quinolizidine iminosugars as hereinbefore defined comprising the above nucleus and having at least 2, 3, 4, 5, 6 or 7 (preferably 3, 4, 5 or 6) hydroxyl groups (or alkyl groups with one or more hydroxy substituent(s)) on the ring system nucleus.

(vii) Nortropanes

Nortropane iminosugars comprise the nucleus:

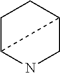

wherein the dotted line represents a bridge containing 2 or 3 carbon atoms between any two different ring carbon atoms.

Preferred are polyhydroxylated nortropane iminosugars as hereinbefore defined comprising the above nucleus and having at least 3 (preferably at least 4) hydroxyl groups (or alkyl groups with one or more hydroxy substituent(s)) on the ring system nucleus.

A preferred class of nortropane iminosugar for use according to the invention are calystegines. These are polyhdroxylated nortropanes which have been reported to inhibit β-glucosidases, β-xylosidases and α-galactosidases (Asano et al., 1997, Glycobiology 7: 1085-1088). The calystegines are common in foods belonging to the Solanaceae that includes potatoes and aubergines (egg plant). The calystegines have been shown to inhibit mammalian glycosidases including human, rat and bovine liver enzymes. Attaching sugars to the calystegines such as in 3-0-β-D-glucopyranoside of 1α,2β,3α,6α-tetrahydroxy-nor-tropane (Calystegine B₁) (Griffiths, et al., 1996, Tetrahedron Letters 37: 3207-3208) can alter the glycosidase inhibition to include α-glucosidases and β-galactosidases.

(viii) 5-7 Fused

These iminosugars comprise the nucleus:

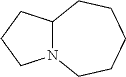

Preferred are polyhydroxylated 5-7 fused iminosugars as hereinbefore defined comprising the above nucleus and having at least 2, 3, 4, 5, 6 or 7 (preferably 3, 4 or 5) hydroxyl groups (or alkyl groups with one or more hydroxy substituent(s)) on the ring system nucleus.

### (ix) Azepanes

Azepane imino sugars comprise the nucleus:

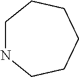

Preferred are polyhydroxylated azepane iminosugars as hereinbefore defined comprising the above nucleus and having at least 2 (preferably at least 3 or 4) hydroxyl groups (or alkyl groups with one or more hydroxy substituent(s)) on the ring system nucleus.

In each of the above iminosugar nuclei described in subsections (i) to (ix), it is to be understood that one or more endocyclic carbon atoms may be substituted with a sulphur, oxygen or nitrogen atom.

It will also be appreciated that iminosugars comprising the various nuclei described in subsections (i) to (ix) comprise compounds having three, four or more rings.

### (x) Ring-Open Iminosugars

Also considered are amino sugars acids formed by the opening of the imino ring such as compound P1 and P2 (found in Cucurbita spp.) and P3. Such compounds may also be the biological precursors of the iminosugar acids.

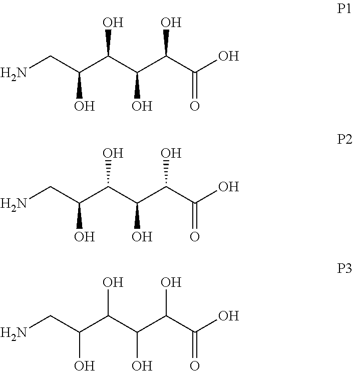

### (b) Iminosugar Structural Subclasses

The principal structural classes described above can be further categorized into various subclasses, for example on the basis of the presence of various functional groups, as described below.

The iminosugars for use according to the invention may therefore be further characterized on the basis of their structural subclass, for example being selected from:

### (i) Iminosugar Acids

The iminosugar acids (ISAs) are mono- or bicyclic analogues of sugar acids in which the ring oxygen is replaced by a nitrogen. Although iminosugars are widely distributed in plants (Watson et al. (2001) Phytochemistry 56: 265-295), the iminosugar acids are much less widely distributed.

Iminosugar acids can be classified structurally on the basis of the configuration of the N-heterocycle. Examples include piperidine, pyrroline, pyrrolidine, pyrrolizidine, indolizidine and nortropanes iminosugar acids (see FIGS. 1-7 of Watson et al. (2001) Phytochemistry 56: 265-295), the disclosure of which is incorporated herein by reference).

Particularly preferred are iminosugar acids selected from the following structural classes:
(a) piperidine ISAs (including (poly)hydroxypipecolic acids);
(b) pyrroline ISAs;
(c) pyrrolidine ISAs (including (poly)hydroxyprolines);
(d) pyrrolizidine ISAs;
(e) indolizidine ISAs; and
(f) nortropane ISAs.

The ISAs for use according to the invention may be N-acid ISAs (as hereinbefore defined).

ISA mixtures or combinations containing two or more different ISAs representative of one or more of the classes listed above may also be used.

Preferred are polyhydroxylated ISAs. Particularly preferred are ISAs having a small molecular weight, since these may exhibit desirable pharmacokinetics. Thus, the ISA may have a molecular weight of 100 to 400 Daltons, preferably 150 to 300 Daltons and most preferably 200 to 250 Daltons.

Also preferred are ISAs, which are analogues of hydroxymethyl-substituted iminosugars in which one or more hydroxymethyl groups are replaced with carboxyl groups.

### Exemplary Piperidine Iminosugar Acids

The ISA of the invention may be a piperidine ISA having at least 3 free hydroxyl (or hydroxyalkyl) groups on the ring system nucleus. Exemplary piperidine ISAs are hydroxypipecolic acids. Particularly preferred hydroxypipecolic acids are polyhydroxypipecolic acids having at least two (e.g. 3) free hydroxyl (or hydroxyalkyl) groups on the ring system nucleus.

### Exemplary Pyrrolidine Iminosugar Acids

The ISA of the invention may be a pyrrolidine ISAs having at least 2 (preferably at least 3) free hydroxyl (or hydroxyalkyl) groups on the ring system nucleus. Preferred pyrrolidine ISAs are hydroxyprolines. Particularly preferred hydroxyprolines are polyhydroxyprolines having at least two (e.g. at least 3) free hydroxyl (or hydroxyalkyl) groups on the ring system nucleus.

### Exemplary Pyrrolizidine Iminosugar Acids

The ISA of the invention may be a pyrrolizidine ISA having at least 2 (preferably at least 3, 4 or 5) free hydroxyl (or hydroxyalkyl) groups on the ring system nucleus.

### Exemplary Indolizidine Iminosugar Acids

The ISA of the invention may be an indolizidine ISA having at least 2 (preferably at least 3, 4 or 5) free hydroxyl (or hydroxyalkyl) groups on the ring system nucleus.

### Exemplary Nortropane Iminosugar Acids

The ISA of the invention may be a nortropane ISA having at least 2 (preferably at least 3) free hydroxyl (or hydroxyalkyl) groups on the ring system nucleus.

### (ii) 1-N-Iminosugars (Isoiminosuaars)

Isoimino sugars are carbohydrate mimics in which the anomeric carbon is replaced by a nitrogen atom and the ring oxygen is repaced by a carbon atom (for example, a methylene group in the case of monocyclic piperidine and pyrrolidine compounds).

(iii) Iminosugar Coniuqates

Carbohydrates are often conjugated to other biomolecules in vivo, including lipids, proteins, nucleosides and phosphate groups. Thus, of particular interest as a subclass of the various principal classes of iminosugar described above are iminosugar conjugates. These include:
Iminosugar-based glycopeptide analogues
Iminosugar phosphonate analogues
Iminosugar nucleotide analogues
Iminosugar glycolipid analogues (e.g. C- or N-alkyl iminosugar derivatives)

### (iv) Iminosugar C-glucosides

Imino-analogues of glycosides in which an aglycone moiety is attached to the anomeric (C-1) carbon via an O-glycosidic bond are of limited utility as drugs due to the lability of the N,O-acetal function. Replacement of the oxygen atom of the N,O-acetal by a methylene group yields iminosugar C-glycosides, which are stable analogues of glycoconjugates. The endocyclic nitrogen is preferably unsubstituted in such C-glycosides, so that the compounds may comprise a nucleus selected from those listed below:

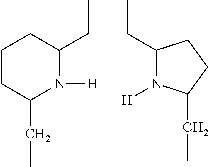

Iminosugars of this structural subclass are described by Compain (2007) in Iminosugars From Synthesis to Therapeutic Applications: Compain, Philippe/Martin, Olivier R. (eds.) ISBN-13: 978-0-470-03391-3-John Wiley & Sons) pages 63-86 (the disclosure of which is hereby incorporated by reference).

### (v) N-Substituted Iminosugars

N-substituted iminosugars may be considered as analogues of the iminosugar C-glycosides described above in which the aglycone moiety is positioned on the endocyclic nitrogen rather than the “anomeric” C-1 carbon atom.

### (vi) Imino-C-Disaccharides and Analogues

Imino-C-disaccharides and analogues for use according to the invention may fall into any one of the three structural subclasses described by Vogel et al. (2007) in Iminosugars From Synthesis to Therapeutic Applications: Compain, Philippe/Martin, Olivier R. (eds.) ISBN-13: 978-0-470-03391-3-John Wiley & Sons) pages 87-130 the disclosure of which is hereby incorporated herein by reference. For example, they may be: (a) linear (1→1)-Clinked; (b) linear (1→ω)-C-linked; or (c) branched (1→n)-C-linked (see FIG. 5.1 of Vogel et al. (2007), op. cit.).

(vii) Iminosugar lactams

Iminosugar lactams for use according to the invention may for example comprise a nucleus selected from:

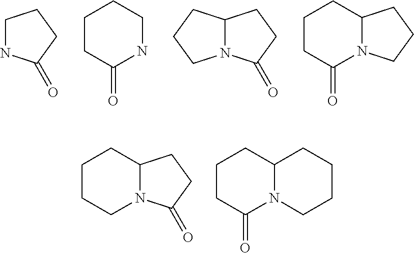

in which the ═O group may be on both rings of the bicyclic nuclei.

In each of the above iminosugar lactam nuclei, it is to be understood that one or more endocyclic carbon atoms may be substituted with a sulphur, oxygen or nitrogen atom.

(viii) Branched Iminosugars

The iminosugars for use according to the invention may be a branched imino sugar. Branched iminosugars are as defined in sections (i) to (x) (above) but are distinguished by the presence of two non-H substituents (e.g. two alkyl groups, two hydroxyalkyl groups, a hydroxy and hydroxyalkyl group or a hydroxy and alkyl group) on any one or more endocyclic carbon atom.

It will be appreciated that iminosugars with features characteristic of two or more of the foregoing subclasses (i) to (x) may also find application according to the invention.

### (c) Iminosugar Carbohydrate Mimetics

As described above, the iminosugars for use according to the invention, may be of any structural class and/or subclass, including the classes and subclasses described above in Sections II(a) and II(b). In addition to this structural classification, the iminosugars for use according to the invention may also be further structurally and/or functionally defined by reference to the carbohydrate(s) they mimic, as described below:

### (i) General Considerations

An iminosugar carbohydrate mimetic is an iminosugar that mimics one or more carbohydrates (for example, a mono- or disaccharide) through replication of one or more structural motifs of the carbohydrate scaffold. Thus, iminosugar carbohydrate mimetics share absolute/relative stereochemical motifs with the carbohydrate(s) they mimic.

This structural mimicry may be associated with functional mimicry: the shared absolute/relative stereochemical motifs may give rise to shared functional attributes. In such cases the compound may be defined as a functional sugar mimetic (as discussed in more detail in Section B, below). However, since the sugar mimics of the carbohydrate may also contain new functional groups, a new scaffold, or both, they may also exhibit functional attributes which are distinct from those of the carbohydrate(s) mimicked.

Thus, iminosugar carbohydrate mimetics correspond structurally to one or more carbohydrates and this structural mimicry may be accompanied by functional mimicry (e.g. at the level of interaction with a biological target in vivo) or other functional attributes related to, but distinct from, those of the carbohydrate they mimic (for example, the ability to competitively inhibit an enzyme for which the carbohydrate mimicked is a substrate in vivo).

For example, and considering the following pentose (3 contiguous chiral centres) and hexose (4 contiguous chiral centres) stereochemistries (Scheme 1, below):

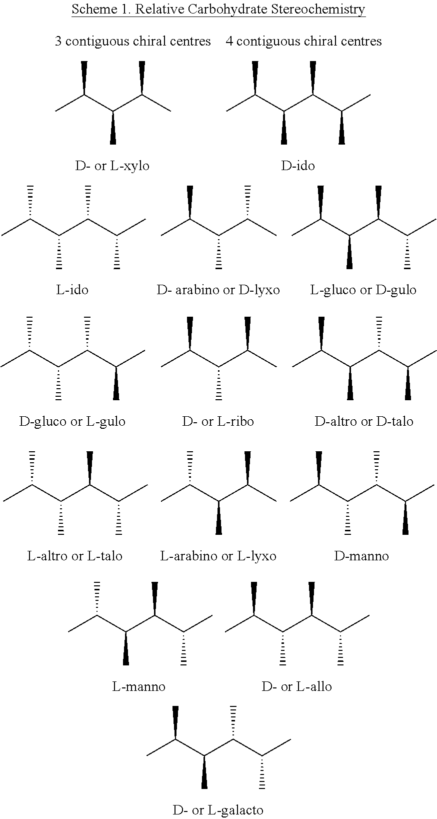

The above analysis is non-limiting, and intended to be illustrative only of a wider principle. A similar analysis can readily be extended to lower sugars (e.g. tetroses) and higher sugars (e.g. heptoses), as well as to ketoses and the like.

An iminosugar can be considered as being a structural mimetic of a particular reference monosaccharide, disaccharide or oligosaccharide unit when stereochemical comparisons between the iminosugar and the relative carbohydrate stereochemistry exhibited by the carbohydrate scaffold reveal shared stereochemical motifs. For the purposes of the analysis, the stereochemical comparison relates to consideration of contiguous C-het stereocentres (these being C—O, C—N etc.)

For example in the case of two simple monocyclic iminosugars IS1 and IS2 (shown below) the relative stereochemical relationship to the reference monosaccharide units (D-arabinose and D-glucose respectively) can be seen:

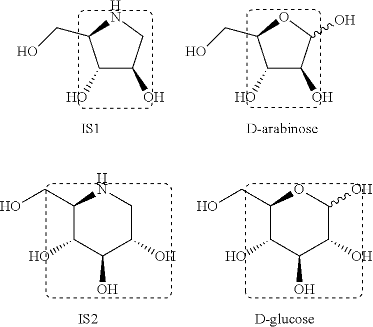

Thus, IS1 is a D-arabinose mimetic while IS2 is a D-glucose mimetic.

However, as monosaccharides can exist in both acyclic and several cyclic forms, the relative stereochemical relationship between the iminosugar and the parent monosaccharide is not necessarily fixed to one structural class or type or to the contiguous sequence depicted.

For example, D-arabinose can exist in the following cyclic forms:

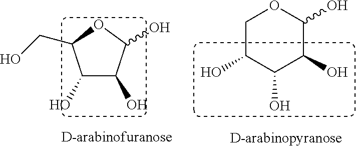

Exemplary iminosugar mimetics include the iminosugars IS1 and 153, respectively, as shown below:

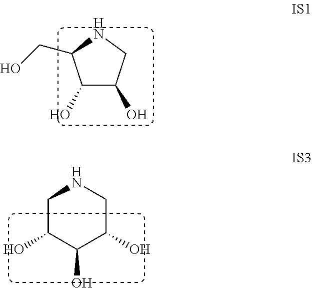

Note that unlike their monosaccharide counterparts these compounds generally cannot interconvert and are chemically distinct from each other. Thus, IS1 is a D-arabinofuranose mimetic while IS3 is a D-arabinopyranose mimetic.

However, in the case of IS3 the stereochemistry represents that not just of D-arabinopyranose but also that of D-lyxose:

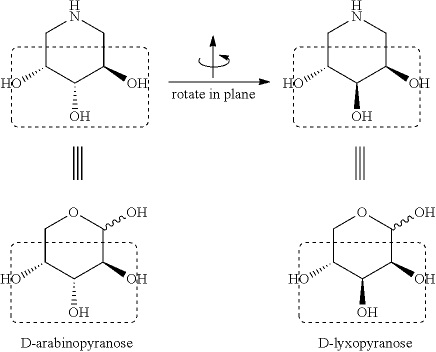

This is a consequence of the stereochemical sequence overlap that exists amongst carbohydrate sequences. For these purposes the carbon backbone with the most contiguous chiral centres is selected primarily. When considering cyclic iminosugars the ring nitrogen is included amongst the primary contiguous chiral centres.

For example, the iminosugar IS4 exhibits the following stereochemical sequences:

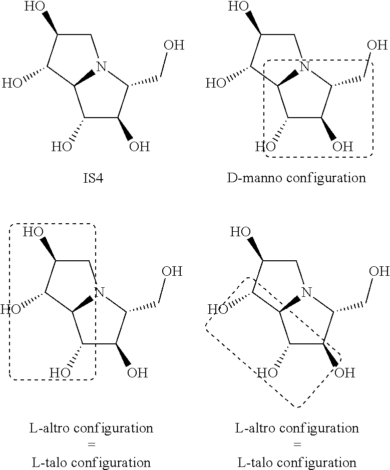

The iminosugar IS5 exhibits the following stereochemical sequences:

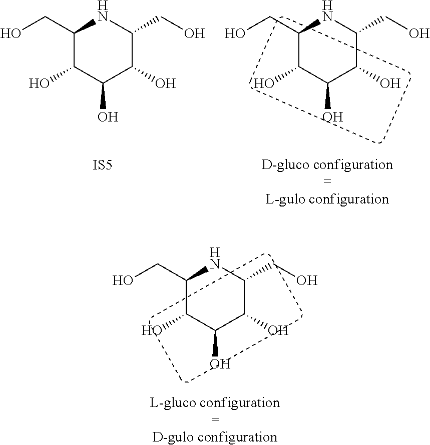

The iminosugar IS6 exhibits the following stereochemical sequences:

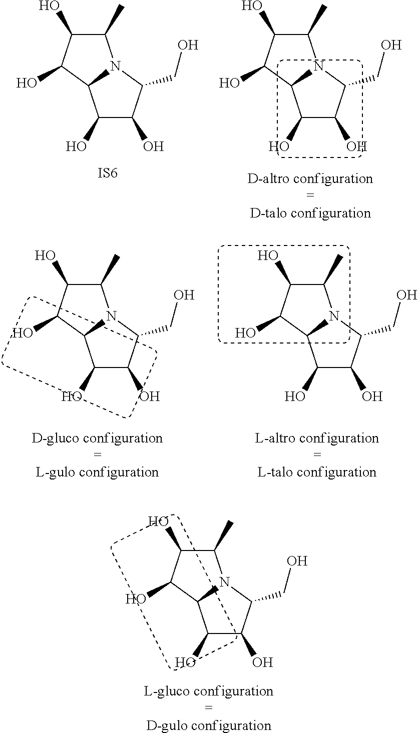

However, although an iminosugar may present more than one stereochemical sequence it is not necessarily a carbohydrate mimetic for each and every stereochemical sequence exhibited.

For example, the 2,5-imino pyrrolidine IS7 exhibits both D-gluco and L-gulo stereochemistry and can be considered as both a glucose and gulose mimetic:

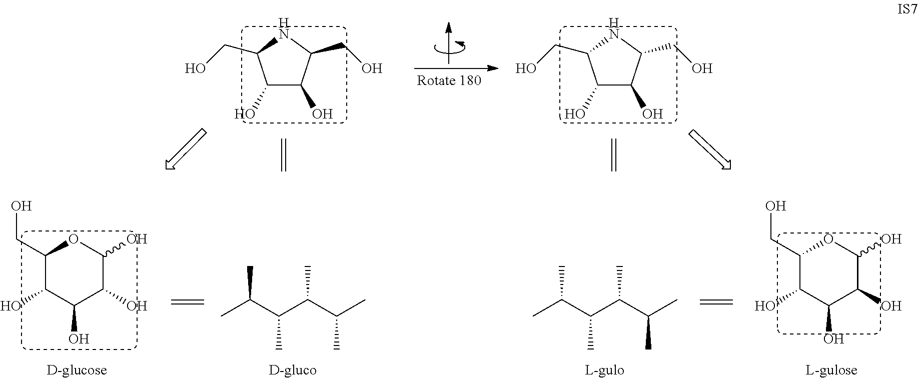

Note that an alternative, but chemically distinct isomer of IS7, not the 2,5-pyrrolidine but the 1,4-pyrrolidine IS8, also exhibits both D-gluco and L-gulo stereochemistries but is considered a D-glucose mimetic only. This is by virtue of the structural constraints enforced by the cyclic nature of IS8 leading to presentation of the structural motifs of D-glucose only. Note that in chemical terms IS7 and IS8 are distinct and cannot interconvert.

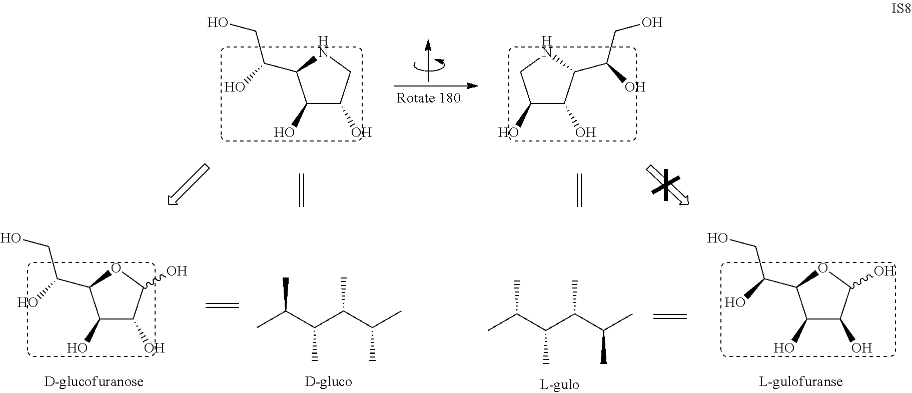

### (ii) Deoxysugar Mimetics and Further Substitution

Where an iminosugar mimics a deoxy sugar, this may also be considered as mimicry (albeit partial) of the cognate (fully oxygenated) monosaccharide. For example, the mimetic properties of iminosugar IS9 can be analysed as follows:

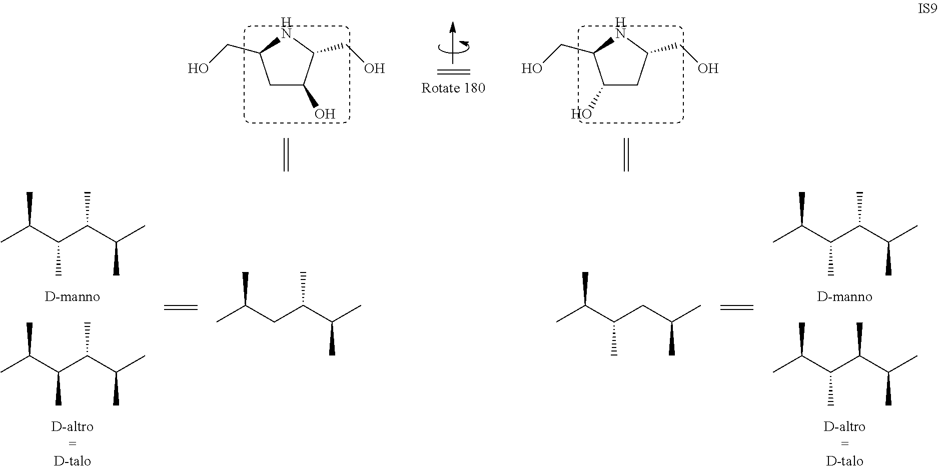

Moreover, replacement of hydroxyl groups with hydroxyl isosteres (e.g. similarly sized atoms or groups such as Me, Cl and F) may also generates iminosugars which are mimetics of a monosaccaride. For example, IS10 is a D-arabinofuranose mimetic, as shown below:

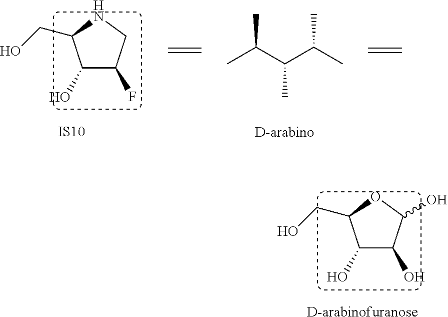

However, it should be noted that where the stereochemical configuration of the iminosugar matches one or more monosaccharides, but the group is not OH or an isostere (e.g. OBn, CO₂H or N₃) this would also be considered a mimetic for the purposes of the present invention. For example, the iminosugar IS11 is considered to be a mimetic of D-arabinofuranose, as shown below:

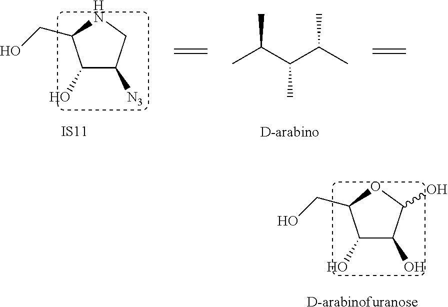

(iii) Quaternary Centres

Where these are present only the stereochemically defined groups on adjacent carbon atoms are considered when assigning matches, as shown below in the case of iminosugar IS12:

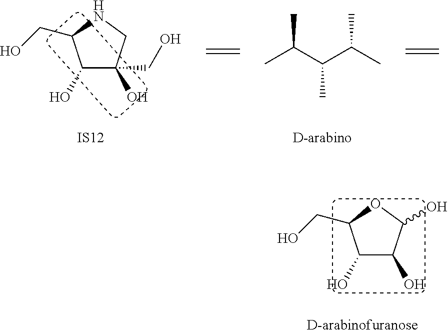

### (iv) Disaccharides and Oligosaccharides

Appropriately substituted iminosugars may also be considered as mimics of di- or oligosaccharides. In the case the same general principles described above are applied, with the caveat being that the iminosugar must contain two or more non-overlapping carbohydrate mimics.

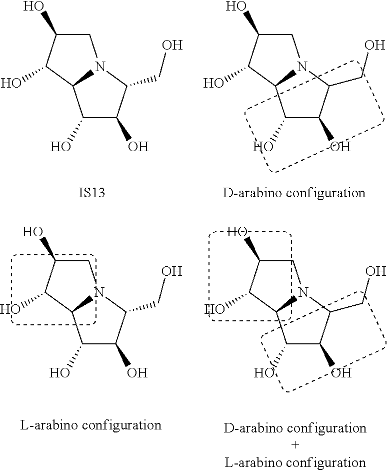

### (v) D- and L-Sugar Mimicry

Iminosugars may mimic either D- or L-forms of sugars. In the example below it can be seen that IS14 is a mimic of D-glucose, whereas its enantiomer IS15 is a mimic of L-glucose. This principle is generally applicable.

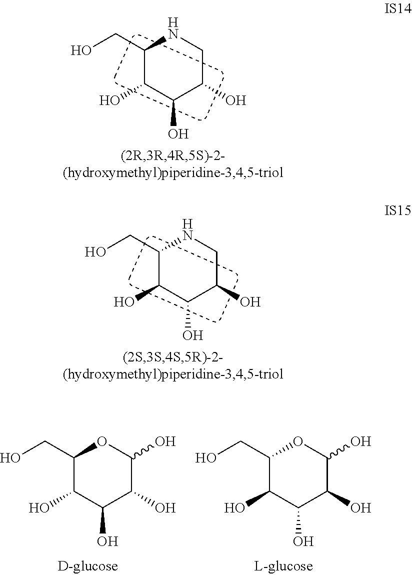

Thus, the iminosugars for use according to the invention may be of any structural class and/or subclass, including the classes and subclasses described above in Sections II(a) and II(b), and may be further characterized on the basis of the stereochemical configuration as follows:
Iminosugars of D- or L-gluco configuration;
Iminosugars of D- or L-galacto configuration;
Iminosugars of D- or L-manno configuration;
Iminosugars of D- or L-allo configuration;
Iminosugars of D- or L-altro configuration;
Iminosugars of D- or L-ido configuration;
Iminosugars of D- or L-gulo configuration;
Iminosugars of D- or L-talo configuration;
Iminosugars of D- or L-arabino configuration;
Iminosugars of D- or L-ribo configuration;
Iminosugars of D- or L-xylo configuration; and/or
Iminosugars of D- or L-lyxo configuration.

Alternatively, or in addition, the iminosugars for use according to the invention may be classified according to their stereochemical configuration in combination with other structural characteristics by reference to the sugars mimicked, as follows:
D- or L-glucose;
D- or L-galactose;
D- or L-mannose;
D- or L-allose;
D- or L-altrose;
D- or L-idose;
D- or L-gulose;
D- or L-talose;
D- or L-arabinose;
D- or L-ribose;
D- or L-deoxyribose;
D- or L-xylose;
D- or L-lyxose;
D- or L-psicose;
D- or L-fructose;
D- or L-sorbose;
D- or L-tagatose;
D- or L-ribulose;
D- or L-xylulose;
D- or L-fucose;
D- or L-fuculose;
D- or L-rhamnose;
D- or L-seduheptulose;
Sucrose;
Lactose;
Trehalose;
Maltose;
Acarbose;
Raffinose;
Melezitose;
Maltotriose;
Stachyose;
Glycogen;
Cellulose;
Chitin;
Starch;
Dextrin;
Glucan;
Glycosaminoglycans; and/or
Other oligosaccharides.

### B. Functional Considerations

The compounds for use according to the invention (including the compounds having the general formulae defined in section A(I) and the iminosugars described in section A(II), above) may have various functional properties. Any such functional properties may or may not contribute to the claimed in vivo activity, therapeutic activity or mode of action.

Thus, in some cases the compound for use according to the present invention may have one or more of the functional characteristics described below, wherein the functional characteristic(s) do not contribute to the claimed therapeutic activity and are purely incidental. In other cases, the compound for use according to the present invention may have one or more of the functional characteristics described below, wherein the functional characteristic(s) are responsible, wholly or partly, for the claimed therapeutic activity.

### (I) Glycosidase Ligands

The compounds for use according to the invention may act as a ligand for one or more enzyme(s) of the following glycosidase classes in vitro and/or in vivo:
α-glucosidases;
β-glucosidases;
α-galactosidases;
β-galactosidases;
α-mannosidases;
α-fucosidases; or
α-iduronidases; or
β-glucuronidases; or
β-mannosidases; or
hexosaminidases; or
α-N-acetylglucosaminidases; or
α-N-acetylgalactosaminidases; or
β-N-acetylglucosaminidases; or
β-N-acetylgalactosaminidases; or
sialidases; or
heparinases; or
neuraminidases; or
hyaluronidase; or
amylases; or
two or more of the foregoing enzyme classes.

The glycosidase ligands for use according to the invention may function as:
Inhibitors (competitive or non-competitive) of the target enzyme (e.g. by binding to the catalytic site of the enzyme);
Activators (e.g. by binding to an allosteric site of the enzyme);
Allosteric site ligands (e.g. acting as inhibitors or activators of enzyme activity);
Catalytic site ligands (e.g. acting as competitive inhibitor);
Pharmacoperones for the target enzyme, for example by binding to: (i) the catalytic site; (ii) an allosteric site; (iii), a site outside the catalytic site; and/or (d) a site outside an allosteric site (see also Section III, below); or
Two or more of the foregoing.

The compounds for use according to the invention preferably do not inhibit enzymes involved in metabolism of xenobiotics as this could lead to drug-drug interactions. Thus, the compounds of the invention preferably do not inhibit one or more of the following enzymes: CYP3A3/4 (most abundant isoenzyme in humans and responsible for metabolism of widest range of drugs), CYP1A, CYP2D6, CYP2C9/10 and CYP2C19.

The compounds for use according to the invention preferably do not inhibit digestive disaccharidases (unless such inhibition is desirable in order to, for example, modify sugar metabolism in the treatment of metabolic disorders).

### (II) Glycosyltransferase Ligands

The compounds for use according to the invention may act as a ligand for a glycosyltransferase. Such compounds may act as a ligand for any glycosyltransferase, but preferred are compounds which are ligands for one or more enzyme(s) of the following glycosyltransferase enzyme classes in vitro and/or in vivo:
Fucosyltransferase;
Chitin synthetase;
Ceramide glucosyltransferase;
β-1,4-galactosyltransferase;
α-1,3-galactosyltransferase;
arabinofuranosyl transferase;
galactofuranosyltransferase; or
two or more of the foregoing enzyme classes.

The glycosyltransferase ligands for use according to the invention may function as:
Inhibitors (competitive or non-competitive) of the target enzyme (e.g. by binding to the catalytic site of the enzyme);
Activators (e.g. by binding to an allosteric site of the enzyme);
Allosteric site ligands (e.g. acting as inhibitors or activators of enzyme activity);
Catalytic site ligands (e.g. acting as competitive inhibitor);
Pharmacoperones for the target enzyme, for example by binding to: (i) the catalytic site; (ii) an allosteric site; (iii), a site outside the catalytic site; and/or (d) a site outside an allosteric site (see also Section III, below); or
Two or more of the foregoing.

### (III) Other Enzyme Ligands

The compounds for use according to the invention may act as a ligand for one or more enzyme(s) of the following classes in vitro and/or in vivo:
Matrix metalloproteinases;
Nucleoside processing enzymes;
UDP Gal mutases;
Glycogen phosphorylases;
ATPases;
GTPases;
Kinases (e.g. protein kinases, for example selected from serine/threonine specific, tyrosine specific, receptor tyrosine, histidine specific, aspartic acid/glutamic acid specific and mixed protein kinase classes);
Phosphatases;
Enzymes involved in nucleic acid synthesis; and
Two or more of the foregoing.

The above enzyme ligands for use according to the invention may function as:
Inhibitors (competitive or non-competitive) of the target enzyme (e.g. by binding to the catalytic site of the enzyme);
Activators (e.g. by binding to an allosteric site of the enzyme);
Allosteric site ligands (e.g. acting as inhibitors or activators of enzyme activity);
Catalytic site ligands (e.g. acting as competitive inhibitor);
Pharmacoperones for the target enzyme, for example by binding to: (i) the catalytic site; (ii) an allosteric site; (iii), a site outside the catalytic site; and/or (d) a site outside an allosteric site (see also Section III, below); or
Two or more of the foregoing.

The compounds for use according to the invention may act as a ligand for one or more G-protein coupled receptor(s) in vitro and/or in vivo.

They may act as ligands for a carbohydrate binding site of any protein (including, for example, any of the lectins hereinbefore described).

### (IV) PRR Ligands

The innate immune response has evolved to recognize a few, highly conserved structures present in diverse groups of microorganisms. These highly conserve structures are known as pathogen-associated molecular patterns (PAMPs). They are recognized by a class of receptors known as pathogen-(or pattern)recognition receptors (PRRs), which are expressed on various effector cells of the innate immune system, including the professional antigen-presenting cells, macrophages and dendritic cells.

The best-studied class of PRR is the Toll-like receptor class (TLRs). Mammalian TLRs comprise at least 10 members, designated TLR1-10, and may be expressed as homodimers or heterodimers (TLR1 plus TLR2 or TLR6 plus TLR2). It seems that different classes of pathogen are recognized by different TLRs. For example, TLR4 appears to be responsible for the detection of Gram-negative bacteria, its cognate PAMP being lipopolysaccharide (LPS). TLR2 appears to have several ligands, including peptidoglycan of Gram-positive bacteria, lipoproteins from Mycobacterium tuberculosis, and certain components of Saccharomyces cerevisiae zymosan, as well as highly purified Porphyromonas gingivalis LPS. TLR3 recognizes dsRNA, while TLR5 binds flagellin and TLR6 cooperates with TLR2 in detecting a subset of bacterial peptidoglycan. TLR7 can be triggered by imidazoquinolines, as well as ssRNA, and may thus be involved in the detection of viral infection. TLR9 detects bacterial and viral DNA sequences containing unmethylated cytosine-guanosine dinucleotides (CpGs). Other members of the mammalian TLR family may be specific for PAMPs characteristic of other classes of pathogens such as fungi (mannan, glucan and mycobacteria (via lipoarabinomannan and/or muramyldipeptide as cognate PAMPs)).

Another major class of PRR are the C-type lectins (reviewed by Figdor et al. (2002) Nature Reviews Immunology 2: 77-84). These PRRs share a conserved domain (the carbohydrate recognition domain or CRD) which was first characterized in animal lectins and which appears to function as a calcium-dependent carbohydrate-recognition domain. This consists of about 110 to 130 residues and contains four cysteines which are involved in two disulfide bonds. This domain may be present in multiple copies in some C-type lectin PRRs (for example, the mannose receptor contains eight CRDs).

Examples of C-type lectins include DC-SIGN (Dendritic Cell Specific ICAM-3 Grabbing Nonintegrin, or CD209), which can signal in response to Mycobacterium tuberculosis, synergising with LPS to induce IL-10 production by monocyte-derived DCs. The mannose receptor (MR) is involved, in recognition of mycobacteria, fungi and protozoa. Dectin-1 acts as a PRR for β-glucan. Other C-type lectins are expressed in DCs (e.g. blood dendritic cell antigen-2 (BDCA-2), dendritic cell immunoactivating receptor (DCAR) and can also act as signalling receptors, though their role in PAMP recognition has yet to be established.

Preferred compounds for use according to the invention are PRR ligands (as defined herein). Such PRR ligands may be readily identified by screening assays which detect: (a) binding to a PRR (for example, TLR, C-type lectin or NOD-protein); and/or (b) the stimulation of PRR (for example, TLR, C-type lectin or NOD-protein) signalling. In the former case, the assays may involve competitive binding assays using an isolated PRR and a known cognate PAMP ligand as test reagents. Such competitive binding assays are routine in the art, and those skilled in the art will readily be able to identify appropriate conditions and formats for such assays. In the latter case, assays for PRR (for example C-type lectin) signalling activity may involve the use of PRR (for example C-type lectin)-bearing immune cells (typically DCs) as test reagent. Those skilled in the art will readily be able to identify appropriate conditions and formats for such assays, including inter alia the nature and number of the dendritic cells, the relative concentrations of compound and cells, the duration of stimulation with the compound and the methods used to detect signalling (for example by immunoassay for cytokine release).

The PRR ligands of the invention may bind any PRR, including any TLR, C-type lectin or NOD-protein. Preferably, the compounds for use according to the invention bind to PRRs displayed on/expressed by neutrophils, though they may bind to PRRs in, on or secreted by other cells including other cells of the innate immune system as well as to PRRs in, on or secreted by, for example, DCs, macrophages and/or T-cells.

### (a) NOD-Protein Ligands

The NOD-proteins (also known as the caterpillar family and NOD-LRR family) are cytosolic proteins that have a role in various innate and adaptive immune responses to cytosolic pathogens. Particularly preferred NOD-protein ligands for use according to the invention are NOD1 and/or NOD2 ligands. These latter proteins bind structures derived from peptidoglycan that are not TLR ligands.

NOD-protein PRRs comprise C-terminal leucine-rich repeats (LRRs), a central nucleotide-binding oligomerization domain (NOD), and N-terminal protein-protein interaction motifs, such as caspase recruitment domains (CARDs), pyrin domains or a TIR domain.

### (b) Toll-Like Receptor (TLR) Ligands

The PRR ligands of the invention may bind to any TLR receptor. Thus, the PRRs of the invention may bind to one or more of TLR1, TLR2, TLR3, TLR4, TLR5, TLR6, TLR7, TLR8, TLR9, TLR10 and TLR11.

Preferably, the TLR ligands for use according to the invention bind to:
(a) a TLR coupled with the MyD88 adaptor signalling pathway; and/or
(b) a TLR coupled with the TRIF adaptor signalling pathway; and/or
(c) a cell-surface TLR; and/or
(d) an endosomal TLR (e.g. TLR7, TLR8 and/or TLR9);
(e) an intracellular TLR (e.g. TLR3).

Particularly preferred are TLR9 or TLR4 ligands.

### (c) Lectin Ligands

As used herein, the term “lectin” defines a proteins which specifically binds (or crosslinks) a carbohydrate. Many lectins are multivalent carbohydrate-binding proteins or glycoproteins (excluding enzymes and antibodies). Preferred compounds for use according to the invention are ligands for C-type lectins. However, the compounds for use according to the invention may bind to any lectin, for example to any of the lectins described in Figdor et al. (2002) Nature Reviews Immunology 2: 77-84 (the disclosure of which relating to the identification of various lectins is incorporated herein by reference). Thus, the compounds of the invention may be ligands for type I and/or type II C-type lectins.

The compounds of the invention may be ligands for lectins selected from:
(a) MMR(CD206, macrophage mannose receptor); and/or
(b) DEC-205; and/or
(c) Dectin 1; and/or
(d) Dectin 2; and/or
(e) Langerin; and/or
(f) DC-SIGN; and/or
(g) BDCA-2; and/or
(h) DCIR; and/or
(i) DLEC; and/or
(j) CLEC; and/or
(k) a rhamnose-binding C-type lectin; and/or
(l) asialoglycoprotein receptor; and/or
(m) collectins; and/or
(n) selectins; and/or
(o) galectins; and/or
(p) annexins; and/or
(q) lecticans; and/or
(r) I-type lectins (for example, siglecs (sialic acid-binding immunoglobulin superfamily lectins); and/or
(s) P-type lectins.

The PRR or lectin (for example C-type lectin) ligands (as defined herein) may be identified by assays for PRR/lectin (for example C-type lectin) binding. These may involve competitive binding assays using an isolated PRR/lectin (for example C-type lectin) and a known cognate PAMP ligand as test reagents. Such competitive binding assays are routine in the art, and those skilled in the art will readily be able to identify appropriate conditions and formats for such assays.

### (d) Other Ligands

The compounds of the invention may be ligands for chaperone proteins. For example, the compounds of the invention may be ligands for calnexin and/or calreticulin.

### (V) Pharmacoperones

### Proteostasis Regulators

The compounds of the invention may be proteostasis regulators, as herein defined.

In embodiments where the compounds of the invention are proteostasis regulators, then they may be proteostasis regulators of any functional class. For example, the compound of the invention may be a: (a) pharmacoperone; (b) UPR signalling regulator; (c) HSR regulator; (d) proteasome inhibitor; (e) upregulator of macromolecular chaperone(s) in the ER; (f) trasncriptional and/or translational upregulators; and/or (g) a calcium homeostasis regulator.

In embodiments where the compounds of the invention are UPR signalling regulators, the compounds may regulate one or more of: (a) the IRE1 arm of the UPR; (b) the ATF6 arm of the UPR; and/or (c) the PERK arm of the UPR.

### (a) Pharmacoperones

It has recently been discovered that certain small molecules can serve as a molecular scaffolding and cause otherwise-misfolded mutant proteins to fold and route correctly within the cell. Such molecules have been dubbed “chemical chaperones”, “pharmaceutical chaperones” or “pharmacoperones”.

The term pharmacoperone is a term of art (from “pharmacological chaperone”) used to define a class of biologically active small molecules (sometimes also referred to in the art as “chemical chaperones”) that serve as molecular scaffolds, causing otherwise misfolded mutant proteins to fold and route correctly within the cell.

The compounds of the invention may be pharmacoperones as defined above.

In particular, it has been recognised that certain iminosugars can act as competitive inhibitors of the mutant enzymes implicated in various lysosomal storage disorder's can, at subinhibitory concentrations, act as “Active-Site-Specific Chaperones” or ASSCs by either inducing or stabilizing the proper conformation of the mutant enzyme by specific binding to the catalytic site (see Fan (2007) Iminosugars as active-site-specific chaperones for the treatment of lysosomal storage disorders, in Iminosugars From Synthesis to Therapeutic Applications: Compain, Philippe/Martin, Olivier R. (eds.) ISBN-13: 978-0-470-03391-3-John Wiley & Sons; pages 225-247). Thus, the compounds for use according to the invention may be ASSCs as defined above.

In other embodiments, the compounds or iminosugars of the invention are pharmacoperones of an enzyme which do not bind to a catalytic site of said enzyme.

Thus, the pharmacoperones of the invention need not be a competitive inhibitor of said enzyme, so removing the problems associated with chaperone:inhibitor ratios associated with known pharmacoperones.

In preferred embodiments, the pharmacoperone for use according to the invention is an activator of said enzyme. In such embodiments, the pharmacoperone may specifically bind an activating allosteric site on the enzyme.

In other embodiments, the pharmacoperone may be a non-competitive inhibitor of said enzyme. In such embodiments, the chaperone:inhibitor ratio may be favourable in view of the availablity of the catalytic site. In such embodiments the pharmacoperone may specifically bind an inhibiting allosteric site on the enzyme.

In yet other embodiments, the pharmacoperone of the invention does not bind to the enzyme at all, but acts as an indirect chaperone via a chaperone effect attendant on binding to a protein (e.g. enzyme) which itself acts as a chaperone or co-chaperone of the enzyme. For example, the pharmacoperones of the invention may bind to chaperone proteins such as calnexin and calreticulin and so influence protein trafficking through the Golgi apparatus.

### (VI) Immunomodulators

### (a) General Considerations

The compounds of the invention may be immunomodulatory. The term immunomodulatory is used in this context in relation to the compounds for use according to the invention to define a compound (e.g. a compound as described in section A(I) above or an iminosugar as described in Section A(II), above) which can stimulate and/or suppress one or more components or activities of the immune system (e.g. the mammalian immune system) in vivo or in vitro. Preferred immunomodulatory compounds for use according to the invention are capable of stimulating the activity of one or more cytokine(s) in a PRR-bearing cell. Such alkaloids are said to exhibit a cytokine stimulation profile in that PRR-bearing cell. Typically, the immunomodulatory alkaloids of the invention are capable of stimulating the activity of one or more cytokines in macrophages and/or dendritic cells. This stimulatory activity may be observable in vitro and/or in vivo. The stimulation may occur directly or indirectly via any mechanism and at any level (e.g. at the level of transcription, translation, post-translational modification, secretion, activation, shedding, stabilization or sequestration). Typically, the stimulation comprises an increase in the production of the cytokine(s) by the PRR-bearing cell. Typically, the one or more cytokine(s) stimulated by the immunomodulatory alkaloids for use according to the invention comprise one or more Th1 cytokines (as herein defined and described). Particularly preferred are immunomodulatory alkaloids that stimulate IL-2 and/or IL-12 in dendritic cells and/or macrophages (in vivo and/or in vitro).

Immunomodulatory compounds for use according to the invention may be readily identified by screening assays designed to detect the induction of one or more cytokine(s) (for example, IL-12 production in dendritic cells) in vitro. Such assays conveniently involve immune assays or microarray analysis (the latter being especially useful in embodiments where immunomodulatory compounds which stimulate a large number of different cytokines or which differentially stimulate a specific subclass of cytokines (e.g. Th1 cytokines) are to be selected). Those skilled in the art will readily be able to identify appropriate conditions for such assays, including inter alia the nature, source and number of the PRR-bearing cell (e.g. macrophages or dendritic cells), the relative concentrations of compound and cells, the duration of stimulation with the compound and the methods used to detect the induction of the cytokine(s).

Immunomodulatory activity may be determined by in vitro cytokine release assays (for example using one or more immune cells, e.g. macrophage, dendritic or spleen cells). Preferred immunomodulatory compounds of the invention stimulate the release of one or more cytokines (e.g. IL-12) in vitro (for example, in spleen cells, macrophages and/or dendritic cells). They may act as PRR ligands, a term used herein in relation to certain preferred compounds for use according to the invention to define compounds which can act as binding partners for a PRR. Such immunomodulatory compounds therefore include those which bind (or directly physically interact) with a PRR in vivo irrespective of the physiological consequences of that binding. Thus, the PRR ligands of the invention may bind a PRR as part of a cellular signalling cascade in which the PRR forms a part. Alternatively, they may bind PRR in the context of some other aspect of cellular physiology. In the latter case, the ligands may for example bind PRR at the cell surface without triggering a signalling cascade, in which case the binding may affect other aspects of cell function. Thus, the ligands of the invention may bind PRRs and thereby effect an increase in the concentration of functional PRR at the cell surface (for example mediated via an increase in PRR stability, absolute receptor numbers and/or PRR activity). Alternatively, the ligands may bind PRR (or PRR precursors) intracellularly, in which case they may act as molecular chaperones to increase the expression of active PRR.

### (b) PRR Agonists

In preferred embodiments, the PRR ligands of the invention are PRR agonists. The term agonist is used herein in relation to the PRR ligands of the invention to define a subclass of ligands which productively bind PRR to trigger the cellular signalling cascade of which the PRR forms a part.

As used herein, the term PRR-bearing cell defines any cell which expresses one or more pathogen-(or pattern) recognition receptors (PRRs). The term PRR is a term of art used to define a class of receptors which are expressed on various cells (e.g. epithelial cells and effector cells of the innate immune system, including the professional antigen-presenting cells, macrophages and dendritic cells) and which recognize a few, highly conserved structures present in diverse groups of microorganisms known as pathogen-associated molecular patterns (PAMPs). Thus, PRR-bearing cells as described herein may comprise epithelial cells, macrophages, neutrophils, dendritic cells or other effector cells of the innate immune system. In preferred embodiments, the PRR-bearing cell for use in relation to the invention are dendritic cells or macrophages. Thus, those functional attributes of the immunomodulatory compounds of the invention that are defined by reference to inter alia a PRR-bearing cell are to be understood to relate to any of a wide variety of different PRR-bearing cells of diverse cytological properties and biological functions, including inter alia epithelial cells, dendritic cells, macrophages, various APCs, natural killer (NK) cells and other cells of the innate immune system (including e.g. neutrophils, granulocytes and monocytes). Preferably, however, the PRR-bearing cells described herein (and used for example to define a parameter of the reference conditions under which the functional properties of the immunomodulatory compound are manifest) are macrophages or dendritic cells.

The term cytokine stimulatory is used herein to define a subclass of immunomodulatory compounds for use according to the invention which are capable of stimulating the activity of one or more cytokine(s) in a PRR-bearing cell. Such compounds are said to exhibit a cytokine stimulation profile in that PRR-bearing cell. Typically, the immunomodulatory compounds of the invention are capable of stimulating the activity of one or more cytokines in macrophages and/or dendritic cells. This stimulatory activity may be observable in vitro and/or in vivo. The stimulation may occur directly or indirectly via any mechanism and at any level (e.g. at the level of transcription, translation, post-translational modification, secretion, activation, shedding, stabilization or sequestration). Preferred cytokine stimulatory compounds for use according to the invention are PRR ligands (as herein defined). Typically, the stimulation comprises an increase in the production of the cytokine(s) by the PRR-bearing cell. Typically, the one or more cytokine(s) stimulated by the immunomodulatory compounds for use according to the invention comprise one or more Th1 cytokines (as herein defined and described). Particularly preferred are immunomodulatory compounds that stimulate IL-2 and/or IL-12 in dendritic cells and/or macrophages (in vivo and/or in vitro).

Some iminosugars have immunomodulatory activity that is independent of any glycosidase inhibitory activity. Examples of such compounds are described, for example, in WO2004/064715, WO2005/070415 and WO2005/070418. It is thought that this immunomodulatory activity may arise from the stimulation of secretion of various cytokines (e.g. IL-12 and/or IL-2) by immune cells (e.g. dendritic cells and/or macrophages). As described in WO2004/064715, WO2005/070415 and WO2005/070418 (the content of which relating to the structure of the various compounds described and their biological activity is hereby incorporated herein by reference), the immunomodulatory activity of such compounds can itself confer antiviral activity.

### (c) Cytokine Stimulation

The compounds for use according to the invention may be cytokine stimulatory compounds capable of stimulating the activity of one or more cytokine(s) in a PRR-bearing cell. In preferred embodiments, the compound may stimulate one or more Th1 cytokine(s) in a PRR-bearing cell, for example IL-12 and/or IL-2.

IL-2 is a Th1 cytokine involved in mediating type-1 responses. It appears to be involved not only in T cell activation but also in the activation of inter alia NK cells, so functioning to regulate and link innate and adaptive immunity. Thus, the induced expression of IL-2 by the compounds for use according to the invention may directly potentiate a Th1 response and so increase the Th1:Th2 response ratio. The induced expression of IL-2 may also indirectly potentiate a Th1 response (and so increase the Th1:Th2 response ratio) by stimulating the activity of endogenous dendritic cells, which cells then trigger responses by other classes of lymphocytes (CTL, B, NK, and NKT cells) and also elicit T cell memory (a critical goal of vaccination).

The induced expression of IL-2 may also indirectly potentiate a Th1 response (and so increase the Th1:Th2 response ratio) by stimulating the activity of endogenous dendritic cells, which cells then trigger responses by other classes of lymphocytes (CTL, B, NK, and NKT cells) and also elicit T cell memory (a critical goal of vaccination).

The compounds for use according to the invention may stimulate the expression of IL-12 in PRR-bearing, cells (for example in dendritic cells and/or macrophages). IL-12 is the primary mediator of type-1 immunity (the Th1 response). It induces natural killer (NK) cells to produce IFN-γ as part of the innate immune response and promotes the expansion of CD4⁺ Th1 cells and cytotoxic CD8⁺ cells which produce IFN-γ. It therefore increases T-cell invasion of tumours as well as the susceptibility of tumour cells to T-cell invasion.

Thus, without wishing to be bound by any theory, the immunomodulatory activity of certain preferred compounds for use according to the invention may arise from the stimulation of one or more cytokines (for example one or more Th1 cytokines, e.g. IL-12 and/or IL-2) in PRR-bearing cells (e.g. neutrophils, macrophages or dendritic cells). This leads to the stimulation of NK cells to produce IFN-γ and induces the development of CD4⁺ Th1 cells. The induced Th1 cells then produce IFN-γ and IL-2. The stimulated cytokine(s) (e.g. IL-12 and/or IL-2) then enhances further proliferation of Th1 cells and the differentiation of pathogen (e.g. tumour and virus)—specific CD8⁺ T cells. The cytokine(s) also stimulate the cytolytic activity of NK cells of the innate immune system.

The term cytokine stimulation profile is used herein to define a functional attribute of certain immunomodulatory compounds for use according to the invention which is characterized by reference to the identity of one or more cytokines stimulated (and optionally the identity of one or more cytokines unstimulated) in a PRR-bearing cell when contacted with the relevant immunomodulatory compound. Preferably, the cytokine stimulation profile is characterized by reference to the presence or absence of stimulation of two or more cytokines, more preferably four or more. Even more preferably, the cytokine stimulation profile is characterized by reference to the presence or absence of stimulation of one or more Th1 cytokines and/or one or more Th2 cytokines. Alternatively, or in addition, the stimulation profiles which functionally define the immunomodulatory compounds may be characterized by the degree of stimulation of one or more reference cytokine(s) (or classes thereof). The degree of stimulation may be expressed as an induction ratio with respect to: (a) the levels of the reference cytokine(s) (or markers thereof, such as encoding nucleic acids) in the PRR-bearing cell in the absence of the relevant test immunomodulatory compound; and/or (b) the level of one or more other cytokine(s) (or classes thereof) also present in the PRR-bearing cell (whether stimulated or not by the immunomodulatory compound). The cytokine stimulation profile of the immunomodulatory compounds for use according to the invention is preferably characterized by the stimulation of one or more Th1 cytokines (and optionally the absence of stimulation of one or more Th2 cytokines).

The term Th1 cytokine (or Type-1 cytokine) is a term of art used to define those cytokines produced by Th1 T-helper cells. Th1 cytokines include, for example, IL2, IFN-γ, IFN-α/β, IL12, IL-18, IL-27 and TNF-β. The term Th2 cytokine (or Type-2 cytokine) is a term of art used to define those cytokines produced by Th2 T-helper cells. Th2 cytokines include, for example, IL-4, IL-5, IL-9, IL-13, IL-25 and TSLP. The term Treg cytokine is a term of art used to define those cytokines produced by regulatory T-cells. Treg cytokines include, for example, IL-10, TGF-β and TSP1.

Immunomodulatory compounds for use according to the invention are preferably cytokine stimulatory compounds capable of stimulating the activity of one or more cytokine(s) in a PRR-bearing cell. In preferred embodiments, the compound may stimulate one or more Th1 cytokine(s) in a PRR-bearing cell, for example IL-12 and/or IL-2.

Immunomodulatory compounds for use according to the invention may also be able to reduce the overproduction of Th 1 cytokines such as IFN-γ via regulating production of IL-2 or IL-12 directly or by stimulating production of Th 2 cytokines such as IL-4. The compounds of the invention may also affect the production of glucosylated cytokines such as IFN-γ such that any overproduction is reduced or IFN-γ produced becomes less active or inactive as proposed for deoxynojirimycin and N-methyl-deoxynojirimycin in isolated splenocyte studies by Kosuge et al. (2000) Biol. Pharm. Bull. 23 (1): 1-5. Therapeutic improvements to iminosugars for therapeutic applications involving reduction of overproduction of IFN-γ would be increased glycosidase specificity to avoid inhibition of off-target glucosidases caused by DNJ and N-methyl-DNJ.

### (VII) Functional Sugar Mimicry

### (a) General Considerations

As described in Section A(II)(c) (above), the iminosugars for use according to the invention may be structural sugar mimetics and in many cases this structural mimicry is reflected in shared functional properties. Such functional sugar mimetics, as defined above, are compounds which share some or all of the functional properties of the sugar mimicked. For example, functional sugar mimetics may share some of the binding properties of the sugar mimicked in vivo (without necessarily sharing all of the attendant functional properties thereof).

Certain sugar mimetics may be identified by assays for saccharase inhibitory activity. Such enzyme assays are routine in the art, and those skilled in the art will readily be able to identify appropriate conditions and formats for such assays. For example, many polyhydroxylated iminosugars are potent and highly selective glycosidase inhibitors. These compounds can mimic the number, position and configuration of hydroxyl groups present in pyranosyl or furanosyl moieties and so bind to the active site of a cognate glycosidase, thereby inhibiting it. This area is reviewed in Legler (1990) Adv. Carbohydr. Chem. Biochem. 48: 319-384 and in Asano et al. (1995) J. Med. Chem. 38: 2349-2356.

In yet other embodiments, the functional sugar mimetic binds to a sugar receptor PRR. Such binding per se need not necessarily trigger a sugar receptor-mediated signalling pathway (i.e. initiate the cellular signalling cascade in which the sugar receptor forms a part): other co-stimulatory events may be required. Moreover, the binding may occur in the context of some other aspect of cellular physiology. In the latter case, the compounds of the invention may act as ligands as hereinbefore defined and may for example bind a sugar receptor at the cell surface without triggering a signalling cascade, in which case the binding may affect other aspects of cell function. Thus, the functional sugar mimetics of the invention may bind to a sugar receptor and thereby effect an increase in the concentration of functional sugar receptor at the cell surface (for example mediated via an increase in receptor stability, absolute receptor numbers and/or receptor activity). Alternatively, the function sugar mimetics may bind a sugar receptors (or a sugar receptor precursor) intracellularly, in which case they may act as molecular chaperones to increase the expression of active PRR.

### (b) Glucose Mimetics

The compounds for use according to the invention may be glucose mimetics. Such compounds may share some or all of the binding properties of glucose in vivo (without necessarily sharing all of the attendant functional properties thereof).

Such glucose mimetics may be identified by assays for glucosidase inhibitory activity. Such enzyme assays are routine in the art, and those skilled in the art will readily be able to identify appropriate conditions and formats for such assays.

Examples of such compounds are described in e.g. WO9929321 (the disclosure of which relating to specific piperidine iminosugars and their structure is hereby incorporated by reference). An example of such a glucose mimetic the iminosugar designated 1,5-dideoxy-1,5-imino-D-glucitol (alternately designated deoxynojirimycin), hereinafter “DNJ.” Numerous DNJ derivatives have been described. DNJ and its alkyl derivatives are potent inhibitors of the N-linked oligosaccharide processing enzymes, alpha-glucosidase I and alpha-glucosidase II (Saunier et al. (1982) J Biol Chem 257:14155-14161; Elbein (1987) Ann Rev Biochem 56:497534). These glucosidases are associated with the endoplasmic reticulum of mammalian cells. The N-butyl and N-nonyl derivatives of DNJ may also inhibit glucosyltransferases associated with the Golgi.

(c) Mannose and/or Rhamnose Mimetics

For example, the compounds of the invention may be mannose and/or rhamnose mimetics. Such compounds may share some or all of the binding properties of mannose and/or rhamnose in vivo (without necessarily sharing all of the attendant functional properties thereof).

Such sugar mimetics may be identified by assays for mannosidase and/or rhamnosidase inhibitory activity. Such enzyme assays are routine in the art, and those skilled in the art will readily be able to identify appropriate conditions and formats for such assays.

Thus, preferred rhamnose mimetics for use according to the invention are iminosugars which exhibit inhibitory activity against one or more rhamnosidase enzyme(s). Similarly, preferred mannose mimetics for use according to the invention are iminosugars which exhibit inhibitory activity against one or more mannosidase enzyme(s).

In yet other embodiments, preferred iminosugars may be rhamnose mimetics which bind to the rhamnose receptor PRR (see Grillon, Monsigny and Kieda (1990) Glycobiology 1(1): 33-8). Such binding per se need not necessarily trigger the rhamnose receptor-mediated signalling pathway (i.e. initiate the cellular signalling cascade in which the rhamnose receptor forms a part): other co-stimulatory events may be required. Moreover, the binding may occur in the context of some other aspect of cellular physiology. In the latter case, the iminosugars may act as ligands as hereinbefore defined and may for example bind rhamnose receptor at the cell surface without triggering a signalling cascade, in which case the binding may affect other aspects of cell function. Thus, the rhamnose mimetics of the invention may bind to the rhamnose receptor and thereby effect an increase in the concentration of functional rhamnose receptor at the cell surface (for example mediated via an increase in receptor stability, absolute receptor numbers and/or receptor activity). Alternatively, the rhamnose mimetics may bind rhamnose receptors (or rhamnose receptor precursors) intracellularly, in which case they may act as molecular chaperones to increase the expression of active PRR.

Similarly, other preferred iminosugars may be mannose mimetics which bind to the mannose receptor PRR. Again, such binding per se need not necessarily trigger the mannose receptor-mediated signalling pathway (i.e. initiate the cellular signalling cascade in which the mannose receptor forms a part): other co-stimulatory events may be required. Moreover, the binding may occur in the context of some other aspect of cellular physiology. In the latter case, the iminosugars may act as ligands as hereinbefore defined and may for example bind mannose receptor at the cell surface without triggering a signalling cascade, in which case the binding may effect other aspects of cell function. Thus, the mannose mimetics of the invention may bind to the mannose receptor and thereby effect an increase in the concentration of functional mannose receptor at the cell surface (for example mediated via an increase in receptor stability, absolute receptor numbers and/or receptor activity). Alternatively, the mannose mimetics may bind mannose receptors (or mannose receptor precursors) intracellularly, in which case they may act as molecular chaperones to increase the expression of active PRR.

### C. General Physicochemical Considerations

The compounds for use according to the invention (including the compounds having the general formulae defined in section A(I) and the iminosugars described in section A(II), above) may have various physicochemical properties.

The compounds for use according to the invention are preferably crystalline materials. Also preferred are compounds which are water soluble, or which are soluble in pharmaceutically acceptable excipients and formulations used in oral or i.v. administration (e.g. those described below). Also preferred are compounds which are subject to efficient passive or active transport to the desired site of action in vivo.

Preferred are iminosugars having a small molecular weight, since these may exhibit desirable pharmacokinetics. Thus, the iminosugar may have a molecular weight of 100 to 400 Daltons, preferably 150 to 300 Daltons and most preferably 200 to 250 Daltons.

Also preferred are non-metabolizable iminosugars. Such sugars may exhibit extended tissue residence durations, and so exhibit favourable pharmacokinetics.

### (VIII) Other Functional Attributes

Without wishing to be bound by any theory, the activity of the compounds of the invention may derive, at least in part, from the induction of feedback mechanisms produced by inhibitory activity leading to increased production of glycosidases. For example, the introduction of the compounds of the invention into cell organelles other than the lysosome may induce a feedback mechanism that leads to more enzyme being produced/released. Such a mechanism may involve direct (or more probably indirect) interaction of the compounds of the invention with calnexin and/or calreticulin

### D. Specific Examples

Particular examples of compounds suitable for use according to the invention are listed in Table 1 (below). References to particular compound numbers herein refer to the numbers in this list.

| | | | Stereochemistry | | | | | | | | | | | |
|---|---|---|---|---|---|---|---|---|---|---|---|---|---|---|
| Compound # | Chemical Name | Compound Class | Allose | Altrose | Arabinose | Galactose | Glucose | Gulose | Idose | Lyxose | Mannose | Ribose | Talose | Xylose |
| 1 | (1R,2R,3S,6S,7R,7aS)- | pyrrolizidine | | y | | | y | y | y | | | | y | |
| | 3- | | | | | | | | | | | | | |
| | (hydroxymethyl)hexahydro- | | | | | | | | | | | | | |
| | 1H-pyrrolizine- | | | | | | | | | | | | | |
| | 1,2,6,7-tetraol | | | | | | | | | | | | | |
| 2 | (2R,3R,4R)-2- | pyrrolidine | | | y | | | | | y | | | | |
| | (hydroxymethyl)pyrrolidine- | | | | | | | | | | | | | |
| | 3,4-diol | | | | | | | | | | | | | |
| 3 | (2R,3R,4R,5S)-2- | piperidine | | | | | y | y | | | | | | |
| | (hydroxymethyl)-1- | | | | | | | | | | | | | |
| | methylpiperidine-3,4,5- | | | | | | | | | | | | | |
| | triol | | | | | | | | | | | | | |
| 4 | (3R,4R)-4-hydroxy-1,1- | pyrrolidine | | | | | | | | | | | | |
| | dimethylpyrrolidinium-3- | | | | | | | | | | | | | |
| | carboxylate | | | | | | | | | | | | | |
| 5 | (2R,3S,4S)-4-hydroxy- | pyrrolidine | | | | | | | | | | | | y |
| | 2-(4- | | | | | | | | | | | | | |
| | methoxybenzyl)pyrrolidin- | | | | | | | | | | | | | |
| | 3-yl acetate | | | | | | | | | | | | | |
| 6 | (2S,4R)-4-hydroxy-1,1- | pyrrolidine | | | | | | | | | | y | | y |
| | dimethylpyrrolidinium-2- | | | | | | | | | | | | | |
| | carboxylate | | | | | | | | | | | | | |
| 7 | (2S,3R,4R,5S)-3,4,5- | piperidine | | | | | y | y | | | | | | |
| | trihydroxypiperidine-2- | | | | | | | | | | | | | |
| | carboxylic acid | | | | | | | | | | | | | |
| 8 | (1R,5S,8R)-1,8- | other | | | y | | | | | y | | | | |
| | dihydroxy-6-oxa-3- | | | | | | | | | | | | | |
| | azabicyclo[3.2.1]octan- | | | | | | | | | | | | | |
| | 2-one | | | | | | | | | | | | | |
| 9 | (3R,4R,5S)-3- | piperidine | | | y | | | | | y | | | | |
| | (hydroxymethyl)piperidine- | | | | | | | | | | | | | |
| | 3,4,5-triol | | | | | | | | | | | | | |
| 10 | (1S,2R,3S,4R,5S)-8- | nortropane | | | | | | | | | | | | y |
| | methyl-8- | | | | | | | | | | | | | |
| | azabicyclo[3.2.1]octane- | | | | | | | | | | | | | |
| | 1,2,3,4-tetraol | | | | | | | | | | | | | |
| 11 | (2R,3R,4R,5R)-2-((R)- | pyrrolidine | | | | | | | | | y | | | |
| | 1,2-dihydroxyethyl)-5- | | | | | | | | | | | | | |
| | (hydroxymethyl)pyrrolidine- | | | | | | | | | | | | | |
| | 3,4-diol | | | | | | | | | | | | | |
| 12 | (1S,7S,8S,8aR)- | indolizidine | | | | y | | | | | y | | | |
| | octahydroindolizine- | | | | | | | | | | | | | |
| | 1,2,7,8-tetraol | | | | | | | | | | | | | |
| 13 | (2R,3R,4R,5R)-2,5- | pyrrolidine | | | | | | | | | y | | | |
| | bis(hydroxymethyl)pyrrolidine- | | | | | | | | | | | | | |
| | 3,4-diol | | | | | | | | | | | | | |
| 14 | (1R,2R,3R,5R,7aR)-3- | pyrrolizidine | | | | | | | | | y | | | |
| | (hydroxymethyl)-5- | | | | | | | | | | | | | |
| | methylhexahydro-1H- | | | | | | | | | | | | | |
| | pyrrolizine-1,2-diol | | | | | | | | | | | | | |
| 15 | (2R,3S,4R,5R,6R)-2,6- | piperidine | | | | | y | y | y | | | | | |
| | bis(hydroxymethyl)piperidine- | | | | | | | | | | | | | |
| | 3,4,5-triol | | | | | | | | | | | | | |
| 16 | (2R,3R,4S,5S,6R)-2- | piperidine | | | | | y | y | y | | | | | |
| | (hydroxymethyl)-6- | | | | | | | | | | | | | |
| | (((2R,3R,4S,5S,6R)- | | | | | | | | | | | | | |
| | 3,4,5-trihydroxy-6- | | | | | | | | | | | | | |
| | (hydroxymethyl)tetrahydro- | | | | | | | | | | | | | |
| | 2H-pyran-2- | | | | | | | | | | | | | |
| | yloxy)methyl)piperidine- | | | | | | | | | | | | | |
| | 3,4,5-triol | | | | | | | | | | | | | |
| 17 | (1R,2R,3R,7S,7aS)-3- | pyrrolizidine | | | | | y | y | | | | | | |
| | (hydroxymethyl)hexahydro- | | | | | | | | | | | | | |
| | 1H-pyrrolizine-1,2,7- | | | | | | | | | | | | | |
| | triol | | | | | | | | | | | | | |
| 18 | (3aR,3a1R,4R,5S,8aS)- | pyrrolizidine | | | | y | y | y | | | | | | |
| | 5-(hydroxymethyl)-2,2- | | | | | | | | | | | | | |
| | dimethylhexahydro- | | | | | | | | | | | | | |
| | 3aH-[1,3]dioxino[4,5,6- | | | | | | | | | | | | | |
| | gh]pyrrolizin-4-ol | | | | | | | | | | | | | |
| 19 | Ioline | pyrrolizidine | | | y | | | | | y | | | | |
| 20 | (1R,2S,3R,5R)-8- | nortropane | | | y | | | | | y | | | | |
| | azabicyclo[3.2.1]octane- | | | | | | | | | | | | | |
| | 1,2,3-triol | | | | | | | | | | | | | |
| 21 | (1R,2S,3R,4S,5R)-8- | nortropane | | | | | | | | | | | | y |
| | azabicyclo[3.2.1]octane- | | | | | | | | | | | | | |
| | 1,2,3,4-tetraol | | | | | | | | | | | | | |
| 22 | (1R,2R,3R,5S,7S,7aR)- | pyrrolizidine | | | | y | | | | | y | | | |
| | 3-(hydroxymethyl)-5- | | | | | | | | | | | | | |
| | methylhexahydro-1H- | | | | | | | | | | | | | |
| | pyrrolizine-1,2,7-triol | | | | | | | | | | | | | |
| 23 | (2S,3R,4S,5S,6S)-2- | piperidine | | | | | y | y | y | | | | | |
| | ethyl-6- | | | | | | | | | | | | | |
| | (hydroxymethyl)piperidine- | | | | | | | | | | | | | |
| | 3,4,5-triol | | | | | | | | | | | | | |
| 24 | (1S,2R,3R,5R,6S,7R,7aR)- | pyrrolizidine | | y | | y | y | y | | | | | y | |
| | 3-(hydroxymethyl)-5- | | | | | | | | | | | | | |
| | methylhexahydro-1H- | | | | | | | | | | | | | |
| | pyrrolizine-1,2,6,7- | | | | | | | | | | | | | |
| | tetraol | | | | | | | | | | | | | |
| 25 | (2R,3R,4R,5R)-1-(2- | pyrrolidine | | | | | | | | | y | | | |
| | hydroxyethyl)-2,5- | | | | | | | | | | | | | |
| | bis(hydroxymethyl)pyrrolidine- | | | | | | | | | | | | | |
| | 3,4-diol | | | | | | | | | | | | | |
| 26 | (2R,3R,4R,5R)-2-(3- | pyrrolidine | | | | | | | | | y | | | |
| | hydroxy-4- | | | | | | | | | | | | | |
| | methoxyphenyl)-5- | | | | | | | | | | | | | |
| | (hydroxymethyl)pyrrolidine- | | | | | | | | | | | | | |
| | 3,4-diol | | | | | | | | | | | | | |
| 27 | (2R,3R,4R,5R)-2- | pyrrolidine | | | | | | | | | y | | | |
| | (hydroxymethyl)-5-(4- | | | | | | | | | | | | | |
| | hydroxyphenyl)pyrrolidine- | | | | | | | | | | | | | |
| | 3,4-diol | | | | | | | | | | | | | |
| 28 | (1R,2R,3R,6S,7S,7aS)- | pyrrolizidine | | y | | | | | | | y | | y | |
| | 3-(hydroxymethyl)-6- | | | | | | | | | | | | | |
| | (3,4,5-trihydroxy-6- | | | | | | | | | | | | | |
| | (hydroxymethyl)tetrahydro- | | | | | | | | | | | | | |
| | 2H-pyran-2- | | | | | | | | | | | | | |
| | yloxy)hexahydro-1H- | | | | | | | | | | | | | |
| | pyrrolizine-1,2,7-triol | | | | | | | | | | | | | |
| 29 | (2S,3S,4R)-1-(2- | pyrrolidine | | | | | | | | | | y | | |
| | hydroxyethyl)-2- | | | | | | | | | | | | | |
| | (hydroxymethyl)pyrrolidine- | | | | | | | | | | | | | |
| | 3,4-diol | | | | | | | | | | | | | |
| 30 | (1R,2S,6R,7R,8R,8aR)- | indolizidine | | | | | | | | | y | | | |
| | octahydroindolizine- | | | | | | | | | | | | | |
| | 1,2,6,7,8-pentaol | | | | | | | | | | | | | |
| 31 | (1R,2R,3R,7aR)-3- | pyrrolizidine | | | | | | | | | y | | | |
| | (hydroxymethyl)-5- | | | | | | | | | | | | | |
| | (3,10,11- | | | | | | | | | | | | | |
| | trihydroxyundecyl)hexahydro- | | | | | | | | | | | | | |
| | 1H-pyrrolizine- | | | | | | | | | | | | | |
| | 1,2,6-triol | | | | | | | | | | | | | |
| 32 | (1S,6S,7R,8R,8aR)-8- | indolizidine | | | | | y | y | | | | | | |
| | (3,4,5-trihydroxy-6- | | | | | | | | | | | | | |
| | (hydroxymethyl)tetrahydro- | | | | | | | | | | | | | |
| | 2H-pyran-2- | | | | | | | | | | | | | |
| | yloxy)octahydroindolizine- | | | | | | | | | | | | | |
| | 1,6,7-triol | | | | | | | | | | | | | |
| 33 | (1R,2S,3R,4S,5R,6R)-8- | nortropane | | | | | | | | | | | | y |
| | azabicyclo[3.2.1]octane- | | | | | | | | | | | | | |
| | 1,2,3,4,6-pentaol | | | | | | | | | | | | | |
| 34 | (2R,3R,4R,6R)-6-butyl- | piperidine | | | y | | | | | y | | | | |
| | 2- | | | | | | | | | | | | | |
| | (hydroxymethyl)piperidine- | | | | | | | | | | | | | |
| | 3,4-diol | | | | | | | | | | | | | |
| 35 | (1R,2R,3S,6S,7R,7aR)- | pyrrolizidine | | | | | y | y | | | y | | | |
| | 3- | | | | | | | | | | | | | |
| | (butyryloxymethyl)hexahydro- | | | | | | | | | | | | | |
| | 1H-pyrrolizine- | | | | | | | | | | | | | |
| | 1,2,6,7-tetrayl | | | | | | | | | | | | | |
| | tetrabutyrate | | | | | | | | | | | | | |
| 36 | (1S,2R,8R,8aR)- | indolizidine | | | | | | | | | y | | | |
| | octahydroindolizine- | | | | | | | | | | | | | |
| | 1,2,8-triol | | | | | | | | | | | | | |
| 37 | (1S,2R,6R,7S)- | pyrrolizidine | | y | | | | | | | y | | y | |
| | hexahydro-1H- | | | | | | | | | | | | | |
| | pyrrolizine-1,2,6,7- | | | | | | | | | | | | | |
| | tetraol | | | | | | | | | | | | | |
| 38 | (1R,2R,3S,6S,7R,7aR)- | pyrrolizidine | | | | | y | y | | | y | | | |
| | 7-amino-3- | | | | | | | | | | | | | |
| | (hydroxymethyl)hexahydro- | | | | | | | | | | | | | |
| | 1H-pyrrolizine-1,2,6- | | | | | | | | | | | | | |
| | triol | | | | | | | | | | | | | |
| 39 | (2R,3R,4R,5R)-2-((1R)- | pyrrolidine | | | | | | | | | y | | | |
| | 2-(3,4-dihydroxy-4- | | | | | | | | | | | | | |
| | (hydroxymethyl)tetrahydrofuran- | | | | | | | | | | | | | |
| | 2-yloxy)-1- | | | | | | | | | | | | | |
| | hydroxyethyl)-5- | | | | | | | | | | | | | |
| | (hydroxymethyl)pyrrolidine- | | | | | | | | | | | | | |
| | 3,4-diol | | | | | | | | | | | | | |
| 40 | (2R,3R,4R)-2- | piperidine | | | y | | | | | y | | | | |
| | (hydroxymethyl)piperidine- | | | | | | | | | | | | | |
| | 3,4-diol | | | | | | | | | | | | | |
| 41 | (1R,2S,6S,7R,8R,8aS)- | indolizidine | | | | | y | y | | | y | | | |
| | 2-(3,4,5-trihydroxy-6- | | | | | | | | | | | | | |
| | (hydroxymethyl)tetrahydro- | | | | | | | | | | | | | |
| | 2H-pyran-2- | | | | | | | | | | | | | |
| | yloxy)octahydroindolizine- | | | | | | | | | | | | | |
| | 1,6,7,8-tetraol | | | | | | | | | | | | | |
| 42 | (2R,3R,4R,5R)-2-((Z)-5- | pyrrolidine | | | | | | | | | y | | | |
| | hydrazono-4- | | | | | | | | | | | | | |
| | iminopentyl)-5- | | | | | | | | | | | | | |
| | (hydroxymethyl)pyrrolidine- | | | | | | | | | | | | | |
| | 3,4-diol | | | | | | | | | | | | | |
| 43 | (1S,2R,3S,5R)-8- | nortropane | | | y | | | | | y | | | | |
| | azabicyclo[3.2.1]octane- | | | | | | | | | | | | | |
| | 1,2,3,6-tetraol | | | | | | | | | | | | | |
| 44 | (1S,3R,4R,5S)-8- | nortropane | | | y | | | | | y | | | | |
| | azabicyclo[3.2.1]octane- | | | | | | | | | | | | | |
| | 1,3,4-triol | | | | | | | | | | | | | |
| 45 | (4R,5R,6S)-4,5- | oxazilidine | | | y | | | | | y | | | | |
| | dihydroxy-6- | | | | | | | | | | | | | |
| | (hydroxymethyl)morpholin- | | | | | | | | | | | | | |
| | 2-ium | | | | | | | | | | | | | |
| 46 | (1S,6S,7S,8R)-1,7,8- | indolizidine | | | | | y | y | | | | | | |
| | trihydroxyoctahydroindolizin- | | | | | | | | | | | | | |
| | 6-yl butyrate | | | | | | | | | | | | | |
| 47 | (1R,2R,3R,6S,7S,7aR)- | pyrrolizidine | | y | | | | | | | y | | y | |
| | 3- | | | | | | | | | | | | | |
| | (acetoxymethyl)hexahydro- | | | | | | | | | | | | | |
| | 1H-pyrrolizine- | | | | | | | | | | | | | |
| | 1,2,6,7-tetrayl | | | | | | | | | | | | | |
| | tetraacetate | | | | | | | | | | | | | |
| 48 | (2R,3R,4S)-2-((R)-1,2- | pyrrolidine | | y | | | | | | | | | y | |
| | dihydroxyethyl)-1-(2- | | | | | | | | | | | | | |
| | hydroxyethyl)pyrrolidine- | | | | | | | | | | | | | |
| | 3,4-diol | | | | | | | | | | | | | |
| 49 | (2R,3R,4R)-1-butyl-2- | pyrrolidine | | | y | | | | | y | | | | |
| | (hydroxymethyl)pyrrolidine- | | | | | | | | | | | | | |
| | 3,4-diol | | | | | | | | | | | | | |
| 50 | 2-((2R,3R,4R)-3- | piperidine | | | y | | | | | y | | | | |
| | hydroxy-2- | | | | | | | | | | | | | |
| | (hydroxymethyl)piperidin- | | | | | | | | | | | | | |
| | 4-yloxy)-6- | | | | | | | | | | | | | |
| | (hydroxymethyl)tetrahydro- | | | | | | | | | | | | | |
| | 2H-pyran-3,4,5-triol | | | | | | | | | | | | | |
| 51 | (2R,3R,4R,5S,6R)-2- | piperidine | | | | | y | y | y | | | | | |
| | (hydroxymethyl)-6- | | | | | | | | | | | | | |
| | methylpiperidine-3,4,5- | | | | | | | | | | | | | |
| | triol | | | | | | | | | | | | | |
| 52 | (2R,3R,4S,5S)-2,5- | piperidine | | | | | | | | | y | | | |
| | bis(hydroxymethyl)piperidine- | | | | | | | | | | | | | |
| | 3,4,5-triol | | | | | | | | | | | | | |
| 53 | 2-((S)-2-((2S,3S,4S,5S)- | pyrrolidine | | | | | | | | | y | | | |
| | 3,4-dihydroxy-5- | | | | | | | | | | | | | |
| | (hydroxymethyl)pyrrolidin- | | | | | | | | | | | | | |
| | 2-yl)-2- | | | | | | | | | | | | | |
| | hydroxyethoxy)tetrahydro- | | | | | | | | | | | | | |
| | 2H-pyran-3,4,5-triol | | | | | | | | | | | | | |
| 54 | (1S,2R,3R,7aR)-3- | pyrrolizidine | | y | | | | | | | | | y | |
| | (hydroxymethyl)hexahydro- | | | | | | | | | | | | | |
| | 1H-pyrrolizine-1,2- | | | | | | | | | | | | | |
| | diol | | | | | | | | | | | | | |
| 55 | (1R,2R,3R,6S,7S,7aR)- | pyrrolizidine | | y | | | | | | | y | | y | |
| | 3-((3,4,5-trihydroxy-6- | | | | | | | | | | | | | |
| | (hydroxymethyl)tetrahydro- | | | | | | | | | | | | | |
| | 2H-pyran-2- | | | | | | | | | | | | | |
| | yloxy)methyl)hexahydro- | | | | | | | | | | | | | |
| | 1H-pyrrolizine-1,2,6,7- | | | | | | | | | | | | | |
| | tetraol | | | | | | | | | | | | | |
| 56 | (1R,2R,3S,7S,7aR)- | pyrrolizidine | | | | y | | | | | y | | | |
| | 1,2,7- | | | | | | | | | | | | | |
| | trihydroxyhexahydro- | | | | | | | | | | | | | |
| | 1H-pyrrolizine-3- | | | | | | | | | | | | | |
| | carboxylic acid | | | | | | | | | | | | | |
| 57 | (2R,3S)-2- | pyrrolidine | | | | | | | | | | | | |
| | (hydroxymethyl)pyrrolidin- | | | | | | | | | | | | | |
| | 3-ol | | | | | | | | | | | | | |
| 58 | (3S,4S,5R,6S)-3,4,5- | piperidine | | | | | y | y | | | | | | |
| | trihydroxy-3,6- | | | | | | | | | | | | | |
| | bis(hydroxymethyl)piperidin- | | | | | | | | | | | | | |
| | 2-one | | | | | | | | | | | | | |
| 59 | (1S,2R,3R,5S,7aR)-5- | pyrrolizidine | | y | | | | | | | | | y | |
| | ((1R)-1,3- | | | | | | | | | | | | | |
| | dihydroxybutyl)-3- | | | | | | | | | | | | | |
| | (hydroxymethyl)hexahydro- | | | | | | | | | | | | | |
| | 1H-pyrrolizine-1,2- | | | | | | | | | | | | | |
| | diol | | | | | | | | | | | | | |
| 60 | (2S,3S,4S,5S)-2-(4- | pyrrolidine | | | | | | | | | y | | | |
| | aminopentyl)-5- | | | | | | | | | | | | | |
| | (hydroxymethyl)pyrrolidine- | | | | | | | | | | | | | |
| | 3,4-diol | | | | | | | | | | | | | |
| 61 | 4-((2S,3S,4R,5R)-3,4- | piperidine | | | | | y | y | | | | | | |
| | dihydroxy-2- | | | | | | | | | | | | | |
| | (hydroxymethyl)-5- | | | | | | | | | | | | | |
| | (3,4,5-trihydroxy-6- | | | | | | | | | | | | | |
| | (hydroxymethyl)tetrahydro- | | | | | | | | | | | | | |
| | 2H-pyran-2- | | | | | | | | | | | | | |
| | yloxy)piperidin-1- | | | | | | | | | | | | | |
| | yl)butanoic acid | | | | | | | | | | | | | |
| 62 | (2R,3R,4R,5R)-2- | pyrrolidine | | | | | | | | | y | | | |
| | (hydroxymethyl)-5-((R)- | | | | | | | | | | | | | |
| | 1- | | | | | | | | | | | | | |
| | hydroxypropyl)pyrrolidine- | | | | | | | | | | | | | |
| | 3,4-diol | | | | | | | | | | | | | |
| 63 | (2R,3R,4R,5R)-2-((1R)- | pyrrolidine | | | | | | | | | y | | | |
| | 1,2-dihydroxypropyl)-5- | | | | | | | | | | | | | |
| | (hydroxymethyl)pyrrolidine- | | | | | | | | | | | | | |
| | 3,4-diol | | | | | | | | | | | | | |
| 64 | (2R,3R,4R,5R)-1-(2- | pyrrolidine | | | | | | | | | y | | | |
| | acetoxyethyl)-2,5- | | | | | | | | | | | | | |
| | bis(acetoxymethyl)pyrrolidine- | | | | | | | | | | | | | |
| | 3,4-diyl diacetate | | | | | | | | | | | | | |
| 65 | (2S,4R)-4-hydroxy-1- | pyrrolidine | | | | | | | | | | y | | y |
| | methylpyrrolidine-2- | | | | | | | | | | | | | |
| | carboxylic acid | | | | | | | | | | | | | |
| 66 | (1S,2R,3R,5R,6S,7aR)- | pyrrolizidine | | | | | | | | | y | | | |
| | 5-(3-hydroxybutyl)-3- | | | | | | | | | | | | | |
| | (hydroxymethyl)hexahydro- | | | | | | | | | | | | | |
| | 1H-pyrrolizine-1,2,6- | | | | | | | | | | | | | |
| | triol | | | | | | | | | | | | | |
| 67 | (1S,2R,3R,5S,7aR)-5- | pyrrolizidine | | | | | | | | | y | | | |
| | (3-hydroxybutyl)-3- | | | | | | | | | | | | | |
| | (hydroxymethyl)hexahydro- | | | | | | | | | | | | | |
| | 1H-pyrrolizine-1,2- | | | | | | | | | | | | | |
| | diol | | | | | | | | | | | | | |
| 68 | (1S,2R,3R,5S,7R,7aR)- | pyrrolizidine | | | | | y | y | | | y | | | |
| | 3,5- | | | | | | | | | | | | | |
| | bis(hydroxymethyl)hexahydro- | | | | | | | | | | | | | |
| | 1H-pyrrolizine- | | | | | | | | | | | | | |
| | 1,2,7-triol | | | | | | | | | | | | | |
| 69 | (2S,3S,4S,5R,6S)-2- | piperidine | | | | | y | y | y | | | | | |
| | (acetoxymethyl)-6- | | | | | | | | | | | | | |
| | ethylpiperidine-3,4,5- | | | | | | | | | | | | | |
| | triyl triacetate | | | | | | | | | | | | | |
| 70 | (2S,3R,4S)-2-((R)-1,2- | pyrrolidine | | | | | | | | | y | | | |
| | dihydroxyethyl)pyrrolidine- | | | | | | | | | | | | | |
| | 3,4-diol | | | | | | | | | | | | | |
| 71 | (2R,3S)-3-hydroxy-1,1- | pyrrolidine | | | | | | | | | | | | |
| | dimethylpyrrolidinium-2- | | | | | | | | | | | | | |
| | carboxylate | | | | | | | | | | | | | |
| 72 | (2S,3S,4S,5R)-2- | piperidine | | | | | y | y | | | | | | |
| | ethylpiperidine-3,4,5- | | | | | | | | | | | | | |
| | triol | | | | | | | | | | | | | |
| 73 | (2S,3S,4R)-1-benzyl-2- | pyrrolidine | y | | | | | | | | | | | |
| | ((R)-1,2- | | | | | | | | | | | | | |
| | dihydroxyethyl)pyrrolidine- | | | | | | | | | | | | | |
| | 3,4-diol | | | | | | | | | | | | | |
| 74 | (2S,3S,4R)-1-butyl-2- | pyrrolidine | | | | | | | | | | | | |
| | (hydroxymethyl)pyrrolidine- | | | | | | | | | | | | | |
| | 3,4-diol | | | | | | | | | | | | | |
| 75 | (2S,3R,4S)-2-(1,2- | pyrrolidine | | | | | | | | | y | | | |
| | dihydroxypropyl)pyrrolidine- | | | | | | | | | | | | | |
| | 3,4-diol | | | | | | | | | | | | | |
| 76 | (2S,3S,4S,5S)-2-(3,6- | pyrrolidine | | | | | | | | | y | | | |
| | dihydroxyheptyl)-5- | | | | | | | | | | | | | |
| | (hydroxymethyl)pyrrolidine- | | | | | | | | | | | | | |
| | 3,4-diol | | | | | | | | | | | | | |
| 77 | (2S,3R,4R,5R,6R)-5- | piperidine | | | | y | | | | | y | | | |
| | (3,4-dihydroxy-2,5- | | | | | | | | | | | | | |
| | bis(hydroxymethyl)tetra | | | | | | | | | | | | | |
| | hydrofuran-2-yloxy)-2,6- | | | | | | | | | | | | | |
| | bis(hydroxymethyl)piperidine- | | | | | | | | | | | | | |
| | 3,4-diol | | | | | | | | | | | | | |
| 78 | (2R,3R,4R,5S)-2- | piperidine | | | | | y | y | | | | | | |
| | ((3,4,5-trihydroxy-6- | | | | | | | | | | | | | |
| | (hydroxymethyl)tetrahydro- | | | | | | | | | | | | | |
| | 2H-pyran-2- | | | | | | | | | | | | | |
| | yloxy)methyl)piperidine- | | | | | | | | | | | | | |
| | 3,4,5-triol | | | | | | | | | | | | | |
| 79 | 2-((2R,3R,4R,5R)-4- | pyrrolidine | | | | | | | | | y | | | |
| | hydroxy-2,5- | | | | | | | | | | | | | |
| | bis(hydroxymethyl)pyrrolidin- | | | | | | | | | | | | | |
| | 3-yloxy)-6- | | | | | | | | | | | | | |
| | (hydroxymethyl)tetrahydro- | | | | | | | | | | | | | |
| | 2H-pyran-3,4,5-triol | | | | | | | | | | | | | |
| 80 | (2S,3R,4R)-3,4- | pyrrolidine | | | y | | | | | y | | | | |
| | dihydroxy-1,1- | | | | | | | | | | | | | |
| | dimethylpyrrolidinium-2- | | | | | | | | | | | | | |
| | carboxylate | | | | | | | | | | | | | |
| 81 | (1R,2R,3S,4R,6S,7R,7aR)- | pyrrolizidine | | | | | y | y | | | y | | | |
| | 1,2,6,7-tetrahydroxy- | | | | | | | | | | | | | |
| | 3- | | | | | | | | | | | | | |
| | (hydroxymethyl)octahydropyrrolizine 4-oxide | | | | | | | | | | | | | |
| 82 | (2S,3R,4S,5R)-2,3- | piperidine | | | | y | | | | | | | | |
| | dimethylpiperidine- | | | | | | | | | | | | | |
| | 3,4,5-triol | | | | | | | | | | | | | |
| 83 | (3R,4R,5S)-3,4,5- | piperidine | | | y | | | | | y | | | | |
| | trihydroxy-3- | | | | | | | | | | | | | |
| | (hydroxymethyl)piperidin- | | | | | | | | | | | | | |
| | 2-one | | | | | | | | | | | | | |
| 84 | (3R,4S)-2,2- | pyrrolidine | | | y | | | | | y | | y | | |
| | bis(hydroxymethyl)pyrrolidine- | | | | | | | | | | | | | |
| | 3,4-diol | | | | | | | | | | | | | |
| 85 | (2S,4S)-4- | pyrrolidine | | | y | | | | | y | | | | |
| | (hydroxymethyl)-1- | | | | | | | | | | | | | |
| | methylpyrrolidine-2- | | | | | | | | | | | | | |
| | carboxylic acid | | | | | | | | | | | | | |
| 86 | (2R,3S,4R)-2-((S)-1,2- | pyrrolidine | | | | | | | | | y | | | |
| | dihydroxyethyl)-4- | | | | | | | | | | | | | |
| | methylpyrrolidine-3,4- | | | | | | | | | | | | | |
| | diol | | | | | | | | | | | | | |
| 87 | (3R,4R,5R)-3,4,5- | piperidine | | | y | | | | | y | | | | |
| | trihydroxypiperidine-3- | | | | | | | | | | | | | |
| | carboxylic acid | | | | | | | | | | | | | |
| 88 | (2R,3S,4S)-2-((S)-1- | pyrrolidine | | | | | y | y | y | | | | | |
| | hydroxyethyl)pyrrolidine- | | | | | | | | | | | | | |
| | 3,4-diol | | | | | | | | | | | | | |
| 89 | (3S,4R)-1-(allyloxy)-2,2- | pyrrolidine | | | y | | | | | y | | y | | |
| | bis(hydroxymethyl)pyrrolidine- | | | | | | | | | | | | | |
| | 3,4-diol | | | | | | | | | | | | | |
| 90 | N-((1S,7aR)-hexahydro- | pyrrolizidine | | | | | | | | | | | | |
| | 1H-pyrrolizin-1-yl)-2- | | | | | | | | | | | | | |
| | methylbutanamide | | | | | | | | | | | | | |
| 91 | (3S,4R)-2,2- | pyrrolidine | | | y | | | | | y | | y | | |
| | bis(hydroxymethyl)-1- | | | | | | | | | | | | | |
| | propoxypyrrolidine-3,4- | | | | | | | | | | | | | |
| | diol | | | | | | | | | | | | | |
| 92 | (2S,3S,4R)-2- | pyrrolidine | | | | | | | | | | y | | |
| | (hydroxymethyl)-2- | | | | | | | | | | | | | |
| | methylpyrrolidine-3,4- | | | | | | | | | | | | | |
| | diol | | | | | | | | | | | | | |
| 93 | (1R,2S,6S,8S,8aS)-6- | indolizidine | | | | | | | | | y | | | |
| | methyloctahydroindolizine- | | | | | | | | | | | | | |
| | 1,2,8-triol | | | | | | | | | | | | | |
| 94 | (2R,3R,4R)-3,4- | pyrrolidine | | | y | | | | | y | | | | |
| | dihydroxy-1-(2- | | | | | | | | | | | | | |
| | hydroxyethyl)-2- | | | | | | | | | | | | | |
| | (hydroxymethyl)pyrrolidine | | | | | | | | | | | | | |
| | 1-oxide | | | | | | | | | | | | | |
| 95 | (2R,3R,4R)-1-butyl-3,4- | pyrrolidine | | | y | | | | | y | | | | |
| | dihydroxy-2- | | | | | | | | | | | | | |
| | (hydroxymethyl)pyrrolidine | | | | | | | | | | | | | |
| | 1-oxide | | | | | | | | | | | | | |
| 96 | (2S,3R,4S)-1-butyl-2- | pyrrolidine | | | | | y | y | | | | | | |
| | ((S)-1,2- | | | | | | | | | | | | | |
| | dihydroxyethyl)pyrrolidine- | | | | | | | | | | | | | |
| | 3,4-diol | | | | | | | | | | | | | |
| 97 | (S)-1-((3aS,4R,6aS)-6a- | pyrrolidine | | | | | | | | | y | | | |
| | (hydroxymethyl)-2,2- | | | | | | | | | | | | | |
| | dimethyltetrahydro-3aH- | | | | | | | | | | | | | |
| | [1,3]dioxolo[4,5-c]pyrrol- | | | | | | | | | | | | | |
| | 4-yl)ethane-1,2-diol | | | | | | | | | | | | | |
| 98 | (2R,3S,4S)-2-((S)-1,2- | pyrrolidine | | | | | | | | | y | | | |
| | dihydroxyethyl)-4- | | | | | | | | | | | | | |
| | (hydroxymethyl)pyrrolidine- | | | | | | | | | | | | | |
| | 3,4-diol | | | | | | | | | | | | | |
| 99 | (2S,3S,4R)-1-(2- | pyrrolidine | | | | | | | | | | y | | |
| | hydroxyethyl)-2- | | | | | | | | | | | | | |
| | (hydroxymethyl)-2- | | | | | | | | | | | | | |
| | methylpyrrolidine-3,4- | | | | | | | | | | | | | |
| | diol | | | | | | | | | | | | | |
| 100 | (2S,3R,4S,5R)-1-butyl- | piperidine | | | | y | | | | | | | | |
| | 2,3-dimethylpiperidine- | | | | | | | | | | | | | |
| | 3,4,5-triol | | | | | | | | | | | | | |
| 101 | N-((3R,5R)-1-benzyl-5- | pyrrolidine | y | y | | | | | | | | | y | |
| | ((S)-1,2- | | | | | | | | | | | | | |
| | dihydroxyethyl)pyrrolidin- | | | | | | | | | | | | | |
| | 3-yl)acetamide | | | | | | | | | | | | | |
| 102 | (3R,4S,5R)-5,6- | piperidine | | | y | | | | | y | | | | |
| | dimethyl-2,3,4,5- | | | | | | | | | | | | | |
| | tetrahydropyridine- | | | | | | | | | | | | | |
| | 3,4,5-triol | | | | | | | | | | | | | |
| 103 | (3R,4r,5S)-piperidine- | piperidine | | | | | | | | | | | | y |
| | 3,4,5-triol | | | | | | | | | | | | | |
| 104 | (1S,6S,7R,8R,8aR)- | indolizidine | | | | | y | y | | | | | | |
| | octahydroindolizine- | | | | | | | | | | | | | |
| | 1,6,7,8-tetraol | | | | | | | | | | | | | |
| 105 | (1R,2S,3S,4S,5R)-4- | nortropane | | | | | | | | | | | | y |
| | (3,4,5-trihydroxy-6- | | | | | | | | | | | | | |
| | (hydroxymethyl)tetrahydro- | | | | | | | | | | | | | |
| | 2H-pyran-2-yloxy)-8- | | | | | | | | | | | | | |
| | azabicyclo[3.2.1]octane- | | | | | | | | | | | | | |
| | 1,2,3-triol | | | | | | | | | | | | | |
| 106 | (1S,2S,3R,4S,5S)-5- | nortropane | | y | | | | | | | y | | y | |
| | methyl-8-oxa-6- | | | | | | | | | | | | | |
| | azabicyclo[3.2.1]octane- | | | | | | | | | | | | | |
| | 2,3,4-triol | | | | | | | | | | | | | |
| 107 | (7aS,7a1R,10aR,18aR, | pyrrolizidine | | y | | | y | y | | | | | y | |
| | 18bS)- | | | | | | | | | | | | | |
| | 2,2,6,6,13,13,17,17- | | | | | | | | | | | | | |
| | octamethyltetradecahydrobis[1, | | | | | | | | | | | | | |
| | 5]dioxecino[2,3- | | | | | | | | | | | | | |
| | b:2′,3′,4′-gh]pyrrolizine- | | | | | | | | | | | | | |
| | 4,15(7aH,7a1H)-dione | | | | | | | | | | | | | |
| 108 | (2S,3S,4R,5R,6R)-2- | piperidine | | | | | y | y | | | | | | |
| | butyl-6- | | | | | | | | | | | | | |
| | (hydroxymethyl)piperidine- | | | | | | | | | | | | | |
| | 3,4,5-triol | | | | | | | | | | | | | |
| 109 | (2R,3R,4S,5R)-2- | piperidine | | y | | | | | | | | | y | |
| | methylpiperidine-3,4,5- | | | | | | | | | | | | | |
| | triol | | | | | | | | | | | | | |
| 110 | 2-((2R,3R,4R,5S,6R)- | piperidine | | | | | y | y | y | | | | | |
| | 4,5-dihydroxy-2,6- | | | | | | | | | | | | | |
| | bis(hydroxymethyl)piperidin- | | | | | | | | | | | | | |
| | 3-yloxy)-6- | | | | | | | | | | | | | |
| | (hydroxymethyl)tetrahydro- | | | | | | | | | | | | | |
| | 2H-pyran-3,4,5-triol | | | | | | | | | | | | | |
| 111 | 1-((2S,3R,4R,5R)-3,4- | pyrrolidine | | | | | y | y | | | | | | |
| | dihydroxy-5- | | | | | | | | | | | | | |
| | (hydroxymethyl)pyrrolidin- | | | | | | | | | | | | | |
| | 2-yl)-2-methoxy-1H- | | | | | | | | | | | | | |
| | imidazole-4,5-diol | | | | | | | | | | | | | |
| 112 | (3S,4S,5R,6R)-3,4,5- | piperidine | | | | | | | | | y | | | |
| | trihydroxy-6- | | | | | | | | | | | | | |
| | (hydroxymethyl)piperidin- | | | | | | | | | | | | | |
| | 2-one | | | | | | | | | | | | | |
| 113 | 2-((1S,5R,6R,7R,7aS)- | pyrrolizidine | | | | y | | | | | y | | | |
| | 6,7-dihydroxy-5- | | | | | | | | | | | | | |
| | (hydroxymethyl)hexahydro- | | | | | | | | | | | | | |
| | 1H-pyrrolizin-1- | | | | | | | | | | | | | |
| | yloxy)-6- | | | | | | | | | | | | | |
| | (hydroxymethyl)tetrahydro- | | | | | | | | | | | | | |
| | 2H-pyran-3,4,5-triol | | | | | | | | | | | | | |
| 114 | (2S,3S,4R,5R,6R)-2- | piperidine | | | | | y | y | | | | | | |
| | pentyl-6-((3,4,5- | | | | | | | | | | | | | |
| | trihydroxytetrahydro-2H- | | | | | | | | | | | | | |
| | pyran-2- | | | | | | | | | | | | | |
| | yloxy)methyl)piperidine- | | | | | | | | | | | | | |
| | 3,4,5-triol | | | | | | | | | | | | | |
| 115 | (2R,4R)-2- | piperidine | | | | | | | | | | y | | y |
| | carboxypiperidinium-4-yl | | | | | | | | | | | | | |
| | sulfate | | | | | | | | | | | | | |
| 116 | (1S,2R,3R,5R,7aR)-3,5- | pyrrolizidine | | y | | | | | | | | | y | |
| | bis(hydroxymethyl)hexahydro- | | | | | | | | | | | | | |
| | 1H-pyrrolizine- | | | | | | | | | | | | | |
| | 1,2-diol | | | | | | | | | | | | | |
| 117 | (2R,3R,4R,5R)-3,4- | pyrrolidine | | | | | | | | | y | | | |
| | dihydroxy-2-methyl-1- | | | | | | | | | | | | | |
| | oxo-5- | | | | | | | | | | | | | |
| | phenylpyrrolidinium | | | | | | | | | | | | | |
| 118 | (2R,3R,4R,5S)-1-butyl- | piperidine | | | | | y | y | | | | | | |
| | 2- | | | | | | | | | | | | | |
| | (hydroxymethyl)piperidine- | | | | | | | | | | | | | |
| | 3,4,5-triol | | | | | | | | | | | | | |
| 119 | (2S,3S,4S,5R)-2- | pyrrolidine | | | | | y | y | | | | | | |
| | (hydroxymethyl)-5- | | | | | | | | | | | | | |
| | methylpyrrolidine-3,4- | | | | | | | | | | | | | |
| | diol | | | | | | | | | | | | | |
| 120 | 2-((2R,3R,4R,5S)-3,5- | piperidine | | | | | y | y | | | | | | |
| | dihydroxy-2- | | | | | | | | | | | | | |
| | (hydroxymethyl)piperidin- | | | | | | | | | | | | | |
| | 4-yloxy)-6- | | | | | | | | | | | | | |
| | (hydroxymethyl)tetrahydro- | | | | | | | | | | | | | |
| | 2H-pyran-3,4,5-triol | | | | | | | | | | | | | |
| 121 | 2-((3S,4S,5R,6R)-4,5- | piperidine | | | | | y | y | | | | | | |
| | dihydroxy-6- | | | | | | | | | | | | | |
| | (hydroxymethyl)piperidin- | | | | | | | | | | | | | |
| | 3-yloxy)-6- | | | | | | | | | | | | | |
| | (hydroxymethyl)tetrahydro- | | | | | | | | | | | | | |
| | 2H-pyran-3,4,5-triol | | | | | | | | | | | | | |
| 122 | 2-((3S,4S,5R,6R)-4,5- | piperidine | | | | | y | y | | | | | | |
| | dihydroxy-6- | | | | | | | | | | | | | |
| | (hydroxymethyl)-1- | | | | | | | | | | | | | |
| | methylpiperidin-3- | | | | | | | | | | | | | |
| | yloxy)-6- | | | | | | | | | | | | | |
| | (hydroxymethyl)tetrahydro- | | | | | | | | | | | | | |
| | 2H-pyran-3,4,5-triol | | | | | | | | | | | | | |
| 123 | 2-((3R,4R,5R)-4- | pyrrolidine | | | y | | | | | y | | | | |
| | hydroxy-5- | | | | | | | | | | | | | |
| | (hydroxymethyl)pyrrolidin- | | | | | | | | | | | | | |
| | 3-yloxy)-6- | | | | | | | | | | | | | |
| | (hydroxymethyl)tetrahydro- | | | | | | | | | | | | | |
| | 2H-pyran-3,4,5-triol | | | | | | | | | | | | | |
| 124 | (2R,3R,4R,5S,6R)-2,6- | piperidine | | | | | y | y | y | | | | | |
| | bis(hydroxymethyl)-1- | | | | | | | | | | | | | |
| | methylpiperidine-3,4,5- | | | | | | | | | | | | | |
| | triol | | | | | | | | | | | | | |
| 125 | (3aR,6S,7R,7aS)- | piperidine | | | | | | | y | | | | | |
| | hexahydrospiro[[1,3]dioxolo[4, | | | | | | | | | | | | | |
| | 5-b]pyridine-2,1′- | | | | | | | | | | | | | |
| | cyclohexane]-6,7-diol | | | | | | | | | | | | | |
| 126 | (3S)-2,3-dihydroxy-3- | pyrrolidine | | | | | | | | | y | | | |
| | ((2R,3R,4R)-2,3,4- | | | | | | | | | | | | | |
| | trihydroxypyrrolidin-2- | | | | | | | | | | | | | |
| | yl)propanoic acid | | | | | | | | | | | | | |
| 127 | (1R,2R,3S,7S,7aR)-3- | pyrrolizidine | | | | y | y | y | | | | | | |
| | (hydroxymethyl)hexahydro- | | | | | | | | | | | | | |
| | 1H-pyrrolizine-1,2,7- | | | | | | | | | | | | | |
| | triol | | | | | | | | | | | | | |
| 128 | (1R,2R,3R,7S,7aR)-3- | pyrrolizidine | | | | y | | | | | y | | | |
| | (hydroxymethyl)hexahydro- | | | | | | | | | | | | | |
| | 1H-pyrrolizine-1,2,7- | | | | | | | | | | | | | |
| | triol | | | | | | | | | | | | | |
| 129 | (1R,2R,3S,6S,7S,7aR)- | pyrrolizidine | | y | | | y | y | | | | | y | |
| | 3- | | | | | | | | | | | | | |
| | (hydroxymethyl)hexahydro- | | | | | | | | | | | | | |
| | 1H-pyrrolizine- | | | | | | | | | | | | | |
| | 1,2,6,7-tetraol | | | | | | | | | | | | | |
| 130 | (1S,2S,6S,7S,8S,8aS)- | indolizidine | | | | | y | y | | | y | | | |
| | octahydroindolizine- | | | | | | | | | | | | | |
| | 1,2,6,7,8-pentaol | | | | | | | | | | | | | |
| 131 | (1R,2R,3S,6R,7R,7aR)- | pyrrolizidine | | | | | y | y | | | | | | |
| | 3- | | | | | | | | | | | | | |
| | (hydroxymethyl)hexahydro- | | | | | | | | | | | | | |
| | 1H-pyrrolizine- | | | | | | | | | | | | | |
| | 1,2,6,7-tetraol | | | | | | | | | | | | | |
| 132 | (1R,2R,3R,6S,7S,7aR)- | pyrrolizidine | | y | | | | | | | y | | y | |
| | 3- | | | | | | | | | | | | | |
| | (butyryloxymethyl)hexahydro- | | | | | | | | | | | | | |
| | 1H-pyrrolizine- | | | | | | | | | | | | | |
| | 1,2,6,7-tetrayl | | | | | | | | | | | | | |
| | tetrabutyrate | | | | | | | | | | | | | |
| 133 | (2R,3S,4S,5S,6R)-2- | piperidine | | | | y | y | y | | | | | | |
| | (hydroxymethyl)-6- | | | | | | | | | | | | | |
| | methylpiperidine-3,4,5- | | | | | | | | | | | | | |
| | triol | | | | | | | | | | | | | |
| 134 | (2R,3R,4R,5R,6S)-2- | piperidine | | | | y | | | | | y | | | |
| | (hydroxymethyl)-6- | | | | | | | | | | | | | |
| | methylpiperidine-3,4,5- | | | | | | | | | | | | | |
| | triol | | | | | | | | | | | | | |
| 135 | (2S,3R,4S,5S)-2,5- | piperidine | | | | | y | y | | | | | | |
| | bis(hydroxymethyl)piperidine- | | | | | | | | | | | | | |
| | 3,4,5-triol | | | | | | | | | | | | | |
| 136 | (1R,2R,3S,6S,7S,7aR)- | pyrrolizidine | | y | | | y | y | | | | | y | |
| | 3- | | | | | | | | | | | | | |
| | (acetoxymethyl)hexahydro- | | | | | | | | | | | | | |
| | 1H-pyrrolizine- | | | | | | | | | | | | | |
| | 1,2,6,7-tetrayl | | | | | | | | | | | | | |
| | tetraacetate | | | | | | | | | | | | | |
| 137 | (1R,2R,3R,6S,7S,7aR)- | pyrrolizidine | | y | | | | | | | y | | y | |
| | 3- | | | | | | | | | | | | | |
| | (hydroxymethyl)hexahydro- | | | | | | | | | | | | | |
| | 1H-pyrrolizine- | | | | | | | | | | | | | |
| | 1,2,6,7-tetraol | | | | | | | | | | | | | |
| 138 | (1R,2R,3R,4S,5R)-8- | nortropane | | | y | | | | | y | | | | |
| | azabicyclo[3.2.1]octane- | | | | | | | | | | | | | |
| | 1,2,3,4-tetraol | | | | | | | | | | | | | |
| 139 | (1S,2R,3R,7S,7aR)-3- | pyrrolizidine | | y | | | y | y | | | | | y | |
| | (hydroxymethyl)hexahydro- | | | | | | | | | | | | | |
| | 1H-pyrrolizine-1,2,7- | | | | | | | | | | | | | |
| | triol | | | | | | | | | | | | | |
| 140 | (2R,3R,4S)-2- | piperidine | | | | | | | | | | y | | |
| | (hydroxymethyl)piperidine- | | | | | | | | | | | | | |
| | 3,4-diol | | | | | | | | | | | | | |
| 141 | (1R,2S,3R,4R,5R)-8- | nortropane | | | y | | | | | y | | | | |
| | azabicyclo[3.2.1]octane- | | | | | | | | | | | | | |
| | 1,2,3,4-tetraol | | | | | | | | | | | | | |
| 142 | (2R,3R,4R)-1-(2- | pyrrolidine | | | y | | | | | y | | | | |
| | hydroxyethyl)-2- | | | | | | | | | | | | | |
| | (hydroxymethyl)pyrrolidine- | | | | | | | | | | | | | |
| | 3,4-diol | | | | | | | | | | | | | |
| 143 | (1S,2R,3R,5R,7R,7aR)- | pyrrolizidine | | y | | | | | | | y | | y | |
| | 3-(hydroxymethyl)-5- | | | | | | | | | | | | | |
| | methylhexahydro-1H- | | | | | | | | | | | | | |
| | pyrrolizine-1,2,7-triol | | | | | | | | | | | | | |
| 144 | (1R,2R,3S,6S,7R,7aR)- | pyrrolizidine | | | | | y | y | | | y | | | |
| | 3- | | | | | | | | | | | | | |
| | (acetoxymethyl)hexahydro- | | | | | | | | | | | | | |
| | 1H-pyrrolizine- | | | | | | | | | | | | | |
| | 1,2,6,7-tetrayl | | | | | | | | | | | | | |
| | tetraacetate | | | | | | | | | | | | | |
| 145 | (2R,3S,4R)-2-((S)-1,2- | pyrrolidine | | | | | | | | | y | | | |
| | dihydroxyethyl)-1-(2- | | | | | | | | | | | | | |
| | hydroxyethyl)pyrrolidine- | | | | | | | | | | | | | |
| | 3,4-diol | | | | | | | | | | | | | |
| 146 | (1R,2R,3S,6S,7R,7aS)- | pyrrolizidine | | y | | | | | y | | | | y | |
| | 3- | | | | | | | | | | | | | |
| | (acetoxymethyl)hexahydro- | | | | | | | | | | | | | |
| | 1H-pyrrolizine- | | | | | | | | | | | | | |
| | 1,2,6,7-tetrayl | | | | | | | | | | | | | |
| | tetraacetate | | | | | | | | | | | | | |
| 147 | (1S,2S,3S,6R,7R,7aS)- | pyrrolizidine | | y | | | | | | | y | | y | |
| | 3- | | | | | | | | | | | | | |
| | (hydroxymethyl)hexahydro- | | | | | | | | | | | | | |
| | 1H-pyrrolizine- | | | | | | | | | | | | | |
| | 1,2,6,7-tetraol | | | | | | | | | | | | | |
| 148 | (1S,2S,3S,6S,7S,7aS)- | pyrrolizidine | | | | | y | y | | | y | | | |
| | 3- | | | | | | | | | | | | | |
| | (hydroxymethyl)hexahydro- | | | | | | | | | | | | | |
| | 1H-pyrrolizine- | | | | | | | | | | | | | |
| | 1,2,6,7-tetraol | | | | | | | | | | | | | |
| 149 | (1S,2R,3R,5S,7R,7aR)- | pyrrolizidine | | y | | | | | | | y | | y | |
| | 3-(hydroxymethyl)-5- | | | | | | | | | | | | | |
| | methylhexahydro-1H- | | | | | | | | | | | | | |
| | pyrrolizine-1,2,7-triol | | | | | | | | | | | | | |
| 150 | (1S,2R,3R,5R,7aR)-3- | pyrrolizidine | | y | | | | | | | | | y | |
| | (hydroxymethyl)-5- | | | | | | | | | | | | | |
| | methylhexahydro-1H- | | | | | | | | | | | | | |
| | pyrrolizine-1,2-diol | | | | | | | | | | | | | |
| 151 | (1R,2S,3R,5R,7aR)-3- | pyrrolizidine | | y | | | | | | | | | y | |
| | (hydroxymethyl)-5- | | | | | | | | | | | | | |
| | methylhexahydro-1H- | | | | | | | | | | | | | |
| | pyrrolizine-1,2-diol | | | | | | | | | | | | | |
| 152 | (1S,2R,3R,5S,6R,7S,7aR)- | pyrrolizidine | y | y | | | | | | | y | | y | |
| | 3-(hydroxymethyl)-5- | | | | | | | | | | | | | |
| | methylhexahydro-1H- | | | | | | | | | | | | | |
| | pyrrolizine-1,2,6,7- | | | | | | | | | | | | | |
| | tetraol | | | | | | | | | | | | | |
| 153 | (1R,2S,8S,8aS)- | indolizidine | | | | | | | | | y | | | |
| | octahydroindolizine- | | | | | | | | | | | | | |
| | 1,2,8-triol | | | | | | | | | | | | | |
| 154 | (2R,3R,4S)-1-(2- | pyrrolidine | | | | | | | | | | y | | |
| | hydroxyethyl)-2- | | | | | | | | | | | | | |
| | (hydroxymethyl)pyrrolidine- | | | | | | | | | | | | | |
| | 3,4-diol | | | | | | | | | | | | | |
| 155 | (2S,3R,4S)-2-((S)-1,2- | pyrrolidine | | | | | y | y | | | | | | |
| | dihydroxyethyl)pyrrolidine- | | | | | | | | | | | | | |
| | 3,4-diol | | | | | | | | | | | | | |
| 156 | (2S,3S,4R)-2-((R)-1,2- | pyrrolidine | y | | | | | | | | | | | |
| | dihydroxyethyl)pyrrolidine- | | | | | | | | | | | | | |
| | 3,4-diol | | | | | | | | | | | | | |
| 157 | (2S,3R,4R)-1-butyl-2- | pyrrolidine | | | | | | | | | | | | y |
| | (hydroxymethyl)pyrrolidine- | | | | | | | | | | | | | |
| | 3,4-diol | | | | | | | | | | | | | |
| 158 | (1R,2S,3R,5S,7S,7aR)- | pyrrolizidine | | y | | | | | | | | | y | |
| | 3-(hydroxymethyl)-5- | | | | | | | | | | | | | |
| | methylhexahydro-1H- | | | | | | | | | | | | | |
| | pyrrolizine-1,2,7-triol | | | | | | | | | | | | | |
| 159 | (2S,3R,4S)-1-benzyl-2- | pyrrolidine | | | | | y | y | | | | | | |
| | ((S)-1,2- | | | | | | | | | | | | | |
| | dihydroxyethyl)pyrrolidine- | | | | | | | | | | | | | |
| | 3,4-diol | | | | | | | | | | | | | |
| 160 | (2S,3S,4S)-2- | pyrrolidine | | | y | | | | | y | | | | |
| | (hydroxymethyl)pyrrolidine- | | | | | | | | | | | | | |
| | 3,4-diol | | | | | | | | | | | | | |
| 161 | (2S,3S,4S)-1-(2- | pyrrolidine | | | y | | | | | y | | | | |
| | hydroxyethyl)-2- | | | | | | | | | | | | | |
| | (hydroxymethyl)pyrrolidine- | | | | | | | | | | | | | |
| | 3,4-diol | | | | | | | | | | | | | |
| 162 | (2S,3R,4S)-1-butyl-2- | pyrrolidine | | | y | | | | | y | | | | |
| | (hydroxymethyl)pyrrolidine- | | | | | | | | | | | | | |
| | 3,4-diol | | | | | | | | | | | | | |
| 163 | (2S,3S,4S)-1-butyl-2- | pyrrolidine | | | y | | | | | y | | | | |
| | (hydroxymethyl)pyrrolidine- | | | | | | | | | | | | | |
| | 3,4-diol | | | | | | | | | | | | | |
| 164 | (1S,2R,3S,5S,7S,7aR)- | pyrrolizidine | | | | y | y | y | | | | | | |
| | 3-(hydroxymethyl)-5- | | | | | | | | | | | | | |
| | methylhexahydro-1H- | | | | | | | | | | | | | |
| | pyrrolizine-1,2,7-triol | | | | | | | | | | | | | |
| 165 | (1S,2R,3S,4S)-1-butyl- | pyrrolidine | | | y | | | | | y | | | | |
| | 3,4-dihydroxy-2- | | | | | | | | | | | | | |
| | (hydroxymethyl)pyrrolidine | | | | | | | | | | | | | |
| | 1-oxide | | | | | | | | | | | | | |
| 166 | (2S,3S,4R)-2- | pyrrolidine | | | | | | | | | | y | | |
| | (hydroxymethyl)pyrrolidine- | | | | | | | | | | | | | |
| | 3,4-diol | | | | | | | | | | | | | |
| 167 | (2S,3R,4S)-2- | pyrrolidine | | | y | | | | | y | | | | |
| | (hydroxymethyl)pyrrolidine- | | | | | | | | | | | | | |
| | 3,4-diol | | | | | | | | | | | | | |
| 168 | (2R,3R,4S,5R)-1-(2- | pyrrolidine | | y | | | | | | | | | y | |
| | hydroxyethyl)-2,5- | | | | | | | | | | | | | |
| | bis(hydroxymethyl)pyrrolidine- | | | | | | | | | | | | | |
| | 3,4-diol | | | | | | | | | | | | | |
| 169 | (1R,2R,3R,6S,7S,7aR)- | pyrrolizidine | | y | | | | | | | y | | y | |
| | 1,2,6,7-tetrahydroxy-3- | | | | | | | | | | | | | |
| | (hydroxymethyl)octahydropyrrolizine | | | | | | | | | | | | | |
| | 4-oxide | | | | | | | | | | | | | |
| 170 | (3R,4R,5R)-3,4,5- | piperidine | | | | | | | | | | y | | |
| | trihydroxy-3- | | | | | | | | | | | | | |
| | (hydroxymethyl)piperidin- | | | | | | | | | | | | | |
| | 2-one | | | | | | | | | | | | | |
| 171 | (2S,3R,4R)-2- | pyrrolidine | | | | | | | | | | | | y |
| | (hydroxymethyl)pyrrolidine- | | | | | | | | | | | | | |
| | 3,4-diol | | | | | | | | | | | | | |
| 172 | (1R,2S,3S,7S,7aR)-3- | pyrrolizidine | y | y | | | | | | | | | y | |
| | (hydroxymethyl)hexahydro- | | | | | | | | | | | | | |
| | 1H-pyrrolizine-1,2,7- | | | | | | | | | | | | | |
| | triol | | | | | | | | | | | | | |
| 173 | (1R,2S,3R,5R,7S,7aR)- | pyrrolizidine | | y | | | | | | | | | y | |
| | 3-(hydroxymethyl)-5- | | | | | | | | | | | | | |
| | methylhexahydro-1H- | | | | | | | | | | | | | |
| | pyrrolizine-1,2,7-triol | | | | | | | | | | | | | |
| 174 | (2S,4R)-4- | pyrrolidine | | | | | | | | | | y | | y |
| | (hydroxymethyl)-1- | | | | | | | | | | | | | |
| | methylpyrrolidine-2- | | | | | | | | | | | | | |
| | carboxylic acid | | | | | | | | | | | | | |
| 175 | (1R,2S,6R,8S,8aS)-6- | indolizidine | | | | | | | | | y | | | |
| | methyloctahydroindolizine- | | | | | | | | | | | | | |
| | 1,2,8-triol | | | | | | | | | | | | | |
| 176 | (2S,3R,4S)-2-((S)-1,2- | pyrrolidine | | | | | y | y | | | | | | |
| | dihydroxyethyl)-1-(2- | | | | | | | | | | | | | |
| | hydroxyethyl)pyrrolidine- | | | | | | | | | | | | | |
| | 3,4-diol | | | | | | | | | | | | | |
| 177 | (1R,2R,3S,6S,7R,7aS)- | pyrrolizidine | | y | | | | | y | | | | y | |
| | 1,2,6,7-tetrahydroxy-3- | | | | | | | | | | | | | |
| | (hydroxymethyl)octahydropyrrolizine | | | | | | | | | | | | | |
| | 4-oxide | | | | | | | | | | | | | |
| 178 | (1R,2S,3R,4R)-1-butyl- | pyrrolidine | | | | | | | | | | | | y |
| | 3,4-dihydroxy-2- | | | | | | | | | | | | | |
| | (hydroxymethyl)pyrrolidine | | | | | | | | | | | | | |
| | 1-oxide | | | | | | | | | | | | | |
| 179 | (2S,3S,4S)-2-((S)-1,2- | pyrrolidine | | y | | | | | | | | | y | |
| | dihydroxyethyl)-4- | | | | | | | | | | | | | |
| | (hydroxymethyl)pyrrolidine- | | | | | | | | | | | | | |
| | 3,4-diol | | | | | | | | | | | | | |
| 180 | (2S,3S,4R,5S)-2,3- | piperidine | | y | | | | | | | | | y | |
| | dimethylpiperidine- | | | | | | | | | | | | | |
| | 3,4,5-triol | | | | | | | | | | | | | |
| 181 | (2R,3S,4R,5S)-2,3- | piperidine | | | | y | | | | | | | | |
| | dimethylpiperidine- | | | | | | | | | | | | | |
| | 3,4,5-triol | | | | | | | | | | | | | |
| 182 | (2R,3R,4S,5R)-2,3- | piperidine | | y | | | | | | | | | y | |
| | dimethylpiperidine- | | | | | | | | | | | | | |
| | 3,4,5-triol | | | | | | | | | | | | | |
| 183 | (2R,3R,4S,5R)-2,5- | pyrrolidine | | y | | | | | | | | | y | |
| | bis(hydroxymethyl)pyrrolidine- | | | | | | | | | | | | | |
| | 3,4-diol | | | | | | | | | | | | | |
| 184 | (2R,3R,4S,5R,6R)-2- | piperidine | | y | | | | | | | y | | y | |
| | (hydroxymethyl)-6- | | | | | | | | | | | | | |
| | methylpiperidine-3,4,5- | | | | | | | | | | | | | |
| | triol | | | | | | | | | | | | | |
| 185 | (1R,2R,3R,7R,7aR)-3- | pyrrolizidine | | y | | | | | | | y | | y | |
| | (hydroxymethyl)hexahydro- | | | | | | | | | | | | | |
| | 1H-pyrrolizine-1,2,7- | | | | | | | | | | | | | |
| | triol | | | | | | | | | | | | | |
| 186 | (1S,6R,7R,8R,8aR)- | indolizidine | | | | y | | | | | y | | | |
| | octahydroindolizine- | | | | | | | | | | | | | |
| | 1,6,7,8-tetraol | | | | | | | | | | | | | |
| 187 | (2R,3R,4S,5S)-2- | piperidine | y | | | | | | | | | | | |
| | (hydroxymethyl)piperidine- | | | | | | | | | | | | | |
| | 3,4,5-triol | | | | | | | | | | | | | |
| 188 | (2R,3R,4R,5S,6S)-2- | piperidine | | | | | y | y | | | | | | |
| | (hydroxymethyl)-6- | | | | | | | | | | | | | |
| | methylpiperidine-3,4,5- | | | | | | | | | | | | | |
| | triol | | | | | | | | | | | | | |
| 189 | (2R,3S,5S,6R)-2,6- | piperidine | | | | y | y | y | | | | | | |
| | bis(hydroxymethyl)piperidine- | | | | | | | | | | | | | |
| | 3,4,5-triol | | | | | | | | | | | | | |
| 190 | (2R,3R,4R,5R)-2- | pyrrolidine | | | | | | | | | y | | | |
| | (hydroxymethyl)-5- | | | | | | | | | | | | | |
| | methylpyrrolidine-3,4- | | | | | | | | | | | | | |
| | diol | | | | | | | | | | | | | |
| 191 | (1R,2R,3R,5R,7R,7aR)- | pyrrolizidine | | y | | | | | | | y | | y | |
| | 3-(hydroxymethyl)-5- | | | | | | | | | | | | | |
| | methylhexahydro-1H- | | | | | | | | | | | | | |
| | pyrrolizine-1,2,7-triol | | | | | | | | | | | | | |
| 192 | (1R,2R,3S,6S,7R,7aR)- | pyrrolizidine | | | | | y | y | | | y | | | |
| | 3- | | | | | | | | | | | | | |
| | (hydroxymethyl)hexahydro- | | | | | | | | | | | | | |
| | 1H-pyrrolizine- | | | | | | | | | | | | | |
| | 1,2,6,7-tetraol | | | | | | | | | | | | | |
| 193 | (2R,3R,4R,5S)-2- | piperidine | | | | | y | y | | | | | | |
| | (hydroxymethyl)piperidine- | | | | | | | | | | | | | |
| | 3,4,5-triol | | | | | | | | | | | | | |
| 194 | (2R,3R,4R,5R)-2-(2- | pyrrolidine | | | | | | | | | y | | | |
| | hydroxyethyl)-5- | | | | | | | | | | | | | |
| | (hydroxymethyl)pyrrolidine- | | | | | | | | | | | | | |
| | 3,4-diol | | | | | | | | | | | | | |
| 195 | (2S,3R,4R,5R)-2-(3- | pyrrolidine | | | | | y | y | | | | | | |
| | hydroxy-4- | | | | | | | | | | | | | |
| | methoxyphenyl)-5- | | | | | | | | | | | | | |
| | (hydroxymethyl)pyrrolidine- | | | | | | | | | | | | | |
| | 3,4-diol | | | | | | | | | | | | | |
| 196 | (2R,3R,4R,5S)-2- | pyrrolidine | | | | | y | y | | | | | | |
| | (hydroxymethyl)-5-(4- | | | | | | | | | | | | | |
| | hydroxyphenyl)pyrrolidine- | | | | | | | | | | | | | |
| | 3,4-diol | | | | | | | | | | | | | |
| 197 | (1R,2S,6S,7S,8R,8aR)- | indolizidine | | | | | y | y | | | y | | | |
| | 6- | | | | | | | | | | | | | |
| | methyloctahydroindolizine- | | | | | | | | | | | | | |
| | 1,2,6,7,8-pentaol | | | | | | | | | | | | | |
| 198 | (1R,2S,6R,7R,8R,8aR)- | indolizidine | | y | | | | | | | y | | y | |
| | 6- | | | | | | | | | | | | | |
| | methyloctahydroindolizine- | | | | | | | | | | | | | |
| | 1,2,6,7,8-pentaol | | | | | | | | | | | | | |
| 199 | (2S,3S,4R)-1-benzyl-2- | pyrrolidine | | | | | | | | | y | | | |
| | ((S)-1,2- | | | | | | | | | | | | | |
| | dihydroxyethyl)pyrrolidine- | | | | | | | | | | | | | |
| | 3,4-diol | | | | | | | | | | | | | |
| 200 | (2S,3S,4R)-2-((R)-1,2- | pyrrolidine | | y | | | | | | | | | y | |
| | dihydroxyethyl)-4- | | | | | | | | | | | | | |
| | (hydroxymethyl)pyrrolidine- | | | | | | | | | | | | | |
| | 3,4-diol | | | | | | | | | | | | | |
| 201 | (2R,3R,4S,5R)-2- | piperidine | | | | | | | | | y | | | |
| | (hydroxymethyl)-5- | | | | | | | | | | | | | |
| | methylpiperidine-3,4,5- | | | | | | | | | | | | | |
| | triol | | | | | | | | | | | | | |
| 202 | (2S,3S,4S)-2,4- | pyrrolidine | | | | | | | | | | y | | |
| | bis(hydroxymethyl)pyrrolidine- | | | | | | | | | | | | | |
| | 3,4-diol | | | | | | | | | | | | | |
| 203 | (2S,3S,4S)-2-((S)-1,2- | pyrrolidine | | y | | | | | | | | | y | |
| | dihydroxyethyl)-4- | | | | | | | | | | | | | |
| | (hydroxymethyl)pyrrolidine- | | | | | | | | | | | | | |
| | 3,4-diol | | | | | | | | | | | | | |
| 204 | 3-((2R,3R,4R,5S)-3,4,5- | piperidine | | | | | y | y | | | | | | |
| | trihydroxy-2- | | | | | | | | | | | | | |
| | (hydroxymethyl)piperidin- | | | | | | | | | | | | | |
| | 1-yl)propanoic acid | | | | | | | | | | | | | |
| 205 | (2S,3R,4R,5S)-butyl 1- | piperidine | | | | | y | y | | | | | | |
| | butyl-3,4,5- | | | | | | | | | | | | | |
| | trihydroxypiperidine-2- | | | | | | | | | | | | | |
| | carboxylate | | | | | | | | | | | | | |
| 206 | (1S,6R,7R,7aS)-7- | pyrrolizidine | | | | y | | | | | | | | |
| | (methylamino)hexahydro- | | | | | | | | | | | | | |
| | 1H-pyrrolizine-1,6-diol | | | | | | | | | | | | | |
| 207 | 2-((2S,3S,4S,5S)-3,4- | pyrrolidine | | | | | | | | | y | | | |
| | dihydroxy-2,5- | | | | | | | | | | | | | |
| | bis(hydroxymethyl)pyrrolidin- | | | | | | | | | | | | | |
| | 1-yl)acetic acid | | | | | | | | | | | | | |
| 208 | (1R,2R)-1-((2R,3R,4S)- | pyrrolidine | | | | y | | | | | y | | | |
| | 3,4-dihydroxypyrrolidin- | | | | | | | | | | | | | |
| | 2-yl)propane-1,2,3-triol | | | | | | | | | | | | | |
| 209 | (2S,3R,4R,5S)-3,4,5- | piperidine | | | | | y | y | | | | | | |
| | trihydroxy-1-(2- | | | | | | | | | | | | | |
| | hydroxyethyl)piperidine- | | | | | | | | | | | | | |
| | 2-carboxylic acid | | | | | | | | | | | | | |
| 210 | 2-((2R,3R,4R)-3,4- | pyrrolidine | | | y | | | | | y | | | | |
| | dihydroxy-2- | | | | | | | | | | | | | |
| | (hydroxymethyl)pyrrolidin- | | | | | | | | | | | | | |
| | 1-yl)acetic acid | | | | | | | | | | | | | |
| 211 | (2S,3S,4R)-2-((R)-1,2- | pyrrolidine | y | | | | | | | | | | | |
| | dihydroxyethyl)-4- | | | | | | | | | | | | | |
| | methylpyrrolidine-3,4- | | | | | | | | | | | | | |
| | diol | | | | | | | | | | | | | |
| 212 | (2S,3S,4R)-2- | pyrrolidine | | | | | | | | | | y | | |
| | (hydroxymethyl)-4- | | | | | | | | | | | | | |
| | methylpyrrolidine-3,4- | | | | | | | | | | | | | |
| | diol | | | | | | | | | | | | | |
| 213 | (1S,5R,8S)-6-oxa-3- | piperidine | | | y | | | | | y | | | | |
| | azabicyclo[3.2.1]octane- | | | | | | | | | | | | | |
| | 1,8-diol | | | | | | | | | | | | | |
| 214 | 2-((2R,3R,4R,5S)-3,4,5- | piperidine | | | | | y | y | | | | | | |
| | trihydroxy-2- | | | | | | | | | | | | | |
| | (hydroxymethyl)piperidin- | | | | | | | | | | | | | |
| | 1-yl)acetic acid | | | | | | | | | | | | | |
| 215 | (2R,3S,4R,5S)-2- | piperidine | | | | y | | | | | | | | |
| | (hydroxymethyl)piperidine- | | | | | | | | | | | | | |
| | 3,4,5-triol | | | | | | | | | | | | | |
| 216 | (2S,3S,4S,5R)-2- | piperidine | | | | | y | y | | | | | | |
| | (hydroxymethyl)piperidine- | | | | | | | | | | | | | |
| | 3,4,5-triol | | | | | | | | | | | | | |
| 217 | (3aS,4R,6aR)-N-benzyl- | pyrrolidine | | | | | | | | | | y | | |
| | 2,2,4- | | | | | | | | | | | | | |
| | trimethyltetrahydro-3aH- | | | | | | | | | | | | | |
| | [1,3]dioxolo[4,5- | | | | | | | | | | | | | |
| | c]pyrrole-4-carboxamide | | | | | | | | | | | | | |
| 218 | (2R,3S,4R)-N-benzyl- | pyrrolidine | | | | | | | | | | y | | |
| | 3,4-dihydroxy-2- | | | | | | | | | | | | | |
| | methylpyrrolidine-2- | | | | | | | | | | | | | |
| | carboxamide | | | | | | | | | | | | | |
| 219 | (3R,4S,5S)-5- | piperidine | | | y | | | | | y | | | | |
| | (hydroxymethyl)piperidine- | | | | | | | | | | | | | |
| | 3,4-diol | | | | | | | | | | | | | |
| 220 | (2S,3S,4R)-1-butyl-2- | pyrrolidine | | | | | | | | | | y | | |
| | (hydroxymethyl)-2- | | | | | | | | | | | | | |
| | methylpyrrolidine-3,4- | | | | | | | | | | | | | |
| | diol | | | | | | | | | | | | | |
| 221 | (2S,3S,4S,5R)-2- | piperidine | | | | | y | y | | | | | | |
| | (hydroxymethyl)piperidine- | | | | | | | | | | | | | |
| | 3,4,5-triol | | | | | | | | | | | | | |
| 222 | (2R,3R,4R,5R)-2-(3,4- | pyrrolidine | | | | | | | | | y | | | |
| | dimethoxyphenyl)-5- | | | | | | | | | | | | | |
| | (hydroxymethyl)pyrrolidine- | | | | | | | | | | | | | |
| | 3,4-diol | | | | | | | | | | | | | |
| 223 | (2S,3R,4S,5R)-2- | piperidine | | | | y | | | | | | | | |
| | (hydroxymethyl)piperidine- | | | | | | | | | | | | | |
| | 3,4,5-triol | | | | | | | | | | | | | |
| 224 | (3R,4R,5R,6S)-2,2- | azepane | | | | | y | y | | | | | | |
| | bis(hydroxymethyl)azepane- | | | | | | | | | | | | | |
| | 3,4,5,6-tetraol | | | | | | | | | | | | | |
| 225 | 1-hydroxy-13- | pyrrolidine | | y | | | | | | | | | y | |
| | ((2R,3R,4S,5R)-4- | | | | | | | | | | | | | |
| | hydroxy-5- | | | | | | | | | | | | | |
| | (hydroxymethyl)-3- | | | | | | | | | | | | | |
| | (3,4,5-trihydroxy-6- | | | | | | | | | | | | | |
| | (hydroxymethyl)tetrahydro- | | | | | | | | | | | | | |
| | 2H-pyran-2- | | | | | | | | | | | | | |
| | yloxy)pyrrolidin-2- | | | | | | | | | | | | | |
| | yl)tridecan-5-one | | | | | | | | | | | | | |
| 226 | (R)-13-((2R,3R,4R,5R)- | pyrrolidine | | | | | | | | | y | | | |
| | 3,4-dihydroxy-5- | | | | | | | | | | | | | |
| | (hydroxymethyl)pyrrolidin- | | | | | | | | | | | | | |
| | 2-yl)-1,13- | | | | | | | | | | | | | |
| | dihydroxytridecan-5-one | | | | | | | | | | | | | |
| 227 | (2R,3S,4R,5S)-2- | piperidine | | | | | | | | | y | | | |
| | (aminomethyl)piperidine- | | | | | | | | | | | | | |
| | 3,4,5-triol | | | | | | | | | | | | | |
| 228 | (4S,5S)-phenyl 3- | piperidine | | | y | | | | | y | | | | |
| | bromo-4,5- | | | | | | | | | | | | | |
| | dihydroxypiperidine-1- | | | | | | | | | | | | | |
| | carboxylate | | | | | | | | | | | | | |
| 229 | (2R,3R,4R,5R)-2- | piperidine | | | | | | | | | y | | | |
| | (hydroxymethyl)piperidine- | | | | | | | | | | | | | |
| | 3,4,5-triol | | | | | | | | | | | | | |
| 230 | (4S,5S)-phenyl 3- | piperidine | | | | | | | | | | y | | |
| | bromo-4,5- | | | | | | | | | | | | | |
| | dihydroxypiperidine-1- | | | | | | | | | | | | | |
| | carboxylate | | | | | | | | | | | | | |
| 231 | (3R,4s,5S)-1- | piperidine | | | | | | | | | | y | | |
| | nonylpiperidine-3,4,5- | | | | | | | | | | | | | |
| | triol | | | | | | | | | | | | | |
| 232 | (3R,4r,5S)-1- | piperidine | | | | | | | | | | | | y |
| | butylpiperidine-3,4,5- | | | | | | | | | | | | | |
| | triol | | | | | | | | | | | | | |
| 233 | (3aS,4R,8R,8aS)-4,8- | azepane | | | | y | | | | | | | | |
| | dihydroxy-2,2- | | | | | | | | | | | | | |
| | dimethyltetrahydro-3aH- | | | | | | | | | | | | | |
| | [1,3]dioxolo[4,5- | | | | | | | | | | | | | |
| | d]azepin-5(4H)-one | | | | | | | | | | | | | |
| 234 | (2S,3S,4S,5S)-4,5- | piperidine | | | | y | | | | | | | | |
| | bis(tert- | | | | | | | | | | | | | |
| | butyldimethylsilyloxy)-2- | | | | | | | | | | | | | |
| | ((tert- | | | | | | | | | | | | | |
| | butyldimethylsilyloxy)methyl)piperidin- | | | | | | | | | | | | | |
| | 3-ol | | | | | | | | | | | | | |
| 235 | 1-((2S,3S,4S)-2-((S)- | pyrrolidine | | | | y | | | | | | | | |
| | 1,2-dihydroxyethyl)-3,4- | | | | | | | | | | | | | |
| | dihydroxypyrrolidin-1- | | | | | | | | | | | | | |
| | yl)ethanone | | | | | | | | | | | | | |
| 236 | (2S,3R)-3,4- | pyrrolidine | | | y | | | | | y | | | | |
| | dihydroxypyrrolidine-2- | | | | | | | | | | | | | |
| | carboxylic acid | | | | | | | | | | | | | |
| 237 | (3aR,6S,7S,7aR)-7- | piperidine | | | | | | | | | y | | | |
| | hydroxy-2,2,6- | | | | | | | | | | | | | |
| | trimethyltetrahydro- | | | | | | | | | | | | | |
| | [1,3]dioxolo[4,5- | | | | | | | | | | | | | |
| | c]pyridin-4(3aH)-one | | | | | | | | | | | | | |
| 238 | (3aS,6R,7R,7aS)-6- | piperidine | | | | | | | | | y | | | |
| | ((tert- | | | | | | | | | | | | | |
| | butyldimethylsilyloxy)methyl)- | | | | | | | | | | | | | |
| | 7-hydroxy-2,2- | | | | | | | | | | | | | |
| | dimethyltetrahydro- | | | | | | | | | | | | | |
| | [1,3]dioxolo[4,5- | | | | | | | | | | | | | |
| | c]pyridin-4(3aH)-one | | | | | | | | | | | | | |
| 239 | (S)-1-((2S,3S,4S)-3,4- | pyrrolidine | | | | y | | | | | | | | |
| | bis(benzyloxy)pyrrolidin- | | | | | | | | | | | | | |
| | 2-yl)ethane-1,2-diol | | | | | | | | | | | | | |
| 240 | 1-((3aS,4S,8R,8aS)-8- | azepane | | y | | | | | | | | | y | |
| | hydroxy-4,7-anhydro- | | | | | | | | | | | | | |
| | 2,2,4-trimethyl-3aH- | | | | | | | | | | | | | |
| | [1,3]dioxolo[4,5- | | | | | | | | | | | | | |
| | c]azepin- | | | | | | | | | | | | | |
| | 5(4H,6H,7H,8H,8aH)- | | | | | | | | | | | | | |
| | yl)ethanone | | | | | | | | | | | | | |
| 241 | (3aS,7R,8R,8aS)-7,8- | azepane | | | | | | | | | y | | | |
| | dihydroxy-2,2- | | | | | | | | | | | | | |
| | dimethyltetrahydro-3aH- | | | | | | | | | | | | | |
| | [1,3]dioxolo[4,5- | | | | | | | | | | | | | |
| | c]azepin-4(5H)-one | | | | | | | | | | | | | |
| 242 | (3S,4S,5R)-3,4- | pyrrolidine | | | | | y | y | | | | | | |
| | bis(benzyloxy)-5-((S)- | | | | | | | | | | | | | |
| | 1,2- | | | | | | | | | | | | | |
| | dihydroxyethyl)pyrrolidin- | | | | | | | | | | | | | |
| | 2-one | | | | | | | | | | | | | |
| 243 | (3S,4S,5R,6R)-3,4,5- | piperidine | | | | | | | | | y | | | |
| | trihydroxy-6- | | | | | | | | | | | | | |
| | methylpiperidin-2-one | | | | | | | | | | | | | |
| 244 | (3aR,4S,7R,7aS)-6- | piperidine | | | | y | y | y | | | | | | |
| | (hydroxymethyl)-2,2,4- | | | | | | | | | | | | | |
| | trimethylhexahydro- | | | | | | | | | | | | | |
| | [1,3]dioxolo[4,5- | | | | | | | | | | | | | |
| | c]pyridin-7-ol | | | | | | | | | | | | | |
| 245 | (2R,3S,4R,5S,6R)-N- | piperidine | | y | | | y | y | | | | | y | |
| | butyl-3,4,5-trihydroxy-6- | | | | | | | | | | | | | |
| | methylpiperidine-2- | | | | | | | | | | | | | |
| | carboxamide | | | | | | | | | | | | | |
| 246 | (2S,3R,4S,5R,6R)- | piperidine | | y | | | | | | | y | | y | |
| | 3,4,5-trihydroxy-6- | | | | | | | | | | | | | |
| | (hydroxymethyl)-N- | | | | | | | | | | | | | |
| | methylpiperidine-2- | | | | | | | | | | | | | |
| | carboxamide | | | | | | | | | | | | | |
| 247 | (2R,3S,4R,5S,6R)-N- | piperidine | | y | | | y | y | | | | | y | |
| | benzyl-3,4,5-trihydroxy- | | | | | | | | | | | | | |
| | 6-methylpiperidine-2- | | | | | | | | | | | | | |
| | carboxamide | | | | | | | | | | | | | |
| 248 | (2S,3R,4S,5R,6R)- | piperidine | | y | | | | | | | y | | y | |
| | 3,4,5-trihydroxy-6- | | | | | | | | | | | | | |
| | (hydroxymethyl)piperidine- | | | | | | | | | | | | | |
| | 2-carboxylic acid | | | | | | | | | | | | | |
| 249 | (2R,3S,4R,5S,6R)- | piperidine | | y | | | y | y | | | | | y | |
| | 3,4,5-trihydroxy-N,6- | | | | | | | | | | | | | |
| | dimethylpiperidine-2- | | | | | | | | | | | | | |
| | carboxamide | | | | | | | | | | | | | |
| 250 | methyl 2-((7R)-7- | piperidine | | | | | | | | | y | | | |
| | hydroxy-2,2-dimethyl-4- | | | | | | | | | | | | | |
| | oxohexahydro- | | | | | | | | | | | | | |
| | [1,3]dioxolo[4,5- | | | | | | | | | | | | | |
| | c]pyridin-6-yl)acetate | | | | | | | | | | | | | |
| 251 | (3aS,4R,8R,8aR,8bS)- | pyrrolizidine | | y | | | | | | | y | | y | |
| | 4-(benzyloxymethyl)-8- | | | | | | | | | | | | | |
| | hydroxy-2,2- | | | | | | | | | | | | | |
| | dimethyltetrahydro-3aH- | | | | | | | | | | | | | |
| | [1,3]dioxolo[4,5- | | | | | | | | | | | | | |
| | a]pyrrolizin-6(4H)-one | | | | | | | | | | | | | |
| 252 | (2S,3R,4R)-1-butyl-2- | piperidine | | | y | | | | | y | | | | |
| | (hydroxymethyl)piperidine- | | | | | | | | | | | | | |
| | 3,4-diol | | | | | | | | | | | | | |
| 253 | (3R,4r,5S)-1- | piperidine | | | | | | | | | | | | y |
| | methylpiperidine-3,4,5- | | | | | | | | | | | | | |
| | triol | | | | | | | | | | | | | |
| 254 | (3R,4r,5S)-1- | piperidine | | | | | | | | | | | | y |
| | nonylpiperidine-3,4,5- | | | | | | | | | | | | | |
| | triol | | | | | | | | | | | | | |
| 255 | (2S,3S,4S)-1-benzyl-2- | pyrrolidine | | | | y | | | | | | | | |
| | ((S)-1,2- | | | | | | | | | | | | | |
| | dihydroxyethyl)pyrrolidine- | | | | | | | | | | | | | |
| | 3,4-diol | | | | | | | | | | | | | |
| 256 | (2S,5S)-2- | piperidine | | y | | | | | | | y | | y | |
| | (hydroxymethyl)-6- | | | | | | | | | | | | | |
| | methylpiperidine-3,4,5- | | | | | | | | | | | | | |
| | triol | | | | | | | | | | | | | |
| 257 | (2S,3R,4S,5R)-2- | piperidine | | | | y | | | | | | | | |
| | methylpiperidine-3,4,5- | | | | | | | | | | | | | |
| | triol | | | | | | | | | | | | | |
| 258 | (2R,3S,4R,5S)-2- | piperidine | | | | y | | | | | | | | |
| | methylpiperidine-3,4,5- | | | | | | | | | | | | | |
| | triol | | | | | | | | | | | | | |
| 259 | (2R,3R,4R,5R)-2- | piperidine | | | | | | | | | y | | | |
| | methylpiperidine-3,4,5- | | | | | | | | | | | | | |
| | triol | | | | | | | | | | | | | |
| 260 | (3S,4S,5R,6S)-3,4,5- | piperidine | | | | | y | y | | | | | | |
| | trihydroxy-6- | | | | | | | | | | | | | |
| | (hydroxymethyl)piperidin- | | | | | | | | | | | | | |
| | 2-one | | | | | | | | | | | | | |
| 261 | (2R,4S,5R)-2-(2- | pyrrolidine | | | | | y | y | | | | | | |
| | hydroxyethyl)-5- | | | | | | | | | | | | | |
| | (hydroxymethyl)pyrrolidine- | | | | | | | | | | | | | |
| | 3,4-diol | | | | | | | | | | | | | |
| 262 | (2S,3R,4R)-3,4- | pyrrolidine | | | y | | | | | y | | | | |
| | dihydroxypyrrolidine-2- | | | | | | | | | | | | | |
| | carboxylic acid | | | | | | | | | | | | | |
| 263 | (2R,3S,4R,5S,6R)-2- | piperidine | | | | y | y | y | | | | | | |
| | (hydroxymethyl)-6- | | | | | | | | | | | | | |
| | methylpiperidine-3,4,5- | | | | | | | | | | | | | |
| | triol | | | | | | | | | | | | | |
| 264 | (2S,3S,4R,5S,6R)-2- | piperidine | | y | | | y | y | | | | | y | |
| | (hydroxymethyl)-6- | | | | | | | | | | | | | |
| | methylpiperidine-3,4,5- | | | | | | | | | | | | | |
| | triol | | | | | | | | | | | | | |
| 265 | (3S,4R,5S,6S)-N-butyl- | piperidine | | y | | | | | | | y | | y | |
| | 3,4,5-trihydroxy-6- | | | | | | | | | | | | | |
| | methylpiperidine-2- | | | | | | | | | | | | | |
| | carboxamide | | | | | | | | | | | | | |
| 266 | (1R,2S,6R,7S,7ar)- | pyrrolizidine | y | | | | | | | | | | | |
| | hexahydro-1H- | | | | | | | | | | | | | |
| | pyrrolizine-1,2,6,7- | | | | | | | | | | | | | |
| | tetraol | | | | | | | | | | | | | |
| 267 | (3S,4R,5S,6S)-N- | piperidine | | y | | | | | | | y | | y | |
| | benzyl-3,4,5-trihydroxy- | | | | | | | | | | | | | |
| | 6-methylpiperidine-2- | | | | | | | | | | | | | |
| | carboxamide | | | | | | | | | | | | | |
| 268 | (2S,3S,4S,5S)-2- | piperidine | | | | | | | | | y | | | |
| | methylpiperidine-3,4,5- | | | | | | | | | | | | | |
| | triol | | | | | | | | | | | | | |
| 269 | (2S,3S,4R,5S,6S)-2- | piperidine | | | | y | | | | | y | | | |
| | (hydroxymethyl)-6- | | | | | | | | | | | | | |
| | methylpiperidine-3,4,5- | | | | | | | | | | | | | |
| | triol | | | | | | | | | | | | | |
| 270 | 1- | azepane | | | y | | | | | y | | | | |
| | ((1R,2S,3S,4S,5S,7R)- | | | | | | | | | | | | | |
| | 2,3,4-trihydroxy-5- | | | | | | | | | | | | | |
| | methyl-7- | | | | | | | | | | | | | |
| | ((2R,3S,4R,5S,6S)- | | | | | | | | | | | | | |
| | 3,4,5-trihydroxy-6- | | | | | | | | | | | | | |
| | (hydroxymethyl)tetrahydro- | | | | | | | | | | | | | |
| | 2H-pyran-2-yloxy)-8- | | | | | | | | | | | | | |
| | oxa-6- | | | | | | | | | | | | | |
| | azabicyclo[3.2.1]octan- | | | | | | | | | | | | | |
| | 6-yl)ethanone | | | | | | | | | | | | | |
| 271 | (1R,2R,3R,6S,7S,7aR)- | pyrrolizidine | | y | | | | | | | y | | y | |
| | 5-gem-dideuterio-3- | | | | | | | | | | | | | |
| | (hydroxymethyl)hexahydro- | | | | | | | | | | | | | |
| | 1H-pyrrolizine- | | | | | | | | | | | | | |
| | 1,2,6,7-tetraol | | | | | | | | | | | | | |
| 272 | (3S,4s,5R)-1- | piperidine | | | | | | | | | | y | | |
| | butylpiperidine-3,4,5- | | | | | | | | | | | | | |
| | triol | | | | | | | | | | | | | |
| 273 | (3R,5R)-piperidine- | piperidine | | | y | | | | | y | | | | |
| | 3,4,5-triol | | | | | | | | | | | | | |
| 274 | ((2S,4S)-4- | pyrrolidine | | | y | | | | | y | | | | |
| | azidopyrrolidin-2- | | | | | | | | | | | | | |
| | yl)methanol | | | | | | | | | | | | | |
| 275 | ((2S,4S)-4-azido-1- | pyrrolidine | | | y | | | | | y | | | | |
| | butylpyrrolidin-2- | | | | | | | | | | | | | |
| | yl)methanol | | | | | | | | | | | | | |
| 276 | (2R,3R,4R,5S)-1-(4- | piperidine | | | | | y | y | | | | | | |
| | hydroxybutyl)-2- | | | | | | | | | | | | | |
| | (hydroxymethyl)piperidine- | | | | | | | | | | | | | |
| | 3,4,5-triol | | | | | | | | | | | | | |
| 277 | 2-((2S,4S)-4-azido-2- | pyrrolidine | | | y | | | | | y | | | | |
| | (hydroxymethyl)pyrrolidin- | | | | | | | | | | | | | |
| | 1-yl)ethanol | | | | | | | | | | | | | |
| 278 | (2R,3R,3aR,5S,6R,7R,7aS)- | piperidine | | y | | | | | | | | | y | |
| | 3-((R)-1- | | | | | | | | | | | | | |
| | hydroxybutyl)-5- | | | | | | | | | | | | | |
| | (hydroxymethyl)octahydrofuro[3, | | | | | | | | | | | | | |
| | 2-b]pyridine- | | | | | | | | | | | | | |
| | 2,6,7-triol | | | | | | | | | | | | | |
| 279 | (3R,4R,5R)-5- | | | | | | | | | | | | | y |
| | (hydroxymethyl)piperidine- | | | | | | | | | | | | | |
| | 3,4-diol | | | | | | | | | | | | | |
| 280 | (3R,5S)-1-(2- | pyrrolidine | | | | | | | | | | y | | y |
| | hydroxyethyl)-5- | | | | | | | | | | | | | |
| | (hydroxymethyl)pyrrolidin- | | | | | | | | | | | | | |
| | 3-ol | | | | | | | | | | | | | |
| 281 | (3R,5R)-3,4,5- | piperidine | | | y | | | | | y | | | | |
| | trihydroxypiperidine-1- | | | | | | | | | | | | | |
| | carbaldehyde | | | | | | | | | | | | | |
| 282 | (3S,5S)-piperidine- | piperidine | | | y | | | | | y | | | | |
| | 3,4,5-triol | | | | | | | | | | | | | |
| 283 | (3R,5S)-5- | pyrrolidine | | | | | | | | | | y | | y |
| | (hydroxymethyl)pyrrolidin- | | | | | | | | | | | | | |
| | 3-ol | | | | | | | | | | | | | |
| 284 | ((2S,4S)-4-azido-1- | pyrrolidine | | | y | | | | | y | | | | |
| | nonylpyrrolidin-2- | | | | | | | | | | | | | |
| | yl)methanol | | | | | | | | | | | | | |
| 285 | (3R,5S)-5- | pyrrolidine | | | | | | | | | | y | | y |
| | (aminomethyl)-1-(2- | | | | | | | | | | | | | |
| | hydroxyethyl)pyrrolidin- | | | | | | | | | | | | | |
| | 3-ol | | | | | | | | | | | | | |
| 286 | (3R,5S)-5- | pyrrolidine | | | | | | | | | | y | | y |
| | (azidomethyl)-1- | | | | | | | | | | | | | |
| | butylpyrrolidin-3-ol | | | | | | | | | | | | | |
| 287 | (3R,5S)-5- | pyrrolidine | | | | | | | | | | y | | y |
| | (azidomethyl)-1-(2- | | | | | | | | | | | | | |
| | hydroxyethyl)pyrrolidin- | | | | | | | | | | | | | |
| | 3-ol | | | | | | | | | | | | | |
| 288 | (2R,3R,4R,5S)-2- | piperidine | | | | | y | y | | | | | | |
| | (hydroxymethyl)-1-(3- | | | | | | | | | | | | | |
| | phenoxypropyl)piperidine- | | | | | | | | | | | | | |
| | 3,4,5-triol | | | | | | | | | | | | | |
| 289 | (3R,5S)-5- | pyrrolidine | | | | | | | | | | y | | y |
| | (aminomethyl)-1- | | | | | | | | | | | | | |
| | butylpyrrolidin-3-ol | | | | | | | | | | | | | |
| 290 | 2-((2S,4S)-4-amino-2- | pyrrolidine | | | y | | | | | y | | | | |
| | (hydroxymethyl)pyrrolidin- | | | | | | | | | | | | | |
| | 1-yl)ethanol | | | | | | | | | | | | | |
| 291 | diethyl 3-((2S,4S)-4- | pyrrolidine | | | y | | | | | y | | | | |
| | azido-2- | | | | | | | | | | | | | |
| | (hydroxymethyl)pyrrolidin- | | | | | | | | | | | | | |
| | 1- | | | | | | | | | | | | | |
| | yl)propylphosphonate | | | | | | | | | | | | | |
| 292 | ((2S,4S)-4-amino-1- | pyrrolidine | | | y | | | | | y | | | | |
| | butylpyrrolidin-2- | | | | | | | | | | | | | |
| | yl)methanol | | | | | | | | | | | | | |
| 293 | (3R,5S)-1-(2- | pyrrolidine | | | | | | | | | | y | | y |
| | hydroxyethyl)-5- | | | | | | | | | | | | | |
| | (morpholinomethyl)pyrrolidin- | | | | | | | | | | | | | |
| | 3-ol | | | | | | | | | | | | | |
| 294 | (3R,5S)-5- | pyrrolidine | | | | | | | | | | y | | y |
| | (hydroxymethyl)-1- | | | | | | | | | | | | | |
| | nonylpyrrolidin-3-ol | | | | | | | | | | | | | |
| 295 | ((2S,4S)-4-amino-1- | pyrrolidine | | | y | | | | | y | | | | |
| | nonylpyrrolidin-2- | | | | | | | | | | | | | |
| | yl)methanol | | | | | | | | | | | | | |
| 296 | (3R,5R)-1- | piperidine | | | y | | | | | y | | | | |
| | butylpiperidine-3,4,5- | | | | | | | | | | | | | |
| | triol | | | | | | | | | | | | | |
| 297 | (3R,5R)-1- | piperidine | | | y | | | | | y | | | | |
| | methylpiperidine-3,4,5- | | | | | | | | | | | | | |
| | triol | | | | | | | | | | | | | |
| 298 | (3S,5S)-1- | piperidine | | | y | | | | | y | | | | |
| | butylpiperidine-3,4,5- | | | | | | | | | | | | | |
| | triol | | | | | | | | | | | | | |
| 299 | (3S,5S)-1- | piperidine | | | y | | | | | y | | | | |
| | methylpiperidine-3,4,5- | | | | | | | | | | | | | |
| | triol | | | | | | | | | | | | | |
| 300 | (2S,3S,4S,5S)-2- | pyrrolidine | | | | | | | | | y | | | |
| | (hydroxymethyl)-5- | | | | | | | | | | | | | |
| | methylpyrrolidine-3,4- | | | | | | | | | | | | | |
| | diol | | | | | | | | | | | | | |
| 301 | (3S,4s,5R)-piperidine- | piperidine | | | | | | | | | | y | | |
| | 3,4,5-triol | | | | | | | | | | | | | |
| 302 | (3S,5S)-1- | piperidine | | | y | | | | | y | | | | |
| | nonylpiperidine-3,4,5- | | | | | | | | | | | | | |
| | triol | | | | | | | | | | | | | |
| 303 | (3R,5R)-tert-butyl 3,4,5- | piperidine | | | y | | | | | y | | | | |
| | trihydroxypiperidine-1- | | | | | | | | | | | | | |
| | carboxylate | | | | | | | | | | | | | |
| 304 | (2R,3S,4S)-1-(2- | pyrrolidine | | | | | | | | | | | | y |
| | hydroxyethyl)-2- | | | | | | | | | | | | | |
| | (hydroxymethyl)pyrrolidine- | | | | | | | | | | | | | |
| | 3,4-diol | | | | | | | | | | | | | |
| 305 | (2R,3S,4S)-2- | pyrrolidine | | | | | | | | | | | | y |
| | (hydroxymethyl)pyrrolidine- | | | | | | | | | | | | | |
| | 3,4-diol | | | | | | | | | | | | | |
| 306 | (2R,3S,4S)-1-butyl-2- | pyrrolidine | | | | | | | | | | | | y |
| | (hydroxymethyl)pyrrolidine- | | | | | | | | | | | | | |
| | 3,4-diol | | | | | | | | | | | | | |
| 307 | (2R,3R,4S)-1-benzyl-2- | pyrrolidine | y | | | | | | | | | | | |
| | ((S)-1,2- | | | | | | | | | | | | | |
| | dihydroxyethyl)pyrrolidine- | | | | | | | | | | | | | |
| | 3,4-diol | | | | | | | | | | | | | |
| 308 | (2S,3S,4S)-4-azido-1- | pyrrolidine | | | y | | | | | y | | | | |
| | benzyl-2- | | | | | | | | | | | | | |
| | (hydroxymethyl)pyrrolidin- | | | | | | | | | | | | | |
| | 3-ol | | | | | | | | | | | | | |
| 309 | N-((3S,4R,5S)-1-benzyl- | pyrrolidine | | | y | | | | | y | | | | |
| | 4-hydroxy-5- | | | | | | | | | | | | | |
| | (hydroxymethyl)pyrrolidin- | | | | | | | | | | | | | |
| | 3-yl)acetamide | | | | | | | | | | | | | |
| 310 | (2R,3R,4S)-2- | pyrrolidine | | | | | | | | | | y | | |
| | (hydroxymethyl)pyrrolidine- | | | | | | | | | | | | | |
| | 3,4-diol | | | | | | | | | | | | | |
| 311 | (2R,3R,4S)-1-benzyl-2- | pyrrolidine | | | | | | | | | | y | | |
| | (hydroxymethyl)pyrrolidine- | | | | | | | | | | | | | |
| | 3,4-diol | | | | | | | | | | | | | |
| 312 | (2S,3R,4S)-4-amino-1- | pyrrolidine | | | y | | | | | y | | | | |
| | benzyl-2- | | | | | | | | | | | | | |
| | (hydroxymethyl)pyrrolidin- | | | | | | | | | | | | | |
| | 3-ol | | | | | | | | | | | | | |
| 313 | (2S,3R,4S)-4- | pyrrolidine | | | y | | | | | y | | | | |
| | acetamido-2- | | | | | | | | | | | | | |
| | (acetoxymethyl)-1- | | | | | | | | | | | | | |
| | benzylpyrrolidin-3-yl | | | | | | | | | | | | | |
| | acetate | | | | | | | | | | | | | |
| 314 | (2S,3S,4R)-1-butyl-2- | pyrrolidine | y | | | | | | | | | | | |
| | ((R)-1,2- | | | | | | | | | | | | | |
| | dihydroxyethyl)pyrrolidine- | | | | | | | | | | | | | |
| | 3,4-diol | | | | | | | | | | | | | |
| 315 | (2R,3R,4S)-2-((S)-1,2- | pyrrolidine | y | | | | | | | | | | | |
| | dihydroxyethyl)pyrrolidine- | | | | | | | | | | | | | |
| | 3,4-diol | | | | | | | | | | | | | |
| 316 | (2S,3S,4R)-2-((R)-1,2- | pyrrolidine | y | | | | | | | | | | | |
| | dihydroxyethyl)-1- | | | | | | | | | | | | | |
| | nonylpyrrolidine-3,4-diol | | | | | | | | | | | | | |
| 317 | (2R,3R,4S)-1-butyl-2- | pyrrolidine | | | | | | | | | | y | | |
| | (hydroxymethyl)pyrrolidine- | | | | | | | | | | | | | |
| | 3,4-diol | | | | | | | | | | | | | |
| 318 | N-((3S,4R,5S)-4- | pyrrolidine | | | y | | | | | y | | | | |
| | hydroxy-5- | | | | | | | | | | | | | |
| | (hydroxymethyl)pyrrolidin- | | | | | | | | | | | | | |
| | 3-yl)acetamide | | | | | | | | | | | | | |
| 319 | N-((3S,4R,5S)-1-butyl- | pyrrolidine | | | y | | | | | y | | | | |
| | 4-hydroxy-5- | | | | | | | | | | | | | |
| | (hydroxymethyl)pyrrolidin- | | | | | | | | | | | | | |
| | 3-yl)acetamide | | | | | | | | | | | | | |
| 320 | (2S,3R,4R)-1- | pyrrolidine | | | | | | | | | | | | y |
| | (cyclohexylmethyl)-2- | | | | | | | | | | | | | |
| | (hydroxymethyl)pyrrolidine- | | | | | | | | | | | | | |
| | 3,4-diol | | | | | | | | | | | | | |
| 321 | (2S,3R,4R)-1-(2- | pyrrolidine | | | | | | | | | | | | y |
| | hydroxyethyl)-2- | | | | | | | | | | | | | |
| | (hydroxymethyl)pyrrolidine- | | | | | | | | | | | | | |
| | 3,4-diol | | | | | | | | | | | | | |
| 322 | (1R,2R)-1-((2R,3R,4S)- | pyrrolidine | | | | y | | | | | y | | | |
| | 1-butyl-3,4- | | | | | | | | | | | | | |
| | dihydroxypyrrolidin-2- | | | | | | | | | | | | | |
| | yl)propane-1,2,3-triol | | | | | | | | | | | | | |
| 323 | (1R,2R)-1-((2R,3R,4S)- | pyrrolidine | | | | y | | | | | y | | | |
| | 3,4-dihydroxypyrrolidin- | | | | | | | | | | | | | |
| | 2-yl)propane-1,2,3-triol | | | | | | | | | | | | | |
| 324 | (1S,2R)-1-((2R,3R,4S)- | pyrrolidine | y | | | | | | | | | | | |
| | 3,4-dihydroxy-1- | | | | | | | | | | | | | |
| | nonylpyrrolidin-2- | | | | | | | | | | | | | |
| | yl)propane-1,2,3-triol | | | | | | | | | | | | | |
| 325 | (1S,2R)-1-((2R,3R,4S)- | pyrrolidine | y | | | | | | | | | | | |
| | 1-butyl-3,4- | | | | | | | | | | | | | |
| | dihydroxypyrrolidin-2- | | | | | | | | | | | | | |
| | yl)propane-1,2,3-triol | | | | | | | | | | | | | |
| 326 | (1S,2R)-1-((2R,3R,4S)- | pyrrolidine | y | | | | | | | | | | | |
| | 3,4-dihydroxy-1-(2- | | | | | | | | | | | | | |
| | hydroxyethyl)pyrrolidin- | | | | | | | | | | | | | |
| | 2-yl)propane-1,2,3-triol | | | | | | | | | | | | | |
| 327 | (1R,2R)-1-((2R,3R,4S)- | pyrrolidine | | | | y | | | | | y | | | |
| | 3,4-dihydroxy-1-(2- | | | | | | | | | | | | | |
| | hydroxyethyl)pyrrolidin- | | | | | | | | | | | | | |
| | 2-yl)propane-1,2,3-triol | | | | | | | | | | | | | |
| 328 | (1R,2R)-1-((2R,3R,4S)- | pyrrolidine | | | | y | | | | | y | | | |
| | 1-benzyl-3,4- | | | | | | | | | | | | | |
| | dihydroxypyrrolidin-2- | | | | | | | | | | | | | |
| | yl)propane-1,2,3-triol | | | | | | | | | | | | | |
| 329 | (1S,2R)-1-((2R,3R,4S)- | pyrrolidine | y | | | | | | | | | | | |
| | 1-benzyl-3,4- | | | | | | | | | | | | | |
| | dihydroxypyrrolidin-2- | | | | | | | | | | | | | |
| | yl)propane-1,2,3-triol | | | | | | | | | | | | | |
| 330 | ((2S,4S)-4-acetamido-1- | pyrrolidine | | | y | | | | | y | | | | |
| | (2- | | | | | | | | | | | | | |
| | acetoxyethyl)pyrrolidin- | | | | | | | | | | | | | |
| | 2-yl)methyl acetate | | | | | | | | | | | | | |
| 331 | ((2S,4S)-4-acetamido-1- | pyrrolidine | | | y | | | | | y | | | | |
| | butylpyrrolidin-2- | | | | | | | | | | | | | |
| | yl)methyl acetate | | | | | | | | | | | | | |
| 332 | ((2S,4S)-4-acetamido-1- | pyrrolidine | | | y | | | | | y | | | | |
| | nonylpyrrolidin-2- | | | | | | | | | | | | | |
| | yl)methyl acetate | | | | | | | | | | | | | |
| 333 | (1R,2S,8R,8aR)- | indolizidine | y | | | | | | | | | | | |
| | octahydroindolizine- | | | | | | | | | | | | | |
| | 1,2,8-triol | | | | | | | | | | | | | |
| 334 | (2S,3S,4R)-2-((R)-1,2- | pyrrolidine | y | | | | | | | | | | | |
| | dihydroxyethyl)-1-(2- | | | | | | | | | | | | | |
| | hydroxyethyl)pyrrolidine- | | | | | | | | | | | | | |
| | 3,4-diol | | | | | | | | | | | | | |
| 335 | N-((3S,4R,5S)-4- | pyrrolidine | | | y | | | | | y | | | | |
| | hydroxy-1-(2- | | | | | | | | | | | | | |
| | hydroxyethyl)-5- | | | | | | | | | | | | | |
| | (hydroxymethyl)pyrrolidin- | | | | | | | | | | | | | |
| | 3-yl)acetamide | | | | | | | | | | | | | |
| 336 | (1S,2R)-1-((2S,3R,4S)- | pyrrolidine | y | | | | | | | | | | | |
| | 3,4-dihydroxypyrrolidin- | | | | | | | | | | | | | |
| | 2-yl)propane-1,2,3-triol | | | | | | | | | | | | | |
| 337 | (1R,2S,8R,8aS)-1,2,8- | indolizidine | y | | | | | | | | | | | |
| | trihydroxyhexahydroindolizin- | | | | | | | | | | | | | |
| | 5(1H)-one | | | | | | | | | | | | | |
| 338 | N-((3S,4R,5S)-4- | pyrrolidine | | | y | | | | | y | | | | |
| | hydroxy-5- | | | | | | | | | | | | | |
| | (hydroxymethyl)-1- | | | | | | | | | | | | | |
| | nonylpyrrolidin-3- | | | | | | | | | | | | | |
| | yl)acetamide | | | | | | | | | | | | | |
| 339 | (2R,3R,4S)-1-butyl-2- | pyrrolidine | y | | | | | | | | | | | |
| | ((S)-1,2- | | | | | | | | | | | | | |
| | dihydroxyethyl)pyrrolidine- | | | | | | | | | | | | | |
| | 3,4-diol | | | | | | | | | | | | | |
| 340 | (2R,3R,4S)-2-((S)-1,2- | pyrrolidine | y | | | | | | | | | | | |
| | dihydroxyethyl)-1-(2- | | | | | | | | | | | | | |
| | hydroxyethyl)pyrrolidine- | | | | | | | | | | | | | |
| | 3,4-diol | | | | | | | | | | | | | |
| 341 | 2-((2S,3S,4R)-2-((R)- | pyrrolidine | y | | | | | | | | | | | |
| | 1,2-dihydroxyethyl)-3,4- | | | | | | | | | | | | | |
| | dihydroxypyrrolidin-1- | | | | | | | | | | | | | |
| | yl)acetic acid | | | | | | | | | | | | | |
| 342 | (2S,3S,4R)-1-benzyl-2- | pyrrolidine | | | | | | | | | | | y | |
| | (hydroxymethyl)pyrrolidine- | | | | | | | | | | | | | |
| | 3,4-diol | | | | | | | | | | | | | |
| 343 | (2R,3R,4R,5R)-2,5- | pyrrolidine | | | | | | | | | y | | | |
| | bis(hydroxymethyl)-1-(3- | | | | | | | | | | | | | |
| | phenoxypropyl)pyrrolidine- | | | | | | | | | | | | | |
| | 3,4-diol | | | | | | | | | | | | | |
| 344 | ((2S,4S)-4- | pyrrolidine | | | y | | | | | y | | | | |
| | aminopyrrolidin-2- | | | | | | | | | | | | | |
| | yl)methanol | | | | | | | | | | | | | |
| 345 | (2S,4S)-4- | pyrrolidine | | | y | | | | | y | | | | |
| | azidopyrrolidine-2- | | | | | | | | | | | | | |
| | carboxylic acid | | | | | | | | | | | | | |
| 346 | N-((3S,5S)-5- | pyrrolidine | | | y | | | | | y | | | | |
| | (hydroxymethyl)pyrrolidin- | | | | | | | | | | | | | |
| | 3-yl)acetamide | | | | | | | | | | | | | |
| 347 | N-((3S,5S)-1-(2- | pyrrolidine | | | y | | | | | y | | | | |
| | hydroxyethyl)-5- | | | | | | | | | | | | | |
| | (hydroxymethyl)pyrrolidin- | | | | | | | | | | | | | |
| | 3-yl)acetamide | | | | | | | | | | | | | |
| 348 | N-((3S,5S)-5- | pyrrolidine | | | y | | | | | y | | | | |
| | (hydroxymethyl)-1- | | | | | | | | | | | | | |
| | nonylpyrrolidin-3- | | | | | | | | | | | | | |
| | yl)acetamide | | | | | | | | | | | | | |
| 349 | (2R,3R,4R)-2- | pyrrolidine | | | y | | | | | y | | | | |
| | (hydroxymethyl)-1-(3- | | | | | | | | | | | | | |
| | phenoxypropyl)pyrrolidine- | | | | | | | | | | | | | |
| | 3,4-diol | | | | | | | | | | | | | |
| 350 | N-((3S,5S)-1-butyl-5- | pyrrolidine | | | y | | | | | y | | | | |
| | (hydroxymethyl)pyrrolidin- | | | | | | | | | | | | | |
| | 3-yl)acetamide | | | | | | | | | | | | | |
| 351 | (2S,3R,4S)-1-benzyl-2- | pyrrolidine | | | | | | | | | y | | | |
| | ((R)-1,2- | | | | | | | | | | | | | |
| | dihydroxyethyl)pyrrolidine- | | | | | | | | | | | | | |
| | 3,4-diol | | | | | | | | | | | | | |
| 352 | (1S,2S,6S,7R,8R,8aR)- | indolizidine | | y | | | y | y | | | | | y | |
| | octahydroindolizine- | | | | | | | | | | | | | |
| | 1,2,6,7,8-pentaol | | | | | | | | | | | | | |
| 353 | N-((3S,4R,5S)-4,5- | piperidine | | | y | | | | | y | | | | |
| | dihydroxypiperidin-3- | | | | | | | | | | | | | |
| | yl)acetamide | | | | | | | | | | | | | |
| 354 | (3R,5R)-benzyl 3,4,5- | piperidine | | | y | | | | | y | | | | |
| | trihydroxypiperidine-1- | | | | | | | | | | | | | |
| | carboxylate | | | | | | | | | | | | | |
| 355 | 2-((2S,4S)-4-azido-2- | pyrrolidine | | | y | | | | | y | | | | |
| | (hydroxymethyl)pyrrolidin- | | | | | | | | | | | | | |
| | 1-yl)acetic acid | | | | | | | | | | | | | |
| 356 | (1R,2S,3S,7R,7aR)-3- | pyrrolizidine | y | | | | | | | | | | | |
| | (hydroxymethyl)hexahydro- | | | | | | | | | | | | | |
| | 1H-pyrrolizine-1,2,7- | | | | | | | | | | | | | |
| | triol | | | | | | | | | | | | | |
| 357 | (2R,3S,4S)-2- | piperidine | | | | | | | | | | | | y |
| | (hydroxymethyl)piperidine- | | | | | | | | | | | | | |
| | 3,4-diol | | | | | | | | | | | | | |
| 358 | 2-((2S,3R,4S)-4- | pyrrolidine | | | y | | | | | y | | | | |
| | acetamido-3-hydroxy-2- | | | | | | | | | | | | | |
| | (hydroxymethyl)pyrrolidin- | | | | | | | | | | | | | |
| | 1-yl)acetic acid | | | | | | | | | | | | | |
| 359 | 2-((2R,3R,4S)-2-((S)- | pyrrolidine | y | | | | | | | | | | | |
| | 1,2-dihydroxyethyl)-3,4- | | | | | | | | | | | | | |
| | dihydroxypyrrolidin-1- | | | | | | | | | | | | | |
| | yl)acetic acid | | | | | | | | | | | | | |
| 360 | (2S,3S,4R)-1-butyl-2- | pyrrolidine | | | | | | | | | | y | | |
| | (hydroxymethyl)pyrrolidine- | | | | | | | | | | | | | |
| | 3,4-diol | | | | | | | | | | | | | |
| 361 | 2-((2R,3R,4S)-3,4- | pyrrolidine | | | | | | | | | | y | | |
| | dihydroxy-2- | | | | | | | | | | | | | |
| | (hydroxymethyl)pyrrolidin- | | | | | | | | | | | | | |
| | 1-yl)acetic acid | | | | | | | | | | | | | |
| 362 | (2R,3S,4R)-4- | pyrrolidine | | | y | | | | | y | | | | |
| | acetamido-2- | | | | | | | | | | | | | |
| | (acetoxymethyl)-1- | | | | | | | | | | | | | |
| | benzylpyrrolidin-3-yl | | | | | | | | | | | | | |
| | acetate | | | | | | | | | | | | | |
| 363 | (2R,3R,4R)-4-azido-1- | pyrrolidine | | | y | | | | | y | | | | |
| | benzyl-2- | | | | | | | | | | | | | |
| | (hydroxymethyl)pyrrolidin- | | | | | | | | | | | | | |
| | 3-ol | | | | | | | | | | | | | |
| 364 | N-((3R,4S,5R)-1-benzyl- | pyrrolidine | | | y | | | | | y | | | | |
| | 4-hydroxy-5- | | | | | | | | | | | | | |
| | (hydroxymethyl)pyrrolidin- | | | | | | | | | | | | | |
| | 3-yl)acetamide | | | | | | | | | | | | | |
| 365 | N-((3R,4S,5R)-4- | pyrrolidine | | | y | | | | | y | | | | |
| | hydroxy-5- | | | | | | | | | | | | | |
| | (hydroxymethyl)pyrrolidin- | | | | | | | | | | | | | |
| | 3-yl)acetamide | | | | | | | | | | | | | |
| 366 | 2-((2R,3S,4R)-4- | pyrrolidine | | | y | | | | | y | | | | |
| | acetamido-3-hydroxy-2- | | | | | | | | | | | | | |
| | (hydroxymethyl)pyrrolidin- | | | | | | | | | | | | | |
| | 1-yl)acetic acid | | | | | | | | | | | | | |
| 367 | N-((3R,4S,5R)-4- | pyrrolidine | | | y | | | | | y | | | | |
| | hydroxy-5- | | | | | | | | | | | | | |
| | (hydroxymethyl)-1- | | | | | | | | | | | | | |
| | isopropylpyrrolidin-3- | | | | | | | | | | | | | |
| | yl)acetamide | | | | | | | | | | | | | |
| 368 | 2-((2S,3S,4R)-3,4- | pyrrolidine | | | | | | | | | | y | | |
| | dihydroxy-2- | | | | | | | | | | | | | |
| | (hydroxymethyl)pyrrolidin- | | | | | | | | | | | | | |
| | 1-yl)acetic acid | | | | | | | | | | | | | |
| 369 | N-((3R,4S,5R)-4- | pyrrolidine | | | y | | | | | y | | | | |
| | hydroxy-1-(2- | | | | | | | | | | | | | |
| | hydroxyethyl)-5- | | | | | | | | | | | | | |
| | (hydroxymethyl)pyrrolidin- | | | | | | | | | | | | | |
| | 3-yl)acetamide | | | | | | | | | | | | | |
| 370 | (2R,3S,4R)-4-amino-1- | pyrrolidine | | | y | | | | | y | | | | |
| | benzyl-2- | | | | | | | | | | | | | |
| | (hydroxymethyl)pyrrolidin- | | | | | | | | | | | | | |
| | 3-ol | | | | | | | | | | | | | |
| 371 | (2S,3S,4S,5S)-3,4- | pyrrolidine | | | | | | | | | y | | | |
| | dihydroxy-2,5- | | | | | | | | | | | | | |
| | bis(hydroxymethyl)pyrrolidine- | | | | | | | | | | | | | |
| | 1-carbaldehyde | | | | | | | | | | | | | |
| 372 | N-((3R,4R,5S)-1-benzyl- | pyrrolidine | | | | | | | | | | y | | |
| | 4-hydroxy-5- | | | | | | | | | | | | | |
| | (hydroxymethyl)pyrrolidin- | | | | | | | | | | | | | |
| | 3-yl)acetamide | | | | | | | | | | | | | |
| 373 | (2S,3R,4R)-4-amino-1- | pyrrolidine | | | | | | | | | | y | | |
| | benzyl-2- | | | | | | | | | | | | | |
| | (hydroxymethyl)pyrrolidin- | | | | | | | | | | | | | |
| | 3-ol | | | | | | | | | | | | | |
| 374 | (2S,3S,4R)-4-azido-1- | pyrrolidine | | | | | | | | | | y | | |
| | benzyl-2- | | | | | | | | | | | | | |
| | (hydroxymethyl)pyrrolidin- | | | | | | | | | | | | | |
| | 3-ol | | | | | | | | | | | | | |
| 375 | N-((3R,4R,5S)-4- | pyrrolidine | | | | | | | | | | y | | |
| | hydroxy-1-(2- | | | | | | | | | | | | | |
| | hydroxyethyl)-5- | | | | | | | | | | | | | |
| | (hydroxymethyl)pyrrolidin- | | | | | | | | | | | | | |
| | 3-yl)acetamide | | | | | | | | | | | | | |
| 376 | (2S,3R,4R)-4- | pyrrolidine | | | | | | | | | | y | | |
| | acetamido-2- | | | | | | | | | | | | | |
| | (acetoxymethyl)-1- | | | | | | | | | | | | | |
| | benzylpyrrolidin-3-yl | | | | | | | | | | | | | |
| | acetate | | | | | | | | | | | | | |
| 377 | 2-((2S,3R,4R)-4- | pyrrolidine | | | | | | | | | | y | | |
| | acetamido-3-hydroxy-2- | | | | | | | | | | | | | |
| | (hydroxymethyl)pyrrolidin- | | | | | | | | | | | | | |
| | 1-yl)acetic acid | | | | | | | | | | | | | |
| 378 | N-((3R,4R,5S)-4- | pyrrolidine | | | | | | | | | | y | | |
| | hydroxy-5- | | | | | | | | | | | | | |
| | (hydroxymethyl)pyrrolidin- | | | | | | | | | | | | | |
| | 3-yl)acetamide | | | | | | | | | | | | | |
| 379 | N-((3R,4R,5S)-1-butyl- | pyrrolidine | | | | | | | | | | y | | |
| | 4-hydroxy-5- | | | | | | | | | | | | | |
| | (hydroxymethyl)pyrrolidin- | | | | | | | | | | | | | |
| | 3-yl)acetamide | | | | | | | | | | | | | |
| 380 | (2R,3R,4S,5S,6S)-2- | piperidine | | | | | y | y | | | | | | |
| | (but-3-enyl)-6- | | | | | | | | | | | | | |
| | (hydroxymethyl)piperidine- | | | | | | | | | | | | | |
| | 3,4,5-triol | | | | | | | | | | | | | |
| 381 | (3R,5S)-5- | pyrrolidine | | | | | | | | | | y | | y |
| | (azidomethyl)pyrrolidin- | | | | | | | | | | | | | |
| | 3-ol | | | | | | | | | | | | | |
| 382 | (2R,3R,4R,5S)-3,4,5- | piperidine | | | | | | | y | | | | | |
| | trihydroxy-N- | | | | | | | | | | | | | |
| | methylpiperidine-2- | | | | | | | | | | | | | |
| | carboxamide | | | | | | | | | | | | | |
| 383 | (5R)-3-hydroxy-5- | pyrrolidine | | | | | | | | | | | | |
| | (hydroxymethyl)pyrrolidine- | | | | | | | | | | | | | |
| | 3-carboxylic acid | | | | | | | | | | | | | |
| 384 | (2R,3S,4R)-2- | pyrrolidine | | | y | | | | | y | | | | |
| | (hydroxymethyl)pyrrolidine- | | | | | | | | | | | | | |
| | 3,4-diol | | | | | | | | | | | | | |
| 385 | (2R,3S,4R)-1-benzyl-2- | pyrrolidine | | | y | | | | | y | | | | |
| | (hydroxymethyl)pyrrolidine- | | | | | | | | | | | | | |
| | 3,4-diol | | | | | | | | | | | | | |
| 386 | (2S,3R,4S)-1-benzyl-2- | pyrrolidine | | | y | | | | | y | | | | |
| | (hydroxymethyl)pyrrolidine- | | | | | | | | | | | | | |
| | 3,4-diol | | | | | | | | | | | | | |
| 387 | (3R,5R)-1- | piperidine | | | y | | | | | y | | | | |
| | nonylpiperidine-3,4,5- | | | | | | | | | | | | | |
| | triol | | | | | | | | | | | | | |
| 388 | (2R,3R,4R,5S)-2- | piperidine | | | | | y | y | | | | | | |
| | methylpiperidine-3,4,5- | | | | | | | | | | | | | |
| | triol | | | | | | | | | | | | | |
| 389 | (2R,3S,4S)-4- | pyrrolidine | | | | | | | | | | y | | |
| | acetamido-2- | | | | | | | | | | | | | |
| | (acetoxymethyl)-1- | | | | | | | | | | | | | |
| | benzylpyrrolidin-3-yl | | | | | | | | | | | | | |
| | acetate | | | | | | | | | | | | | |
| 390 | (2R,3S,4S)-4-amino-1- | pyrrolidine | | | | | | | | | | y | | |
| | benzyl-2- | | | | | | | | | | | | | |
| | (hydroxymethyl)pyrrolidin- | | | | | | | | | | | | | |
| | 3-ol | | | | | | | | | | | | | |
| 391 | N-((3S,4S,5R)-1-butyl- | pyrrolidine | | | | | | | | | | y | | |
| | 4-hydroxy-5- | | | | | | | | | | | | | |
| | (hydroxymethyl)pyrrolidin- | | | | | | | | | | | | | |
| | 3-yl)acetamide | | | | | | | | | | | | | |
| 392 | 2-((2R,3S,4S)-4- | pyrrolidine | | | | | | | | | | y | | |
| | acetamido-3-hydroxy-2- | | | | | | | | | | | | | |
| | (hydroxymethyl)pyrrolidin- | | | | | | | | | | | | | |
| | 1-yl)acetic acid | | | | | | | | | | | | | |
| 393 | (R)-5-((1R,2S,3S)- | pyrrolidine | | | | | y | y | | | | | | |
| | 1,2,3,4- | | | | | | | | | | | | | |
| | tetrahydroxybutyl)pyrrolidin- | | | | | | | | | | | | | |
| | 2-one | | | | | | | | | | | | | |
| 394 | (2R,3R,4S)-4-azido-1- | pyrrolidine | | | | | | | | | | y | | |
| | benzyl-2- | | | | | | | | | | | | | |
| | (hydroxymethyl)pyrrolidin- | | | | | | | | | | | | | |
| | 3-ol | | | | | | | | | | | | | |
| 395 | N-((3S,4S,5R)-4- | pyrrolidine | | | | | | | | | | y | | |
| | hydroxy-5- | | | | | | | | | | | | | |
| | (hydroxymethyl)pyrrolidin- | | | | | | | | | | | | | |
| | 3-yl)acetamide | | | | | | | | | | | | | |
| 396 | N-((3S,4S,5R)-1-benzyl- | pyrrolidine | | | | | | | | | | y | | |
| | 4-hydroxy-5- | | | | | | | | | | | | | |
| | (hydroxymethyl)pyrrolidin- | | | | | | | | | | | | | |
| | 3-yl)acetamide | | | | | | | | | | | | | |
| 397 | N-((3S,4S,5R)-4- | pyrrolidine | | | | | | | | | | y | | |
| | hydroxy-1-(2- | | | | | | | | | | | | | |
| | hydroxyethyl)-5- | | | | | | | | | | | | | |
| | (hydroxymethyl)pyrrolidin- | | | | | | | | | | | | | |
| | 3-yl)acetamide | | | | | | | | | | | | | |
| 398 | 2-((2S,3R,4S)-2-((R)- | pyrrolidine | | | | | | | | | y | | | |
| | 1,2-dihydroxyethyl)-3,4- | | | | | | | | | | | | | |
| | dihydroxypyrrolidin-1- | | | | | | | | | | | | | |
| | yl)acetic acid | | | | | | | | | | | | | |
| 399 | (1R,2S,5S,8R,8aS)-5- | indolizidine | y | | | | | | | | | | | |
| | methyloctahydroindolizine- | | | | | | | | | | | | | |
| | 1,2,8-triol | | | | | | | | | | | | | |
| 400 | (1R,2S,6S,7R,8R,8aR)- | indolizidine | | | | | y | y | | | y | | | |
| | octahydroindolizine- | | | | | | | | | | | | | |
| | 1,2,6,7,8-pentaol | | | | | | | | | | | | | |
| 401 | (1R,2S,6R,7S,8R,8aR)- | indolizidine | | | | | y | y | | | y | | | |
| | 1,2,6,7,8- | | | | | | | | | | | | | |
| | pentahydroxyhexahydroindolizin- | | | | | | | | | | | | | |
| | 5(1H)-one | | | | | | | | | | | | | |
| 402 | (1R,2S,8S,8aS)-1,2,8- | indolizidine | | | | | | | | | y | | | |
| | trihydroxyhexahydroindolizin- | | | | | | | | | | | | | |
| | 5(1H)-one | | | | | | | | | | | | | |
| 403 | (2S,3R,4S)-1-butyl-2- | pyrrolidine | | | | | | | | | y | | | |
| | ((R)-1,2- | | | | | | | | | | | | | |
| | dihydroxyethyl)pyrrolidine- | | | | | | | | | | | | | |
| | 3,4-diol | | | | | | | | | | | | | |
| 404 | (1R,2R)-1-((2R,3R,4S)- | pyrrolidine | | | | y | | | | | y | | | |
| | 3,4-dihydroxy-1- | | | | | | | | | | | | | |
| | nonylpyrrolidin-2- | | | | | | | | | | | | | |
| | yl)propane-1,2,3-triol | | | | | | | | | | | | | |
| 405 | (2S,3R,4S)-2-((R)-1,2- | pyrrolidine | | | | | | | | | y | | | |
| | dihydroxyethyl)-1-(2- | | | | | | | | | | | | | |
| | hydroxyethyl)pyrrolidine- | | | | | | | | | | | | | |
| | 3,4-diol | | | | | | | | | | | | | |
| 406 | (1R,2S,5R,8S,8aS)-5- | indolizidine | | | | | | | | | y | | | |
| | methyloctahydroindolizine- | | | | | | | | | | | | | |
| | 1,2,8-triol | | | | | | | | | | | | | |
| 407 | (S)-5-((1S,2R,3R)- | pyrrolidine | | | | | y | y | | | | | | |
| | 1,2,3,4- | | | | | | | | | | | | | |
| | tetrahydroxybutyl)pyrrolidin- | | | | | | | | | | | | | |
| | 2-one | | | | | | | | | | | | | |
| 408 | (S)-4-((2S,3R,4S)-1- | pyrrolidine | | | | | | | | | y | | | |
| | benzyl-3,4- | | | | | | | | | | | | | |
| | dihydroxypyrrolidin-2- | | | | | | | | | | | | | |
| | yl)-4- | | | | | | | | | | | | | |
| | hydroxybutanenitrile | | | | | | | | | | | | | |
| 409 | (3aS,6R,9S,9aS,9bR)- | indolizidine | | | | | | | | | y | | | |
| | 2,2-diethyl-6- | | | | | | | | | | | | | |
| | methyloctahydro- | | | | | | | | | | | | | |
| | [1,3]dioxolo[4,5- | | | | | | | | | | | | | |
| | a]indolizin-9-ol | | | | | | | | | | | | | |
| 410 | (1R,2S,5R,8S,8aR)-8- | indolizidine | | | | | | | | | y | | | |
| | methoxy-5- | | | | | | | | | | | | | |
| | methyloctahydroindolizine- | | | | | | | | | | | | | |
| | 1,2-diol | | | | | | | | | | | | | |
| 411 | (1R,2S,3S)-1-((R)-1- | pyrrolidine | | | | | y | y | | | | | | |
| | butylpyrrolidin-2- | | | | | | | | | | | | | |
| | yl)butane-1,2,3,4-tetraol | | | | | | | | | | | | | |
| 412 | (3aR,5R,6R,6aS)-4- | pyrrolidine | | y | | | | | | | | | y | |
| | benzyl-6-hydroxy-5- | | | | | | | | | | | | | |
| | (hydroxymethyl)hexahydro- | | | | | | | | | | | | | |
| | 2H-furo[3,2-b]pyrrol- | | | | | | | | | | | | | |
| | 2-one | | | | | | | | | | | | | |
| 413 | (1R,2S,3S)-1-((R)- | pyrrolidine | | | | | y | y | | | | | | |
| | pyrrolidin-2-yl)butane- | | | | | | | | | | | | | |
| | 1,2,3,4-tetraol | | | | | | | | | | | | | |
| 414 | (1R,2S,3S)-1-((R)-1-(2- | pyrrolidine | | | | | y | y | | | | | | |
| | hydroxyethyl)pyrrolidin- | | | | | | | | | | | | | |
| | 2-yl)butane-1,2,3,4- | | | | | | | | | | | | | |
| | tetraol | | | | | | | | | | | | | |
| 415 | (S)-5-((1R,2S,3S)- | pyrrolidine | | | | | | | y | | | | | |
| | 1,2,3,4- | | | | | | | | | | | | | |
| | tetrahydroxybutyl)pyrrolidin- | | | | | | | | | | | | | |
| | 2-one | | | | | | | | | | | | | |
| 416 | (1R,2S,3S)-1-((S)-1-(2- | pyrrolidine | | | | | | | y | | | | | |
| | hydroxyethyl)pyrrolidin- | | | | | | | | | | | | | |
| | 2-yl)butane-1,2,3,4- | | | | | | | | | | | | | |
| | tetraol | | | | | | | | | | | | | |
| 417 | N-((3S,4R,5S)-1-benzyl- | pyrrolidine | | | y | | | | | y | | | | |
| | 4-hydroxy-5- | | | | | | | | | | | | | |
| | (hydroxymethyl)pyrrolidin- | | | | | | | | | | | | | |
| | 3-yl)-2,2,2- | | | | | | | | | | | | | |
| | trifluoroacetamide | | | | | | | | | | | | | |
| 418 | (3S,4S,5S)-1-butyl-5- | piperidine | | | | | | | | | | | | y |
| | (hydroxymethyl)piperidine- | | | | | | | | | | | | | |
| | 3,4-diol | | | | | | | | | | | | | |
| 419 | (3R,4R,5R)-1-butyl-5- | piperidine | | | | | | | | | | | | y |
| | (hydroxymethyl)piperidine- | | | | | | | | | | | | | |
| | 3,4-diol | | | | | | | | | | | | | |
| 420 | (3R,4R,5R)-1-(2- | piperidine | | | | | | | | | | | | y |
| | hydroxyethyl)-5- | | | | | | | | | | | | | |
| | (hydroxymethyl)piperidine- | | | | | | | | | | | | | |
| | 3,4-diol | | | | | | | | | | | | | |
| 421 | (1R,2S,3S)-1-((S)- | pyrrolidine | | | | | | | y | | | | | |
| | pyrrolidin-2-yl)butane- | | | | | | | | | | | | | |
| | 1,2,3,4-tetraol | | | | | | | | | | | | | |
| 422 | (1S,2R,3R)-1-((S)- | pyrrolidine | | | | | y | y | | | | | | |
| | pyrrolidin-2-yl)butane- | | | | | | | | | | | | | |
| | 1,2,3,4-tetraol | | | | | | | | | | | | | |
| 423 | (2S,3S,4R)-1-benzyl-2- | pyrrolidine | | y | | | | | | | | | y | |
| | ((S)-1,2- | | | | | | | | | | | | | |
| | dihydroxyethyl)pyrrolidine- | | | | | | | | | | | | | |
| | 3,4-diol | | | | | | | | | | | | | |
| 424 | (2R,3R,4R,5S)-tert-butyl | piperidine | | | | | y | y | | | | | | |
| | 3,4,5-trihydroxy-2- | | | | | | | | | | | | | |
| | (hydroxymethyl)piperidine- | | | | | | | | | | | | | |
| | 1-carboxylate | | | | | | | | | | | | | |
| 425 | (2S,3S,4R)-2-((S)-1,2- | pyrrolidine | | y | | | | | | | | | y | |
| | dihydroxyethyl)-1-(2- | | | | | | | | | | | | | |
| | hydroxyethyl)pyrrolidine- | | | | | | | | | | | | | |
| | 3,4-diol | | | | | | | | | | | | | |
| 426 | (2S,3S,4R)-1-butyl-2- | pyrrolidine | | y | | | | | | | | | y | |
| | ((S)-1,2- | | | | | | | | | | | | | |
| | dihydroxyethyl)pyrrolidine- | | | | | | | | | | | | | |
| | 3,4-diol | | | | | | | | | | | | | |
| 427 | (2S,3S,4R)-2-((S)-1,2- | pyrrolidine | | y | | | | | | | | | y | |
| | dihydroxyethyl)-1- | | | | | | | | | | | | | |
| | nonylpyrrolidine-3,4-diol | | | | | | | | | | | | | |
| 428 | (2S,3S,4R)-2-((S)-1,2- | pyrrolidine | | y | | | | | | | | | y | |
| | dihydroxyethyl)-1- | | | | | | | | | | | | | |
| | methylpyrrolidine-3,4- | | | | | | | | | | | | | |
| | diol | | | | | | | | | | | | | |
| 429 | (2S,3S,4R)-2-((S)-1,2- | pyrrolidine | | y | | | | | | | | | y | |
| | dihydroxyethyl)pyrrolidine- | | | | | | | | | | | | | |
| | 3,4-diol | | | | | | | | | | | | | |
| 430 | (3aR,4R,6aS)-4- | pyrrolidine | | | | | | | | | | y | | |
| | (azidomethyl)-5-benzyl- | | | | | | | | | | | | | |
| | 2,2-dimethyltetrahydro- | | | | | | | | | | | | | |
| | 3aH-[1,3]dioxolo[4,5- | | | | | | | | | | | | | |
| | c]pyrrole | | | | | | | | | | | | | |
| 431 | N-((3S,4R,5S)-1-benzyl- | pyrrolidine | | | y | | | | | y | | | | |
| | 4-hydroxy-5- | | | | | | | | | | | | | |
| | (hydroxymethyl)pyrrolidin- | | | | | | | | | | | | | |
| | 3-yl)butyramide | | | | | | | | | | | | | |
| 432 | (2R,3R,4S)-2- | pyrrolidine | | | | | | | | | | y | | |
| | (azidomethyl)-1- | | | | | | | | | | | | | |
| | benzylpyrrolidine-3,4- | | | | | | | | | | | | | |
| | diol | | | | | | | | | | | | | |
| 433 | (2S,3S,4R)-2-((S)-1,2- | pyrrolidine | | y | | | | | | | | | y | |
| | dihydroxyethyl)-1-(9- | | | | | | | | | | | | | |
| | hydroxynonyl)pyrrolidine- | | | | | | | | | | | | | |
| | 3,4-diol | | | | | | | | | | | | | |
| 434 | 2-((2S,3S,4R)-2-((S)- | pyrrolidine | | y | | | | | | | | | y | |
| | 1,2-dihydroxyethyl)-3,4- | | | | | | | | | | | | | |
| | dihydroxypyrrolidin-1- | | | | | | | | | | | | | |
| | yl)acetonitrile | | | | | | | | | | | | | |
| 435 | (2S,3S,4R)-2-((S)-1,2- | pyrrolidine | | y | | | | | | | | | y | |
| | dihydroxyethyl)-1-(2-(2- | | | | | | | | | | | | | |
| | methoxyethoxy)ethyl)pyrrolidine- | | | | | | | | | | | | | |
| | 3,4-diol | | | | | | | | | | | | | |
| 436 | (3R,4R,4aR,7S,8R,8aR)- | piperidine | | | | | y | y | | | | | | |
| | octahydro-2H- | | | | | | | | | | | | | |
| | pyrano[3,2-b]pyridine- | | | | | | | | | | | | | |
| | 3,4,7,8-tetrol | | | | | | | | | | | | | |
| 437 | 6-[(2S,4R)-4-hydroxy-2- | pyrrolidine | | | | | | | | | | y | | y |
| | (hydroxymethyl)pyrrolidin- | | | | | | | | | | | | | |
| | 1-yl]hexanoic acid | | | | | | | | | | | | | |
| 438 | 2-{[(2S,3S,4R,5S)-4- | pyrrolidine | | y | | | y | y | | | | | y | |
| | hydroxy-5- | | | | | | | | | | | | | |
| | (hydroxymethyl)-2- | | | | | | | | | | | | | |
| | [(2S,4Z)-undec-4-ene- | | | | | | | | | | | | | |
| | 1,2,11-triol]pyrrolidin-3- | | | | | | | | | | | | | |
| | yl]oxy}-6- | | | | | | | | | | | | | |
| | (hydroxymethyl)tetrahydro- | | | | | | | | | | | | | |
| | 2H-pyran-3,4,5-triol | | | | | | | | | | | | | |
| 439 | 3-[(2R,3R,4R)-3,4- | pyrrolidine | | | y | | | | | y | | | | |
| | dihydroxy-2- | | | | | | | | | | | | | |
| | (hydroxymethyl)pyrrolidin- | | | | | | | | | | | | | |
| | 1-yl]propanoic acid | | | | | | | | | | | | | |
| 440 | [(2S,3R,4R)-3,4- | pyrrolidine | | | | | | | | | | | | y |
| | dihydroxy-2- | | | | | | | | | | | | | |
| | (hydroxymethyl)pyrrolidin- | | | | | | | | | | | | | |
| | 1-yl]acetic acid | | | | | | | | | | | | | |
| 441 | (2S,4S,5S)-4,5- | piperidine | | | | y | y | y | | | | | | |
| | dihydroxypiperidine-2- | | | | | | | | | | | | | |
| | carboxylic acid | | | | | | | | | | | | | |
| 442 | (2R,3R,4R,5S)-3,4,5- | piperidine | | | | | | | y | | | | | |
| | trihydroxypiperidine-2- | | | | | | | | | | | | | |
| | carboxylic acid | | | | | | | | | | | | | |
| 443 | (3aR,6R,7R,8R,8aS,8bS)- | pyrrolizidine | | | | | y | y | y | | | | | |
| | 7,8-dihydroxy-6- | | | | | | | | | | | | | |
| | (hydroxymethyl)-2,2- | | | | | | | | | | | | | |
| | dimethylhexahydro-4H- | | | | | | | | | | | | | |
| | [1,3]dioxolo[4,5- | | | | | | | | | | | | | |
| | a]pyrrolizin-4-one | | | | | | | | | | | | | |
| 444 | (2S,5S,6S,7S,8R,8aS)- | indolizidine | | | | | y | y | | | | | | |
| | 6,7,8-trihydroxy-5- | | | | | | | | | | | | | |
| | (hydroxymethyl)-3- | | | | | | | | | | | | | |
| | oxooctahydroindolizine- | | | | | | | | | | | | | |
| | 2-carboxylic acid | | | | | | | | | | | | | |
| 445 | (2S,5R,6R,7R,8R,8aR)- | indolizidine | | | | | | | | | y | | | |
| | 6,7,8-trihydroxy-5- | | | | | | | | | | | | | |
| | (hydroxymethyl)-3- | | | | | | | | | | | | | |
| | oxooctahydroindolizine- | | | | | | | | | | | | | |
| | 2-carboxylic acid | | | | | | | | | | | | | |
| 446 | (3S,4R,5S,6R)-3,4,5- | piperidine | | | | | | | y | | | | | |
| | trihydroxy-6- | | | | | | | | | | | | | |
| | (hydroxymethyl)piperidin- | | | | | | | | | | | | | |
| | 2-one | | | | | | | | | | | | | |
| 447 | (2R,3S,4S,5R)-2- | piperidine | | | | | | | y | | | | | |
| | (hydroxymethyl)piperidine- | | | | | | | | | | | | | |
| | 3,4,5-triol | | | | | | | | | | | | | |
| 448 | (1R,2S,5R,6S,7S,7aS)- | pyrrolizidine | | | | | | | y | | | | | |
| | 1,2,6,7-tetrahydroxy-5- | | | | | | | | | | | | | |
| | (hydroxymethyl)hexahydro- | | | | | | | | | | | | | |
| | 3H-pyrrolizin-3-one | | | | | | | | | | | | | |
| 449 | (2R,3R,4R,5S)-benzyl | piperidine | | | | | y | y | | | | | | |
| | 3,4,5-trihydroxy-2- | | | | | | | | | | | | | |
| | (hydroxymethyl)piperidine- | | | | | | | | | | | | | |
| | 1-carboxylate | | | | | | | | | | | | | |
| 450 | 2-((3R,4R,5R)-3,4- | piperidine | | | | | | | | | | | | y |
| | dihydroxy-5- | | | | | | | | | | | | | |
| | (hydroxymethyl)piperidin- | | | | | | | | | | | | | |
| | 1-yl)acetic acid | | | | | | | | | | | | | |
| 451 | 2-((3S,4S,5S)-3,4- | piperidine | | | | | | | | | | | | y |
| | dihydroxy-5- | | | | | | | | | | | | | |
| | (hydroxymethyl)piperidin- | | | | | | | | | | | | | |
| | 1-yl)acetic acid | | | | | | | | | | | | | |
| 452 | (2R,3S,4R)-3,4- | pyrrolidine | | | | | | | | | | y | | |
| | dihydroxy-2- | | | | | | | | | | | | | |
| | methylpyrrolidine-2- | | | | | | | | | | | | | |
| | carboxylic acid | | | | | | | | | | | | | |
| 453 | 8-Aza- | nortropane | | | | | | | | | | | | |
| | bicyclo[3,2,1]octan-3-ol | | | | | | | | | | | | | |
| 454 | (R)-3-Hydroxypiperidine | piperidine | | | | | | | | | | | | |
| 455 | 4-Hydroxypiperidine | piperidine | | | | | | | | | | | | |
| 456 | cis-L-3-Hydroxyproline | pyrrolidine | | | | | | | | | | | | |
| 457 | (R)-3- | pyrrolidine | | | | | | | | | | | | |
| | Hydroxypyrrolidine | | | | | | | | | | | | | |
| 458 | cis-4-Hydroxy-D-proline | pyrrolidine | | | y | | | | | y | | | | |
| 459 | 4-hydroxy-2- | pyrrolidine | | | | | | | | | | | | |
| | Pyrrolidinecarboxamide | | | | | | | | | | | | | |
| 460 | 2-methyl-4-Piperidinol | piperidine | | | | | | | | | | | | |
| 461 | L-beta- | pyrrolidine | | | | | | | | | | y | | y |
| | Homohydroxyproline | | | | | | | | | | | | | |
| 462 | (R)-5-Hydroxy-piperidin- | piperidine | | | | | | | | | | | | |
| | 2-one | | | | | | | | | | | | | |
| 463 | (S)-(−)-4-Hydroxy-2- | pyrrolidine | | | | | | | | | | | | |
| | pyrrolidinone | | | | | | | | | | | | | |
| 464 | Nojirimycin-1-Sulfonic | piperidine | | | | | y | y | | | | | | |
| | Acid | | | | | | | | | | | | | |
| 465 | Siastatin B microbial | piperidine | y | | | | | | | | | | | |
| 466 | D-Glucaro-delta-lactam | piperidine | | | | | y | y | | | | | | |
| 467 | 4-hydroxy-4- | piperidine | | | | | | | | | | | | |
| | Piperidinecarboxylic | | | | | | | | | | | | | |
| | acid | | | | | | | | | | | | | |
| 468 | Labumine | pyrrolizidine | | | | | | | | | | | | |
| 469 | 1-Deoxy-L- | piperidine | | | | | | | y | | | | | |
| | idonojirimycin | | | | | | | | | | | | | |
| 470 | 2,5-Anhydro-2,5-imino- | pyrrolidine | | | | | y | y | | | | | | |
| | D-glucitol | | | | | | | | | | | | | |
| 471 | 1,4-Dideoxy-1,4-imino- | pyrrolidine | | | | | | | | | y | | | |
| | D-mannitol | | | | | | | | | | | | | |
| 472 | (2S,5S)- | pyrrolidine | | | | | | | y | | | | | |
| | Bishydroxymethyl- | | | | | | | | | | | | | |
| | (3R,4R)- | | | | | | | | | | | | | |
| | bishydroxypyrrolidine | | | | | | | | | | | | | |
| 473 | 4-hydroxy-2- | pyrrolidine | | | | | | | | | | | | |
| | Pyrrolidinemethanol | | | | | | | | | | | | | |
| 474 | (R)-3-Hydroxypiperidine | piperidine | | | | | | | | | | | | |
| 475 | cis-L-3-Hydroxyproline | pyrrolidine | | | | | | | | | | | | |
| 476 | (S)-3-Hydroxypyrrolidine | pyrrolidine | | | | | | | | | | | | |
| 477 | trans-4-Hydroxy-D- | pyrrolidine | | | | | | | | | | y | | y |
| | proline | | | | | | | | | | | | | |
| 478 | trans-4-Hydroxy-D- | pyrrolidine | | | | | | | | | | y | | y |
| | proline | | | | | | | | | | | | | |
| 479 | (R)-(+)-4-Hydroxy-2- | pyrrolidine | | | | | | | | | | | | |
| | pyrrolidinone | | | | | | | | | | | | | |
| 480 | S)-3-Hydroxy-pyrrolidin- | pyrrolidine | | | | | | | | | | | | |
| | 2-one | | | | | | | | | | | | | |
| 481 | N-(((3aR,4R,6aS)-5- | pyrrolidine | | | | | | | | | | y | | |
| | benzyl-2,2- | | | | | | | | | | | | | |
| | dimethyltetrahydro-3aH- | | | | | | | | | | | | | |
| | [1,3]dioxolo[4,5-c]pyrrol- | | | | | | | | | | | | | |
| | 4-yl)methyl)acetamide | | | | | | | | | | | | | |
| 482 | N-(((2R,3R,4S)-1- | pyrrolidine | | | | | | | | | | y | | |
| | benzyl-3,4- | | | | | | | | | | | | | |
| | dihydroxypyrrolidin-2- | | | | | | | | | | | | | |
| | yl)methyl)acetamide | | | | | | | | | | | | | |
| 483 | ((3aR,4R,6aS)-5- | pyrrolidine | | | | | | | | | | y | | |
| | benzyl-2,2- | | | | | | | | | | | | | |
| | dimethyltetrahydro-3aH- | | | | | | | | | | | | | |
| | [1,3]dioxolo[4,5-c]pyrrol- | | | | | | | | | | | | | |
| | 4-yl)methanamine | | | | | | | | | | | | | |
| 484 | N-(((2R,3R,4S)-3,4- | pyrrolidine | | | | | | | | | | y | | |
| | dihydroxypyrrolidin-2- | | | | | | | | | | | | | |
| | yl)methyl)acetamide | | | | | | | | | | | | | |
| 485 | N-((3S,4R,5S)-1-benzyl- | pyrrolidine | | | y | | | | | y | | | | |
| | 4-hydroxy-5- | | | | | | | | | | | | | |
| | (hydroxymethyl)pyrrolidin- | | | | | | | | | | | | | |
| | 3-yl)benzamide | | | | | | | | | | | | | |
| 486 | (2R,3R,4S)-2- | pyrrolidine | | | | | | | | | | y | | |
| | (aminomethyl)-1- | | | | | | | | | | | | | |
| | benzylpyrrolidine-3,4- | | | | | | | | | | | | | |
| | diol | | | | | | | | | | | | | |
| 487 | 2-((2S,3S,4R)-2-((S)- | pyrrolidine | | y | | | | | | | | | y | |
| | 1,2-dihydroxyethyl)-3,4- | | | | | | | | | | | | | |
| | dihydroxypyrrolidin-1- | | | | | | | | | | | | | |
| | yl)acetic acid | | | | | | | | | | | | | |
| 488 | (2R,3R,4S,5R,6R)-2- | piperidine | | y | | | | | | | y | | y | |
| | butyl-6- | | | | | | | | | | | | | |
| | (hydroxymethyl)piperidine- | | | | | | | | | | | | | |
| | 3,4,5-triol | | | | | | | | | | | | | |
| 489 | (1R,2S,8R,8aR)-1,2,8- | indolizidine | y | | | | | | | | | | | |
| | trihydroxy-6-(2- | | | | | | | | | | | | | |
| | hydroxyethyl)hexahydro | | | | | | | | | | | | | |
| | indolizin-5(1H)-one | | | | | | | | | | | | | |
| 490 | (1R,2S,8R,8aR)-1,2,8- | indolizidine | y | | | | | | | | | | | |
| | trihydroxy-6- | | | | | | | | | | | | | |
| | methylhexahydroindolizin- | | | | | | | | | | | | | |
| | 5(1H)-one | | | | | | | | | | | | | |
| 491 | 5-[(2R,3R,4R)-3,4- | pyrrolidine | | | y | | | | | y | | | | |
| | dihydroxy-2- | | | | | | | | | | | | | |
| | (hydroxymethyl)pyrrolidin- | | | | | | | | | | | | | |
| | 1-yl]pentanoic acid | | | | | | | | | | | | | |
| 492 | (1R,2S,6R,8R,8aR)-6- | indolizidine | y | | | | | | | | | | | |
| | (2- | | | | | | | | | | | | | |
| | hydroxyethyl)octahydroindolizine- | | | | | | | | | | | | | |
| | 1,2,8-triol | | | | | | | | | | | | | |
| 493 | (1R,2S,6S,8R,8aR)-6- | indolizidine | y | | | | | | | | | | | |
| | (2- | | | | | | | | | | | | | |
| | hydroxyethyl)octahydroindolizine- | | | | | | | | | | | | | |
| | 1,2,8-triol | | | | | | | | | | | | | |
| 494 | 3-[(2R,3R,4R,5R)-3,4- | pyrrolidine | | | | | | | | | y | | | |
| | dihydroxy-2,5- | | | | | | | | | | | | | |
| | bis(hydroxymethyl)pyrrolidin- | | | | | | | | | | | | | |
| | 1-yl]propanoic acid | | | | | | | | | | | | | |
| 495 | (2S,3S,3aS,6S,7S,7aS)- | pyrrolidine | | | | y | y | y | | | y | | | |
| | 2-(hydroxymethyl)-1- | | | | | | | | | | | | | |
| | (methylsulfonyl)octahydropyrano[3, | | | | | | | | | | | | | |
| | 2-b]pyrrole- | | | | | | | | | | | | | |
| | 3,6,7-triol | | | | | | | | | | | | | |
| 496 | (3S,3aS,5S,6R,7R,7aS)- | pyrrolidine | y | y | | | | | | | | | y | |
| | 5-(hydroxymethyl)-1- | | | | | | | | | | | | | |
| | (methylsulfonyl)octahydropyrano[3, | | | | | | | | | | | | | |
| | 2-b]pyrrole- | | | | | | | | | | | | | |
| | 3,6,7-triol | | | | | | | | | | | | | |
| 497 | 3-[(2S,4R)-4-hydroxy-2- | pyrrolidine | | | | | | | | | | y | | y |
| | (hydroxymethyl)pyrrolidin- | | | | | | | | | | | | | |
| | 1-yl]propanoic acid | | | | | | | | | | | | | |
| 498 | [(2S,4R)-4-hydroxy-2- | pyrrolidine | | | | | | | | | | y | | y |
| | (hydroxymethyl)pyrrolidin- | | | | | | | | | | | | | |
| | 1-yl]acetic acid | | | | | | | | | | | | | |
| 499 | 4-[(2R,3R,4R)-3,4- | pyrrolidine | | | y | | | | | y | | | | |
| | dihydroxy-2- | | | | | | | | | | | | | |
| | (hydroxymethyl)pyrrolidin- | | | | | | | | | | | | | |
| | 1-yl]butanoic acid | | | | | | | | | | | | | |
| 500 | (2S,3S,4S)-1-benzyl-2- | pyrrolidine | | | y | | | | | y | | | | |
| | (hydroxymethyl)pyrrolidine- | | | | | | | | | | | | | |
| | 3,4-diol | | | | | | | | | | | | | |
| 501 | (2S,3S,4S)-2- | pyrrolidine | | | y | | | | | y | | | | |
| | (hydroxymethyl)-2- | | | | | | | | | | | | | |
| | methylpyrrolidine-3,4- | | | | | | | | | | | | | |
| | diol | | | | | | | | | | | | | |
| 502 | (2R,3S,4S)-N-benzyl- | pyrrolidine | | | y | | | | | y | | | | |
| | 3,4-dihydroxy-2- | | | | | | | | | | | | | |
| | methylpyrrolidine-2- | | | | | | | | | | | | | |
| | carboxamide | | | | | | | | | | | | | |
| 503 | N-{[(3S,4S,5R)-1- | piperidine | | | | | | | | | | y | | |
| | benzyl-4,5- | | | | | | | | | | | | | |
| | dihydroxypiperidin-3- | | | | | | | | | | | | | |
| | yl]methyl}acetamide | | | | | | | | | | | | | |
| 504 | (2R,3S,4S)-3,4- | pyrrolidine | | | y | | | | | y | | | | |
| | dihydroxy-2- | | | | | | | | | | | | | |
| | methylpyrrolidine-2- | | | | | | | | | | | | | |
| | carboxylic acid | | | | | | | | | | | | | |
| 505 | (3aR,4S,6aS)-4- | pyrrolidine | | | y | | | | | y | | | | |
| | (azidomethyl)-5-benzyl- | | | | | | | | | | | | | |
| | 2,2-dimethyltetrahydro- | | | | | | | | | | | | | |
| | 3aH-[1,3]dioxolo[4,5- | | | | | | | | | | | | | |
| | c]pyrrole | | | | | | | | | | | | | |
| 506 | (2S,3R,4S)-2- | pyrrolidine | | | y | | | | | y | | | | |
| | (azidomethyl)-1- | | | | | | | | | | | | | |
| | benzylpyrrolidine-3,4- | | | | | | | | | | | | | |
| | diol | | | | | | | | | | | | | |
| 507 | ((3aR,4S,6aS)-5-benzyl- | pyrrolidine | | | y | | | | | y | | | | |
| | 2,2-dimethyltetrahydro- | | | | | | | | | | | | | |
| | 3aH-[1,3]dioxolo[4,5- | | | | | | | | | | | | | |
| | c]pyrrol-4- | | | | | | | | | | | | | |
| | yl)methanamine | | | | | | | | | | | | | |
| 508 | N-(((3aR,4S,6aS)-5- | pyrrolidine | | | y | | | | | y | | | | |
| | benzyl-2,2- | | | | | | | | | | | | | |
| | dimethyltetrahydro-3aH- | | | | | | | | | | | | | |
| | [1,3]dioxolo[4,5-c]pyrrol- | | | | | | | | | | | | | |
| | 4-yl)methyl)acetamide | | | | | | | | | | | | | |
| 509 | (2R,3R,4R,5S)-2- | piperidine | | | | | y | y | | | | | | |
| | (hydroxymethyl)-1-(2- | | | | | | | | | | | | | |
| | morpholinoethyl)piperidine- | | | | | | | | | | | | | |
| | 3,4,5-triol | | | | | | | | | | | | | |
| 510 | (2R,3R,4R,5S)-1- | piperidine | | | | | y | y | | | | | | |
| | benzyl-2- | | | | | | | | | | | | | |
| | (hydroxymethyl)piperidine- | | | | | | | | | | | | | |
| | 3,4,5-triol | | | | | | | | | | | | | |
| 511 | N-(((2R,3R,4S)-3,4- | pyrrolidine | | | | | | | | | | y | | |
| | dihydroxy-1-(9- | | | | | | | | | | | | | |
| | hydroxynonyl)pyrrolidin- | | | | | | | | | | | | | |
| | 2-yl)methyl)acetamide | | | | | | | | | | | | | |
| 512 | N-(((2R,3R,4S)-3,4- | pyrrolidine | | | | | | | | | | y | | |
| | dihydroxy-1- | | | | | | | | | | | | | |
| | nonylpyrrolidin-2- | | | | | | | | | | | | | |
| | yl)methyl)acetamide | | | | | | | | | | | | | |
| 513 | (2R,3R,4S)-2- | pyrrolidine | | | | | | | | | | y | | |
| | (aminomethyl)pyrrolidine- | | | | | | | | | | | | | |
| | 3,4-diol | | | | | | | | | | | | | |
| 514 | (2R,3R,4R,5R)-1- | pyrrolidine | | | | | | | | | y | | | |
| | benzyl-2,5- | | | | | | | | | | | | | |
| | bis(hydroxymethyl)pyrrolidine- | | | | | | | | | | | | | |
| | 3,4-diol | | | | | | | | | | | | | |
| 515 | (2R,3R,4R,5R)-2,5- | pyrrolidine | | | | | | | | | y | | | |
| | bis(hydroxymethyl)-1- | | | | | | | | | | | | | |
| | methylpyrrolidine-3,4- | | | | | | | | | | | | | |
| | diol | | | | | | | | | | | | | |
| 516 | N-(((2R,3R,4S)-3,4- | pyrrolidine | | | | | | | | | | y | | |
| | dihydroxy-1-(2-(2- | | | | | | | | | | | | | |
| | methoxyethoxy)ethyl)pyrrolidin- | | | | | | | | | | | | | |
| | 2- | | | | | | | | | | | | | |
| | yl)methyl)acetamide | | | | | | | | | | | | | |
| 517 | N-(((2R,3R,4S)-3,4- | pyrrolidine | | | | | | | | | | y | | |
| | dihydroxy-1-(2- | | | | | | | | | | | | | |
| | hydroxyethyl)pyrrolidin- | | | | | | | | | | | | | |
| | 2-yl)methyl)acetamide | | | | | | | | | | | | | |
| 518 | N-(((2R,3R,4S)-1- | pyrrolidine | | | | | | | | | | y | | |
| | (biphenyl-4-ylmethyl)- | | | | | | | | | | | | | |
| | 3,4-dihydroxypyrrolidin- | | | | | | | | | | | | | |
| | 2-yl)methyl)acetamide | | | | | | | | | | | | | |
| 519 | N-(((2R,3R,4S)-1-butyl- | pyrrolidine | | | | | | | | | | y | | |
| | 3,4-dihydroxypyrrolidin- | | | | | | | | | | | | | |
| | 2-yl)methyl)acetamide | | | | | | | | | | | | | |
| 520 | N-(((2R,3R,4S)-3,4- | pyrrolidine | | | | | | | | | | y | | |
| | dihydroxy-1-(2- | | | | | | | | | | | | | |
| | morpholinoethyl)pyrrolidin- | | | | | | | | | | | | | |
| | 2- | | | | | | | | | | | | | |
| | yl)methyl)acetamide | | | | | | | | | | | | | |
| 521 | 3-((2R,3R,4S)-2- | pyrrolidine | | | | | | | | | | y | | |
| | (acetamidomethyl)-3,4- | | | | | | | | | | | | | |
| | dihydroxypyrrolidin-1- | | | | | | | | | | | | | |
| | yl)propanamide | | | | | | | | | | | | | |
| 522 | (1R,2S,3S)-1- | pyrrolidine | | y | | | y | y | | | | | y | |
| | [(2R,3S,4S)-3,4- | | | | | | | | | | | | | |
| | dihydroxypyrrolidin-2- | | | | | | | | | | | | | |
| | yl]butane-1,2,3,4-tetrol | | | | | | | | | | | | | |
| 523 | (2R,3R,4R,5S)-2- | piperidine | | | | | y | y | | | | | | |
| | (hydroxymethyl)-1-(2- | | | | | | | | | | | | | |
| | (piperidin-1- | | | | | | | | | | | | | |
| | yl)ethyl)piperidine-3,4,5- | | | | | | | | | | | | | |
| | triol | | | | | | | | | | | | | |
| 524 | (2R,3R,4R,5S)-1- | piperidine | | | | | y | y | | | | | | |
| | (biphenyl-4-ylmethyl)-2- | | | | | | | | | | | | | |
| | (hydroxymethyl)piperidine- | | | | | | | | | | | | | |
| | 3,4,5-triol | | | | | | | | | | | | | |
| 525 | (1R,2S,5R,8S,8aS)-5- | indolizidine | | | | | | | | | y | | | |
| | methyloctahydroindolizine- | | | | | | | | | | | | | |
| | 1,2,8-triyl triacetate | | | | | | | | | | | | | |
| 526 | (1R,2S,3R)-1- | pyrrolidine | | | | y | y | y | | | y | | | |
| | ((2R,3R,4R)-1-benzyl- | | | | | | | | | | | | | |
| | 3,4-dihydroxypyrrolidin- | | | | | | | | | | | | | |
| | 2-yl)butane-1,2,3,4- | | | | | | | | | | | | | |
| | tetraol | | | | | | | | | | | | | |
| 527 | (1R,2S,3R)-1- | pyrrolidine | | | | y | | | | | y | | | |
| | ((2R,3R,4S)-3,4- | | | | | | | | | | | | | |
| | dihydroxy-1- | | | | | | | | | | | | | |
| | nonylpyrrolidin-2- | | | | | | | | | | | | | |
| | yl)butane-1,2,3,4-tetraol | | | | | | | | | | | | | |
| 528 | (1R,2S,3R)-1- | pyrrolidine | | | | y | | | | | y | | | |
| | ((2R,3R,4S)-1- | | | | | | | | | | | | | |
| | (biphenyl-4-ylmethyl)- | | | | | | | | | | | | | |
| | 3,4-dihydroxypyrrolidin- | | | | | | | | | | | | | |
| | 2-yl)butane-1,2,3,4- | | | | | | | | | | | | | |
| | tetraol | | | | | | | | | | | | | |
| 529 | (1R,2S,3R)-1- | pyrrolidine | | | | y | | | | | y | | | |
| | ((2R,3R,4S)-3,4- | | | | | | | | | | | | | |
| | dihydroxy-1-(9- | | | | | | | | | | | | | |
| | hydroxynonyl)pyrrolidin- | | | | | | | | | | | | | |
| | 2-yl)butane-1,2,3,4- | | | | | | | | | | | | | |
| | tetraol | | | | | | | | | | | | | |
| 530 | 2-((2R,3R,4S)-3,4- | pyrrolidine | | | | y | | | | | y | | | |
| | dihydroxy-2- | | | | | | | | | | | | | |
| | ((1R,2S,3R)-1,2,3,4- | | | | | | | | | | | | | |
| | tetrahydroxybutyl)pyrrolidin- | | | | | | | | | | | | | |
| | 1-yl)acetic acid | | | | | | | | | | | | | |
| 531 | (1R,2S,3R)-1- | pyrrolidine | | | | y | | | | | y | | | |
| | ((2R,3R,4S)-1butyl-3,4- | | | | | | | | | | | | | |
| | dihydroxypyrrolidin-2- | | | | | | | | | | | | | |
| | yl)butane-1,2,3,4-tetraol | | | | | | | | | | | | | |
| 532 | (1R,2S,3R)-1- | pyrrolidine | | | | y | | | | | y | | | |
| | ((2R,3R,4S)-1-benzyl- | | | | | | | | | | | | | |
| | 3,4-dihydroxypyrrolidin- | | | | | | | | | | | | | |
| | 2-yl)butane-1,2,3,4- | | | | | | | | | | | | | |
| | tetraol | | | | | | | | | | | | | |
| 533 | (1R,2S,3R)-1- | pyrrolidine | | | | y | | | | | y | | | |
| | ((2R,3R,4S)-3,4- | | | | | | | | | | | | | |
| | dihydroxy-1-(2- | | | | | | | | | | | | | |
| | hydroxyethyl)pyrrolidin- | | | | | | | | | | | | | |
| | 2-yl)butane-1,2,3,4- | | | | | | | | | | | | | |
| | tetraol | | | | | | | | | | | | | |
| 534 | (1R,2S,5R,6R,7S,8R,8aR)- | indolizidine | | y | | | | | | | y | | y | |
| | 5- | | | | | | | | | | | | | |
| | methyloctahydroindolizine- | | | | | | | | | | | | | |
| | 1,2,6,7,8-pentaol | | | | | | | | | | | | | |
| 535 | (1R,2S,3R)-1- | pyrrolidine | | | | y | | | | | y | | | |
| | ((2R,3R,4S)-3,4- | | | | | | | | | | | | | |
| | dihydroxypyrrolidin-2- | | | | | | | | | | | | | |
| | yl)butane-1,2,3,4-tetraol | | | | | | | | | | | | | |
| 536 | (1R,2S,3R)-1- | pyrrolidine | | | | y | | | | | y | | | |
| | ((2R,3R,4S)-3,4- | | | | | | | | | | | | | |
| | dihydroxy-1-(2-(2- | | | | | | | | | | | | | |
| | methoxyethoxy)ethyl)pyrrolidin- | | | | | | | | | | | | | |
| | 2-yl)butane- | | | | | | | | | | | | | |
| | 1,2,3,4-tetraol | | | | | | | | | | | | | |
| 537 | N-(((2R,3R,4S)-3,4- | pyrrolidine | | | | | | | | | | y | | |
| | dihydroxy-1-(2- | | | | | | | | | | | | | |
| | (piperidin-1- | | | | | | | | | | | | | |
| | yl)ethyl)pyrrolidin-2- | | | | | | | | | | | | | |
| | yl)methyl)acetamide | | | | | | | | | | | | | |
| 538 | N-butyl-2- | pyrrolidine | | y | | | | | | | | | y | |
| | ((2R,3S,4R,5R)-3,4- | | | | | | | | | | | | | |
| | dihydroxy-5- | | | | | | | | | | | | | |
| | (hydroxymethyl)-1- | | | | | | | | | | | | | |
| | nonylpyrrolidin-2- | | | | | | | | | | | | | |
| | yl)acetamide | | | | | | | | | | | | | |
| 539 | 2-((2R,3S,4R,5R)-1- | pyrrolidine | | y | | | | | | | | | y | |
| | benzyl-3,4-dihydroxy-5- | | | | | | | | | | | | | |
| | (hydroxymethyl)pyrrolidin- | | | | | | | | | | | | | |
| | 2-yl)-N- | | | | | | | | | | | | | |
| | butylacetamide | | | | | | | | | | | | | |
| 540 | N-butyl-2- | pyrrolidine | | y | | | | | | | | | y | |
| | ((2R,3S,4R,5R)-3,4- | | | | | | | | | | | | | |
| | dihydroxy-1-(2- | | | | | | | | | | | | | |
| | hydroxyethyl)-5- | | | | | | | | | | | | | |
| | (hydroxymethyl)pyrrolidin- | | | | | | | | | | | | | |
| | 2-yl)acetamide | | | | | | | | | | | | | |
| 541 | N-butyl-2- | pyrrolidine | | y | | | | | | | | | y | |
| | ((2R,3S,4R,5R)-1-butyl- | | | | | | | | | | | | | |
| | 3,4-dihydroxy-5- | | | | | | | | | | | | | |
| | (hydroxymethyl)pyrrolidin- | | | | | | | | | | | | | |
| | 2-yl)acetamide | | | | | | | | | | | | | |
| 542 | N-(((2R,3R,4S)-1-(2- | pyrrolidine | | | | | | | | | | y | | |
| | (dimethylamino)ethyl)- | | | | | | | | | | | | | |
| | 3,4-dihydroxypyrrolidin- | | | | | | | | | | | | | |
| | 2-yl)methyl)acetamide | | | | | | | | | | | | | |
| 543 | N-butyl-2- | pyrrolidine | | | | | | | | | y | | | |
| | ((2R,3S,4R,5R)-3,4- | | | | | | | | | | | | | |
| | dihydroxy-5- | | | | | | | | | | | | | |
| | (hydroxymethyl)pyrrolidin- | | | | | | | | | | | | | |
| | 2-yl)acetamide | | | | | | | | | | | | | |
| 544 | 2-((2R,3R,4S)-2- | pyrrolidine | | | | | | | | | | y | | |
| | (acetamidomethyl)-3,4- | | | | | | | | | | | | | |
| | dihydroxypyrrolidin-1- | | | | | | | | | | | | | |
| | yl)acetic acid | | | | | | | | | | | | | |
| 545 | (3R,5S)-5- | pyrrolidine | | | | | | | | | | y | | y |
| | (acetamidomethyl)-1-(2- | | | | | | | | | | | | | |
| | acetoxyethyl)pyrrolidin- | | | | | | | | | | | | | |
| | 3-yl acetate | | | | | | | | | | | | | |
| 546 | (3R,5S)-5- | pyrrolidine | | | | | | | | | | y | | y |
| | (acetamidomethyl)-1- | | | | | | | | | | | | | |
| | butylpyrrolidin-3-yl | | | | | | | | | | | | | |
| | acetate | | | | | | | | | | | | | |
| 547 | (3R,5S)-5- | pyrrolidine | | | | | | | | | | y | | y |
| | (acetamidomethyl)-1- | | | | | | | | | | | | | |
| | nonylpyrrolidin-3-yl | | | | | | | | | | | | | |
| | acetate | | | | | | | | | | | | | |
| 548 | N-(((2S,4R)-4-hydroxy- | pyrrolidine | | | | | | | | | | | | |
| | 1-(2- | | | | | | | | | | | | | |
| | hydroxyethyl)pyrrolidin- | | | | | | | | | | | | | |
| | 2-yl)methyl)acetamide | | | | | | | | | | | | | |
| 549 | N-(((2S,4R)-1-butyl-4- | pyrrolidine | | | | | | | | | | y | | y |
| | hydroxypyrrolidin-2- | | | | | | | | | | | | | |
| | yl)methyl)acetamide | | | | | | | | | | | | | |
| 550 | (2R,3R,4R,5S)-2- | piperidine | | | | | y | y | | | | | | |
| | (hydroxymethyl)-1- | | | | | | | | | | | | | |
| | nonylpiperidine-3,4,5- | | | | | | | | | | | | | |
| | triol | | | | | | | | | | | | | |
| 551 | azetidin-3-ol | other | | | | | | | | | | | | |
| 552 | (3S,4S)-tert-butyl 4- | piperidine | | | | | | | | | | | | |
| | bromo-3- | | | | | | | | | | | | | |
| | hydroxypiperidine-1- | | | | | | | | | | | | | |
| | carboxylate | | | | | | | | | | | | | |
| 553 | (R)-tert-butyl 3- | piperidine | | | | | | | | | | | | |
| | (hydroxymethyl)piperidine- | | | | | | | | | | | | | |
| | 1-carboxylate | | | | | | | | | | | | | |
| 554 | (S)-tert-butyl 3- | piperidine | | | | | | | | | | | | |
| | (hydroxymethyl)piperidine- | | | | | | | | | | | | | |
| | 1-carboxylate | | | | | | | | | | | | | |
| 555 | (2R,3R,4R,5R)-2,5- | pyrrolidine | | | | | | | | | y | | | |
| | bis(hydroxymethyl)-1- | | | | | | | | | | | | | |
| | nonylpyrrolidine-3,4-diol | | | | | | | | | | | | | |
| 556 | (2R,3R,4R,5R)-1-(2- | pyrrolidine | | | | | | | | | y | | | |
| | (benzyloxy)ethyl)-2,5- | | | | | | | | | | | | | |
| | bis(hydroxymethyl)pyrrolidine- | | | | | | | | | | | | | |
| | 3,4-diol | | | | | | | | | | | | | |
| 557 | (2R,3R,4R,5R)-2,5- | pyrrolidine | | | | | | | | | y | | | |
| | bis(hydroxymethyl)-1-(9- | | | | | | | | | | | | | |
| | hydroxynonyl)pyrrolidine- | | | | | | | | | | | | | |
| | 3,4-diol | | | | | | | | | | | | | |
| 558 | (2R,3R,4R,5R)-1- | pyrrolidine | | | | | | | | | y | | | |
| | (biphenyl-4-ylmethyl)- | | | | | | | | | | | | | |
| | 2,5- | | | | | | | | | | | | | |
| | bis(hydroxymethyl)pyrrolidine- | | | | | | | | | | | | | |
| | 3,4-diol | | | | | | | | | | | | | |
| 559 | (2R,3R,4R,5R)-2,5- | pyrrolidine | | | | | | | | | y | | | |
| | bis(hydroxymethyl)-1-(2- | | | | | | | | | | | | | |
| | morpholinoethyl)pyrrolidine- | | | | | | | | | | | | | |
| | 3,4-diol | | | | | | | | | | | | | |
| 560 | (2R,3R,4R,5R)-2,5- | pyrrolidine | | | | | | | | | y | | | |
| | bis(hydroxymethyl)-1-(2- | | | | | | | | | | | | | |
| | (piperidin-1- | | | | | | | | | | | | | |
| | yl)ethyl)pyrrolidine-3,4- | | | | | | | | | | | | | |
| | diol | | | | | | | | | | | | | |
| 561 | (2S,3S,4S,5S)-2-((R)-4- | pyrrolidine | | | | | | | | | y | | | |
| | aminopentyl)-5- | | | | | | | | | | | | | |
| | (hydroxymethyl)pyrrolidine- | | | | | | | | | | | | | |
| | 3,4-diol | | | | | | | | | | | | | |
| 562 | (2S,3S,4S,5S)-2-((S)-4- | pyrrolidine | | | | | | | | | y | | | |
| | aminopentyl)-5- | | | | | | | | | | | | | |
| | (hydroxymethyl)pyrrolidine- | | | | | | | | | | | | | |
| | 3,4-diol | | | | | | | | | | | | | |
| 563 | N-((3R,4S,5R)-4,5- | piperidine | | | y | | | | | y | | | | |
| | dihydroxypiperidin-3- | | | | | | | | | | | | | |
| | yl)acetamide | | | | | | | | | | | | | |
| 564 | (2R,3R,4R)-1-(biphenyl- | pyrrolidine | | | y | | | | | y | | | | |
| | 4-ylmethyl)-2- | | | | | | | | | | | | | |
| | (hydroxymethyl)pyrrolidine- | | | | | | | | | | | | | |
| | 3,4-diol | | | | | | | | | | | | | |
| 565 | (R)-piperidin-3- | piperidine | | | | | | | | | | | | |
| | ylmethanol | | | | | | | | | | | | | |
| 566 | (2R,3R,4R)-1-benzyl-2- | pyrrolidine | | | y | | | | | y | | | | |
| | (hydroxymethyl)pyrrolidine- | | | | | | | | | | | | | |
| | 3,4-diol | | | | | | | | | | | | | |
| 567 | (2R,3R,4R,5R)-2,5- | pyrrolidine | | | | | | | | | y | | | |
| | bis(hydroxymethyl)-1-(2- | | | | | | | | | | | | | |
| | (2- | | | | | | | | | | | | | |
| | methoxyethoxy)ethyl)pyrrolidine- | | | | | | | | | | | | | |
| | 3,4-diol | | | | | | | | | | | | | |
| 568 | (2R,3R,4R)-2- | pyrrolidine | | | y | | | | | y | | | | |
| | (hydroxymethyl)-1- | | | | | | | | | | | | | |
| | nonylpyrrolidine-3,4-diol | | | | | | | | | | | | | |
| 569 | (2R,3R,4R)-2- | pyrrolidine | | | y | | | | | y | | | | |
| | (hydroxymethyl)-1-(9- | | | | | | | | | | | | | |
| | hydroxynonyl)pyrrolidine- | | | | | | | | | | | | | |
| | 3,4-diol | | | | | | | | | | | | | |
| 570 | ((3aS,4S,6aR)-5-benzyl- | pyrrolidine | | | | | | | | | | y | | |
| | 2,2-dimethyltetrahydro- | | | | | | | | | | | | | |
| | 3aH-[1,3]dioxolo[4,5- | | | | | | | | | | | | | |
| | c]pyrrol-4-yl)methanol | | | | | | | | | | | | | |
| 571 | (2R,3R,4R)-2- | pyrrolidine | | | y | | | | | y | | | | |
| | (hydroxymethyl)-1-(2-(2- | | | | | | | | | | | | | |
| | methoxyethoxy)ethyl)pyrrolidine- | | | | | | | | | | | | | |
| | 3,4-diol | | | | | | | | | | | | | |
| 572 | (2R,3R,4R)-2- | pyrrolidine | | | y | | | | | y | | | | |
| | (hydroxymethyl)-1-(2- | | | | | | | | | | | | | |
| | morpholinoethyl)pyrrolidine- | | | | | | | | | | | | | |
| | 3,4-diol | | | | | | | | | | | | | |
| 573 | (2R,3R,4R)-2- | pyrrolidine | | | y | | | | | y | | | | |
| | (hydroxymethyl)-1-(2- | | | | | | | | | | | | | |
| | (piperidin-1- | | | | | | | | | | | | | |
| | yl)ethyl)pyrrolidine-3,4- | | | | | | | | | | | | | |
| | diol | | | | | | | | | | | | | |
| 574 | 3-((2R,3R,4R)-3,4- | pyrrolidine | | | y | | | | | y | | | | |
| | dihydroxy-2- | | | | | | | | | | | | | |
| | (hydroxymethyl)pyrrolidin- | | | | | | | | | | | | | |
| | 1-yl)propanamide | | | | | | | | | | | | | |
| 575 | (3aR,7R,7aR)-7- | piperidine | | | | | | | | | | y | | |
| | hydroxy-3a- | | | | | | | | | | | | | |
| | (hydroxymethyl)-2,2- | | | | | | | | | | | | | |
| | dimethyltetrahydro- | | | | | | | | | | | | | |
| | [1,3]dioxolo[4,5- | | | | | | | | | | | | | |
| | c]pyridin-4(3aH)-one | | | | | | | | | | | | | |
| 576 | (3aS,4R,7R,7aR)-4- | piperidine | | y | | | | | | | | | y | |
| | (hydroxymethyl)-2,2- | | | | | | | | | | | | | |
| | dimethylhexahydro- | | | | | | | | | | | | | |
| | [1,3]dioxolo[4,5- | | | | | | | | | | | | | |
| | c]pyridin-7-ol | | | | | | | | | | | | | |
| 577 | (3aR,4S,6aS)-N-benzyl- | pyrrolidine | | | | | | | | | | y | | |
| | 2,2,4,6a- | | | | | | | | | | | | | |
| | tetramethyltetrahydro- | | | | | | | | | | | | | |
| | 3aH-[1,3]dioxolo[4,5- | | | | | | | | | | | | | |
| | c]pyrrole-4-carboxamide | | | | | | | | | | | | | |
| 578 | (3aS,7S,7aR)-7- | piperidine | | | | | | | | | | y | | |
| | (azidomethyl)-5-benzyl- | | | | | | | | | | | | | |
| | 2,2,3a- | | | | | | | | | | | | | |
| | trimethylhexahydro- | | | | | | | | | | | | | |
| | [1,3]dioxolo[4,5- | | | | | | | | | | | | | |
| | c]pyridine | | | | | | | | | | | | | |
| 579 | (3aS,4R,7R,7aR)-tert- | piperidine | | y | | | | | | | | | y | |
| | butyl 7-hydroxy-2,2,4- | | | | | | | | | | | | | |
| | trimethyltetrahydro- | | | | | | | | | | | | | |
| | [1,3]dioxolo[4,5- | | | | | | | | | | | | | |
| | c]pyridine-5(6H)- | | | | | | | | | | | | | |
| | carboxylate | | | | | | | | | | | | | |
| 580 | tert-butyl 5-hydroxy-5,6- | piperidine | | | | | | | | | | | | |
| | dihydropyridine-1(2H)- | | | | | | | | | | | | | |
| | carboxylate | | | | | | | | | | | | | |
| 581 | N-butyl-2- | pyrrolidine | | y | | | | | | | | | y | |
| | ((2R,3S,4R,5R)-3,4- | | | | | | | | | | | | | |
| | dihydroxy-5- | | | | | | | | | | | | | |
| | (hydroxymethyl)-1-(2-(2- | | | | | | | | | | | | | |
| | methoxyethoxy)ethyl)pyrrolidin- | | | | | | | | | | | | | |
| | 2-yl)acetamide | | | | | | | | | | | | | |
| 582 | N-butyl-2- | pyrrolidine | | y | | | | | | | | | y | |
| | ((2R,3S,4R,5R)-3,4- | | | | | | | | | | | | | |
| | dihydroxy-5- | | | | | | | | | | | | | |
| | (hydroxymethyl)-1-(9- | | | | | | | | | | | | | |
| | hydroxynonyl)pyrrolidin- | | | | | | | | | | | | | |
| | 2-yl)acetamide | | | | | | | | | | | | | |
| 583 | N-(((2S,3R,4S)-3,4- | pyrrolidine | | | y | | | | | y | | | | |
| | dihydroxy-1-(2-(2- | | | | | | | | | | | | | |
| | methoxyethoxy)ethyl)pyrrolidin- | | | | | | | | | | | | | |
| | 2- | | | | | | | | | | | | | |
| | yl)methyl)acetamide | | | | | | | | | | | | | |
| 584 | N-(((2S,3R,4S)-1- | pyrrolidine | | | y | | | | | y | | | | |
| | (biphenyl-4-ylmethyl)- | | | | | | | | | | | | | |
| | 3,4-dihydroxypyrrolidin- | | | | | | | | | | | | | |
| | 2-yl)methyl)acetamide | | | | | | | | | | | | | |
| 585 | 2-((2R,3S,4R,5R)-1- | pyrrolidine | | y | | | | | | | | | y | |
| | (biphenyl-4-ylmethyl)- | | | | | | | | | | | | | |
| | 3,4-dihydroxy-5- | | | | | | | | | | | | | |
| | (hydroxymethyl)pyrrolidin- | | | | | | | | | | | | | |
| | 2-yl)-N- | | | | | | | | | | | | | |
| | butylacetamide | | | | | | | | | | | | | |
| 586 | N-(((2R,3R,4S)-1- | pyrrolidine | | | | | | | | | | y | | |
| | benzyl-3,4- | | | | | | | | | | | | | |
| | dihydroxypyrrolidin-2- | | | | | | | | | | | | | |
| | yl)methyl)benzamide | | | | | | | | | | | | | |
| 587 | N-(((2S,3R,4S)-3,4- | pyrrolidine | | | y | | | | | y | | | | |
| | dihydroxy-1- | | | | | | | | | | | | | |
| | nonylpyrrolidin-2- | | | | | | | | | | | | | |
| | yl)methyl)acetamide | | | | | | | | | | | | | |
| 588 | ((3aS,4S,6aR)-2,2- | pyrrolidine | | | | | | | | | | y | | |
| | dimethyltetrahydro-3aH- | | | | | | | | | | | | | |
| | [1,3]dioxolo[4,5-c]pyrrol- | | | | | | | | | | | | | |
| | 4-yl)methanol | | | | | | | | | | | | | |
| 589 | (1R,2S,3R)-1-((3R,4R)- | pyrrolidine | | | | y | y | y | | | y | | | |
| | 3,4-dihydroxy-1-(2- | | | | | | | | | | | | | |
| | hydroxyethyl)pyrrolidin- | | | | | | | | | | | | | |
| | 2-yl)butane-1,2,3,4- | | | | | | | | | | | | | |
| | tetraol | | | | | | | | | | | | | |
| 590 | (1R,2S,3R)-1-((3R,4R)- | pyrrolidine | | | | y | y | y | | | y | | | |
| | 1-(biphenyl-4-ylmethyl)- | | | | | | | | | | | | | |
| | 3,4-dihydroxypyrrolidin- | | | | | | | | | | | | | |
| | 2-yl)butane-1,2,3,4- | | | | | | | | | | | | | |
| | tetraol | | | | | | | | | | | | | |
| 591 | (1R,2S,3R)-1-((3R,4R)- | pyrrolidine | | | | y | y | y | | | y | | | |
| | 3,4-dihydroxy-1-(9- | | | | | | | | | | | | | |
| | hydroxynonyl)pyrrolidin- | | | | | | | | | | | | | |
| | 2-yl)butane-1,2,3,4- | | | | | | | | | | | | | |
| | tetraol | | | | | | | | | | | | | |
| 592 | (1R,2S,3R)-1-((3R,4R)- | pyrrolidine | | | | y | y | y | | | y | | | |
| | 3,4-dihydroxy-1-(2-(2- | | | | | | | | | | | | | |
| | methoxyethoxy)ethyl)pyrrolidin- | | | | | | | | | | | | | |
| | 2-yl)butane- | | | | | | | | | | | | | |
| | 1,2,3,4-tetraol | | | | | | | | | | | | | |
| 593 | (1R,2S,3R)-1-((3R,4R)- | pyrrolidine | | | | y | y | y | | | y | | | |
| | 3,4-dihydroxypyrrolidin- | | | | | | | | | | | | | |
| | 2-yl)butane-1,2,3,4- | | | | | | | | | | | | | |
| | tetraol | | | | | | | | | | | | | |
| 594 | (1R,2S,3R)-1-((3R,4R)- | pyrrolidine | | | | y | y | y | | | y | | | |
| | 1-butyl-3,4- | | | | | | | | | | | | | |
| | dihydroxypyrrolidin-2- | | | | | | | | | | | | | |
| | yl)butane-1,2,3,4-tetraol | | | | | | | | | | | | | |
| 595 | (1R,2S,3R)-1-((3R,4R)- | pyrrolidine | | | | y | y | y | | | y | | | |
| | 3,4-dihydroxy-1- | | | | | | | | | | | | | |
| | nonylpyrrolidin-2- | | | | | | | | | | | | | |
| | yl)butane-1,2,3,4-tetraol | | | | | | | | | | | | | |
| 596 | (5R,6R,7S,8R)-5- | piperidine | | | | | | | | | y | | | |
| | methyl-5,6,7,8- | | | | | | | | | | | | | |
| | tetrahydrotetrazolo[1,5- | | | | | | | | | | | | | |
| | a]pyridine-6,7,8-triol | | | | | | | | | | | | | |
| 597 | N-(((2S,3R,4S)-1- | pyrrolidine | | | y | | | | | y | | | | |
| | benzyl-3,4- | | | | | | | | | | | | | |
| | dihydroxypyrrolidin-2- | | | | | | | | | | | | | |
| | yl)methyl)benzamide | | | | | | | | | | | | | |
| 598 | N-(((2S,3R,4S)-1- | pyrrolidine | | | y | | | | | y | | | | |
| | benzyl-3,4- | | | | | | | | | | | | | |
| | dihydroxypyrrolidin-2- | | | | | | | | | | | | | |
| | yl)methyl)acetamide | | | | | | | | | | | | | |
| 599 | N-(((3aR,4S,6aS)-5- | pyrrolidine | | | y | | | | | y | | | | |
| | benzyl-2,2- | | | | | | | | | | | | | |
| | dimethyltetrahydro-3aH- | | | | | | | | | | | | | |
| | [1,3]dioxolo[4,5-c]pyrrol- | | | | | | | | | | | | | |
| | 4-yl)methyl)benzamide | | | | | | | | | | | | | |
| 600 | N-(((2S,3R,4S)-3,4- | pyrrolidine | | | y | | | | | y | | | | |
| | dihydroxy-1-(2- | | | | | | | | | | | | | |
| | hydroxyethyl)pyrrolidin- | | | | | | | | | | | | | |
| | 2-yl)methyl)acetamide | | | | | | | | | | | | | |
| 601 | N-(((2S,3R,4S)-3,4- | pyrrolidine | | | y | | | | | y | | | | |
| | dihydroxypyrrolidin-2- | | | | | | | | | | | | | |
| | yl)methyl)acetamide | | | | | | | | | | | | | |
| 602 | N-(((2S,3R,4S)-3,4- | pyrrolidine | | | y | | | | | y | | | | |
| | dihydroxypyrrolidin-2- | | | | | | | | | | | | | |
| | yl)methyl)benzamide | | | | | | | | | | | | | |
| 603 | N-(((2S,3R,4S)-1-butyl- | pyrrolidine | | | y | | | | | y | | | | |
| | 3,4-dihydroxypyrrolidin- | | | | | | | | | | | | | |
| | 2-yl)methyl)acetamide | | | | | | | | | | | | | |
| 604 | (2S,3R,4S)-2- | pyrrolidine | | | y | | | | | y | | | | |
| | (aminomethyl)-1- | | | | | | | | | | | | | |
| | benzylpyrrolidine-3,4- | | | | | | | | | | | | | |
| | diol | | | | | | | | | | | | | |
| 605 | ((3aS,4S,6aR)-5- | pyrrolidine | | | | | | | | | | y | | |
| | (biphenyl-4-ylmethyl)- | | | | | | | | | | | | | |
| | 2,2-dimethyltetrahydro- | | | | | | | | | | | | | |
| | 3aH-[1,3]dioxolo[4,5- | | | | | | | | | | | | | |
| | c]pyrrol-4-yl)methanol | | | | | | | | | | | | | |
| 606 | (2S,3S,4R)-1-(biphenyl- | pyrrolidine | | y | | | | | | | | | y | |
| | 4-ylmethyl)-2-((S)-1,2- | | | | | | | | | | | | | |
| | dihydroxyethyl)pyrrolidine- | | | | | | | | | | | | | |
| | 3,4-diol | | | | | | | | | | | | | |
| 607 | N-(((2S,3R,4S)-1-butyl- | pyrrolidine | | | y | | | | | y | | | | |
| | 3,4-dihydroxypyrrolidin- | | | | | | | | | | | | | |
| | 2-yl)methyl)benzamide | | | | | | | | | | | | | |
| 608 | N-(((3aR,4S,6aS)-5- | pyrrolidine | | | y | | | | | y | | | | |
| | benzyl-2,2- | | | | | | | | | | | | | |
| | dimethyltetrahydro-3aH- | | | | | | | | | | | | | |
| | [1,3]dioxolo[4,5-c]pyrrol- | | | | | | | | | | | | | |
| | 4-yl)methyl)-2,2,2- | | | | | | | | | | | | | |
| | trifluoroacetamide | | | | | | | | | | | | | |
| 609 | N-(((3aR,4S,6aS)-2,2- | pyrrolidine | | | y | | | | | y | | | | |
| | dimethyltetrahydro-3aH- | | | | | | | | | | | | | |
| | [1,3]dioxolo[4,5-c]pyrrol- | | | | | | | | | | | | | |
| | 4-yl)methyl)acetamide | | | | | | | | | | | | | |
| 610 | N-(((3aR,4S,6aS)-5- | pyrrolidine | | | y | | | | | y | | | | |
| | (biphenyl-4-ylmethyl)- | | | | | | | | | | | | | |
| | 2,2-dimethyltetrahydro- | | | | | | | | | | | | | |
| | 3aH-[1,3]dioxolo[4,5- | | | | | | | | | | | | | |
| | c]pyrrol-4- | | | | | | | | | | | | | |
| | yl)methyl)acetamide | | | | | | | | | | | | | |
| 611 | N-(((3aR,4S,6aS)-2,2- | pyrrolidine | | | y | | | | | y | | | | |
| | dimethyltetrahydro-3aH- | | | | | | | | | | | | | |
| | [1,3]dioxolo[4,5-c]pyrrol- | | | | | | | | | | | | | |
| | 4-yl)methyl)-2,2,2- | | | | | | | | | | | | | |
| | trifluoroacetamide | | | | | | | | | | | | | |
| 612 | N-(((2S,3R,4S)-3,4- | pyrrolidine | | | y | | | | | y | | | | |
| | dihydroxy-1-(2- | | | | | | | | | | | | | |
| | (piperidin-1- | | | | | | | | | | | | | |
| | yl)ethyl)pyrrolidin-2- | | | | | | | | | | | | | |
| | yl)methyl)benzamide | | | | | | | | | | | | | |
| 613 | N-(((2S,3R,4S)-3,4- | pyrrolidine | | | y | | | | | y | | | | |
| | dihydroxy-1-(9- | | | | | | | | | | | | | |
| | hydroxynonyl)pyrrolidin- | | | | | | | | | | | | | |
| | 2-yl)methyl)benzamide | | | | | | | | | | | | | |
| 614 | N-(((2R,3R,4S)-3,4- | pyrrolidine | | | | | | | | | | y | | |
| | dihydroxypyrrolidin-2- | | | | | | | | | | | | | |
| | yl)methyl)benzamide | | | | | | | | | | | | | |
| 615 | N-(((2S,3R,4S)-1-(2- | pyrrolidine | | | y | | | | | y | | | | |
| | (dimethylamino)ethyl)- | | | | | | | | | | | | | |
| | 3,4-dihydroxypyrrolidin- | | | | | | | | | | | | | |
| | 2-yl)methyl)acetamide | | | | | | | | | | | | | |
| 616 | N-(((2S,3R,4S)-3,4- | pyrrolidine | | | y | | | | | y | | | | |
| | dihydroxy-1-(2- | | | | | | | | | | | | | |
| | (piperidin-1- | | | | | | | | | | | | | |
| | yl)ethyl)pyrrolidin-2- | | | | | | | | | | | | | |
| | yl)methyl)acetamide | | | | | | | | | | | | | |
| 617 | N-(((2R,3R,4S)-1-butyl- | pyrrolidine | | | | | | | | | | y | | |
| | 3,4-dihydroxypyrrolidin- | | | | | | | | | | | | | |
| | 2-yl)methyl)benzamide | | | | | | | | | | | | | |
| 618 | N-(((2S,3R,4S)-3,4- | pyrrolidine | | | y | | | | | y | | | | |
| | dihydroxy-1-(2- | | | | | | | | | | | | | |
| | morpholinoethyl)pyrrolidin- | | | | | | | | | | | | | |
| | 2- | | | | | | | | | | | | | |
| | yl)methyl)acetamide | | | | | | | | | | | | | |
| 619 | N-(((2R,3R,4S)-1- | pyrrolidine | | | | | | | | | | y | | |
| | benzyl-3,4- | | | | | | | | | | | | | |
| | dihydroxypyrrolidin-2- | | | | | | | | | | | | | |
| | yl)methyl)-2,2,2- | | | | | | | | | | | | | |
| | trifluoroacetamide | | | | | | | | | | | | | |
| 620 | N-butyl-2- | pyrrolidine | | y | | | | | | | | | y | |
| | ((2R,3S,4R,5R)-3,4- | | | | | | | | | | | | | |
| | dihydroxy-5- | | | | | | | | | | | | | |
| | (hydroxymethyl)-1-(2- | | | | | | | | | | | | | |
| | morpholinoethyl)pyrrolidin- | | | | | | | | | | | | | |
| | 2-yl)acetamide | | | | | | | | | | | | | |
| 621 | N-(((2S,3R,4S)-3,4- | pyrrolidine | | | y | | | | | y | | | | |
| | dihydroxy-1-(2-(2- | | | | | | | | | | | | | |
| | methoxyethoxy)ethyl)pyrrolidin- | | | | | | | | | | | | | |
| | 2- | | | | | | | | | | | | | |
| | yl)methyl)benzamide | | | | | | | | | | | | | |
| 622 | N-(((2S,3R,4S)-3,4- | pyrrolidine | | | y | | | | | y | | | | |
| | dihydroxy-1- | | | | | | | | | | | | | |
| | nonylpyrrolidin-2- | | | | | | | | | | | | | |
| | yl)methyl)benzamide | | | | | | | | | | | | | |
| 623 | (2S,3S,4R)-1-(biphenyl- | pyrrolidine | | | | | | | | | | y | | |
| | 4-ylmethyl)-2- | | | | | | | | | | | | | |
| | (hydroxymethyl)pyrrolidine- | | | | | | | | | | | | | |
| | 3,4-diol | | | | | | | | | | | | | |
| 624 | (1R,2S,3R)-1-((3R,4S)- | pyrrolidine | | | | y | | | | | y | | | |
| | 3,4-dihydroxy-1- | | | | | | | | | | | | | |
| | methylpyrrolidin-2- | | | | | | | | | | | | | |
| | yl)butane-1,2,3,4-tetraol | | | | | | | | | | | | | |
| 625 | (1R,2S,3R)-1-((3R,4R)- | pyrrolidine | | | | y | y | y | | | y | | | |
| | 3,4-dihydroxy-1- | | | | | | | | | | | | | |
| | methylpyrrolidin-2- | | | | | | | | | | | | | |
| | yl)butane-1,2,3,4-tetraol | | | | | | | | | | | | | |
| 626 | (1R,2S,3R)-1-((3R,4R)- | pyrrolidine | | | | y | y | y | | | y | | | |
| | 3,4-dihydroxy-1-(2- | | | | | | | | | | | | | |
| | morpholinoethyl)pyrrolidin- | | | | | | | | | | | | | |
| | 2-yl)butane-1,2,3,4- | | | | | | | | | | | | | |
| | tetraol | | | | | | | | | | | | | |
| 627 | (1R,2S,3R)-1-((3R,4R)- | pyrrolidine | | | | y | y | y | | | y | | | |
| | 3,4-dihydroxy-1-(2- | | | | | | | | | | | | | |
| | (piperidin-1- | | | | | | | | | | | | | |
| | yl)ethyl)pyrrolidin-2- | | | | | | | | | | | | | |
| | yl)butane-1,2,3,4-tetraol | | | | | | | | | | | | | |
| 628 | N-(((2R,3R,4S)-3,4- | pyrrolidine | | | | | | | | | | y | | |
| | dihydroxy-1- | | | | | | | | | | | | | |
| | methylpyrrolidin-2- | | | | | | | | | | | | | |
| | yl)methyl)benzamide | | | | | | | | | | | | | |
| 629 | N-(((3aR,4S,6aS)-2,2- | pyrrolidine | | | y | | | | | y | | | | |
| | dimethyltetrahydro-3aH- | | | | | | | | | | | | | |
| | [1,3]dioxolo[4,5-c]pyrrol- | | | | | | | | | | | | | |
| | 4-yl)methyl)benzamide | | | | | | | | | | | | | |
| 630 | N-(((3aR,4S,6aS)-2,2- | pyrrolidine | | | y | | | | | y | | | | |
| | dimethyl-5- | | | | | | | | | | | | | |
| | nonyltetrahydro-3aH- | | | | | | | | | | | | | |
| | [1,3]dioxolo[4,5-c]pyrrol- | | | | | | | | | | | | | |
| | 4-yl)methyl)acetamide | | | | | | | | | | | | | |
| 631 | 2,2,2-trifluoro-N- | pyrrolidine | | | y | | | | | y | | | | |
| | (((3aR,4S,6aS)-5-(2-(2- | | | | | | | | | | | | | |
| | methoxyethoxy)ethyl)- | | | | | | | | | | | | | |
| | 2,2-dimethyltetrahydro- | | | | | | | | | | | | | |
| | 3aH-[1,3]dioxolo[4,5- | | | | | | | | | | | | | |
| | c]pyrrol-4- | | | | | | | | | | | | | |
| | yl)methyl)acetamide | | | | | | | | | | | | | |
| 632 | N-(((3aR,4S,6aS)-5- | pyrrolidine | | | y | | | | | y | | | | |
| | (biphenyl-4-ylmethyl)- | | | | | | | | | | | | | |
| | 2,2-dimethyltetrahydro- | | | | | | | | | | | | | |
| | 3aH-[1,3]dioxolo[4,5- | | | | | | | | | | | | | |
| | c]pyrrol-4-yl)methyl)- | | | | | | | | | | | | | |
| | 2,2,2-trifluoroacetamide | | | | | | | | | | | | | |
| 633 | N-(((2S,3R,4S)-3,4- | pyrrolidine | | | y | | | | | y | | | | |
| | dihydroxy-1-(2- | | | | | | | | | | | | | |
| | morpholinoethyl)pyrrolidin- | | | | | | | | | | | | | |
| | 2- | | | | | | | | | | | | | |
| | yl)methyl)benzamide | | | | | | | | | | | | | |
| 634 | N-(((2S,3R,4S)-3,4- | pyrrolidine | | | y | | | | | y | | | | |
| | dihydroxy-1-(9- | | | | | | | | | | | | | |
| | hydroxynonyl)pyrrolidin- | | | | | | | | | | | | | |
| | 2-yl)methyl)acetamide | | | | | | | | | | | | | |
| 635 | (1S,2S,3S,6R,7R,7aR)- | pyrrolizidine | | | | | y | y | y | | | | | |
| | 1,6,7-trihydroxy-3- | | | | | | | | | | | | | |
| | (hydroxymethyl)hexahydro- | | | | | | | | | | | | | |
| | 1H-pyrrolizin-2-yl | | | | | | | | | | | | | |
| | methanesulfonate | | | | | | | | | | | | | |
| 636 | (3R,4S,5S)-5- | piperidine | | | | | | | | | | y | | |
| | (aminomethyl)piperidine- | | | | | | | | | | | | | |
| | 3,4-diol | | | | | | | | | | | | | |
| 637 | N-{[(3R,4S,5R)-4,5- | piperidine | | | | | | | | | | y | | |
| | dihydroxypiperidin-3- | | | | | | | | | | | | | |
| | yl]methyl}acetamide | | | | | | | | | | | | | |
| 638 | (2S,4R)-4-hydroxy-1,1- | pyrrolidine | | | | | | | | | | | | |
| | dimethylpyrrolidinium-2- | | | | | | | | | | | | | |
| | carboxylate | | | | | | | | | | | | | |
| 639 | N-((3R,4S,5R)-5- | piperidine | | | | | | | | | | | | |
| | (benzyloxy)-4- | | | | | | | | | | | | | |
| | hydroxypiperidin-3- | | | | | | | | | | | | | |
| | yl)acetamide | | | | | | | | | | | | | |
| 640 | (3S,4S,5S)-1-(2- | piperidine | | | | | | | | | | | | |
| | hydroxyethyl)-5- | | | | | | | | | | | | | |
| | (hydroxymethyl)piperidine- | | | | | | | | | | | | | |
| | 3,4-diol | | | | | | | | | | | | | |
| 641 | (3S,4S,5S)-5- | piperidine | | | | | | | | | | | | |
| | (hydroxymethyl)piperidine- | | | | | | | | | | | | | |
| | 3,4-diol | | | | | | | | | | | | | |
| 642 | (1S,2R,3S,4R,5R)- | other | | | | | Y | Y | Y | | | | | |
| | 2,3,4-trihydroxy-N-(N′- | | | | | | | | | | | | | |
| | octylthiocarbamoyl)-6- | | | | | | | | | | | | | |
| | oxa-nor-tropane | | | | | | | | | | | | | |
| 643 | (5R,6R,7S,8R,8aR)- | other | | | | | Y | Y | Y | | | | | |
| | 5,6,7,8-Tetrahydroxy-3- | | | | | | | | | | | | | |
| | octylimino-2- | | | | | | | | | | | | | |
| | oxaindolizidine | | | | | | | | | | | | | |
| 644 | (1S,2R,3S,4R,5R)-N- | other | | | | | Y | Y | Y | | | | | |
| | (N′-Butylthiocarbamoyl)- | | | | | | | | | | | | | |
| | 2,3,4-trihydroxy-6-oxa- | | | | | | | | | | | | | |
| | nor-tropane | | | | | | | | | | | | | |
| 645 | (3Z,5R,6R,7S,8R,8aR)- | other | | | | | Y | Y | Y | | | | | |
| | 3- | | | | | | | | | | | | | |
| | (octylimino)hexahydro[1, | | | | | | | | | | | | | |
| | 3]thiazolo[3,4- | | | | | | | | | | | | | |
| | a]pyridine-5,6,7,8-tetrol | | | | | | | | | | | | | |
| 646 | N-(((2R,3R,4S)-3,4- | pyrrolidine | | | | | | | | | | Y | | |
| | dihydroxy-1- | | | | | | | | | | | | | |
| | nonylpyrrolidin-2- | | | | | | | | | | | | | |
| | yl)methyl)benzamide | | | | | | | | | | | | | |
| 647 | N-(((2R,3R,4S)-3,4- | pyrrolidine | | | | | | | | | | Y | | |
| | dihydroxy-1-(2- | | | | | | | | | | | | | |
| | hydroxyethyl)pyrrolidin- | | | | | | | | | | | | | |
| | 2-yl)methyl)benzamide | | | | | | | | | | | | | |
| 648 | N-(((2R,3R,4S)-3,4- | pyrrolidine | | | | | | | | | | Y | | |
| | dihydroxy-1-(9- | | | | | | | | | | | | | |
| | hydroxynonyl)pyrrolidin- | | | | | | | | | | | | | |
| | 2-yl)methyl)benzamide | | | | | | | | | | | | | |
| 649 | N-(((2R,3R,4S)-1- | pyrrolidine | | | | | | | | | | Y | | |
| | (biphenyl-4-ylmethyl)- | | | | | | | | | | | | | |
| | 3,4-dihydroxypyrrolidin- | | | | | | | | | | | | | |
| | 2-yl)methyl)benzamide | | | | | | | | | | | | | |
| 650 | N-(((2R,3R,4S)-3,4- | pyrrolidine | | | | | | | | | | Y | | |
| | dihydroxy-1-(2-(2- | | | | | | | | | | | | | |
| | methoxyethoxy)ethyl)pyrrolidin- | | | | | | | | | | | | | |
| | 2- | | | | | | | | | | | | | |
| | yl)methyl)benzamide | | | | | | | | | | | | | |
| 651 | N-(((2R,3R,4S)-3,4- | pyrrolidine | | | | | | | | | | Y | | |
| | dihydroxy-1-(2- | | | | | | | | | | | | | |
| | morpholinoethyl)pyrrolidin- | | | | | | | | | | | | | |
| | 2- | | | | | | | | | | | | | |
| | yl)methyl)benzamide | | | | | | | | | | | | | |
| 652 | N-(((3aR,4S,6aS)-5- | pyrrolidine | | | Y | | | | | Y | | | | |
| | benzyl-2,2- | | | | | | | | | | | | | |
| | dimethyltetrahydro-3aH- | | | | | | | | | | | | | |
| | [1,3]dioxolo[4,5-c]pyrrol- | | | | | | | | | | | | | |
| | 4-yl)methyl)biphenyl-4- | | | | | | | | | | | | | |
| | carboxamide | | | | | | | | | | | | | |
| 653 | N-(((2R,3R,4S)-3,4- | pyrrolidine | | | | | | | | | | Y | | |
| | dihydroxy-1-(2- | | | | | | | | | | | | | |
| | (piperidin-1- | | | | | | | | | | | | | |
| | yl)ethyl)pyrrolidin-2- | | | | | | | | | | | | | |
| | yl)methyl)benzamide | | | | | | | | | | | | | |
| 654 | N-(((2R,3R,4S)-1-(2- | pyrrolidine | | | | | | | | | | Y | | |
| | (dimethylamino)ethyl)- | | | | | | | | | | | | | |
| | 3,4-dihydroxypyrrolidin- | | | | | | | | | | | | | |
| | 2-yl)methyl)benzamide | | | | | | | | | | | | | |
| 655 | 2-((2R,3R,4S)-2- | pyrrolidine | | | | | | | | | | Y | | |
| | (benzamidomethyl)-3,4- | | | | | | | | | | | | | |
| | dihydroxypyrrolidin-1- | | | | | | | | | | | | | |
| | yl)acetic acid | | | | | | | | | | | | | |
| 656 | N-(((2R,3R,4S)-3,4- | pyrrolidine | | | | | | | | | | Y | | |
| | dihydroxy-1- | | | | | | | | | | | | | |
| | methylpyrrolidin-2- | | | | | | | | | | | | | |
| | yl)methyl)acetamide | | | | | | | | | | | | | |
| 657 | N-(((2S,3R,4S)-1- | pyrrolidine | | | Y | | | | | Y | | | | |
| | benzyl-3,4- | | | | | | | | | | | | | |
| | dihydroxypyrrolidin-2- | | | | | | | | | | | | | |
| | yl)methyl)biphenyl-4- | | | | | | | | | | | | | |
| | carboxamide | | | | | | | | | | | | | |
| 658 | N-(((2S,3R,4S)-1-butyl- | pyrrolidine | | | Y | | | | | Y | | | | |
| | 3,4-dihydroxypyrrolidin- | | | | | | | | | | | | | |
| | 2-yl)methyl)biphenyl-4- | | | | | | | | | | | | | |
| | carboxamide | | | | | | | | | | | | | |
| 659 | N-(((2S,3R,4S)-3,4- | pyrrolidine | | | Y | | | | | Y | | | | |
| | dihydroxy-1- | | | | | | | | | | | | | |
| | nonylpyrrolidin-2- | | | | | | | | | | | | | |
| | yl)methyl)biphenyl-4- | | | | | | | | | | | | | |
| | carboxamide | | | | | | | | | | | | | |
| 660 | N-(((2S,3R,4S)-3,4- | pyrrolidine | | | Y | | | | | Y | | | | |
| | dihydroxy-1-(9- | | | | | | | | | | | | | |
| | hydroxynonyl)pyrrolidin- | | | | | | | | | | | | | |
| | 2-yl)methyl)biphenyl-4- | | | | | | | | | | | | | |
| | carboxamide | | | | | | | | | | | | | |
| 661 | N-(((3aR,4S,6aS)-5-(9- | pyrrolidine | | | Y | | | | | Y | | | | |
| | hydroxynonyl)-2,2- | | | | | | | | | | | | | |
| | dimethyltetrahydro-3aH- | | | | | | | | | | | | | |
| | [1,3]dioxolo[4,5-c]pyrrol- | | | | | | | | | | | | | |
| | 4-yl)methyl)benzamide | | | | | | | | | | | | | |
| 662 | N-(((3aR,4S,6aS)-5- | pyrrolidine | | | Y | | | | | Y | | | | |
| | benzyl-2,2- | | | | | | | | | | | | | |
| | dimethyltetrahydro-3aH- | | | | | | | | | | | | | |
| | [1,3]dioxolo[4,5-c]pyrrol- | | | | | | | | | | | | | |
| | 4-yl)methyl)-2,2,2- | | | | | | | | | | | | | |
| | trifluoroacetamide | | | | | | | | | | | | | |
| 663 | 2,2,2-trifluoro-N- | pyrrolidine | | | Y | | | | | Y | | | | |
| | (((3aR,4S,6aS)-5-(9- | | | | | | | | | | | | | |
| | hydroxynonyl)-2,2- | | | | | | | | | | | | | |
| | dimethyltetrahydro-3aH- | | | | | | | | | | | | | |
| | [1,3]dioxolo[4,5-c]pyrrol- | | | | | | | | | | | | | |
| | 4-yl)methyl)acetamide | | | | | | | | | | | | | |
| 664 | N-(((2S,3R,4S)-1- | pyrrolidine | | | Y | | | | | Y | | | | |
| | (biphenyl-4-ylmethyl)- | | | | | | | | | | | | | |
| | 3,4-dihydroxypyrrolidin- | | | | | | | | | | | | | |
| | 2-yl)methyl)benzamide | | | | | | | | | | | | | |
| 665 | 3-((2S,3R,4S)-2- | pyrrolidine | | | Y | | | | | Y | | | | |
| | (acetamidomethyl)-3,4- | | | | | | | | | | | | | |
| | dihydroxypyrrolidin-1- | | | | | | | | | | | | | |
| | yl)propanamide | | | | | | | | | | | | | |
| 666 | N-(((2S,3R,4S)-1-(3- | pyrrolidine | | | Y | | | | | Y | | | | |
| | amino-3-oxopropyl)-3,4- | | | | | | | | | | | | | |
| | dihydroxypyrrolidin-2- | | | | | | | | | | | | | |
| | yl)methyl)benzamide | | | | | | | | | | | | | |
| 667 | N-(((2S,3R,4S)-1-(2- | pyrrolidine | | | Y | | | | | Y | | | | |
| | (dimethylamino)ethyl)- | | | | | | | | | | | | | |
| | 3,4-dihydroxypyrrolidin- | | | | | | | | | | | | | |
| | 2-yl)methyl)benzamide | | | | | | | | | | | | | |
| 668 | N-(((3aR,4R,6aS)-5- | pyrrolidine | | | | | | | | | | Y | | |
| | benzyl-2,2- | | | | | | | | | | | | | |
| | dimethyltetrahydro-3aH- | | | | | | | | | | | | | |
| | [1,3]dioxolo[4,5-c]pyrrol- | | | | | | | | | | | | | |
| | 4-yl)methyl)butyramide | | | | | | | | | | | | | |
| 669 | N-(((2R,3R,4S)-1- | pyrrolidine | | | | | | | | | | Y | | |
| | benzyl-3,4- | | | | | | | | | | | | | |
| | dihydroxypyrrolidin-2- | | | | | | | | | | | | | |
| | yl)methyl)butyramide | | | | | | | | | | | | | |
| 670 | N-(((2S,3R,4S)-3,4- | pyrrolidine | | | Y | | | | | Y | | | | |
| | dihydroxypyrrolidin-2- | | | | | | | | | | | | | |
| | yl)methyl)biphenyl-4- | | | | | | | | | | | | | |
| | carboxamide | | | | | | | | | | | | | |
| 671 | (3aS,4R,6aR)-4- | pyrrolidine | | | Y | | | | | Y | | | | |
| | (azidomethyl)-2,2- | | | | | | | | | | | | | |
| | dimethyltetrahydro-3aH- | | | | | | | | | | | | | |
| | [1,3]dioxolo[4,5- | | | | | | | | | | | | | |
| | c]pyrrole | | | | | | | | | | | | | |
| 672 | N-(((3aR,4R,6aS)-5- | pyrrolidine | | | | | | | | | | Y | | |
| | benzyl-2,2- | | | | | | | | | | | | | |
| | dimethyltetrahydro-3aH- | | | | | | | | | | | | | |
| | [1,3]dioxolo[4,5-c]pyrrol- | | | | | | | | | | | | | |
| | 4-yl)methyl)biphenyl-4- | | | | | | | | | | | | | |
| | carboxamide | | | | | | | | | | | | | |
| 673 | N-(((2R,3R,4S)-1- | pyrrolidine | | | | | | | | | | Y | | |
| | benzyl-3,4- | | | | | | | | | | | | | |
| | dihydroxypyrrolidin-2- | | | | | | | | | | | | | |
| | yl)methyl)biphenyl-4- | | | | | | | | | | | | | |
| | carboxamide | | | | | | | | | | | | | |
| 674 | N-(((2S,3R,4S)-3,4- | pyrrolidine | | | Y | | | | | Y | | | | |
| | dihydroxypyrrolidin-2- | | | | | | | | | | | | | |
| | yl)methyl)-2,2,2- | | | | | | | | | | | | | |
| | trifluoroacetamide | | | | | | | | | | | | | |
| 675 | N-(((3aR,4S,6aS)-2,2- | pyrrolidine | | | Y | | | | | Y | | | | |
| | dimethyl-5-(2- | | | | | | | | | | | | | |
| | morpholinoethyl)tetrahydro- | | | | | | | | | | | | | |
| | 3aH- | | | | | | | | | | | | | |
| | [1,3]dioxolo[4,5-c]pyrrol- | | | | | | | | | | | | | |
| | 4-yl)methyl)-2,2,2- | | | | | | | | | | | | | |
| | trifluoroacetamid | | | | | | | | | | | | | |
| 676 | N-(((3aR,4S,6aS)-5- | pyrrolidine | | | Y | | | | | Y | | | | |
| | butyl-2,2- | | | | | | | | | | | | | |
| | dimethyltetrahydro-3aH- | | | | | | | | | | | | | |
| | [1,3]dioxolo[4,5-c]pyrrol- | | | | | | | | | | | | | |
| | 4-yl)methyl)-2,2,2- | | | | | | | | | | | | | |
| | trifluoroacetamide | | | | | | | | | | | | | |
| 677 | N-(((3aR,4S,6aS)-2,2- | pyrrolidine | | | Y | | | | | Y | | | | |
| | dimethyl-5-(2-(piperidin- | | | | | | | | | | | | | |
| | 1-yl)ethyl)tetrahydro- | | | | | | | | | | | | | |
| | 3aH-[1,3]dioxolo[4,5- | | | | | | | | | | | | | |
| | c]pyrrol-4-yl)methyl)- | | | | | | | | | | | | | |
| | 2,2,2-trifluoroacetamide | | | | | | | | | | | | | |
| 678 | N-(((3aR,4S,6aS)-5-(2- | pyrrolidine | | | Y | | | | | Y | | | | |
| | (dimethylamino)ethyl)- | | | | | | | | | | | | | |
| | 2,2-dimethyltetrahydro- | | | | | | | | | | | | | |
| | 3aH-[1,3]dioxolo[4,5- | | | | | | | | | | | | | |
| | c]pyrrol-4-yl)methyl)- | | | | | | | | | | | | | |
| | 2,2,2-trifluoroacetamide | | | | | | | | | | | | | |
| 679 | N-(((2S,3R,4S)-3,4- | pyrrolidine | | | Y | | | | | Y | | | | |
| | dihydroxy-1- | | | | | | | | | | | | | |
| | methylpyrrolidin-2- | | | | | | | | | | | | | |
| | yl)methyl)acetamide | | | | | | | | | | | | | |
| 680 | (2S,3R,4R)-2- | pyrrolidine | | | | Y | | | | | Y | | | |
| | [(2R,3S,4R)-3,4- | | | | | | | | | | | | | |
| | dihydroxytetrahydrofuran- | | | | | | | | | | | | | |
| | 2-yl]pyrrolidine-3,4- | | | | | | | | | | | | | |
| | diol | | | | | | | | | | | | | |
| 681 | (2R,3R,4R)-1-butyl-2- | piperidine | | | Y | | | | | | | | | Y |
| | (hydroxymethyl)piperidine- | | | | | | | | | | | | | |
| | 3,4-diol | | | | | | | | | | | | | |
| 682 | N-(((2S,3R,4S)-1-butyl- | pyrrolidine | | | Y | | | | | Y | | | | |
| | 3,4-dihydroxypyrrolidin- | | | | | | | | | | | | | |
| | 2-yl)methyl)-2,2,2- | | | | | | | | | | | | | |
| | trifluoroacetamide | | | | | | | | | | | | | |
| 683 | N-(((2S,3R,4S)-3,4- | pyrrolidine | | | Y | | | | | Y | | | | |
| | dihydroxy-1-(2- | | | | | | | | | | | | | |
| | (piperidin-1- | | | | | | | | | | | | | |
| | yl)ethyl)pyrrolidin-2- | | | | | | | | | | | | | |
| | yl)methyl)-2,2,2- | | | | | | | | | | | | | |
| | trifluoroacetamide | | | | | | | | | | | | | |
| 684 | tert-butyl | pyrrolidine | | | Y | | | | | Y | | | | |
| | ((3aR,4S,6aS)-5-(2- | | | | | | | | | | | | | |
| | hydroxyethyl)-2,2- | | | | | | | | | | | | | |
| | dimethyltetrahydro-3aH- | | | | | | | | | | | | | |
| | [1,3]dioxolo[4,5-c]pyrrol- | | | | | | | | | | | | | |
| | 4-yl)methylcarbamate | | | | | | | | | | | | | |
| 685 | dimethyl 1-(((2S,3R,4S)- | pyrrolidine | | | Y | | | | | Y | | | | |
| | 1-benzyl-3,4- | | | | | | | | | | | | | |
| | dihydroxypyrrolidin-2- | | | | | | | | | | | | | |
| | yl)methyl)-1H-1,2,3- | | | | | | | | | | | | | |
| | triazole-4,5- | | | | | | | | | | | | | |
| | dicarboxylate | | | | | | | | | | | | | |
| 686 | (2S,3R,4S)-2- | pyrrolidine | | | Y | | | | | Y | | | | |
| | (aminomethyl)-1- | | | | | | | | | | | | | |
| | (biphenyl-4- | | | | | | | | | | | | | |
| | ylmethyl)pyrrolidine-3,4- | | | | | | | | | | | | | |
| | diol | | | | | | | | | | | | | |
| 687 | (2R,3S,4R)-2- | pyrrolidine | | | | | | | | Y | | | | |
| | (aminomethyl)-1- | | | | | | | | | | | | | |
| | benzylpyrrolidine-3,4- | | | | | | | | | | | | | |
| | diol | | | | | | | | | | | | | |
| 688 | N-(((2S,3R,4S)-3,4- | pyrrolidine | | | | | | | | Y | | | | |
| | dihydroxy-1-(2-(2- | | | | | | | | | | | | | |
| | methoxyethoxy)ethyl)pyrrolidin- | | | | | | | | | | | | | |
| | 2- | | | | | | | | | | | | | |
| | yl)methyl)biphenyl-4- | | | | | | | | | | | | | |
| | carboxamide | | | | | | | | | | | | | |
| 689 | N-(((2R,3R,4S)-1-butyl- | pyrrolidine | | | | | | | | | | Y | | |
| | 3,4-dihydroxypyrrolidin- | | | | | | | | | | | | | |
| | 2-yl)methyl)butyramide | | | | | | | | | | | | | |
| 690 | (2R,3S,4R)-2- | pyrrolidine | | | Y | | | | | Y | | | | |
| | (aminomethyl)pyrrolidine- | | | | | | | | | | | | | |
| | 3,4-diol | | | | | | | | | | | | | |
| 691 | N-((2R,3R)-3- | pyrrolidine | | | | | | | | | | | | Y |
| | ((2R,3R,4R)-3,4- | | | | | | | | | | | | | |
| | dihydroxypyrrolidin-2- | | | | | | | | | | | | | |
| | yl)-2,3- | | | | | | | | | | | | | |
| | dihydroxypropyl)acetamide | | | | | | | | | | | | | |
| 692 | (1R,2R)-1-((2R,3R,4R)- | pyrrolidine | | | | Y | Y | Y | | | | | | |
| | 3,4-dihydroxy-1- | | | | | | | | | | | | | |
| | nonylpyrrolidin-2- | | | | | | | | | | | | | |
| | yl)propane-1,2,3-triol | | | | | | | | | | | | | |
| 693 | (1R,2R)-1-((2R,3R,4R)- | pyrrolidine | | | | Y | Y | Y | | | | | | |
| | 3,4-dihydroxy-1-(2-(2- | | | | | | | | | | | | | |
| | methoxyethoxy)ethyl)pyrrolidin- | | | | | | | | | | | | | |
| | 2-yl)propane- | | | | | | | | | | | | | |
| | 1,2,3-triol | | | | | | | | | | | | | |
| 694 | tert-butyl 4- | pyrrolidine | | | | Y | | | | | Y | | | |
| | (((2R,3R,4S)-3,4- | | | | | | | | | | | | | |
| | dihydroxy-2- | | | | | | | | | | | | | |
| | ((1R,2S,3R)-1,2,3,4- | | | | | | | | | | | | | |
| | tetrahydroxybutyl)pyrrolidin- | | | | | | | | | | | | | |
| | 1- | | | | | | | | | | | | | |
| | yl)methyl)piperidine-1- | | | | | | | | | | | | | |
| | carboxylate | | | | | | | | | | | | | |
| 695 | tert-butyl 4-(((3R,4R)- | pyrrolidine | | | | Y | | Y | | | Y | | Y | |
| | 3,4-dihydroxy-2- | | | | | | | | | | | | | |
| | ((1R,2S,3R)-1,2,3,4- | | | | | | | | | | | | | |
| | tetrahydroxybutyl)pyrrolidin- | | | | | | | | | | | | | |
| | 1- | | | | | | | | | | | | | |
| | yl)methyl)piperidine-1- | | | | | | | | | | | | | |
| | carboxylate | | | | | | | | | | | | | |
| 696 | (1R,2S,3R)-1-((3R,4R)- | pyrrolidine | | | | Y | | Y | | | Y | | Y | |
| | 1-(2- | | | | | | | | | | | | | |
| | (dimethylamino)ethyl)- | | | | | | | | | | | | | |
| | 3,4-dihydroxypyrrolidin- | | | | | | | | | | | | | |
| | 2-yl)butane-1,2,3,4- | | | | | | | | | | | | | |
| | tetraol | | | | | | | | | | | | | |
| 697 | N-((2R,3R)-3- | pyrrolidine | | | | Y | Y | Y | | | | | | |
| | ((2S,3R,4R)-1-benzyl- | | | | | | | | | | | | | |
| | 3,4-dihydroxypyrrolidin- | | | | | | | | | | | | | |
| | 2-yl)-2,3- | | | | | | | | | | | | | |
| | dihydroxypropyl)acetamide | | | | | | | | | | | | | |
| 698 | (2S,3R,4R)-1-benzyl-2- | pyrrolidine | | | | Y | Y | Y | | | | | | |
| | ((1R,2R)-3- | | | | | | | | | | | | | |
| | (benzylamino)-1,2- | | | | | | | | | | | | | |
| | dihydroxypropyl)pyrrolidine- | | | | | | | | | | | | | |
| | 3,4-diol | | | | | | | | | | | | | |
| 699 | (1R,2R)-1-((2R,3R,4R)- | pyrrolidine | | | | Y | Y | Y | | | | | | |
| | 3,4-dihydroxypyrrolidin- | | | | | | | | | | | | | |
| | 2-yl)propane-1,2,3-triol | | | | | | | | | | | | | |
| 700 | (2S,3R,4S)-1-benzyl-2- | pyrrolidine | | | | | | | | | Y | | | |
| | ((S)-2-(benzylamino)-1- | | | | | | | | | | | | | |
| | hydroxyethyl)pyrrolidine- | | | | | | | | | | | | | |
| | 3,4-diol | | | | | | | | | | | | | |
| 701 | N-(((2S,3R,4S)-1- | pyrrolidine | | | Y | | | | | Y | | | | |
| | (biphenyl-4-ylmethyl)- | | | | | | | | | | | | | |
| | 3,4-dihydroxypyrrolidin- | | | | | | | | | | | | | |
| | 2-yl)methyl)biphenyl-4- | | | | | | | | | | | | | |
| | carboxamide | | | | | | | | | | | | | |
| 702 | N-(((2S,3R,4S)-3,4- | pyrrolidine | | | Y | | | | | Y | | | | |
| | dihydroxy-1-(2- | | | | | | | | | | | | | |
| | morpholinoethyl)pyrrolidin- | | | | | | | | | | | | | |
| | 2-yl)methyl)biphenyl- | | | | | | | | | | | | | |
| | 4-carboxamide | | | | | | | | | | | | | |
| 703 | (1R,2R)-1-((2R,3R,4R)- | pyrrolidine | | | | Y | Y | Y | | | | | | |
| | 1-benzyl-3,4- | | | | | | | | | | | | | |
| | dihydroxypyrrolidin-2- | | | | | | | | | | | | | |
| | yl)propane-1,2,3-triol | | | | | | | | | | | | | |
| 704 | (2R,3R,4R)-1-benzyl-2- | pyrrolidine | | | | Y | Y | Y | | | | | | |
| | ((4R,5R)-5- | | | | | | | | | | | | | |
| | ((benzylamino)methyl)- | | | | | | | | | | | | | |
| | 2,2-dimethyl-1,3- | | | | | | | | | | | | | |
| | dioxolan-4- | | | | | | | | | | | | | |
| | yl)pyrrolidine-3,4-diol | | | | | | | | | | | | | |
| 705 | (1R,2R)-1-((2R,3R,4R)- | pyrrolidine | | | | Y | Y | Y | | | | | | |
| | 1-(2- | | | | | | | | | | | | | |
| | (dimethylamino)ethyl)- | | | | | | | | | | | | | |
| | 3,4-dihydroxypyrrolidin- | | | | | | | | | | | | | |
| | 2-yl)propane-1,2,3-triol | | | | | | | | | | | | | |
| 706 | (1R,2R)-1-((2R,3R,4R)- | pyrrolidine | | | | Y | Y | Y | | | | | | |
| | 3,4-dihydroxy-1-(2- | | | | | | | | | | | | | |
| | hydroxyethyl)pyrrolidin- | | | | | | | | | | | | | |
| | 2-yl)propane-1,2,3-triol | | | | | | | | | | | | | |
| 707 | (2S,2′S,3R,3′R,4S,4′S)- | pyrrolidine | | | | Y | | | | | Y | | | |
| | 2,2′-((1R,1′R,2R,2′R)- | | | | | | | | | | | | | |
| | 3,3′-azanediylbis(1,2- | | | | | | | | | | | | | |
| | dihydroxypropane-3,1- | | | | | | | | | | | | | |
| | diyl))bis(1- | | | | | | | | | | | | | |
| | benzylpyrrolidine-3,4- | | | | | | | | | | | | | |
| | diol) | | | | | | | | | | | | | |
| 708 | (2S,3R,4S)-2-((S)-2- | pyrrolidine | | | | | | | | | Y | | | |
| | amino-1- | | | | | | | | | | | | | |
| | hydroxyethyl)pyrrolidine- | | | | | | | | | | | | | |
| | 3,4-diol | | | | | | | | | | | | | |
| 709 | N-((S)-2-((2S,3R,4S)- | pyrrolidine | | | | | | | | | Y | | | |
| | 3,4-dihydroxypyrrolidin- | | | | | | | | | | | | | |
| | 2-yl)-2- | | | | | | | | | | | | | |
| | hydroxyethyl)acetamide | | | | | | | | | | | | | |
| 710 | (2R,4S)-methyl 4- | pyrrolidine | | | | | | | | | | Y | | Y |
| | hydroxypyrrolidine-2- | | | | | | | | | | | | | |
| | carboxylate | | | | | | | | | | | | | |
| 711 | (3R,4R,5R,6R)- | azepane | | | | | | | | | Y | | | |
| | azepane-3,4,5,6-tetraol | | | | | | | | | | | | | |
| 712 | N-butyl-2- | pyrrolidine | | Y | | | | | | | | | Y | |
| | ((2R,3S,4R,5R)-3,4- | | | | | | | | | | | | | |
| | dihydroxy-5- | | | | | | | | | | | | | |
| | (hydroxymethyl)-1-(2- | | | | | | | | | | | | | |
| | (piperidin-1- | | | | | | | | | | | | | |
| | yl)ethyl)pyrrolidin-2- | | | | | | | | | | | | | |
| | yl)acetamide | | | | | | | | | | | | | |
| 713 | (2R,4S)-1-tert-butyl 2- | pyrrolidine | | | | | | | | | | Y | | Y |
| | methyl 4- | | | | | | | | | | | | | |
| | hydroxypyrrolidine-1,2- | | | | | | | | | | | | | |
| | dicarboxylate | | | | | | | | | | | | | |
| 714 | 2-{[(2R,3R,6R)-6-ethyl- | piperidine | | | | | | | | | | | | |
| | 3-hydroxypiperidin-2- | | | | | | | | | | | | | |
| | yl]methoxy}-6- | | | | | | | | | | | | | |
| | (hydroxymethyl)tetrahydro- | | | | | | | | | | | | | |
| | 2H-pyran-3,4,5-triol | | | | | | | | | | | | | |
| 715 | (1S,6R,7R,8S,8aR)- | indolizidine | | | | | | | | | | | | |
| | octahydroindolizine- | | | | | | | | | | | | | |
| | 1,6,7,8-tetraol | | | | | | | | | | | | | |
| 716 | 3-((2S,4S)-4-azido-2- | pyrrolidine | | | Y | | | | | Y | | | | |
| | (hydroxymethyl)pyrrolidin- | | | | | | | | | | | | | |
| | 1-yl)propan-1-ol | | | | | | | | | | | | | |
| 717 | 3-((2R,4R)-4-azido-2- | pyrrolidine | | | Y | | | | | Y | | | | |
| | (hydroxymethyl)pyrrolidin- | | | | | | | | | | | | | |
| | 1-yl)propan-1-ol | | | | | | | | | | | | | |
| 718 | N-(((3aR,4R,6aS)-2,2- | pyrrolidine | | | | | | | | | | Y | | |
| | dimethyltetrahydro-3aH- | | | | | | | | | | | | | |
| | [1,3]dioxolo[4,5-c]pyrrol- | | | | | | | | | | | | | |
| | 4-yl)methyl)butyramide | | | | | | | | | | | | | |
| 719 | (7S,8R,8aS)-methyl 7,8- | other | | | Y | | | | | Y | | | | |
| | dihydroxy-4-oxo- | | | | | | | | | | | | | |
| | 4,6,7,8,8a,9- | | | | | | | | | | | | | |
| | hexahydropyrrolo[1,2- | | | | | | | | | | | | | |
| | d][1,2,3]triazolo[1,5- | | | | | | | | | | | | | |
| | a]pyrazine-3- | | | | | | | | | | | | | |
| | carboxylate | | | | | | | | | | | | | |
| 720 | (2S,3R,4S)-2- | pyrrolidine | | | Y | | | | | Y | | | | |
| | (aminomethyl)-1-(2- | | | | | | | | | | | | | |
| | hydroxyethyl)pyrrolidine- | | | | | | | | | | | | | |
| | 3,4-diol | | | | | | | | | | | | | |
| 721 | (2R,3R,4R)-4-azido-1- | pyrrolidine | | | Y | | | | | Y | | | | |
| | (2-hydroxyethyl)-2- | | | | | | | | | | | | | |
| | (hydroxymethyl)pyrrolidin- | | | | | | | | | | | | | |
| | 3-ol | | | | | | | | | | | | | |
| 722 | (2S,3S,4S)-4-azido-1- | pyrrolidine | | | Y | | | | | Y | | | | |
| | (2-hydroxyethyl)-2- | | | | | | | | | | | | | |
| | (hydroxymethyl)pyrrolidin- | | | | | | | | | | | | | |
| | 3-ol | | | | | | | | | | | | | |
| 723 | 2-((2R,4S)-4-azido-2- | pyrrolidine | | | | | | | | | | Y | | Y |
| | (hydroxymethyl)pyrrolidin- | | | | | | | | | | | | | |
| | 1-yl)ethanol | | | | | | | | | | | | | |
| 724 | (2R,3R,4R,5S)-2- | piperidine | | | | | Y | Y | | | | | | |
| | (hydroxymethyl)-1-(9- | | | | | | | | | | | | | |
| | hydroxynonyl)piperidine- | | | | | | | | | | | | | |
| | 3,4,5-triol | | | | | | | | | | | | | |
| 725 | (2R,3R,4R,5S)-2- | piperidine | | | | | Y | Y | | | | | | |
| | (hydroxymethyl)-1-(2-(2- | | | | | | | | | | | | | |
| | methoxyethoxy)ethyl)piperidine- | | | | | | | | | | | | | |
| | 3,4,5-triol | | | | | | | | | | | | | |
| 726 | (2R,3R,4R,5S)-1-(2- | piperidine | | | | | Y | Y | | | | | | |
| | (dimethylamino)ethyl)-2- | | | | | | | | | | | | | |
| | (hydroxymethyl)piperidine- | | | | | | | | | | | | | |
| | 3,4,5-triol | | | | | | | | | | | | | |
| 727 | N-((3S,5S)-3,5- | piperidine | | | Y | | | | | Y | | | | |
| | dihydroxypiperidin-4- | | | | | | | | | | | | | |
| | yl)acetamide | | | | | | | | | | | | | |
| 728 | (2S,3S,4S,5S)-2-butyl- | pyrrolidine | | | | | Y | Y | | | | | | |
| | 5- | | | | | | | | | | | | | |
| | (hydroxymethyl)pyrrolidine- | | | | | | | | | | | | | |
| | 3,4-diol | | | | | | | | | | | | | |
| 729 | (2S,3S,4S,5S)-2,5- | pyrrolidine | | | | | Y | Y | | | | | | |
| | bis(hydroxymethyl)pyrrolidine- | | | | | | | | | | | | | |
| | 3,4-diol | | | | | | | | | | | | | |
| 730 | N-((3R,4S,5R)-4,5- | piperidine | | | Y | | | | | Y | | | | |
| | dihydroxy-1- | | | | | | | | | | | | | |
| | methylpiperidin-3- | | | | | | | | | | | | | |
| | yl)acetamide | | | | | | | | | | | | | |
| 731 | N-((3R,4S,5R)-4,5- | piperidine | | | Y | | | | | Y | | | | |
| | dihydroxy-1- | | | | | | | | | | | | | |
| | nonylpiperidin-3- | | | | | | | | | | | | | |
| | yl)acetamide | | | | | | | | | | | | | |
| 732 | (2S,3S,4S,5S)-2-ethyl- | pyrrolidine | | | | | | | | | Y | | | |
| | 5- | | | | | | | | | | | | | |
| | (hydroxymethyl)pyrrolidine- | | | | | | | | | | | | | |
| | 3,4-diol | | | | | | | | | | | | | |
| 733 | N-((3S,4R,5R)-4,5- | piperidine | | | | | | | | | | | | Y |
| | dihydroxypiperidin-3- | | | | | | | | | | | | | |
| | yl)acetamide | | | | | | | | | | | | | |
| 734 | (3R,4S,5R,6S)-1-(2- | azepane | | | | Y | | | | | | | | |
| | hydroxyethyl)azepane- | | | | | | | | | | | | | |
| | 3,4,5,6-tetraol | | | | | | | | | | | | | |
| 735 | (3R,4S,5R,6S)-1- | azepane | | | | Y | | | | | | | | |
| | butylazepane-3,4,5,6- | | | | | | | | | | | | | |
| | tetraol | | | | | | | | | | | | | |
| 736 | (3R,4S,5R,6S)-1- | azepane | | | | Y | | | | | | | | |
| | nonylazepane-3,4,5,6- | | | | | | | | | | | | | |
| | tetraol | | | | | | | | | | | | | |
| 737 | (3R,4S,5R,6S)-1-(9- | azepane | | | | Y | | | | | | | | |
| | hydroxynonyl)azepane- | | | | | | | | | | | | | |
| | 3,4,5,6-tetraol | | | | | | | | | | | | | |
| 738 | (3R,4S,5R,6S)-1- | azepane | | | | Y | | | | | | | | |
| | (biphenyl-4- | | | | | | | | | | | | | |
| | ylmethyl)azepane- | | | | | | | | | | | | | |
| | 3,4,5,6-tetraol | | | | | | | | | | | | | |
| 739 | (3R,4S,5R,6S)-1-(2- | azepane | | | | Y | | | | | | | | |
| | (dimethylamino)ethyl)azepane- | | | | | | | | | | | | | |
| | 3,4,5,6-tetraol | | | | | | | | | | | | | |
| 740 | (3R,4S,5R,6S)-1- | azepane | | | | Y | | | | | | | | |
| | benzylazepane-3,4,5,6- | | | | | | | | | | | | | |
| | tetraol | | | | | | | | | | | | | |
| 741 | (R)-(1-butylpiperidin-3- | azepane | | | | | | | | | | | | |
| | yl)methanol | | | | | | | | | | | | | |
| 742 | (3R,4S,5R,6S)- | azepane | | | | Y | | | | | | | | |
| | azepane-3,4,5,6-tetraol | | | | | | | | | | | | | |
| 743 | (2R,3R,4R,5R)-1-(2- | pyrrolidine | | | | | | | | | Y | | | |
| | (dimethylamino)ethyl)- | | | | | | | | | | | | | |
| | 2,5- | | | | | | | | | | | | | |
| | bis(hydroxymethyl)pyrrolidine- | | | | | | | | | | | | | |
| | 3,4-diol | | | | | | | | | | | | | |
| 744 | (R)-2-(3- | piperidine | | | | | | | | | | | | |
| | (hydroxymethyl)piperidin- | | | | | | | | | | | | | |
| | 1-yl)ethanol | | | | | | | | | | | | | |
| 745 | (2R,3R,4R,5R)-1-butyl- | pyrrolidine | | | | | | | | | Y | | | |
| | 2,5- | | | | | | | | | | | | | |
| | bis(hydroxymethyl)pyrrolidine- | | | | | | | | | | | | | |
| | 3,4-diol | | | | | | | | | | | | | |
| 746 | (R)-(1-nonylpiperidin-3- | piperidine | | | | | | | | | | | | |
| | yl)methanol | | | | | | | | | | | | | |
| 747 | (R)-(1-(2-(2- | piperidine | | | | | | | | | | | | |
| | methoxyethoxy)ethyl)piperidin- | | | | | | | | | | | | | |
| | 3-yl)methanol | | | | | | | | | | | | | |
| 748 | 1-(biphenyl-4- | other | | | | | | | | | | | | |
| | ylmethyl)azetidin-3-ol | | | | | | | | | | | | | |
| 749 | 1-(9- | other | | | | | | | | | | | | |
| | hydroxynonyl)azetidin- | | | | | | | | | | | | | |
| | 3-ol | | | | | | | | | | | | | |
| 750 | (1R,4S,7R)-2-oxa-5- | pyrrolidine | | | Y | | | | | Y | | | | |
| | azabicyclo[2.2.1]heptan- | | | | | | | | | | | | | |
| | 7-ol | | | | | | | | | | | | | |
| 751 | (7S,8R,8aR)- | pyrrolidine | | | | | | | | | | Y | | |
| | octahydropyrrolo[1,2- | | | | | | | | | | | | | |
| | a]pyrazine-7,8-diol | | | | | | | | | | | | | |
| 752 | (2S,3R,4S,5R)-1,2- | piperidine | | | | Y | | | | | | | | |
| | dimethylpiperidine- | | | | | | | | | | | | | |
| | 3,4,5-triol | | | | | | | | | | | | | |
| 753 | (6S,7R,8R,8aR)-6,7,8- | piperidine | | | | | Y | Y | | | | | | |
| | trihydroxytetrahydro-1H- | | | | | | | | | | | | | |
| | oxazolo[3,4-a]pyridin- | | | | | | | | | | | | | |
| | 3(5H)-one | | | | | | | | | | | | | |
| 754 | (2R,3R,4R)-1-(2- | piperidine | | | Y | | | | | Y | | | | |
| | hydroxyethyl)-2- | | | | | | | | | | | | | |
| | (hydroxymethyl)piperidine- | | | | | | | | | | | | | |
| | 3,4-diol | | | | | | | | | | | | | |
| 755 | (2R,3R,4R)-2- | piperidine | | | Y | | | | | | | | | |
| | (hydroxymethyl)-1-(2- | | | | | | | | | | | | | |
| | methoxyethyl)piperidine- | | | | | | | | | | | | | |
| | 3,4-diol | | | | | | | | | | | | | |
| 756 | (2R,3R,4R,5S)-1-ethyl- | piperidine | | | | | Y | Y | | | | | | |
| | 2- | | | | | | | | | | | | | |
| | (hydroxymethyl)piperidine- | | | | | | | | | | | | | |
| | 3,4,5-triol | | | | | | | | | | | | | |
| 757 | (1R,2R,3R,4R)-1-butyl- | piperidine | | | Y | | | | | Y | | | | |
| | 3,4-dihydroxy-2- | | | | | | | | | | | | | |
| | (hydroxymethyl)piperidine | | | | | | | | | | | | | |
| | 1-oxide | | | | | | | | | | | | | |
| 758 | (2S,4S,5S)-4,5- | piperidine | | | | Y | Y | Y | | | | | | |
| | dihydroxy-1- | | | | | | | | | | | | | |
| | methylpiperidine-2- | | | | | | | | | | | | | |
| | carboxylic acid | | | | | | | | | | | | | |
| 759 | (4aR,7S,8R,8aR)-5- | piperidine | | | | | Y | Y | | | | | | |
| | benzyl-2,2- | | | | | | | | | | | | | |
| | dimethylhexahydro-4H- | | | | | | | | | | | | | |
| | [1,3]dioxino[5,4- | | | | | | | | | | | | | |
| | b]pyridine-7,8-diol | | | | | | | | | | | | | |
| 760 | (2R,4aR,7S,8R,8aR)- | piperidine | | | | | Y | Y | | | | | | |
| | benzyl 7,8-dihydroxy-2- | | | | | | | | | | | | | |
| | phenyltetrahydro-4H- | | | | | | | | | | | | | |
| | [1,3]dioxino[5,4- | | | | | | | | | | | | | |
| | b]pyridine-5(4aH)- | | | | | | | | | | | | | |
| | carboxylate | | | | | | | | | | | | | |
| 761 | (3R,4R,5R,6R)-1-(2-(2- | azepane | | | | | | | | | Y | | | |
| | methoxyethoxy)ethyl)azepane- | | | | | | | | | | | | | |
| | 3,4,5,6-tetraol | | | | | | | | | | | | | |
| 762 | (3R,4R,5R,6R)-1- | azepane | | | | | | | | | Y | | | |
| | (biphenyl-4- | | | | | | | | | | | | | |
| | ylmethyl)azepane- | | | | | | | | | | | | | |
| | 3,4,5,6-tetraol | | | | | | | | | | | | | |
| 763 | (3R,4R,5R,6R)-1-(9- | azepane | | | | | | | | | Y | | | |
| | hydroxynonyl)azepane- | | | | | | | | | | | | | |
| | 3,4,5,6-tetraol | | | | | | | | | | | | | |
| 764 | (3R,4R,5R,6R)-1- | azepane | | | | | | | | | Y | | | |
| | butylazepane-3,4,5,6- | | | | | | | | | | | | | |
| | tetraol | | | | | | | | | | | | | |
| 765 | 2-((3R,4R,5R,6R)- | azepane | | | | | | | | | Y | | | |
| | 3,4,5,6- | | | | | | | | | | | | | |
| | tetrahydroxyazepan-1- | | | | | | | | | | | | | |
| | yl)acetic acid | | | | | | | | | | | | | |
| 766 | (3R,4R,5R,6R)-1-(5- | azepane | | | | | | | | | Y | | | |
| | (adamantan-1-yl- | | | | | | | | | | | | | |
| | methoxy)- | | | | | | | | | | | | | |
| | pentyl)azepane-3,4,5,6- | | | | | | | | | | | | | |
| | tetraol | | | | | | | | | | | | | |
| 767 | ((2R,4S)-4- | pyrrolidine | | | | | | | | | | Y | | Y |
| | azidopyrrolidin-2- | | | | | | | | | | | | | |
| | yl)methanol | | | | | | | | | | | | | |
| 768 | (2R,4S)-tert-butyl 4- | pyrrolidine | | | | | | | | | | Y | | Y |
| | azido-2- | | | | | | | | | | | | | |
| | (hydroxymethyl)pyrrolidine- | | | | | | | | | | | | | |
| | 1-carboxylate | | | | | | | | | | | | | |
| 769 | (3R,4R,5S,6R)-3,4,5,6- | azepane | | | | | Y | Y | | | | | | |
| | tetrahydroxyazepan-2- | | | | | | | | | | | | | |
| | one | | | | | | | | | | | | | |
| 770 | (3R,4S,5S,6S)- | azepane | | | | | Y | Y | | | | | | |
| | azepane-3,4,5,6-tetraol | | | | | | | | | | | | | |
| 771 | N-((3R,5R)-3,5- | piperidine | | | Y | | | | | Y | | | | |
| | dihydroxypiperidin-4- | | | | | | | | | | | | | |
| | yl)acetamide | | | | | | | | | | | | | |
| 772 | N-((3R,4S,5S)-4,5- | piperidine | | | | | | | | | | | | Y |
| | dihydroxy-1- | | | | | | | | | | | | | |
| | methylpiperidin-3- | | | | | | | | | | | | | |
| | yl)acetamide | | | | | | | | | | | | | |
| 773 | N-((3R,4S,5S)-1-butyl- | piperidine | | | | | | | | | | | | Y |
| | 4,5-dihydroxypiperidin- | | | | | | | | | | | | | |
| | 3-yl)acetamide | | | | | | | | | | | | | |
| 774 | N-((3R,4S,5S)-4,5- | piperidine | | | | | | | | | | | | Y |
| | dihydroxy-1- | | | | | | | | | | | | | |
| | nonylpiperidin-3- | | | | | | | | | | | | | |
| | yl)acetamide | | | | | | | | | | | | | |
| 775 | N-((3S,5S)-3,5- | piperidine | | | Y | | | | | Y | | | | |
| | dihydroxy-1- | | | | | | | | | | | | | |
| | methylpiperidin-4- | | | | | | | | | | | | | |
| | yl)acetamide | | | | | | | | | | | | | |
| 776 | N-((3S,5S)-1-butyl-3,5- | piperidine | | | Y | | | | | Y | | | | |
| | dihydroxypiperidin-4- | | | | | | | | | | | | | |
| | yl)acetamide | | | | | | | | | | | | | |
| 777 | N-((3S,5S)-3,5- | piperidine | | | Y | | | | | Y | | | | |
| | dihydroxy-1- | | | | | | | | | | | | | |
| | nonylpiperidin-4- | | | | | | | | | | | | | |
| | yl)acetamide | | | | | | | | | | | | | |
| 778 | N-((3S,4R,5R)-4,5- | piperidine | | | | | | | | | | | | Y |
| | dihydroxy-1- | | | | | | | | | | | | | |
| | methylpiperidin-3- | | | | | | | | | | | | | |
| | yl)acetamide | | | | | | | | | | | | | |
| 779 | N-((3S,4R,5R)-1-butyl- | piperidine | | | | | | | | | | | | Y |
| | 4,5-dihydroxypiperidin- | | | | | | | | | | | | | |
| | 3-yl)acetamide | | | | | | | | | | | | | |
| 780 | N-((3S,4R,5R)-4,5- | piperidine | | | | | | | | | | | | Y |
| | dihydroxy-1- | | | | | | | | | | | | | |
| | nonylpiperidin-3- | | | | | | | | | | | | | |
| | yl)acetamide | | | | | | | | | | | | | |
| 781 | N-((3R,5R)-3,5- | piperidine | | | Y | | | | | Y | | | | |
| | dihydroxy-1- | | | | | | | | | | | | | |
| | methylpiperidin-4- | | | | | | | | | | | | | |
| | yl)acetamide | | | | | | | | | | | | | |
| 782 | N-((3R,5R)-1-butyl-3,5- | piperidine | | | Y | | | | | Y | | | | |
| | dihydroxypiperidin-4- | | | | | | | | | | | | | |
| | yl)acetamide | | | | | | | | | | | | | |
| 783 | N-((3R,5R)-3,5- | piperidine | | | Y | | | | | Y | | | | |
| | dihydroxy-1- | | | | | | | | | | | | | |
| | nonylpiperidin-4- | | | | | | | | | | | | | |
| | yl)acetamide | | | | | | | | | | | | | |
| 784 | N-((3R,4S,5R)-1-butyl- | piperidine | | | Y | | | | | Y | | | | |
| | 4,5-dihydroxypiperidin- | | | | | | | | | | | | | |
| | 3-yl)acetamide | | | | | | | | | | | | | |
| 785 | N-((3S,4r,5R)-3,5- | piperidine | | | | | | | | | | | | Y |
| | dihydroxypiperidin-4- | | | | | | | | | | | | | |
| | yl)acetamide | | | | | | | | | | | | | |
| 786 | N-((3S,4r,5R)-3,5- | piperidine | | | | | | | | | | | | Y |
| | dihydroxy-1- | | | | | | | | | | | | | |
| | methylpiperidin-4- | | | | | | | | | | | | | |
| | yl)acetamide | | | | | | | | | | | | | |
| 787 | N-((3S,4r,5R)-1-butyl- | piperidine | | | | | | | | | | | | Y |
| | 3,5-dihydroxypiperidin- | | | | | | | | | | | | | |
| | 4-yl)acetamide | | | | | | | | | | | | | |
| 788 | (2R,3S,4R,5R)-2- | pyrrolidine | | | | Y | | | | | | | | |
| | (hydroxymethyl)-5- | | | | | | | | | | | | | |
| | methylpyrrolidine-3,4- | | | | | | | | | | | | | |
| | diol | | | | | | | | | | | | | |
| 789 | N-((3S,4r,5R)-3,5- | piperidine | | | | | | | | | | | | Y |
| | dihydroxy-1- | | | | | | | | | | | | | |
| | nonylpiperidin-4- | | | | | | | | | | | | | |
| | yl)acetamide | | | | | | | | | | | | | |
| 790 | N-((3R,4R,5S)-3- | piperidine | | | | | | | | | | | | Y |
| | (benzyloxy)-1-butyl-5- | | | | | | | | | | | | | |
| | hydroxypiperidin-4- | | | | | | | | | | | | | |
| | yl)acetamide | | | | | | | | | | | | | |
| 791 | (2S,3R,4S,5S)-2- | piperidine | | Y | | | | | | | | | Y | |
| | (hydroxymethyl)piperidine- | | | | | | | | | | | | | |
| | 3,4,5-triol | | | | | | | | | | | | | |
| 792 | (2R,3R,4R,5S)-1-(5- | piperidine | | | | | Y | Y | | | | | | |
| | (adamantan-1-yl- | | | | | | | | | | | | | |
| | methoxy)-pentyl)2- | | | | | | | | | | | | | |
| | (hydroxymethyl)- | | | | | | | | | | | | | |
| | piperidine-3,4,5-triol | | | | | | | | | | | | | |
| 793 | (3R,4R,5R,6R)-3,4,5,6- | azepane | Y | | | | | | | | | | | |
| | tetrahydroxyazepan-2- | | | | | | | | | | | | | |
| | one | | | | | | | | | | | | | |
| 794 | (3R,4R,5R,6R)-1- | azepane | | | | | | | | | Y | | | |
| | nonylazepane-3,4,5,6- | | | | | | | | | | | | | |
| | tetraol | | | | | | | | | | | | | |
| 795 | (2R,3R,4S,5R)-2- | pyrrolidine | | Y | | | | | | | | | Y | |
| | benzyl-5- | | | | | | | | | | | | | |
| | (hydroxymethyl)pyrrolidine- | | | | | | | | | | | | | |
| | 3,4-diol | | | | | | | | | | | | | |
| 796 | (2S,3S,4R)-2-((R)-1,2- | pyrrolidine | Y | | | | | | | | | | | |
| | dihydroxyethyl)-1- | | | | | | | | | | | | | |
| | methylpyrrolidine-3,4- | | | | | | | | | | | | | |
| | diol | | | | | | | | | | | | | |
| 797 | (2S,3R,4S,5R,6R)- | piperidine | | Y | | | Y | Y | | | | | Y | |
| | 3,4,5-trihydroxy-2,6- | | | | | | | | | | | | | |
| | bis(hydroxymethyl)piperidinium | | | | | | | | | | | | | |
| | chloride | | | | | | | | | | | | | |
| 798 | (2S,3R,4R,5R,6R)-2- | piperidine | | | | Y | | | | | Y | | | |
| | ethyl-6- | | | | | | | | | | | | | |
| | (hydroxymethyl)piperidine- | | | | | | | | | | | | | |
| | 3,4,5-triol | | | | | | | | | | | | | |
| 799 | (2R,3S,4R)-1-benzyl-2- | pyrrolidine | | | | | | | | | Y | | | |
| | ((S)-1,2- | | | | | | | | | | | | | |
| | dihydroxyethyl)pyrrolidine- | | | | | | | | | | | | | |
| | 3,4-diol | | | | | | | | | | | | | |
| 800 | (1S,2R,7R,7aR)- | pyrrolizidine | | | | | | | | | Y | | | |
| | hexahydro-1H- | | | | | | | | | | | | | |
| | pyrrolizine-1,2,7-triol | | | | | | | | | | | | | |
| 801 | (2R,3S,4R)-2-((R)-1- | pyrrolidine | | | | | | | | | Y | | | |
| | hydroxyethyl)pyrrolidine- | | | | | | | | | | | | | |
| | 3,4-diol | | | | | | | | | | | | | |
| 802 | N-((3S,4R,5R,6R)-4,5- | piperidine | | | | | Y | Y | | | | | | |
| | dihydroxy-6- | | | | | | | | | | | | | |
| | (hydroxymethyl)piperidin- | | | | | | | | | | | | | |
| | 3-yl)acetamide | | | | | | | | | | | | | |
| 803 | (2S,3S,4R)-2-((S)-2- | pyrrolidine | | | | | | | | | Y | | | |
| | fluoro-1- | | | | | | | | | | | | | |
| | hydroxyethyl)pyrrolidine- | | | | | | | | | | | | | |
| | 3,4-diol | | | | | | | | | | | | | |
| 804 | (2S,3R,4R,5S)-1- | pyrrolidine | | | | | | | Y | | | | | |
| | benzyl-2,5- | | | | | | | | | | | | | |
| | bis(hydroxymethyl)pyrrolidine- | | | | | | | | | | | | | |
| | 3,4-diol | | | | | | | | | | | | | |
| 805 | (1S,2S,3S,5R,8aS)-3- | indolizidine | | | | | | | | | Y | | | |
| | (hydroxymethyl)-5- | | | | | | | | | | | | | |
| | methyloctahydroindolizine- | | | | | | | | | | | | | |
| | 1,2-diol | | | | | | | | | | | | | |
| 806 | (2S,3R,4R,5R)-2- | piperidine | | | | | Y | | Y | | | | | |
| | (hydroxymethyl)piperidine- | | | | | | | | | | | | | |
| | 3,4,5-triol | | | | | | | | | | | | | |
| 807 | (2R,3R,4R)-1-(4- | pyrrolidine | | | Y | | | | | Y | | | | |
| | chlorobenzyl)-2- | | | | | | | | | | | | | |
| | (hydroxymethyl)pyrrolidine- | | | | | | | | | | | | | |
| | 3,4-diol | | | | | | | | | | | | | |
| 808 | (2R,3R,4R)-2- | pyrrolidine | | | Y | | | | | Y | | | | |
| | (hydroxymethyl)-1-(3- | | | | | | | | | | | | | |
| | phenylpropyl)pyrrolidine- | | | | | | | | | | | | | |
| | 3,4-diol | | | | | | | | | | | | | |
| 809 | (2R,3R,4S)-2-((R)-1,2- | pyrrolidine | | Y | | | | | | | | | Y | |
| | dihydroxyethyl)pyrrolidine- | | | | | | | | | | | | | |
| | 3,4-diol | | | | | | | | | | | | | |
| 810 | (2R,3R,4S)-1-benzyl-2- | pyrrolidine | | Y | | | | | | | | | Y | |
| | ((R)-1,2- | | | | | | | | | | | | | |
| | dihydroxyethyl)pyrrolidine- | | | | | | | | | | | | | |
| | 3,4-diol | | | | | | | | | | | | | |
| 811 | (2R,3R,4R,5S)-2- | piperidine | Y | | | | | | | | | | | |
| | (hydroxymethyl)-5- | | | | | | | | | | | | | |
| | methylpiperidine-3,4,5- | | | | | | | | | | | | | |
| | triol | | | | | | | | | | | | | |
| 812 | (2S,3R,4R)-1-benzyl-2- | pyrrolidine | | | | | | | Y | | | | | |
| | ((S)-1,2- | | | | | | | | | | | | | |
| | dihydroxyethyl)pyrrolidine- | | | | | | | | | | | | | |
| | 3,4-diol | | | | | | | | | | | | | |
| 813 | (2S,3R,4R)-2-((S)-1,2- | pyrrolidine | | | | | | | Y | | | | | |
| | dihydroxyethyl)pyrrolidine- | | | | | | | | | | | | | |
| | 3,4-diol | | | | | | | | | | | | | |
| 814 | (1R,4S,7R)-2-benzyl- | pyrrolidine | | | Y | | | | | Y | | | | |
| | 2,5- | | | | | | | | | | | | | |
| | diazabicyclo[2.2.1]heptan- | | | | | | | | | | | | | |
| | 7-ol | | | | | | | | | | | | | |
| 815 | (3S,4S,5S,6S)- | azepane | | | | | | | | | Y | | | |
| | azepane-3,4,5,6-tetraol | | | | | | | | | | | | | |
| 816 | (2R,3S,4S)-1-butyl-2- | piperidine | | | | | | | | | | | | Y |
| | (hydroxymethyl)piperidine- | | | | | | | | | | | | | |
| | 3,4-diol | | | | | | | | | | | | | |
| 817 | (2R,3S,4R)-2-((R)-1,2- | pyrrolidine | | | | | Y | Y | | | | | | |
| | dihydroxyethyl)pyrrolidine- | | | | | | | | | | | | | |
| | 3,4-diol | | | | | | | | | | | | | |
| 818 | (3R,4S,5R,6R)- | azepane | | Y | | | | | | | | | Y | |
| | azepane-3,4,5,6-tetraol | | | | | | | | | | | | | |
| 819 | (2S,3S,4S)-2- | pyrrolidine | | | Y | | | | | Y | | | | |
| | (hydroxymethyl)-1-(3- | | | | | | | | | | | | | |
| | phenylpropyl)pyrrolidine- | | | | | | | | | | | | | |
| | 3,4-diol | | | | | | | | | | | | | |
| 820 | (2S,3R,4R)-1-butyl-2- | pyrrolidine | | | | | | | Y | | | | | |
| | ((S)-1,2- | | | | | | | | | | | | | |
| | dihydroxyethyl)pyrrolidine- | | | | | | | | | | | | | |
| | 3,4-diol | | | | | | | | | | | | | |
| 821 | (2S,3R,4R)-2-((S)-1,2- | pyrrolidine | | | | | | | Y | | | | | |
| | dihydroxyethyl)-1-(2- | | | | | | | | | | | | | |
| | hydroxyethyl)pyrrolidine- | | | | | | | | | | | | | |
| | 3,4-diol | | | | | | | | | | | | | |
| 822 | (2R,3R,4R,5S)-1-hexyl- | piperidine | | | | | Y | Y | | | | | | |
| | 2- | | | | | | | | | | | | | |
| | (hydroxymethyl)piperidine- | | | | | | | | | | | | | |
| | 3,4,5-triol | | | | | | | | | | | | | |
| 823 | (2S,3R,4S)-2- | pyrrolidine | | | Y | | | | | Y | | | | |
| | (hydroxymethyl)-1-(4- | | | | | | | | | | | | | |
| | methoxybenzyl)pyrrolidine- | | | | | | | | | | | | | |
| | 3,4-diol | | | | | | | | | | | | | |
| 824 | (3R,4S,5S,6R)- | azepane | | | | | | | Y | | | | | |
| | azepane-3,4,5,6-tetraol | | | | | | | | | | | | | |
| 825 | (2R,3S,4S)-1-benzyl-2- | pyrrolidine | | | | | | | Y | | | | | |
| | ((R)-1,2- | | | | | | | | | | | | | |
| | dihydroxyethyl)pyrrolidine- | | | | | | | | | | | | | |
| | 3,4-diol | | | | | | | | | | | | | |
| 826 | (2R,3S,4S,5R)-2,5- | pyrrolidine | | | | | | | Y | | | | | |
| | bis(hydroxymethyl)pyrrolidine- | | | | | | | | | | | | | |
| | 3,4-diol | | | | | | | | | | | | | |
| 827 | N-((3S,4S,5R)-4,5- | piperidine | | | | | | | | | | Y | | |
| | dihydroxypiperidin-3- | | | | | | | | | | | | | |
| | yl)acetamide | | | | | | | | | | | | | |
| 828 | N-((3R,4R,5S)-4,5- | piperidine | | | | | | | | | | Y | | |
| | dihydroxypiperidin-3- | | | | | | | | | | | | | |
| | yl)acetamide | | | | | | | | | | | | | |
| 829 | (1S,2S,3R,6S,9aS)-6- | quinolizidine | | | | | Y | Y | | | | | | |
| | methyloctahydro-1H- | | | | | | | | | | | | | |
| | quinolizine-1,2,3-triol | | | | | | | | | | | | | |
| 830 | N-((3S,4S,5R)-4,5- | piperidine | | | | | | | | | | Y | | |
| | dihydroxy-1- | | | | | | | | | | | | | |
| | methylpiperidin-3- | | | | | | | | | | | | | |
| | yl)acetamide | | | | | | | | | | | | | |
| 831 | N-((3S,4S,5R)-4,5- | piperidine | | | | | | | | | | Y | | |
| | dihydroxy-1- | | | | | | | | | | | | | |
| | nonylpiperidin-3- | | | | | | | | | | | | | |
| | yl)acetamide | | | | | | | | | | | | | |
| 832 | N-((3R,4R,5S)-4,5- | piperidine | | | | | | | | | | Y | | |
| | dihydroxy-1- | | | | | | | | | | | | | |
| | methylpiperidin-3- | | | | | | | | | | | | | |
| | yl)acetamide | | | | | | | | | | | | | |
| 833 | N-((3R,4R,5S)-1-butyl- | piperidine | | | | | | | | | | Y | | |
| | 4,5-dihydroxypiperidin- | | | | | | | | | | | | | |
| | 3-yl)acetamide | | | | | | | | | | | | | |
| 834 | N-((3S,4S,5R)-1-butyl- | piperidine | | | | | | | | | | Y | | |
| | 4,5-dihydroxypiperidin- | | | | | | | | | | | | | |
| | 3-yl)acetamide | | | | | | | | | | | | | |
| 835 | (3R,4S,5S)-5- | piperidine | | | | | | | | | | Y | | |
| | aminopiperidine-3,4-diol | | | | | | | | | | | | | |
| 836 | 2-((3R,4r,5S)-3,4,5- | piperidine | | | | | | | | | | | | Y |
| | trihydroxypiperidin-1- | | | | | | | | | | | | | |
| | yl)acetonitrile | | | | | | | | | | | | | |
| 837 | (3R,4r,5S)-1-(2- | piperidine | | | | | | | | | | | | Y |
| | hydroxyethyl)piperidine- | | | | | | | | | | | | | |
| | 3,4,5-triol | | | | | | | | | | | | | |
| 838 | (3R,4r,5S)-1-(2-(2- | piperidine | | | | | | | | | | | | Y |
| | methoxyethoxy)ethyl)piperidine- | | | | | | | | | | | | | |
| | 3,4,5-triol | | | | | | | | | | | | | |
| 839 | (2R,3R,4R,5R)-2-((R)- | piperidine | | | | Y | | | | | Y | | | |
| | 1,2- | | | | | | | | | | | | | |
| | dihydroxyethyl)piperidine- | | | | | | | | | | | | | |
| | 3,4,5-triol | | | | | | | | | | | | | |
| 840 | (2R,3R,4S,5R)-2-((R)- | piperidine | | Y | | | | | | | | | Y | |
| | 1,2- | | | | | | | | | | | | | |
| | dihydroxyethyl)piperidine- | | | | | | | | | | | | | |
| | 3,4,5-triol | | | | | | | | | | | | | |
| 841 | (2R,3R,4R,5S)-2- | pyrrolidine | | | | | Y | Y | | | | | | |
| | (hydroxymethyl)-5- | | | | | | | | | | | | | |
| | methylpyrrolidine-3,4- | | | | | | | | | | | | | |
| | diol | | | | | | | | | | | | | |
| 842 | (2R,3S,4R)-2-((S)-1,2- | pyrrolidine | | | | | | | | | Y | | | |
| | dihydroxyethyl)-1- | | | | | | | | | | | | | |
| | methylpyrrolidine-3,4- | | | | | | | | | | | | | |
| | diol | | | | | | | | | | | | | |
| 843 | (3R,4R,5R)-3- | piperidine | | | Y | | | | | Y | | | | |
| | (hydroxymethyl)piperazine- | | | | | | | | | | | | | |
| | 4,5-diol | | | | | | | | | | | | | |
| 844 | (4R,5R,6R)-6- | piperidine | | | Y | | | | | Y | | | | |
| | (hydroxymethyl)-1- | | | | | | | | | | | | | |
| | methylpiperazine-4,5- | | | | | | | | | | | | | |
| | diol | | | | | | | | | | | | | |
| 845 | retronecine N-oxide | pyrrolizidine | | | | | | | | | | | | |
| 846 | 1-((3R,4R,5R)-4,5- | piperidine | | | Y | | | | | Y | | | | |
| | dihydroxy-3- | | | | | | | | | | | | | |
| | (hydroxymethyl)piperazin- | | | | | | | | | | | | | |
| | 1-yl)ethanone | | | | | | | | | | | | | |
| 847 | (2S,3R,4R,5R)-2-((R)- | piperidine | | | | | Y | Y | | | | | | |
| | 1,2- | | | | | | | | | | | | | |
| | dihydroxyethyl)piperidine- | | | | | | | | | | | | | |
| | 3,4,5-triol | | | | | | | | | | | | | |
| 848 | (2R,3S,4S)-2-((R)-1,2- | pyrrolidine | | | | | | | Y | | | | | |
| | dihydroxyethyl)pyrrolidine- | | | | | | | | | | | | | |
| | 3,4-diol | | | | | | | | | | | | | |
| 849 | (1S,2S,8R,8aS)- | indolizidine | | | | Y | | | | | | | | |
| | octahydroindolizine- | | | | | | | | | | | | | |
| | 1,2,8-triol | | | | | | | | | | | | | |
| 850 | N-((3R,4R,5R,6R)-4,5- | piperidine | | | | | Y | Y | | | | | | |
| | dihydroxy-6- | | | | | | | | | | | | | |
| | (hydroxymethyl)-2- | | | | | | | | | | | | | |
| | oxopiperidin-3- | | | | | | | | | | | | | |
| | yl)acetamide | | | | | | | | | | | | | |
| 851 | (2R,3S,4R,5R)-2-((S)- | pyrrolidine | | Y | | | | | | | Y | | Y | |
| | 1,2-dihydroxyethyl)-5- | | | | | | | | | | | | | |
| | (hydroxymethyl)pyrrolidine- | | | | | | | | | | | | | |
| | 3,4-diol | | | | | | | | | | | | | |
| 852 | (3R,5R)-1- | piperidine | | | Y | | | | | Y | | | | |
| | hexylpiperidine-3,4,5- | | | | | | | | | | | | | |
| | triol | | | | | | | | | | | | | |
| 853 | (3R,4r,5S)-1- | piperidine | | | | | | | | | | | | Y |
| | hexylpiperidine-3,4,5- | | | | | | | | | | | | | |
| | triol | | | | | | | | | | | | | |
| 854 | (1R,2R,3R,7S,7aR)-3- | pyrrolizidine | | Y | Y | | | | | Y | Y | Y | Y | |
| | ((allylamino)methyl)hexahydro- | | | | | | | | | | | | | |
| | 1H-pyrrolizine- | | | | | | | | | | | | | |
| | 1,2,7-triol | | | | | | | | | | | | | |
| 855 | 2-((1R,2R,3R,7S,7aR)- | pyrrolizidine | | Y | Y | | | | | Y | Y | Y | Y | |
| | 1,2,7- | | | | | | | | | | | | | |
| | trihydroxyhexahydro- | | | | | | | | | | | | | |
| | 1H-pyrrolizin-3- | | | | | | | | | | | | | |
| | yl)acetonitrile | | | | | | | | | | | | | |
| 856 | (3S,5S)-1- | piperidine | | | Y | | | | | Y | | | | |
| | hexylpiperidine-3,4,5- | | | | | | | | | | | | | |
| | triol | | | | | | | | | | | | | |
| 857 | (1R,2R,3R,7S,7aR)-3- | pyrrolizidine | | Y | Y | | | | | Y | Y | Y | Y | |
| | ((benzylamino)methyl)hexahydro- | | | | | | | | | | | | | |
| | 1H- | | | | | | | | | | | | | |
| | pyrrolizine-1,2,7-triol | | | | | | | | | | | | | |
| 858 | (2R,3S,4R,5S)-1-(2- | piperidine | | | | Y | | | | | | | | |
| | hydroxyethyl)-2- | | | | | | | | | | | | | |
| | methylpiperidine-3,4,5- | | | | | | | | | | | | | |
| | triol | | | | | | | | | | | | | |
| 859 | (2R,3S,4R,5S)-1-butyl- | piperidine | | | | Y | | | | | | | | |
| | 2-methylpiperidine- | | | | | | | | | | | | | |
| | 3,4,5-triol | | | | | | | | | | | | | |
| 860 | (2R,3S,4R,5S)-1-(2-(2- | piperidine | | | | Y | | | | | | | | |
| | methoxyethoxy)ethyl)-2- | | | | | | | | | | | | | |
| | methylpiperidine-3,4,5- | | | | | | | | | | | | | |
| | triol | | | | | | | | | | | | | |
| 861 | 2-((2R,3S,4R,5S)-3,4,5- | piperidine | | | | Y | | | | | | | | |
| | trihydroxy-2- | | | | | | | | | | | | | |
| | methylpiperidin-1- | | | | | | | | | | | | | |
| | yl)acetic acid | | | | | | | | | | | | | |
| 862 | (2R,3S,4R,5S)-1-(6- | piperidine | | | | Y | | | | | | | | |
| | hydroxyhexyl)-2- | | | | | | | | | | | | | |
| | methylpiperidine-3,4,5- | | | | | | | | | | | | | |
| | triol | | | | | | | | | | | | | |
| 863 | (2R,3S,4R,5S)-2- | piperidine | | | | Y | | | | | | | | |
| | methyl-1-(2- | | | | | | | | | | | | | |
| | morpholinoethyl)piperidine- | | | | | | | | | | | | | |
| | 3,4,5-triol | | | | | | | | | | | | | |
| 864 | (2R,3S,4R,5S)-2- | piperidine | | | | Y | | | | | | | | |
| | methyl-1-(2-(piperidin-1- | | | | | | | | | | | | | |
| | yl)ethyl)piperidine-3,4,5- | | | | | | | | | | | | | |
| | triol | | | | | | | | | | | | | |
| 865 | (2R,3S,4R,5S)-1-(2- | piperidine | | | | Y | | | | | | | | |
| | (dimethylamino)ethyl)-2- | | | | | | | | | | | | | |
| | methylpiperidine-3,4,5- | | | | | | | | | | | | | |
| | triol | | | | | | | | | | | | | |
| 866 | (2R,3S,4R,5S)-1-(6- | piperidine | | | | Y | | | | | | | | |
| | (2,5- | | | | | | | | | | | | | |
| | dimethylphenoxy)hexyl)- | | | | | | | | | | | | | |
| | 2-methylpiperidine- | | | | | | | | | | | | | |
| | 3,4,5-triol | | | | | | | | | | | | | |
| 867 | (2R,3S,4R,5S)-2- | piperidine | | | | Y | | | | | | | | |
| | methyl-1-(6-((1r,4R)-4- | | | | | | | | | | | | | |
| | methylcyclohexyloxy)hexyl)piperidine-3,4,5-triol | | | | | | | | | | | | | |
| 868 | 2-((3R,4r,5S)-3,4,5- | piperidine | | | | | | | | | | | | Y |
| | trihydroxypiperidin-1- | | | | | | | | | | | | | |
| | yl)acetic acid | | | | | | | | | | | | | |
| 869 | N-((3R,4S,5S)-4,5- | piperidine | | | | | | | | | | | | Y |
| | dihydroxypiperidin-3- | | | | | | | | | | | | | |
| | yl)acetamide | | | | | | | | | | | | | |
| 870 | N-((3S,4R,5S)-4,5- | piperidine | | | Y | | | | | Y | | | | |
| | dihydroxy-1- | | | | | | | | | | | | | |
| | methylpiperidin-3- | | | | | | | | | | | | | |
| | yl)acetamide | | | | | | | | | | | | | |
| 871 | N-((3S,4R,5S)-1-butyl- | piperidine | | | Y | | | | | Y | | | | |
| | 4,5-dihydroxypiperidin- | | | | | | | | | | | | | |
| | 3-yl)acetamide | | | | | | | | | | | | | |
| 872 | (2S,3S,4S,5S)-2-(4- | piperidine | | | | | | | | | y | | | |
| | methoxyphenyl)piperidine- | | | | | | | | | | | | | |
| | 3,4,5-triol | | | | | | | | | | | | | |
| 873 | (2S,3S,4S,5S)-2-(4- | piperidine | | | | | | | | | y | | | |
| | hydroxyphenyl)piperidine- | | | | | | | | | | | | | |
| | 3,4,5-triol | | | | | | | | | | | | | |
| 874 | (2S,3S,4S,5S)-2- | piperidine | | | | | | | | | y | | | |
| | phenylpiperidine-3,4,5- | | | | | | | | | | | | | |
| | triol | | | | | | | | | | | | | |
| 875 | (2S,4S,5S)-1-butyl-4,5- | piperidine | | | | y | y | y | | | | | | |
| | dihydroxypiperidine-2- | | | | | | | | | | | | | |
| | carboxylic acid | | | | | | | | | | | | | |
| 876 | (2S,4S,5S)-4,5- | piperidine | | | | y | y | y | | | | | | |
| | dihydroxy-1- | | | | | | | | | | | | | |
| | nonylpiperidine-2- | | | | | | | | | | | | | |
| | carboxylic acid | | | | | | | | | | | | | |
| 877 | (2R,3S,4S,5S)-3,4- | pyrrolidine | | | | | | | | | y | | | |
| | dihydroxy-5- | | | | | | | | | | | | | |
| | (hydroxymethyl)-1- | | | | | | | | | | | | | |
| | methylpyrrolidine-2- | | | | | | | | | | | | | |
| | carboxylic acid | | | | | | | | | | | | | |
| 878 | (2R,3S,4S,5S)-1-butyl- | pyrrolidine | | | | | | | | | y | | | |
| | 3,4-dihydroxy-5- | | | | | | | | | | | | | |
| | (hydroxymethyl)pyrrolidine- | | | | | | | | | | | | | |
| | 2-carboxylic acid | | | | | | | | | | | | | |
| 879 | (2R,3S,4S,5S)-3,4- | pyrrolidine | | | | | | | | | y | | | |
| | dihydroxy-5- | | | | | | | | | | | | | |
| | (hydroxymethyl)-1- | | | | | | | | | | | | | |
| | nonylpyrrolidine-2- | | | | | | | | | | | | | |
| | carboxylic acid | | | | | | | | | | | | | |
| 880 | (2S,3R,4R,5S)-3,4,5- | piperidine | | | | | y | y | | | | | | |
| | trihydroxy-1- | | | | | | | | | | | | | |
| | nonylpiperidine-2- | | | | | | | | | | | | | |
| | carboxylic acid | | | | | | | | | | | | | |
| 881 | (2S,3S,4S,5S)-2,5- | pyrrolidine | | | | | | | | | y | | | |
| | bis(hydroxymethyl)-1- | | | | | | | | | | | | | |
| | methylpyrrolidine-3,4- | | | | | | | | | | | | | |
| | diol | | | | | | | | | | | | | |
| 882 | (2S,3S,4S,5S)-1-butyl- | pyrrolidine | | | | | | | | | y | | | |
| | 2,5- | | | | | | | | | | | | | |
| | bis(hydroxymethyl)pyrrolidine- | | | | | | | | | | | | | |
| | 3,4-diol | | | | | | | | | | | | | |
| 883 | (2S,3S,4S,5S)-2,5- | pyrrolidine | | | | | | | | | y | | | |
| | bis(hydroxymethyl)-1- | | | | | | | | | | | | | |
| | nonylpyrrolidine-3,4-diol | | | | | | | | | | | | | |
| 884 | (2S,3R,4R,5S)-1-ethyl- | piperidine | | | | | y | y | | | | | | |
| | 3,4,5- | | | | | | | | | | | | | |
| | trihydroxypiperidine-2- | | | | | | | | | | | | | |
| | carboxylic acid | | | | | | | | | | | | | |
| 885 | (2S,3R,4R,5S)-3,4,5- | piperidine | | | | | y | y | | | | | | |
| | trihydroxy-1- | | | | | | | | | | | | | |
| | propylpiperidine-2- | | | | | | | | | | | | | |
| | carboxylic acid | | | | | | | | | | | | | |
| 886 | (2S,3R,4R,5S)-3,4,5- | piperidine | | | | | y | y | | | | | | |
| | trihydroxy-1- | | | | | | | | | | | | | |
| | pentylpiperidine-2- | | | | | | | | | | | | | |
| | carboxylic acid | | | | | | | | | | | | | |
| 887 | (3R,4R,5S)-1-(6- | piperidine | | | | | | | | | | | | y |
| | ((1r,4R)-4- | | | | | | | | | | | | | |
| | methylcyclohexyloxy)hexyl)piperidine-3,4,5-triol | | | | | | | | | | | | | |
| 888 | (2S,3R,4R,5S)-3,4,5- | piperidine | | | | | y | y | | | | | | |
| | trihydroxy-1- | | | | | | | | | | | | | |
| | methylpiperidine-2- | | | | | | | | | | | | | |
| | carboxylic acid | | | | | | | | | | | | | |
| | hydrochloride | | | | | | | | | | | | | |
| 889 | (2S,3R,4R,5S)-1-butyl- | piperidine | | | | | y | y | | | | | | |
| | 3,4,5- | | | | | | | | | | | | | |
| | trihydroxypiperidine-2- | | | | | | | | | | | | | |
| | carboxylic acid | | | | | | | | | | | | | |
| | hydrochloride | | | | | | | | | | | | | |
| 890 | (2S,3R,4R,5S)-3,4,5- | piperidine | | | | | y | y | | | | | | |
| | trihydroxypiperidine-2- | | | | | | | | | | | | | |
| | carboxamide | | | | | | | | | | | | | |
| 891 | (2S,3R,4R,5S)-3,4,5- | piperidine | | | | | y | y | | | | | | |
| | trihydroxy-N- | | | | | | | | | | | | | |
| | methylpiperidine-2- | | | | | | | | | | | | | |
| | carboxamide | | | | | | | | | | | | | |
| 892 | (1R,2S,3R,5R,8aR)-3- | indolizidine | | y | | | | | | | y | | y | |
| | (hydroxymethyl)-5- | | | | | | | | | | | | | |
| | methyloctahydroindolizine- | | | | | | | | | | | | | |
| | 1,2-diol | | | | | | | | | | | | | |

### E. Chemical Synthesis

I. General considerations

Generally applicable strategies for the synthesis of iminosugars and iminosugar libraries are described by La Feria et al. (2007) in “Iminosugars: From synthesis to therapeutic applications”, Wiley ISBN 978-0-470-03391-3; Compain and Martin (Eds.) pages 25-61. These general techniques find application in the synthesis of a wide range of compounds for use according to the invention, including monocyclics, 1-N-iminosugars, bicyclic compounds and iminosugar conjugates. This disclosure is hereby incorporated herein by reference.

### II. Synthesis of Iminosugar C-glycosides

Generally applicable strategies for the synthesis of iminosugar C-glycosides are described by Compain (2007) in “Iminosugars: From synthesis to therapeutic applications”, Wiley ISBN 978-0-470-03391-3; Compain and Martin (Eds.) pages 63-86. These general techniques find application in the synthesis of a wide range of iminosugar C-glycosides for use according to the invention and the disclosure is hereby incorporated herein by reference.

### III. Synthesis of Imino-C-disaccharides and Analogues

Generally applicable strategies for the synthesis of imino-C-disaccharides and various analogues are described by Vogel et al. (2007) in “Iminosugars: From synthesis to therapeutic applications”, Wiley ISBN 978-0-470-03391-3; Compain and Martin (Eds.) pages 87-130 the disclosure of which is hereby incorporated herein by reference.

### IV. Synthesis of Polyhydroxylated Iminosugars

The synthesis of polyhydroxylated iminosugars can be carried out by protecting or differentiating the reactivity of the oxygen functions. Bell et al. (1997) Tetrahedron Lett. 38(33): 5869-72 describe the synthesis of four diastereoisomers of casuarine from eight carbon sugar lactones by reduction of open chain azidodimesylates by Suzuki-Takaoka reduction to allow the formation of the pyrrolizidine nucleus by bicyclisation (Bell et al. (1997) Tetrahedron Lett. 38(33): 5869-72).

Another approach is based on tandem [4+2]/[3+2] nitroalkene cycloadditions. It has been used for the synthesis of several pyrrolizidine and indolizidines iminosugars with up to four contiguous stereogenic centres (see Denmark and Hurd (1999) Organic Lett. 1(8): 1311-14). The method was later extended by the same workers to the synthesis of (+)-casuarine by the intermolecular [3+2] cycloaddition of a suitable substituted dipolarophile and a flexible, heavily substituted nitronate.

WO2006/008493 (the content of which relating to synthetic schemes for producing iminosugars is hereby incorporated by reference) describes the synthesis of polyhydroxylated pyrrolizidine and indolizidine compounds without protecting all of the free hydroxyl groups, so achieving considerably shortened synthetic schemes. Moreover, the use of intermediates having free hydroxyl groups provides a mechanism for controlling the product distribution, stereospecificity and yield via complex formation at the free hydroxyl groups. According to WO2006/008493, polyhydroxylated bicyclic (for example pyrrolizidine, indolizidine or quinolizidine) iminosugars can be produced by cyclisation of a pyrrolidine or piperidine intermediate having three or more free hydroxyl groups. The application of a cyclisation step to an intermediate having three or more free hydroxyl groups eliminates the need for selective protection, deprotection and/or activation at these sites.

### V. Synthesis of Iminosudar Acids

The ISAs described herein may be made by conventional methods. Methods of making heteroaromatic ring systems are well known in the art. In particular, methods of synthesis are discussed in Taylor et al. (2005) Tetrahedron: 61(40) 9611-9617 and in Comprehensive Heterocyclic Chemistry, Vol. 1 (Eds.: A R Katritzky, C W Rees), Pergamon Press, Oxford, 1984 and Comprehensive Heterocyclic Chemistry II: A Review of the Literature 1982-1995 The Structure, Reactions, Synthesis, and Uses of Heterocyclic Compounds, Alan R. Katritzky (Editor), Charles W. Rees (Editor), E. F. V. Scriven (Editor), Pergamon Pr, June 1996. Other general resources which would aid synthesis of the compounds of interest include March's Advanced Organic Chemistry: Reactions, Mechanisms, and Structure, Wiley-Interscience; 5th edition (Jan. 15, 2001). Some exemplary synthetic schemes for producing ISAs for use according to the invention are shown below:

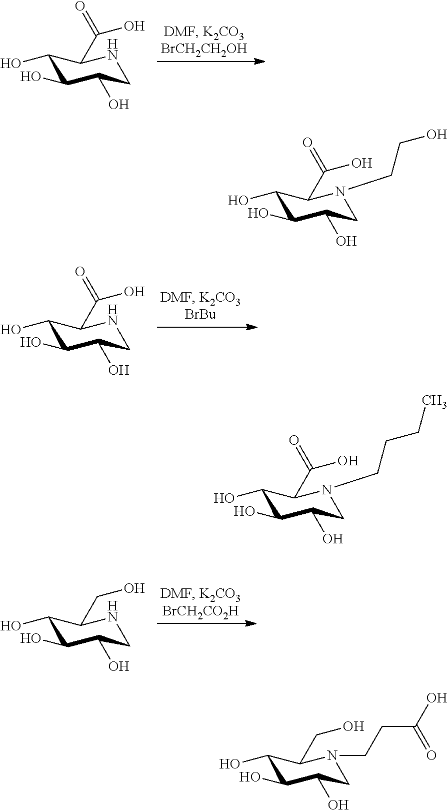

### VI. Synthesis of Nortropanes

Generally applicable strategies for the synthesis of nortropanes are described by Skaanderup and Madsen (2003) J. Org. Chem. 68(6): 2115-2122 the disclosure of which is hereby incorporated herein by reference.

### VII. Synthesis of Azepanes

Generally applicable strategies for the synthesis of azepanes are described by Li et al. (2007) Chem. Comm. (Cambridge, United Kingdom) (2): 183-185 the disclosure of which is hereby incorporated herein by reference.

### VIII. Synthesis of Pyrrolidines

Generally applicable strategies for the synthesis of pyrrolidines are described by Rountree et al. (2007) Tetrahedron Lett. 48: 4287-4291 and Behr and Guillerm (2007) Tetrahedron Lett. 48(13), 2369-2372 the disclosure of which is hereby incorporated herein by reference.

### IX. Synthesis of Piperidines

Generally applicable strategies for the synthesis of piperidines are described by Mane et al. (2008) J. Org. Chem. 73 (8): 3284-3287 and Rengasamy et al. (2008) J. Org. Chem. 73(7): 2898-2901 the disclosure of which is hereby incorporated herein by reference.

### X. Synthesis of Pyrrolizidines

Generally applicable strategies for the synthesis of pyrrolizidines are described in Pyrrolizidine Alkaloids, in The Way of Synthesis, Tomas Hudlicky and Josephine W. Reed, 2007, Wiley, ISBN: 978-3-527-31444-7, pages 617-653 and by Van Ameijde et al. (2006) Tetrahedron: Asymm. 17: 2702-2713, the disclosure of which is hereby incorporated herein by reference.

### XI. Synthesis of Indolizidines

Generally applicable strategies for the synthesis of indolizidines are described in Abrams et al. (2008) J. Org. Chem. 73 (5): 1935-1940 and Kumar et al. (2008) Org. Biomol. Chem. 6(4): 703-711, the disclosure of which is hereby incorporated herein by reference.

### XII. Synthesis of Quinolizidines

Generally applicable strategies for the synthesis of quinolizidines are described in Pasniczek et al. (2007) J. Carbohydrate Chem. 26(3): 195-211 and Kumar et al. (2008) Org. Biomol. Chem. 6(4): 703-711, the disclosure of which is hereby incorporated herein by reference.

### XIII. Synthesis of 4-Membered Monocycles

Generally applicable strategies for the synthesis of 4-membered monocycles are described in Evans et al. (2008) J. Med. Chem. 51(4): 948-956, the disclosure of which is hereby incorporated herein by reference.

### XIV. Synthesis of 9-Membered Monocycles

Generally applicable strategies for the synthesis of 9-membered monocycles are described in Leonard and Swann (1952) J. Am. Chem. Soc. 74: 4620-4, the disclosure of which is hereby incorporated herein by reference.

### XV. Synthesis of 10-Membered Monocycles

Generally applicable strategies for the synthesis of 10-membered monocycles are described by Arata and Kobayashi (1972) Chem. Pharm. Bull. 20(2): 325-9, the disclosure of which is hereby incorporated herein by reference.

### XVI. Synthesis of 4,6 Fused Bicyclics

Generally applicable strategies for the synthesis of 4,6 fused bicyclics are described in Pandey et al. (2006) Tetrahedron Lett. 47(45): 7923-7926, the disclosure of which is hereby incorporated herein by reference.

### XVII. Synthesis of 4,7 Fused Bicyclics

Generally applicable strategies for the synthesis of 4,7 fused bicyclics are described in Alcaide and Saez (2005) Eur. J. Org. Chem. (Issue 8): 1680-1693, the disclosure of which is hereby incorporated herein by reference.

### XVIII. Synthesis of 5,7 Fused Bicyclics

Generally applicable strategies for the synthesis of 5,7 fused bicyclics are described in Bande et al. (2007) Tetrahedron: Asymm. 18(10): 1176-1182, the disclosure of which is hereby incorporated herein by reference.

### XIX. Synthesis of 1,2 Piperazines

Generally applicable strategies for the synthesis of 1,2-piperazines are described in Ernholt et al. (1999) Synlett. 701-704, Liang et al (1999) J. Org. Chem., 64 (23), 8485-8488, Ernholt et al. (2000) Chem. Eur. J., 6(2) 278-287, Jensen et al. (2001) J. Chem. Soc., Perkin Trans. 1, 905-909 and Jensen et al. (2002) J. Chem. Soc., Perkin Trans. 1, 1190-1198 the disclosure of which is hereby incorporated herein by reference.

### F. Purification from Botanic Sources

### I. General

Botanic and microbial sources for a wide range of different iminosugars are described in Watson et al., (2001) Phytochemistry 56: 265-295. Iminosugar acids also have a wide distribution in plants such as in Stevia, Gymnema, Citrus, Lycium species, leguminous spp. e.g. Aspalanthus linearis (Rooibos), Lotus species and Castanospermum australe (Fabaceae), Cucurbitaceae species and Andrographis paniculata (Acanthaceae). The distribution of iminosugar acids in microorganisms is not known but they are likely to be present.

II. Purification of Iminosugars and Iminosugar Acids from Botanic Sources

The compounds described herein for use according to the invention may be isolated from natural sources. For example, plant material from botanic sources such as Stevia species can be used as starting material for the isolation and purification of both iminosugars and iminosugar acids for use according to the invention. Microorganisms such as Bacillus, Streptomyces and Metarrhizium species can be used for isolation of iminosugars. The natural iminosugars and iminosugar acids of the invention are water-soluble and can be concentrated by using strongly acidic cation exchange resins to which they bind with the iminosugar acids then concentrated subsequently by binding them to strongly basic anion exchange resins. The iminosugars are not strongly retained on the anion exchange resins whereas the iminosugar acids are. Purification of the iminosugars and iminosugar acids can then be achieved by using a series of cation and anion exchange resins selected by those experienced in the art. Size exclusion methods can also be used to concentrate them. Thus, it will be appreciated that those skilled in the art can readily purify and isolate the iminosugar and iminosugar acids of the invention using standard techniques.

### G. Adjunctive Agents for Use With the Compounds of the Invention

Thus, the invention provides compositions comprising the compound of the invention in combination with one or more adjunctive agents selected from: (a) an enzyme or protein (e.g. a lysosomal enzyme); (b) an ancillary proteostasis regulator (e.g. a pharmacoperone); (c) an inhibitor of a lysosomal enzyme; (d) nucleic acid (e.g. siRNA, cDNA, or DNA encoding an enzyme); and (e) a chaperone or cochaperone.

The term “ancillary proteostasis regulator” is a proteostasis regulator as defined herein which is not an imino sugar as herein defined or which does not have the formula (1), (2) or (3) as hereinbefore defined.

Exemplary proteostasis regulators include: (a) pharmacoperones; (b) UPR signalling regulators; (c) HSR regulators; (d) proteasome inhibitors; (e) upregulators of macromolecular chaperones in the ER; (f) transcriptional and/or translational upregulators; and/or (g) calcium homeostasis regulators.

In embodiments where the ancillary proteostasis regulator for use with the compounds of the invention is a UPR signalling regulator, the ancillary regulator may regulate one or more of: (a) the IRE1 arm of the UPR; (b) the ATF6 arm of the UPR; and/or (c) the PERK arm of the UPR.

In embodiments where the compound of the invention is a pharmacoperone as herein defined, preferred may be adjunctive use with (or combinations of the compound of the invention with a proteostasis regulator of a different functional class. For example, preferred are combination comprising a pharmacoperone compound of the invention in combination (or for adjunctive use with) a proteostasis regulator selected from: (a) UPR signalling regulators; (b) HSR regulators; (c) proteasome inhibitors; (d) upregulators of macromolecular chaperones in the ER; (e) trasncriptional and/or translational upregulators; and/or (f) calcium homeostasis regulators.

Particularly preferred may be adjunctive use with (or combinations of the compound of the invention with) a UPR signalling regulator, HSR regulator and/or proteasome inhibitor. Preferred UPR signalling and/or HSR regulators for use in the combinations/adjuntive uses of the invention include: (a) salubrinol; (b) celastrol; (c) indomethacin; and (d) sodium salicyclate. Preferred proteasome inhibitors for use in the combinations/adjuntive uses of the invention include: (a) MG-132; (b) lactacystin; (c) PS I; (d) PS IV and (e) tyropeptin A.

### Posology

The compounds of the present invention can be administered by oral or parenteral routes, including intravenous, intramuscular, intraperitoneal, subcutaneous, transdermal, airway (aerosol), rectal, vaginal and topical (including buccal and sublingual) administration.

The amount administered can vary widely according to the particular dosage unit employed, the period of treatment, the age and sex of the patient treated, the nature and extent of the disorder treated, and the particular compound selected.

Moreover, the compounds of the invention can be used in conjunction with other agents known to be useful in the treatment of diseases or disorders arising from protein folding abnormalities (as described infra) and in such embodiments the dose may be adjusted accordingly.

In general, the effective amount of the compound administered will generally range from about 0.01 mg/kg to 500 mg/kg daily. A unit dosage may contain from 0.05 to 500 mg of the compound, and can be taken one or more times per day. The compound can be administered with a pharmaceutical carrier using conventional dosage unit forms either orally, parenterally, or topically, as described below.

The preferred route of administration is oral administration. In general a suitable dose will be in the range of 0.01 to 500 mg per kilogram body weight of the recipient per day, preferably in the range of 0.1 to 50 mg per kilogram body weight per day and most preferably in the range 1 to 5 mg per kilogram body weight per day.

The desired dose is preferably presented as a single dose for daily administration. However, two, three, four, five or six or more sub-doses administered at appropriate intervals throughout the day may also be employed. These sub-doses may be administered in unit dosage forms, for example, containing 0.001 to 100 mg, preferably 0.01 to 10 mg, and most preferably 0.5 to 1.0 mg of active ingredient per unit dosage form.

### Formulation

The compound for use according to the invention may take any form. It may be synthetic, purified or isolated from natural sources.

When isolated from a natural source, the compound may be purified. In embodiments where the compound is formulated together with a pharmaceutically acceptable excipient, any suitable excipient may be used, including for example inert diluents, disintegrating agents, binding agents, lubricating agents, sweetening agents, flavouring agents, colouring agents and preservatives. Suitable inert diluents include sodium and calcium carbonate, sodium and calcium phosphate, and lactose, while corn starch and alginic acid are suitable disintegrating agents. Binding agents may include starch and gelatin, while the lubricating agent, if present, will generally be magnesium stearate, stearic acid or talc.

The pharmaceutical compositions may take any suitable form, and include for example tablets, elixirs, capsules, solutions, suspensions, powders, granules and aerosols.

The pharmaceutical composition may take the form of a kit of parts, which kit may comprise the composition of the invention together with instructions for use and/or a plurality of different components in unit dosage form.

Tablets for oral use may include the compound for use according to the invention, mixed with pharmaceutically acceptable excipients, such as inert diluents, disintegrating agents, binding agents, lubricating agents, sweetening agents, flavouring agents, colouring agents and preservatives. Suitable inert diluents include sodium and calcium carbonate, sodium and calcium phosphate, and lactose, while corn starch and alginic acid are suitable disintegrating agents. Binding agents may include starch and gelatin, while the lubricating agent, if present, will generally be magnesium stearate, stearic acid or talc. If desired, the tablets may be coated with a material such as glyceryl monostearate or glyceryl distearate, to delay absorption in the gastrointestinal tract. Capsules for oral use include hard gelatin capsules in which the compound for use according to the invention is mixed with a solid diluent, and soft gelatin capsules wherein the active ingredient is mixed with water or an oil such as peanut oil, liquid paraffin or olive oil.

Formulations for rectal administration may be presented as a suppository with a suitable base comprising for example cocoa butter or a salicylate. Formulations suitable for vaginal administration may be presented as pessaries, tampons, creams, gels, pastes, foams or spray formulations containing in addition to the active ingredient such carriers as are known in the art to be appropriate.

For intramuscular, intraperitoneal, subcutaneous and intravenous use, the compounds of the invention will generally be provided in sterile aqueous solutions or suspensions, buffered to an appropriate pH and isotonicity. Suitable aqueous vehicles include Ringer's solution and isotonic sodium chloride. Aqueous suspensions according to the invention may include suspending agents such as cellulose derivatives, sodium alginate, polyvinylpyrrolidone and gum tragacanth, and a wetting agent such as lecithin. Suitable preservatives for aqueous suspensions include ethyl and n-propyl p-hydroxybenzoate.

The compounds of the invention may also be presented as liposome formulations.

For oral administration the compound can be formulated into solid or liquid preparations such as capsules, pills, tablets, troches, lozenges, melts, powders, granules, solutions, suspensions, dispersions or emulsions (which solutions, suspensions dispersions or emulsions may be aqueous or non-aqueous). The solid unit dosage forms can be a capsule which can be of the ordinary hard- or soft-shelled gelatin type containing, for example, surfactants, lubricants, and inert fillers such as lactose, sucrose, calcium phosphate, and cornstarch.

In another embodiment, the compounds of the invention are tableted with conventional tablet bases such as lactose, sucrose, and cornstarch in combination with binders such as acacia, cornstarch, or gelatin, disintegrating agents intended to assist the break-up and dissolution of the tablet following administration such as potato starch, alginic acid, corn starch, and guar gum, lubricants intended to improve the flow of tablet granulations and to prevent the adhesion of tablet material to the surfaces of the tablet dies and punches, for example, talc, stearic acid, or magnesium, calcium, or zinc stearate, dyes, coloring agents, and flavoring agents intended to enhance the aesthetic qualities of the tablets and make them more acceptable to the patient.

Suitable excipients for use in oral liquid dosage forms include diluents such as water and alcohols, for example, ethanol, benzyl alcohol, and the polyethylene alcohols, either with or without the addition of a pharmaceutically acceptably surfactant, suspending agent or emulsifying agent.

The compounds of the invention may also be administered parenterally, that is, subcutaneously, intravenously, intramuscularly, or interperitoneally.

In such embodiments, the compound is provided as injectable doses in a physiologically acceptable diluent together with a pharmaceutical carrier (which can be a sterile liquid or mixture of liquids). Suitable liquids include water, saline, aqueous dextrose and related sugar solutions, an alcohol (such as ethanol, isopropanol, or hexadecyl alcohol), glycols (such as propylene glycol or polyethylene glycol), glycerol ketals (such as 2,2-dimethyl-1,3-dioxolane-4-methanol), ethers (such as poly(ethylene-glycol) 400), an oil, a fatty acid, a fatty acid ester or glyceride, or an acetylated fatty acid glyceride with or without the addition of a pharmaceutically acceptable surfactant (such as a soap or a detergent), suspending agent (such as pectin, carhomers, methylcellulose, hydroxypropylmethylcellulose, or carboxymethylcellulose), or emulsifying agent and other pharmaceutically adjuvants. Suitable oils which can be used in the parenteral formulations of this invention are those of petroleum, animal, vegetable, or synthetic origin, for example, peanut oil, soybean oil, sesame oil, cottonseed oil, corn oil, olive oil, petrolatum, and mineral oil. Suitable fatty acids include oleic acid, stearic acid, and isostearic acid. Suitable fatty acid esters are, for example, ethyl oleate and isopropyl myristate.

Suitable soaps include fatty alkali metal, ammonium, and triethanolamine salts and suitable detergents include cationic detergents, for example, dimethyl dialkyl ammonium halides, alkyl pyridinium halides, and alkylamines acetates; anionic detergents, for example, alkyl, aryl, and olefin sulphonates, alkyl, olefin, ether, and monoglyceride sulphates, and sulphosuccinates; nonionic detergents, for example, fatty amine oxides, fatty acid alkanolamides, and polyoxyethylenepolypropylene copolymers; and amphoteric detergents, for example, alkyl-beta-aminopropionates, and 2-alkylimidazoline quarternary ammonium salts, as well as mixtures.

The parenteral compositions of this invention will typically contain from about 0.5 to about 25% by weight of the compound for use according to the invention in solution. Preservatives and buffers may also be used. In order to minimize or eliminate irritation at the site of injection, such compositions may contain a non-ionic surfactant having a hydrophile-lipophile balance (HLB) of from about 12 to about 17. The quantity of surfactant in such formulations, ranges from about 5 to about 15% by weight. The surfactant can be a single component having the above HLB or can be a mixture of two or more components having the desired HLB. Illustrative of surfactants used in parenteral formulations are the class of polyethylene sorbitan fatty acid esters, for example, sorbitan monooleate and the high molecular weight adducts of ethylene oxide with a hydrophobic base, formed by the condensation of propylene oxide with propylene glycol.

The compound for use according to the invention may also be administered topically, and when done so the carrier may suitably comprise a solution, ointment or gel base. The base, for example, may comprise one or more of the following: petrolatum, lanolin, polyethylene glycols, bee wax, mineral oil, diluents such as water and alcohol, and emulsifiers and stabilizers. Topical formulations may contain a concentration of the compound from about 0.1 to about 10% w/v (weight per unit volume).

When used adjunctively, the compound for use according to the invention may be formulated for use with one or more other drug(s). In particular, the compounds may be used in combination with lysosomal enzymes adjunctive to enzyme replacement therapy. Thus, adjunctive use may be reflected in a specific unit dosage designed to be compatible (or to synergize) with the other drug(s), or in formulations in which the compound is admixed with one or more enzymes. Adjunctive uses may also be reflected in the composition of the pharmaceutical kits of the invention, in which the compounds of the invention is co-packaged (e.g. as part of an array of unit doses) with the enzymes. Adjunctive use may also be reflected in information and/or instructions relating to the co-administration of the compound and/or enzyme.

### EXEMPLIFICATION

The invention will now be described with reference to specific Examples. These are merely exemplary and for illustrative purposes only: they are not intended to be limiting in any way to the scope of the monopoly claimed or to the invention described. These examples constitute the best mode currently contemplated for practicing the invention.

### Example 1

### Identification of Pharmacoperones for α-Mannosidase

Solutions of 0.2M Mcllvane buffer at pH 4.5 and 5 mM p-nitrophenyl-α-D-mannopyranoside (Sigma, N2127), dissolved in the buffer, were prepared. A solution of Jack bean α-D-mannosidase enzyme (Sigma, M7257, 22 Units/mg, 6.2 mg/ml.) was also prepared at 0.6 Units/ml in the buffer.

The incubation mixture consisted of 10 μl enzyme solution, 10 μl of 1 mg/ml aqueous iminosugar solution and 50 μl of 5 mM substrate made up in buffer at the optimum pH for the enzyme. The reactions were stopped by addition of 70 μl 0.4M glycine (pH 10.4) during the exponential phase of the reaction, which had been determined at the beginning using uninhibited assays in which water replaced inhibitor. Final absorbances were read at 405 nm using a Versamax microplate reader (Molecular Devices). Assays were carried out in triplicate, and the values given are means of the three replicates. Results were expressed as a percentage of uninhibited assays in which water replaced inhibitor.

Several compounds of the invention have been found to increase the activity of the enzyme by between 49% and 124% at the top concentration used (˜0.8 mM). The stimulation was so great in some cases that the absorbance values were above the linear range and so the compounds were repeated at 0.08 mM and absorbance values were within range and still showed stimulation from 7% to 30% for the diluted samples.

An assay was set up in which (1R,2S,3S,4S,5R,6S)-5-amino-6-hydroxymethyl)cyclohexane-1,2,3,4-tetraol (Compound A, which shows strong stimulation) was mixed with an equal concentration of swainsonine and compared with swainsonine alone and as Compound A alone. The swainsonine plus Compound A and the swainsonine alone both gave 100% inhibition whereas Compound A alone gave 90% stimulation.

Other compounds showing promotion of the mannosidase activity in this experiment were 87, 170, 200 and 201. In the example given here we found that Jack Bean α-mannosidase activity (using p-nitrophenyl-α-D-mannopyranoside as the substrate) was greatly increased by certain iminosugars with a mannose configuration. These compounds did not cause inhibition of the mannosidase and swainsonine, a known inhibitor of this mannosidase, caused total inhibition of the promoted activity. This study indicates that the catalytic site is free for binding of swainsonine and so we presume that the increased activity of the mannosidase is due to binding to another site on the enzyme. Swainsonine does not cause promotion of the mannosidase at any concentration tested.

### Example 2

### Identification of Pharmacoperones for Other Glycosidases

The inventors have also observed stimulation of other glycosidase activities such as α-glucosidase, α-galactosidase and hexosaminidases by a range of other iminosugars without them being inhibitory to other glycosidases. Such compounds might therefore have utility in diseases such as Pompe's disease, Sandhoffs and Fabry's for example.

The assays were conducted as described by Watson et al. 1997, Phytochemistry 46: 255-259 using p-nitrophenyl-substrates. Assays were carried out in microtitre plates. Enzymes were assayed in 0.1M citric acid/0.2M di-sodium hydrogen phosphate (Mcllvaine) buffers at the optimum pH for the enzyme. All assays were carried out at 20° C. All enzymes and substrates were purchased from Sigma Aldrich Chemicals Limited. The incubation mixture consisted of 10 μl of enzyme solution, 10 μl of the iminosugars solution (made up in deionised water at 5 mM) and 50 μl of the appropriate 5 mM p-nitrophenyl substrate made up in McIlvaine buffer at the optimum pH for the enzyme. The reactions were stopped with 0.4M glycine (pH 10.4) during the exponential phase of the reaction, which was determined at the beginning of the assay using blanks with water, which were incubated for a range of time periods to measure the reaction rate using 5 mM substrate solution. Absorbances were read at 405 nm. Water was substituted for the inhibitors in the blanks.

The glycosidases used were α-D-glucosidase (yeast), α-D-glucosidase (Bacillus sterothermophilus), α-D-glucosidase (rice), amyloglucosidase (Aspergillus niger), β-D-glucosidase (almond), α-D-galactosidase (green coffee beans), β-D-galactosidase (bovine liver), α-L-fucosidase (bovine kidney), α-D-mannosidase (Jack bean), β-D-mannosidase (Cellullomonas fimi), Naringinase (Penicillium decumbens), N-acetyl-β-D-glucosaminidase (Bovine kidney), N-acetyl-β-D-glucosaminidase (Aspergillus oryzae) and N-acetyl-β-D-glucosaminidase (Jack bean)

TABLE 2
Promotion of glycosidase activity by some iminosugars of the invention is shown as
% increase in absorbance compared to the water controls

| | α-D-glucosidase | | | α-D- | N-acetyl-β-D- |
|---|---|---|---|---|---|
| Compound | (Bacillus | α-D-galactosidase | α-L-fucosidase | mannosidase | gluc |
| number | sterothermophilus) | (Green coffee bean) | (Bovine kidney) | (Jack bean) | (Bovine kidney) |
| 95 | 53 | 35 | | 86 | 46 |
| 165 | 72 | | | 43 | 76 |
| 178 | 161 | | | 35 | 85 |
| 205 | 60 | | | | |
| 238 | 127 | 30 | | | 31 |
| 242 | 65 | 38 | | | 59 |
| 253 | 56 | | | | |
| 291 | 49 | | | 43 | |
| 292 | | | | 34 | |
| 299 | | | | 35 | |
| 312 | 52 | 30 | | | |
| 316 | 167 | 42 | | | 94 |
| 324 | 150 | 42 | 42 | | |
| 338 | 170 | 35 | | | |
| 344 | 71 | | | | |
| 347 | 68 | | | | |
| 350 | 160 | 38 | | | |
| 370 | 81 | 43 | | | |

### Example 3

### Identification of Pharmacoperones that Bind to the Catalytic Site

The compounds of the invention can also function as chaperones through being specific inhibitors of glycosidases that are deficient in lysosomal storage disorders. An example is Compound 56 which potently inhibited alpha-L-iduronidase but none of the other 14 glycosidases described in Example 2. This compound, for example, might therefore show therapeutic activity for mucopolysaccharidosis type 1. Similarly Compound 7 is a specific inhibitor of beta-glucuronidase and might therefore be a treatment for mucopolysaccharidosis VII.

### Example 4

### Identification of Pharmacoperones for Beta-Glucocerebrosidase

### I. Beta-Glucocerebrosidase Activity Assay

Human Caucasian promyelocytic leukaemia cells (HL60, ECACC No. 98070106) were cultured using a standard sub-culture routine and lysed. The lysates were used as a source for wild type (wt) beta-glucocerebrosidase and used in an assay to determine the enzyme activity and conduct inhibition studies.

### i) Cell Lysate Preparation

HL60 cells were cultured to confluency and washed twice with PBS. Cells were lysed by the addition of lysis buffer (citric phosphate buffer (pH5.2), 0.1% Triton X-100, 0.25% taucholate) at 10×10⁶ cells/ml and incubated at 25° C. for 5 min. Lysates were cleared by centrifugation (400 g, 25° C., 5 min) and protein concentration was determined by using QuantiPro BCA assay kit (Sigma-Aldrich). Lysates were stored in aliquots at −80° C.

### ii) Beta-Glucocerebrosidase Activity Assay

4-Methylumbelliferyl β-D-glucopyranoside (4MU-β-D-glc) (Sigma) was used as a substrate to measure beta-glucocerebrosidase activity in HL60 lysate. Enzyme assays were performed in 96-well microtitre plates. Thawed cell lysate and 0.5 mM 4MU-β-D-glc in lysis buffer (50 μl final reaction volume) were mixed and incubated at 37° C. The reaction was quenched with 150 μl 0.5M sodium carbonate. The activity was measured by determining the rate of product (4MU) released using a fluorometer (OPTIMA, BMG) using excitation 360 nm, emission 450 nm filters. For detailed inhibition kinetic studies, various concentrations of iminosugars (1 nM-1 mM) and 4MU-β-D-Glc (100 μM-4 mM) were used.

In this assay the following compounds showed greater than 50% inhibition at a concentration of 100 μM:

| | Compound |
|---|---|
| Compound Name | No |
| (2R,3R,4R,5S)-2-(hydroxymethyl)-1-methylpiperidine-3,4,5-triol | 3 |
| (1R,2R,3R,7S,7aS)-3-(hydroxymethyl)hexahydro-1H-pyrrolizine-1,2,7-triol | 17 |
| (1R,2S,3R,5R)-8-azabicyclo[3.2.1]octane-1,2,3-triol | 20 |
| (1R,2S,3R,4S,5R)-8-azabicyclo[3.2.1]octane-1,2,3,4-tetraol | 21 |
| (1R,2S,3R,4S,5R,6R)-8-azabicyclo[3.2.1]octane-1,2,3,4,6-pentaol | 33 |
| (1S,2R,3S,5R)-8-azabicyclo[3.2.1]octane-1,2,3,6-tetraol | 43 |
| (1R,2S,3R,5R,8aR)-3-(hydroxymethyl)-5-methyloctahydroindolizine-1,2-diol | 60 |
| (1S,2R,3R,5R,6S,7aR)-5-(3-hydroxybutyl)-3-(hydroxymethyl)hexahydro-1H-pyrrolizine- | 66 |
| 1,2,6-triol | |
| (2S,3R,4R,5R,6R)-5-(3,4-dihydroxy-2,5-bis(hydroxymethyl)tetrahydrofuran-2-yloxy)- | 77 |
| 2,6-bis(hydroxymethyl)piperidine-3,4-diol | |
| (3R,4r,5S)-piperidine-3,4,5-triol | 103 |
| (1S,6S,7R,8R,8aR)-octahydroindolizine-1,6,7,8-tetraol | 104 |
| (1R,2S,3S,4S,5R)-4-(3,4,5-trihydroxy-6-(hydroxymethyl)tetrahydro-2H-pyran-2-yloxy)- | 105 |
| 8-azabicyclo[3.2.1]octane-1,2,3-triol | |
| (2R,3R,4R,5S)-1-butyl-2-(hydroxymethyl)piperidine-3,4,5-triol | 118 |
| (1R,2R,3S,6S,7S,7aR)-3-(hydroxymethyl)hexahydro-1H-pyrrolizine-1,2,6,7-tetraol | 129 |
| (2S,3S,4S)-2-(hydroxymethyl)pyrrolidine-3,4-diol | 160 |
| (3R,4S,5S)-5-(hydroxymethyl)piperidine-3,4-diol | 219 |
| (2S,3S,4S,5R)-2-(hydroxymethyl)piperidine-3,4,5-triol | 221 |
| (R)-13-((2R,3R,4R,5R)-3,4-dihydroxy-5-(hydroxymethyl)pyrrolidin-2-yl)-1,13- | 226 |
| dihydroxytridecan-5-one | |
| (3R,4r,5S)-1-butylpiperidine-3,4,5-triol | 232 |
| (3R,4r,5S)-1-nonylpiperidine-3,4,5-triol | 254 |
| (3S,4R,5S,6S)-N-butyl-3,4,5-trihydroxy-6-methylpiperidine-2-carboxamide | 265 |
| (3S,4R,5S,6S)-N-benzyl-3,4,5-trihydroxy-6-methylpiperidine-2-carboxamide | 267 |
| (3R,4R,5R)-5-(Hydroxymethyl)-3,4-piperidinediol | 279 |
| (2R,3R,4R,5S)-2-(hydroxymethyl)-1-(3-phenoxypropyl)piperidine-3,4,5-triol | 288 |
| (2S,3R,4R)-1-(cyclohexylmethyl)-2-(hydroxymethyl)pyrrolidine-3,4-diol | 320 |
| (2R,3R,4R,5R)-2,5-bis(hydroxymethyl)-1-(3-phenoxypropyl)pyrrolidine-3,4-diol | 343 |
| (2R,3R,4R)-2-(hydroxymethyl)-1-(3-phenoxypropyl)pyrrolidine-3,4-diol | 349 |
| (3R,4R,5R)-1-butyl-5-(hydroxymethyl)piperidine-3,4-diol | 419 |
| (3R,4R,5R)-1-(2-hydroxyethyl)-5-(hydroxymethyl)piperidine-3,4-diol | 420 |
| 2-((3R,4R,5R)-3,4-dihydroxy-5-(hydroxymethyl)piperidin-1-yl)acetic acid | 450 |
| 2-((3S,4S,5S)-3,4-dihydroxy-5-(hydroxymethyl)piperidin-1-yl)acetic acid | 451 |
| Nojirimycin-1-Sulfonic Acid | 464 |
| (2R,3R,4R,5S)-1-(biphenyl-4-ylmethyl)-2-(hydroxymethyl)piperidine-3,4,5-triol | 524 |
| (2R,3R,4R,5S)-2-(hydroxymethyl)-1-nonylpiperidine-3,4,5-triol | 550 |
| (2R,3R,4R,5R)-2,5-bis(hydroxymethyl)-1-nonylpyrrolidine-3,4-diol | 555 |
| (2R,3R,4R,5R)-1-(2-(benzyloxy)ethyl)-2,5-bis(hydroxymethyl)pyrrolidine-3,4-diol | 556 |
| (2R,3R,4R,5R)-2,5-bis(hydroxymethyl)-1-(9-hydroxynonyl)pyrrolidine-3,4-diol | 557 |
| (2R,3R,4R)-2-(hydroxymethyl)-1-nonylpyrrolidine-3,4-diol | 568 |
| (3S,4S,5S)-1-(2-hydroxyethyl)-5-(hydroxymethyl)piperidine-3,4-diol | 640 |
| (3S,4S,5S)-5-(hydroxymethyl)piperidine-3,4-diol | 641 |
| N-(((2R,3R,4S)-1-(biphenyl-4-ylmethyl)-3,4-dihydroxypyrrolidin-2-yl)methyl)benzamide | 649 |
| (2S,3R,4S)-2-(aminomethyl)-1-(biphenyl-4-ylmethyl)pyrrolidine-3,4-diol | 686 |
| N-(((2S,3R,4S)-1-(biphenyl-4-ylmethyl)-3,4-dihydroxypyrrolidin-2-yl)methyl)biphenyl-4- | 701 |
| carboxamide | |
| (2R,3R,4R,5S)-2-(hydroxymethyl)-1-(9-hydroxynonyl)piperidine-3,4,5-triol | 724 |
| (3R,4S,5R,6S)-1-nonylazepane-3,4,5,6-tetraol | 736 |
| (3R,4R,5R,6R)-1-(5-(adamantan-1-yl-methoxy)-pentyl)azepane-3,4,5,6-tetraol | 766 |

### II. Enzyme Enhancement Assay—Cell Based Screening for Chaperones

Lymphoblasts derived from Gaucher's patients can be used for the cell based screening assays. EBV transformed B-lymphocytes from Gaucher's patients such as cell lines homozygous for the N370S mutation (GM01873) and L444P mutation (GM08752) in beta-glucocerebrosidase, were obtained from Coriell Institute for Medical Research. Cells were cultured in RMPI 1640 (Sigma) supplemented with 15% FBS (PAA), 2 mM L-glutamine and penicillin-streptomycin (PAA) as described in the culturing protocol.

Cells were seeded (8×10⁴ cells/well) and dosed (0.3-100 μM) in white 96-well plates (NUNC) to a final volume of 300 μL, and incubated for 72 hr at 37° C. in a 5% CO₂ incubator. Cells (200 μL) were transferred to 96-well Multiscreen harvester plates (Millipore) and harvested under vacuum. Cells were washed twice with PBS and lysed (and the enzyme reaction started) by the addition of 100 μL lysis buffer containing 5 mM 4MU-β-D-glc. Cell debris was removed by filtering through and collecting the cleared lysates. Lysates were incubated at 37° C. for a total time of 2 hrs. The enzyme reaction was quenched by addition of 150 μL 0.5M sodium carbonate to 50 μl of reaction mix. Fluorescence was measured as described above. QuantiPro BCA assay kit (Sigma) was used to determine the protein concentration in the cell lysates. Cell viability was measured using CellTiter-Glo® luminescent cell viability assay (Promega) on the remaining 100 μL unlysed cells. All experiments were performed in triplicates. The fold beta-glucocerebrosidase enzyme activity was determined relative to the vehicle (water or 1% DMSO) control, and normalised against total protein amount per well.

In this assay the following compounds increased enzyme activity by greater than 20%:

| | Compound |
|---|---|
| Compound Name | No |
| (2S,4R)-4-hydroxy-1,1-dimethylpyrrolidinium-2-carboxylate | 6 |
| (1R,2S,3R,5R)-8-azabicyclo[3.2.1]octane-1,2,3-triol | 20 |
| (1R,2S,3R,4S,5R)-8-azabicyclo[3.2.1]octane-1,2,3,4-tetraol | 21 |
| (1R,2S,3R,4S,5R,6R)-8-azabicyclo[3.2.1]octane-1,2,3,4,6-pentaol | 33 |
| (1S,2R,3S,5R)-8-azabicyclo[3.2.1]octane-1,2,3,6-tetraol | 43 |
| (1S,6S,7S,8R)-1,7,8-trihydroxyoctahydroindolizin-6-yl butyrate | 46 |
| (1S,2R,3R,5S,7aR)-5-(3-hydroxybutyl)-3-(hydroxymethyl)hexahydro-1H-pyrrolizine-1,2- | 67 |
| diol | |
| (2S,3S,4R)-1-(2-hydroxyethyl)-2-(hydroxymethyl)-2-methylpyrrolidine-3,4-diol | 99 |
| (3R,4S,5R)-5,6-dimethyl-2,3,4,5-tetrahydropyridine-3,4,5-triol | 102 |
| (3R,4r,5S)-piperidine-3,4,5-triol | 103 |
| (1S,6S,7R,8R,8aR)-octahydroindolizine-1,6,7,8-tetraol | 104 |
| (1R,2S,3S,4S,5R)-4-(3,4,5-trihydroxy-6-(hydroxymethyl)tetrahydro-2H-pyran-2-yloxy)- | 105 |
| 8-azabicyclo[3.2.1]octane-1,2,3-triol | |
| (1S,2S,3R,4S,5S)-5-methyl-8-oxa-6-azabicyclo[3.2.1]octane-2,3,4-triol | 106 |
| (2R,3R,4S,5R)-2-methylpiperidine-3,4,5-triol | 109 |
| 2-((2R,3R,4R,5S,6R)-4,5-dihydroxy-2,6-bis(hydroxymethyl)piperidin-3-yloxy)-6- | 110 |
| (hydroxymethyl)tetrahydro-2H-pyran-3,4,5-triol | |
| 1-((2S,3R,4R,5R)-3,4-dihydroxy-5-(hydroxymethyl)pyrrolidin-2-yl)-2-methoxy-1H- | 111 |
| imidazole-4,5-diol | |
| (3S,4S,5R,6R)-3,4,5-trihydroxy-6-(hydroxymethyl)piperidin-2-one | 112 |
| (2R,3R,4R,5S)-1-butyl-2-(hydroxymethyl)piperidine-3,4,5-triol | 118 |
| 2-((3S,4S,5R,6R)-4,5-dihydroxy-6-(hydroxymethyl)piperidin-3-yloxy)-6- | 121 |
| (hydroxymethyl)tetrahydro-2H-pyran-3,4,5-triol | |
| (2R,3R,4R,5S,6R)-2,6-bis(hydroxymethyl)-1-methylpiperidine-3,4,5-triol | 124 |
| (2S,3R,4R)-1-butyl-2-(hydroxymethyl)pyrrolidine-3,4-diol | 157 |
| (2S,3S,4S)-1-(2-hydroxyethyl)-2-(hydroxymethyl)pyrrolidine-3,4-diol | 161 |
| (2S,3S,4S)-1-butyl-2-(hydroxymethyl)pyrrolidine-3,4-diol | 163 |
| (2S,3R,4R)-2-(hydroxymethyl)pyrrolidine-3,4-diol | 171 |
| (2S,3S,4S)-2-((S)-1,2-dihydroxyethyl)-4-(hydroxymethyl)pyrrolidine-3,4-diol | 179 |
| (2S,3S,4R,5S)-2,3-dimethylpiperidine-3,4,5-triol | 180 |
| (2R,3S,4R,5S)-2,3-dimethylpiperidine-3,4,5-triol | 181 |
| (1R,2R,3R,7R,7aR)-3-(hydroxymethyl)hexahydro-1H-pyrrolizine-1,2,7-triol | 185 |
| (1S,6R,7R,8R,8aR)-octahydroindolizine-1,6,7,8-tetraol | 186 |
| (2R,3R,4S,5S)-2-(hydroxymethyl)piperidine-3,4,5-triol | 187 |
| (2R,3R,4R,5R)-2-(hydroxymethyl)-5-methylpyrrolidine-3,4-diol | 190 |
| (2R,3R,4R,5R)-2-(2-hydroxyethyl)-5-(hydroxymethyl)pyrrolidine-3,4-diol | 194 |
| (2R,3R,4R,5S)-2-(hydroxymethyl)-5-(4-hydroxyphenyl)pyrrolidine-3,4-diol | 196 |
| (2R,3R,4S,5R)-2-(hydroxymethyl)-5-methylpiperidine-3,4,5-triol | 201 |
| (3R,4r,5S)-1-butylpiperidine-3,4,5-triol | 232 |
| (3R,4r,5S)-1-nonylpiperidine-3,4,5-triol | 254 |
| ((2S,4S)-4-azido-1-butylpyrrolidin-2-yl)methanol | 275 |
| 2-((2S,4S)-4-azido-2-(hydroxymethyl)pyrrolidin-1-yl)ethanol | 277 |
| (3R,4R,5R)-5-(Hydroxymethyl)-3,4-piperidinediol | 279 |
| (2R,3R,4R,5S)-2-(hydroxymethyl)-1-(3-phenoxypropyl)piperidine-3,4,5-triol | 288 |
| (2S,3S,4S,5S)-2-(hydroxymethyl)-5-methylpyrrolidine-3,4-diol | 300 |
| (3S,5S)-1-nonylpiperidine-3,4,5-triol | 302 |
| (2R,3S,4S)-1-butyl-2-(hydroxymethyl)pyrrolidine-3,4-diol | 306 |
| N-((3S,4R,5S)-1-butyl-4-hydroxy-5-(hydroxymethyl)pyrrolidin-3-yl)acetamide | 319 |
| N-((3R,4R,5S)-4-hydroxy-1-(2-hydroxyethyl)-5-(hydroxymethyl)pyrrolidin-3- | 375 |
| yl)acetamide | |
| N-((3S,4S,5R)-1-butyl-4-hydroxy-5-(hydroxymethyl)pyrrolidin-3-yl)acetamide | 391 |
| (S)-4-((2S,3R,4S)-1-benzyl-3,4-dihydroxypyrrolidin-2-yl)-4-hydroxybutanenitrile | 408 |
| (S)-5-((1R,2S,3S)-1,2,3,4-tetrahydroxybutyl)pyrrolidin-2-one | 415 |
| (2S,3S,4R)-2-((S)-1,2-dihydroxyethyl)-1-(9-hydroxynonyl)pyrrolidine-3,4-diol | 433 |
| (1R,2S,8R,8aR)-1,2,8-trihydroxy-6-methylhexahydroindolizin-5(1H)-one | 490 |
| (1R,2S,6S,8R,8aR)-6-(2-hydroxyethyl)octahydroindolizine-1,2,8-triol | 493 |
| (2S,3S,3aS,6S,7S,7aS)-2-(hydroxymethyl)-1-(methylsulfonyl)octahydropyrano[3,2- | 495 |
| b]pyrrole-3,6,7-triol | |
| (2S,3S,4S)-2-(hydroxymethyl)-2-methylpyrrolidine-3,4-diol | 501 |
| N-{[(3S,4S,5R)-1-benzyl-4,5-dihydroxypiperidin-3-yl]methyl}acetamide | 503 |
| (2R,3R,4R,5R)-2,5-bis(hydroxymethyl)-1-methylpyrrolidine-3,4-diol | 515 |
| N-(((2S,4R)-1-butyl-4-hydroxypyrrolidin-2-yl)methyl)acetamide | 549 |
| (2R,3R,4R,5R)-1-(2-(benzyloxy)ethyl)-2,5-bis(hydroxymethyl)pyrrolidine-3,4-diol | 556 |
| (2R,3R,4R)-2-(hydroxymethyl)-1-(2-(2-methoxyethoxy)ethyl)pyrrolidine-3,4-diol | 571 |
| (2R,3R,4R)-2-(hydroxymethyl)-1-(2-morpholinoethyl)pyrrolidine-3,4-diol | 572 |
| (2R,3R,4R)-2-(hydroxymethyl)-1-(2-(piperidin-1-yl)ethyl)pyrrolidine-3,4-diol | 573 |
| (3aS,4R,7R,7aR)-tert-butyl 7-hydroxy-2,2,4-trimethyltetrahydro-[1,3]dioxolo[4,5- | 579 |
| c]pyridine-5(6H)-carboxylate | |
| tert-butyl 5-hydroxy-5,6-dihydropyridine-1(2H)-carboxylate | 580 |
| N-(((2R,3R,4S)-1-benzyl-3,4-dihydroxypyrrolidin-2-yl)methyl)benzamide | 586 |
| (1R,2S,3R)-1-((3R,4R)-3,4-dihydroxy-1-(2-hydroxyethyl)pyrrolidin-2-yl)butane-1,2,3,4- | 589 |
| tetraol | |
| N-(((2S,3R,4S)-1-benzyl-3,4-dihydroxypyrrolidin-2-yl)methyl)benzamide | 597 |
| N-(((2S,3R,4S)-1-benzyl-3,4-dihydroxypyrrolidin-2-yl)methyl)acetamide | 598 |
| N-(((3aR,4S,6aS)-5-benzyl-2,2-dimethyltetrahydro-3aH-[1,3]dioxolo[4,5-c]pyrrol-4- | 599 |
| yl)methyl)benzamide | |
| (2S,3S,4R)-1-(biphenyl-4-ylmethyl)-2-((S)-1,2-dihydroxyethyl)pyrrolidine-3,4-diol | 606 |
| N-(((2S,3R,4S)-1-butyl-3,4-dihydroxypyrrolidin-2-yl)methyl)benzamide | 607 |
| N-(((2R,3R,4S)-3,4-dihydroxypyrrolidin-2-yl)methyl)benzamide | 614 |
| N-(((2R,3R,4S)-1-butyl-3,4-dihydroxypyrrolidin-2-yl)methyl)benzamide | 617 |
| N-(((2S,3R,4S)-3,4-dihydroxy-1-(2-morpholinoethyl)pyrrolidin-2-yl)methyl)acetamide | 618 |
| N-(((2R,3R,4S)-1-benzyl-3,4-dihydroxypyrrolidin-2-yl)methyl)-2,2,2-trifluoroacetamide | 619 |
| N-(((3aR,4S,6aS)-2,2-dimethyltetrahydro-3aH-[1,3]dioxolo[4,5-c]pyrrol-4- | 629 |
| yl)methyl)benzamide | |
| N-(((2R,3R,4S)-3,4-dihydroxy-1-nonylpyrrolidin-2-yl)methyl)benzamide | 646 |
| N-(((2R,3R,4S)-3,4-dihydroxy-1-(9-hydroxynonyl)pyrrolidin-2-yl)methyl)benzamide | 648 |
| N-(((2S,3R,4S)-1-butyl-3,4-dihydroxypyrrolidin-2-yl)methyl)-2,2,2-trifluoroacetamide | 682 |
| N-(((2R,3R,4S)-1-butyl-3,4-dihydroxypyrrolidin-2-yl)methyl)butyramide | 689 |
| N-(((2S,3R,4S)-1-(biphenyl-4-ylmethyl)-3,4-dihydroxypyrrolidin-2-yl)methyl)biphenyl-4- | 701 |
| carboxamide | |
| (2R,3R,4R,5S)-2-(hydroxymethyl)-1-(9-hydroxynonyl)piperidine-3,4,5-triol | 724 |
| (2R,3R,4R,5S)-2-(hydroxymethyl)-1-(2-(2-methoxyethoxy)ethyl)piperidine-3,4,5-triol | 725 |
| N-((3S,4R,5R)-4,5-dihydroxypiperidin-3-yl)acetamide | 733 |
| (3R,4S,5R,6S)-1-nonylazepane-3,4,5,6-tetraol | 736 |
| (3R,4S,5R,6S)-1-(9-hydroxynonyl)azepane-3,4,5,6-tetraol | 737 |
| (R)-(1-butylpiperidin-3-yl)methanol | 741 |

### III. Identification of Non-Active Site Chaperones of Beta-Glucocerebrosidases

Compounds that demonstrated a significant increase in cellular beta-glucocerebrosidase activity (protocol II) but showed no competitive inhibition of beta-glucocerebrosidase enzyme activity (protocol I) were considered to be non-active site chaperones.

Compounds identified according to the methods described above (I-III) find utility in the treatment of Gaucher's disease.

### Example 5

### Identification of Pharmacoperones for Alpha-Galactosidase

### I. Alpha-Galactosidase Activity Assay

Human Caucasian promyelocytic leukaemia cells (HL60, ECACC No. 98070106) were cultured using a standard sub-culture routine and lysed. The lysates were used as a source for wild type (wt) alpha-galactosidase and used in an assay to determine the enzyme activity and conduct inhibition studies.

### i) Cell Lysate Preparation

Cell lysates were prepared as described above (Gaucher's I.i)

### ii) Alpha-Galactosidase Activity Assay

4-Methylumbelliferyl alpha-galactopyranoside (4MU-α-D-gal) (Sigma) was used as a substrate to measure alpha-galactosidase activity in HL60 lysate. Enzyme assays were performed in 96-well microtitre plates. Thawed cell lysate and 0.5 mM 4MU-α-D-gal in citric phosphate buffer (pH 4.5) containing 0.1 M N-acetylgalactosamine (50 μl final reaction volume) were mixed and incubated at 37° C. The reaction was quenched with 150 μl 0.5M sodium carbonate. The activity was measured by determining the rate of product (4MU) released using a fluorometer (OPTIMA, BMG) using excitation 360 nm, emission 450 nm filters. For detailed inhibition kinetic studies, various concentrations of iminosugars (1 nM-1 mM) and 4MU-α-D-Gal (100 μM-4 mM) were used.

In this assay, the following compounds showed greater than 50% inhibition at a concentration of 100 uM:

| | Compound |
|---|---|
| Compound Name | No |
| (1R,2S,3R,4S,5R)-8-azabicyclo[3.2.1]octane-1,2,3,4-tetraol | 21 |
| (2R,3R,4S,5R)-1-(2-hydroxyethyl)-2,5-bis(hydroxymethyl)pyrrolidine-3,4-diol | 168 |
| (2R,3R,4S,5R)-2,5-bis(hydroxymethyl)pyrrolidine-3,4-diol | 183 |
| (3S,4S,5R,6R)-3,4,5-trihydroxy-6-(hydroxymethyl)piperidin-2-one | 112 |
| (2R,3S,5S,6R)-2,6-bis(hydroxymethyl)piperidine-3,4,5-triol | 189 |
| (2R,3S,4R,5S)-2-(hydroxymethyl)piperidine-3,4,5-triol | 215 |
| (2R,3S,4R,5S)-2-methylpiperidine-3,4,5-triol | 258 |
| (2R,3S,4R,5S,6R)-2-(hydroxymethyl)-6-methylpiperidine-3,4,5-triol | 263 |
| (2S,3S,4R,5S,6R)-2-(hydroxymethyl)-6-methylpiperidine-3,4,5-triol | 264 |
| (2S,3R,4S,5R,6R)-3,4,5-trihydroxy-6-(hydroxymethyl)-N-methylpiperidine-2- | 246 |
| carboxamide | |
| (2S,3S,4R,5S,6S)-2-(hydroxymethyl)-6-methylpiperidine-3,4,5-triol | 269 |
| (2R,3R,4S,5S,6S)-2-(but-3-enyl)-6-(hydroxymethyl)piperidine-3,4,5-triol | 380 |
| (2R,3S,4R)-2-(hydroxymethyl)pyrrolidine-3,4-diol | 384 |
| (2R,3S,4S,5R)-2-(hydroxymethyl)piperidine-3,4,5-triol | 447 |

### II. Enzyme Enhancement Assay—Cell Based Screening for Chaperones

Lymphoblasts derived from Fabry's patients can be used for the cell based screening assays. EBV transformed B-lymphocytes from Fabry's patient (GM04391) were obtained from Coriell Institute for Medical Research. Cells were cultured in RMPI 1640 (Sigma) supplemented with 15% FBS(PAA), 2 mM L-glutamine and penicillin-streptomycin (PAA) as described in the culturing protocol.

Cells were seeded (8×10⁴ cells/well) and dosed (0.3-100 μM) in white 96-well plates (NUNC) to a final volume of 300 μL, and incubated for 72 hr at 37° C. in a 5% CO₂ incubator. Cells (200 μL) were transferred to 96-well Multiscreen harvester plates (Millipore) and harvested under vacuum. Cells were washed twice with PBS and lysed (and the enzyme reaction started) by the addition of 100 μL 5 mM 4MU-α-D-gal in citric phosphate buffer (pH4.5) with 0.1% Triton X-100 and 0.1M N-acetylgalactosamine (Sigma). Cell debris was removed by filtering through and collecting the cleared lysates, and the lysate was incubated at 37° C. for 2 hrs The enzyme reaction was quenched by addition of 150 μL 0.5M sodium carbonate to 50 μl of reaction mix. Fluorescence was measured as described above. Cell viability was measured using CellTiter-Glo® luminescent cell viability assay (Promega) on the remaining 100 μL unlysed cells. All experiments were performed in triplicates. The fold alpha-galactosidase enzyme activity was determined relative to the vehicle (water or 1% DMSO) control.

In this assay the following compounds increased enzyme activity by greater than 20%:

| | Compound |
|---|---|
| Compound Name | No |
| (1R,2S,3R,5R)-8-azabicyclo[3.2.1]octane-1,2,3-triol | 20 |
| (1R,2S,3R,4S,5R)-8-azabicyclo[3.2.1]octane-1,2,3,4-tetraol | 21 |
| (2S,3S,4R)-1-butyl-2-(hydroxymethyl)pyrrolidine-3,4-diol | 74 |
| (1S,2R,3R,7S,7aR)-3-(hydroxymethyl)hexahydro-1H-pyrrolizine-1,2,7-triol | 139 |
| (2S,3S,4S)-1-butyl-2-(hydroxymethyl)pyrrolidine-3,4-diol | 163 |
| (2R,3S,4R,5S)-2,3-dimethylpiperidine-3,4,5-triol | 181 |
| (2R,3R,4S,5R)-2,5-bis(hydroxymethyl)pyrrolidine-3,4-diol | 183 |
| (2R,3S,5S,6R)-2,6-bis(hydroxymethyl)piperidine-3,4,5-triol | 189 |
| (2R,3R,4S,5R)-2-(hydroxymethyl)-5-methylpiperidine-3,4,5-triol | 201 |
| (2R,3S,4R,5S)-2-(hydroxymethyl)piperidine-3,4,5-triol | 215 |
| (2R,3S,4R,5S)-2-(aminomethyl)piperidine-3,4,5-triol | 227 |
| (2S,3R,4S,5R,6R)-3,4,5-trihydroxy-6-(hydroxymethyl)-N-methylpiperidine-2- | 246 |
| carboxamide | |
| (2R,3S,4R,5S)-2-methylpiperidine-3,4,5-triol | 258 |
| (2R,3S,4R,5S,6R)-2-(hydroxymethyl)-6-methylpiperidine-3,4,5-triol | 263 |
| (2S,3S,4R,5S,6R)-2-(hydroxymethyl)-6-methylpiperidine-3,4,5-triol | 264 |
| (2S,3S,4R,5S,6S)-2-(hydroxymethyl)-6-methylpiperidine-3,4,5-triol | 269 |
| ((2S,4S)-4-amino-1-butylpyrrolidin-2-yl)methanol | 292 |
| (3R,5R)-1-butylpiperidine-3,4,5-triol | 296 |
| (3R,5R)-tert-butyl 3,4,5-trihydroxypiperidine-1-carboxylate | 303 |
| (2R,3S,4S)-1-butyl-2-(hydroxymethyl)pyrrolidine-3,4-diol | 306 |
| N-((3S,4R,5S)-1-benzyl-4-hydroxy-5-(hydroxymethyl)pyrrolidin-3-yl)acetamide | 309 |
| (2R,3R,4S)-2-((S)-1,2-dihydroxyethyl)pyrrolidine-3,4-diol | 315 |
| N-((3S,4R,5S)-1-butyl-4-hydroxy-5-(hydroxymethyl)pyrrolidin-3-yl)acetamide | 319 |
| (1R,2R)-1-((2R,3R,4S)-3,4-dihydroxy-1-(2-hydroxyethyl)pyrrolidin-2-yl)propane-1,2,3- | 327 |
| triol | |
| (1R,2S,8R,8aR)-octahydroindolizine-1,2,8-triol | 333 |
| (2S,3S,4R)-2-((R)-1,2-dihydroxyethyl)-1-(2-hydroxyethyl)pyrrolidine-3,4-diol | 334 |
| N-((3S,4R,5S)-4-hydroxy-1-(2-hydroxyethyl)-5-(hydroxymethyl)pyrrolidin-3- | 335 |
| yl)acetamide | |
| (1S,2R)-1-((2S,3R,4S)-3,4-dihydroxypyrrolidin-2-yl)propane-1,2,3-triol | 336 |
| N-((3S,4R,5S)-4-hydroxy-5-(hydroxymethyl)-1-nonylpyrrolidin-3-yl)acetamide | 338 |
| (2S,3S,4R)-1-benzyl-2-(hydroxymethyl)pyrrolidine-3,4-diol | 342 |
| ((2S,4S)-4-aminopyrrolidin-2-yl)methanol | 344 |
| (2S,4S)-4-azidopyrrolidine-2-carboxylic acid | 345 |
| 2((2R,3R,4S)-3,4-dihydroxy-2-(hydroxymethyl)pyrrolidin-1-yl)acetic acid | 361 |
| N-((3R,4S,5R)-1-benzy1-4-hydroxy-5-(hydroxymethyl)pyrrolidin-3-yl)acetamide | 364 |
| N-((3R,4S,5R)-4-hydroxy-5-(hydroxymethyl)-1-isopropylpyrrolidin-3-yl)acetamide | 367 |
| N-((3R,4S,5R)-4-hydroxy-1-(2-hydroxyethyl)-5-(hydroxymethyl)pyrrolidin-3- | 369 |
| yl)acetamide | |
| (2R,3S,4R)-4-amino-1-benzyl-2-(hydroxymethyl)pyrrolidin-3-ol | 370 |
| 2-acetamido-1,2,4-trideoxy-1,4-imino-L-ribitol | 378 |
| N-((3R,4R,5S)-1-butyl-4-hydroxy-5-(hydroxymethyl)pyrrolidin-3-yl)acetamide | 379 |
| (2R,3S,4S)-4-amino-1-benzyl-2-(hydroxymethyl)pyrrolidin-3-ol | 390 |
| (2S,3R,4S)-2-((R)-1,2-dihydroxyethyl)-1-(2-hydroxyethyl)pyrrolidine-3,4-diol | 405 |
| N-(((2R,3R,4S)-1-benzyl-3,4-dihydroxypyrrolidin-2-yl)methyl)acetamide | 482 |
| N-(((2R,3R,4S)-3,4-dihydroxy-1-(2-(2-methoxyethoxy)ethyl)pyrrolidin-2- | 516 |
| yl)methyl)acetamide | |
| N-butyl-2-((2R,3S,4R,5R)-1-butyl-3,4-dihydroxy-5-(hydroxymethyl)pyrrolidin-2- | 541 |
| yl)acetamide | |
| (2R,3R,4R)-2-(hydroxymethyl)-1-(2-(2-methoxyethoxy)ethyl)pyrrolidine-3,4-diol | 571 |
| N-(((2S,3R,4S)-3,4-dihydroxy-1-(2-(2-methoxyethoxy)ethyl)pyrrolidin-2- | 583 |
| yl)methyl)acetamide | |
| N-(((2S,3R,4S)-1-butyl-3,4-dihydroxypyrrolidin-2-yl)methyl)benzamide | 607 |
| N-(((2S,3R,4S)-1-(2-(dimethylamino)ethyl)-3,4-dihydroxypyrrolidin-2- | 615 |
| yl)methyl)acetamide | |
| N-(((2S,3R,4S)-3,4-dihydroxy-1-(2-(piperidin-1-yl)ethyl)pyrrolidin-2-yl)methyl)acetamide | 616 |
| N-(((2R,3R,4S)-1-butyl-3,4-dihydroxypyrrolidin-2-yl)methyl)benzamide | 617 |
| N-(((2S,3R,4S)-3,4-dihydroxy-1-methylpyrrolidin-2-yl)methyl)acetamide | 679 |
| (2R,3R,4R)-1-butyl-2-(hydroxymethyl)piperidine-3,4-diol | 681 |

### III. Identification of Non-Active Site Chaperones of Alpha-Galactosidase

Compounds that demonstrated a significant increase in cellular alpha-galactosidase activity (protocol II) but showed no competitive inhibition of alpha-galactosidase enzyme activity (protocol I) were considered to be non-active site chaperones.

Compounds identified according to the methods described above (I-III) find utility in the treatment of Fabry's disease.

### Example 6

### Identification of Pharmacoperones for Alpha-Glucosidase

### I. Alpha-Glucosidase Activity Assay

Human Caucasian promyelocytic leukaemia cells (HL60, ECACC No. 98070106) were cultured using a standard sub-culture routine and lysed. The lysates were used as a source for wild type (wt) lysosomal alpha-glucosidase and used in an assay to determine the enzyme activity and conduct inhibition studies.

### i) Cell Lysate Preparation

Cell lysates were prepared as described above (Gaucher's I.i)

### ii) Alpha-Glucosidase Activity Assay

4-methylumbelliferyl alpha-glucopyranoside (4MU-α-D-glc) (Sigma) was used as a substrate to measure alpha-glucosidase activity in HL60 lysate. Enzyme assays were performed in 96-well microtitre plates. Thawed cell lysate and 0.5 mM 4MU-α-D-glc in citric phosphate buffer (pH 4.5) (50 μl final reaction volume) were mixed and incubated at 37° C. The reaction was quenched with 150 μl 0.5M sodium carbonate. The activity was measured by determining the rate of product (4MU) released using a fluorometer (OPTIMA, BMG) using excitation 360 nm, emission 450 nm filters. For detailed inhibition kinetic studies, various concentrations of iminosugars (1 nM-1 mM) and 4MU-α-D-Glc (100 μM-4 mM) were used.

In this assay, the following compounds showed greater than 50% inhibition at a concentration of 100 uM:

| | Compound |
|---|---|
| Compound Name | No |
| (2R,3R,4R,5S)-2-(hydroxymethyl)-1-methylpiperidine-3,4,5-triol | 3 |
| (2R,3S,4R,5R,6R)-2,6-bis(hydroxymethyl)piperidine-3,4,5-triol | 15 |
| (2S,3R,4S,5S,6S)-2-ethyl-6-(hydroxymethyl)piperidine-3,4,5-triol | 23 |
| (1R,2S,6S,7R,8R,8aS)-2-(3,4,5-trihydroxy-6-(hydroxymethyl)tetrahydro-2H-pyran-2- | 41 |
| yloxy)octahydroindolizine-1,6,7,8-tetraol | |
| (1S,6S,7S,8R)-1,7,8-trihydroxyoctahydroindolizin-6-yl butyrate | 46 |
| (2R,3R,4R,5S,6R)-2-(hydroxymethyl)-6-methylpiperidine-3,4,5-triol | 51 |
| (2R,3R,4R,5S)-2-((3,4,5-trihydroxy-6-(hydroxymethyl)tetrahydro-2H-pyran-2- | 78 |
| yloxy)methyl)piperidine-3,4,5-triol | |
| 2-((2R,3R,4R,5R)-4-hydroxy-2,5-bis(hydroxymethyl)pyrrolidin-3-yloxy)-6- | 79 |
| (hydroxymethyl)tetrahydro-2H-pyran-3,4,5-triol | |
| (2S,3R,4R)-3,4-dihydroxy-1,1-dimethylpyrrolidinium-2-carboxylate | 80 |
| (1S,6S,7R,8R,8aR)-octahydroindolizine-1,6,7,8-tetraol | 104 |
| (2R,3R,4R,5S)-1-butyl-2-(hydroxymethyl)piperidine-3,4,5-triol | 118 |
| 2-((3S,4S,5R,6R)-4,5-dihydroxy-6-(hydroxymethyl)piperidin-3-yloxy)-6- | 121 |
| (hydroxymethyl)tetrahydro-2H-pyran-3,4,5-triol | |
| (2R,3R,4R,5S,6R)-2,6-bis(hydroxymethyl)-1-methylpiperidine-3,4,5-triol | 124 |
| (1R,2R,3R,6S,7S,7aR)-3-(hydroxymethyl)hexahydro-1H-pyrrolizine-1,2,6,7-tetraol | 137 |
| (1S,2R,3R,5R,7aR)-3-(hydroxymethyl)-5-methylhexahydro-1H-pyrrolizine-1,2-diol | 150 |
| (2S,3S,4S)-2-(hydroxymethyl)pyrrolidine-3,4-diol | 160 |
| (1R,2R,3R,6S,7S,7aR)-1,2,6,7-tetrahydroxy-3-(hydroxymethyl)octahydropyrrolizine | 169 |
| 4-oxide | |
| (2S,3S,4S)-2-((S)-1,2-dihydroxyethyl)-4-(hydroxymethyl)pyrrolidine-3,4-diol | 179 |
| (2R,3R,4R,5S)-2-(hydroxymethyl)piperidine-3,4,5-triol | 193 |
| (2S,3S,4S)-2,4-bis(hydroxymethyl)pyrrolidine-3,4-diol | 202 |
| 2-((2R,3R,4R,5S)-3,4,5-trihydroxy-2-(hydroxymethyl)piperidin-1-yl)acetic acid | 214 |
| (2S,3S,4S,5R)-2-(hydroxymethyl)piperidine-3,4,5-triol | 221 |
| (1R,2R,3R,6S,7S,7aR)-5-gem-dideuterio-3-(hydroxymethyl)hexahydro-1H- | 271 |
| pyrrolizine-1,2,6,7-tetraol | |
| (2R,3R,4R,5S)-1-(4-hydroxybutyl)-2-(hydroxymethyl)piperidine-3,4,5-triol | 276 |
| (2R,3R,4R,5S)-2-(hydroxymethyl)-1-(3-phenoxypropyl)piperidine-3,4,5-triol | 288 |
| (2S,3S,4S,5S)-2-(hydroxymethyl)-5-methylpyrrolidine-3,4-diol | 300 |
| (2R,3R,4R,5R)-2,5-bis(hydroxymethyl)-1-(3-phenoxypropyl)pyrrolidine-3,4-diol | 343 |
| (1S,2S,6S,7R,8R,8aR)-octahydroindolizine-1,2,6,7,8-pentaol | 352 |
| (2R,3R,4R,5S)-2-methylpiperidine-3,4,5-triol | 388 |
| (3S,4R,5S,6R)-3,4,5-trihydroxy-6-(hydroxymethyl)piperidin-2-one | 446 |
| Nojirimycin-1-Sulfonic Acid | 464 |
| (2R,3R,4S,5R,6R)-2-butyl-6-(hydroxymethyl)piperidine-3,4,5-triol | 488 |
| (2S,3S,4S)-1-benzyl-2-(hydroxymethyl)pyrrolidine-3,4-diol | 500 |
| (2S,3S,4S)-2-(hydroxymethyl)-2-methylpyrrolidine-3,4-diol | 501 |
| (2R,3R,4R,5S)-2-(hydroxymethyl)-1-(2-morpholinoethyl)piperidine-3,4,5-triol | 509 |
| (2R,3R,4R,5S)-1-benzyl-2-(hydroxymethyl)piperidine-3,4,5-triol | 510 |
| (2R,3R,4R,5R)-2,5-bis(hydroxymethyl)-1-methylpyrrolidine-3,4-diol | 515 |
| (2R,3R,4R,5S)-1-(biphenyl-4-ylmethyl)-2-(hydroxymethyl)piperidine-3,4,5-triol | 524 |
| (1R,2S,3R)-1-((2R,3R,4S)-1butyl-3,4-dihydroxypyrrolidin-2-yl)butane-1,2,3,4-tetraol | 531 |
| (2R,3R,4R,5S)-2-(hydroxymethyl)-1-nonylpiperidine-3,4,5-triol | 550 |
| (2R,3R,4R,5R)-1-(2-(benzyloxy)ethyl)-2,5-bis(hydroxymethyl)pyrrolidine-3,4-diol | 556 |
| (2R,3R,4R,5R)-1-(biphenyl-4-ylmethyl)-2,5-bis(hydroxymethyl)pyrrolidine-3,4-diol | 558 |
| (2R,3R,4R,5S)-2-(hydroxymethyl)-1-(9-hydroxynonyl)piperidine-3,4,5-triol | 724 |
| (2R,3R,4R,5S)-2-(hydroxymethyl)-1-(2-(2-methoxyethoxy)ethyl)piperidine-3,4,5-triol | 725 |
| (2S,3S,4S,5S)-2-butyl-5-(hydroxymethyl)pyrrolidine-3,4-diol | 728 |
| (2S,3S,4S,5S)-2,5-bis(hydroxymethyl)pyrrolidine-3,4-diol | 729 |
| (2S,3S,4S,5S)-2-ethyl-5-(hydroxymethyl)pyrrolidine-3,4-diol | 732 |
| (R)-(1-butylpiperidin-3-yl)methanol | 741 |

### II. Enzyme Enhancement Assay—Cell Based Screening for Chaperones

Lymphoblasts derived from Pompe's patients can be used for the cell based screening assays. EBV transformed B-lymphocytes from Pompe's patient such as (GM013963) and (GM06314) were obtained from Coriell Institute for Medical Research. Cells were cultured in RMPI 1640 (Sigma) supplemented with 15% FBS(PAA), 2 mM L-glutamine and penicillin-streptomycin (PAA) as described in the culturing protocol.

Cells were seeded (8×10⁴ cells/well) and dosed (0.3-100 μM) in white 96-well plates (NUNC) to a final volume of 300 μL, and incubated for 72 hr at 37° C. in a 5% CO₂ incubator. Cells (200 μL) were transferred to 96-well Multiscreen harvester plates (Millipore) and harvested under vacuum. Cells were washed twice with PBS and lysed (and the enzyme reaction started) by the addition of 100 μL 5 mM 4MU-α-D-glc in citric phosphate buffer (pH4.5) with 0.1% Triton X-100 (Sigma). Cell debris was removed by filtering through and collecting the cleared lysates, and the lysate was incubated at 37° C. for 2 hrs. The enzyme reaction was quenched by addition of 150 μL 0.5M sodium carbonate to 50 μl of reaction mix. Fluorescence was measured as described above. Cell viability was measured using CellTiter-Glo® luminescent cell viability assay (Promega) on the remaining 100 μL unlysed cells. All experiments were performed in triplicates. The fold alpha-glucosidase enzyme activity was determined relative to the vehicle (water or 1% DMSO) control.

### III. Identification of Non-Active Site Chaperones of Alpha-Glucosidase

Compounds that demonstrated a significant increase in cellular alpha-glucosidase activity (protocol II) but showed no competitive inhibition of alpha-glucosidase enzyme activity (protocol I) were considered to be non-ASSCs.

Compounds identified according to the methods described above (I-III) find utility in the treatment of Pompe's disease.

### EQUIVALENTS

The foregoing description details presently preferred embodiments of the present invention. Numerous modifications and variations in practice thereof are expected to occur to those skilled in the art upon consideration of these descriptions. Those modifications and variations are intended to be encompassed within the claims appended hereto.

1-66. (canceled)

67. A method of treating a proteostatic disease in a subject, comprising administering to a subject in need thereof an effective amount of an β-N-acetylglucosaminidase inhibitor of Formula (1) 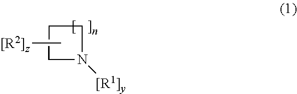
in which
n represents an integer from 1 to 7, provided that where n>1 the ring may also contain at least one unsaturated C—C bond
z represents an integer from 1 to (n+2)
y represents 1 or 2
R¹ represents H; C1-15 alkyl, C1-15 alkenyl or C1-15 alkynyl, optionally substituted with one or more R²; oxygen or an oxygen containing group such that the compound is an N-oxide; C(O)OR³; C(O)NR³R⁴; SO₂NR³; OH, OR³, or formyl
R² represents OH; OR³; ═O; NH₂; N₃; SH; SOₓR³; halo; CN; NO₂; NR³R⁴; (NR³)NR³R⁴; NH(NR³)NR³R⁴; CO₂R⁴; OC(O)R³; CONR³R⁴; NR⁴C(O)R³; NR⁴SO₂R³; P(O)(OR³)₂; C1-15 alkyl or alkenyl optionally substituted with one or more OH, OR³, ═O, NH₂, N₃, SH, SOₓR³, halo, CN, NO₂, NR³R⁴, (NR³)NR³R⁴, NH(NR³)NR³R⁴, CO₂R⁴, OC(O)R³, CONR³R⁴, NR⁴C(O)R³, NR⁴SO₂R³, P(O)(OR³)₂, aryl or carbocyclyl groups; carbocyclyl or aryl, either of which is optionally substituted with one or more OH, OR³, ═O, NH₂, N₃, SH, SOₓR³ halo, CN, NO₂, NR³R⁴, (NR³)NR³R⁴, NH(NR³)NR³R⁴, CO₂R⁴, OC(O)R³, CONR³R⁴, NR⁴C(O)R³, NR⁴SO₂R³, P(O)(OR³)₂, C1-9 alkyl optionally substituted with one or more OH, OR³, ═O, NH₂, N₃, halo, CN, NO₂, NR³R⁴, CO₂R⁴, CONR³R⁴, aryl or carbocyclyl groups; O-glycosyl; C-glycosyl; O-sulfate; O-phosphate or a group which together with the endocyclic carbon forms a spiro ring, with the provisos that: (a) two OH groups may not be attached to the same endocyclic carbon atom; (b) where there is only one R² substituent it contains an oxygen atom directly bonded to an endocyclic carbon atom; and (c) where z>1 any two R² substituents may together form an optionally heterocyclic ring (for example a carbocycle, cyclic ether or acetal)
R³ represents H; C1-6 alkyl, optionally substituted with one or more OH; aryl or C1-3 alkyl optionally substituted with aryl; SiR⁴₃ and
R⁴ represents H; C1-6 alkyl, optionally substituted with one or more OH
R³ and R⁴ may optionally form a 4 to 8 membered ring, containing one or more O, SOₓ or NR³ groups
x represents an integer from 0 to 2
or a pharmaceutically acceptable salt thereof.

68. The method of claim 67 wherein n=2 or 3.

69. The method of claim 67 wherein z=2 to (n+2).

70. The method of claim 67 wherein z=n+2.

71. The method of claim 67 wherein one or more endocyclic carbon atom(s) is replaced with a sulphur, oxygen or nitrogen heteroatom.

72. The method of claim 67 wherein the inhibitor has at least two R² substituents, one being OH and the other being hydroxymethyl.

73. The method of claim 67 wherein said inhibitor is selected from compounds 1 to 892 of Table 1, or a pharmaceutically acceptable salt thereof.

74. The method of claim 67 wherein the proteostatic disease is an aggregative proteostatic disease.

75. The method of claim 67 wherein the aggregative proteostatic disease is selected from: amyloidosis, synucleinopathy, expanded CAG repeat disease and tauopathy.

76. The method of claim 67 wherein the proteostatic disease is a protein conformational (folding) disease.

77. The method of claim 67 wherein the protein conformational (folding) disease is a genetic disease or an acquired disease.

78. The method of claim 67 wherein the protein conformational (folding) disease is selected from: lysosomal storage disease, cystic fibrosis and emphysema.

79. The method of claim 67 wherein the proteostatic disease is an ER stress-induced disease.

80. The method of claim 67 wherein the proteostatic disease is selected from diabetes (e.g. type 2 diabetes), insulin resistance and metabolic syndrome.

81. The method of claim 67 wherein the proteostatic disease is an age-onset disease, for example selected from dementia, neurodegenerative disease (e.g. AD, PD, ALS and HD), cancer, heart disease and autoimmune diseases (e.g. rheumatoid arthritis and diabetes).
